(19) United States
(12) Patent Application Publication
(10) Pub. No.: US 2026/0262445 A1
(43) Pub. Date: Sep. 3, 2026

(54) ORGANIC COMPOUND AND LIGHT-EMITTING DEVICE

(71) Applicant: SEMICONDUCTOR ENERGY LABORATORY CO., LTD., Atsugi-shi (JP)

(72) Inventors: Anna TADA, Isehara (JP); Sachiko KAWAKAMI, Atsugi (JP); Naoaki HASHIMOTO, Sagamihara (JP); Tsunenori SUZUKI, Yokohama (JP)

(21) Appl. No.: 19/546,223

(22) Filed: Feb. 20, 2026

(30) Foreign Application Priority Data
Feb. 21, 2025 (JP) 2025-027127

(51) Int. Cl. H10K 85/60; C07D 405/10; H10K 50/15; H10K 50/17

(52) U.S. Cl. CPC H10K 85/636; C07D 405/10; H10K 50/15; H10K 50/17; H10K 85/615; H10K 85/6572; H10K 85/6574

(57) ABSTRACT

A novel organic compound and a light-emitting device using the organic compound are provided. The organic compound is represented by General Formula (G1). In General Formula (G1), Ar¹ represents any one of General Formulae (g1-1) to (g1-3). Each of R¹ to R⁸ independently represents hydrogen, an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, or a substituted or unsubstituted heteroaryl group having 1 to 30 carbon atoms. Any one of R¹ to R⁴ represents General Formula (g2-1) or (g2-2). Each of Ar² and Ar³ independently represents a substituted or unsubstituted aryl group having 6 to 60 carbon atoms or a substituted or unsubstituted heteroaryl group having 1 to 60 carbon atoms.

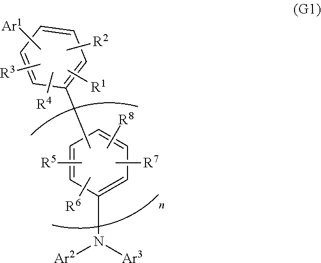

### BACKGROUND OF THE INVENTION

### 1. Field of the Invention

One embodiment of the present invention relates to an organic compound, an organic semiconductor element, a light-emitting device, a photodiode sensor, a display module, a lighting module, a display apparatus, an electronic appliance, alighting device, and an electronic appliance. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Thus, specific examples of the technical field of one embodiment of the present invention disclosed in this specification include a semiconductor device, a display apparatus, a liquid crystal display apparatus, a lighting device, a power storage device, a memory device, an image capturing device, a driving method thereof, and a manufacturing method thereof.

### 2. Description of the Related Art

A light-emitting device (also referred to as an organic EL element) including an organic compound that is a light-emitting substance between a pair of electrodes has characteristics such as thinness, light weight, high-speed response, and low voltage driving. Thus, displays including such light-emitting devices have been developed.

Since a continuous light-emitting layer can be formed two-dimensionally for such light-emitting devices, planar light emission can be achieved. This feature is difficult to achieve with point light sources typified by incandescent lamps and LEDs or linear light sources typified by fluorescent lamps; thus, the light-emitting devices also have great potential as planar light sources which can be used for lighting devices and the like.

For example, a functional panel in which a pixel provided in a display region includes a light-emitting element (light-emitting device) and a photoelectric conversion element (light-receiving device) is known (Patent Document 1).

Although displays or lighting devices including light-emitting devices can be suitably used for a variety of electronic appliances as described above, their performance and cost competitiveness have plenty of room to improve. Therefore, a material that is more excellent in characteristics and easier to handle and an easy method for synthesizing the material are required.

Although the characteristics of organic EL devices have been improved considerably, advanced requirements for various characteristics including efficiency and durability are not yet satisfied. In particular, to solve a problem such as burn-in, which is an issue peculiar to EL, it is preferable to inhibit a reduction in efficiency due to deterioration as much as possible.

Deterioration largely depends on an emission center substance and its surrounding materials; therefore, organic compound materials having favorable characteristics have been actively developed.

In order to obtain a higher-resolution light-emitting apparatus using an organic EL device, patterning a layer formed of an organic compound by a photolithography method using a photoresist or the like, instead of an evaporation method using a metal mask, has been studied. By using the photolithography method, a high-resolution display apparatus in which the distance between EL layers is several micrometers can be obtained (see Patent Document 2, for example).

### REFERENCES

### Patent Documents

[Patent Document 1] PCT International Publication No. WO2020/152556.
[Patent Document 2] Japanese Translation of PCT International Application No. 2018-521459

### SUMMARY OF THE INVENTION

An object of one embodiment of the present invention is to provide a novel organic compound. Another object of one embodiment of the present invention is to provide a method for synthesizing a novel organic compound. Another object of one embodiment of the present invention is to provide an organic compound that can be used for a light-emitting device. Another object of one embodiment of the present invention is to provide an organic compound that can be used for a light-receiving device. Another object of one embodiment of the present invention is to provide a light-emitting device with high emission efficiency. Another object of one embodiment of the present invention is to provide a light-emitting device with high color purity. Another object of one embodiment of the present invention is to provide a light-emitting device with high reliability. Another object of one embodiment of the present invention is to provide any of a low-power-consumption display apparatus, a low-power-consumption electronic appliance, and a low-power-consumption lighting device. Another object of one embodiment of the present invention is to provide any of a highly reliable display apparatus, a highly reliable electronic appliance, and a highly reliable lighting device. Another object of one embodiment of the present invention is to provide any of ahigh-color-purity display apparatus, a high-color-purity electronic appliance, and a high-color-purity lighting device.

It is acceptable that at least one of the above-described objects be achieved in the present invention.

One embodiment of the present invention is an organic compound represented by General Formula (G1).

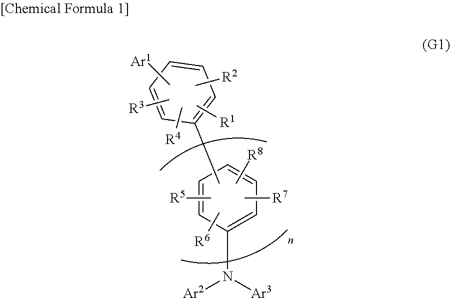

In General Formula (G1), Ar¹ represents any one of General Formulae (g1-1) to (g1-3) below, and each of R¹ to R⁸ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, or a substituted or unsubstituted heteroaryl group having 1 to 30 carbon atoms. Any one of R¹ to R⁴ represents General Formula (g2-1) or General Formula (g2-2) below. Furthermore, n is greater than or equal to 0 and less than or equal to 3. In the case where n is greater than or equal to 2, R⁵s may be the same or different from each other, and the same applies to R⁶s, R⁷s, and R⁸s. Each of Ar² and Ar³ independently represents a substituted or unsubstituted aryl group having 6 to 60 carbon atoms or a substituted or unsubstituted heteroaryl group having 1 to 60 carbon atoms.

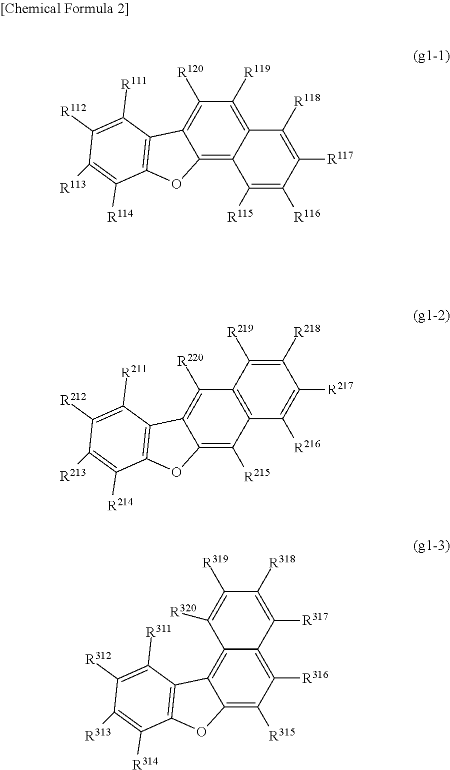

In General Formulae (g1-1) to (g1-3), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹ to R¹²⁰, any one of R²¹¹ to R²²⁰, and any one of R³¹¹ to R³²⁰ each represent a bond with General Formula (G1).

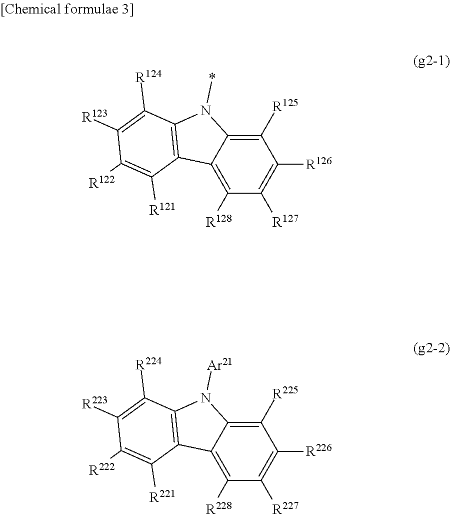

In General Formulae (g2-1) and (g2-2), each of R¹²¹ to R¹²⁸ and R²²¹ to R²²⁸ independently represents hydrogen (including deuterium), an alkyl group having 1 to 6 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. An asterisk (*) and any one of R²²¹ to R²²⁸ represent a bond with General Formula (G1). Ar²¹ represents a substituted or unsubstituted aryl group having 6 to 60 carbon atoms or a substituted or unsubstituted heteroaryl group having 1 to 60 carbon atoms.

One embodiment of the present invention is an organic compound represented by General Formula (G2).

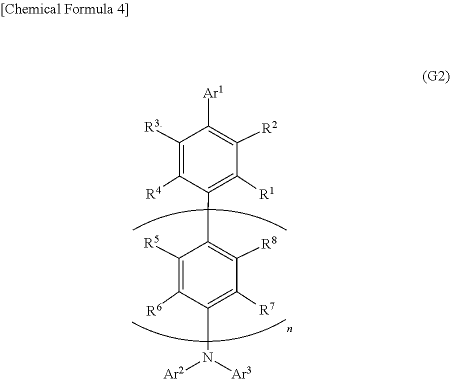

In General Formula (G2), Ar¹ represents any one of General Formulae (g1-1) to (g1-3) above, and each of R¹ to R⁸ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, or a heteroaryl group having 1 to 30 carbon atoms. Any one of R¹ to R⁴ represents General Formula (g2-1) or General Formula (g2-2) above. Furthermore, n is greater than or equal to 0 and less than or equal to 3. Each of Ar² and Ar³ independently represents a substituted or unsubstituted aryl group having 6 to 60 carbon atoms or a substituted or unsubstituted heteroaryl group having 1 to 60 carbon atoms.

In General Formula (G2), Ar¹ represents any one of General Formulae (g1-1) to (g1-3) above, and each of R¹ to R⁸ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, or a heteroaryl group having 1 to 30 carbon atoms. R² represents General Formula (g2-1) or General Formula (g2-2) above. Furthermore, n is greater than or equal to 0 and less than or equal to 3. Each of Ar² and Ar³ independently represents a substituted or unsubstituted aryl group having 6 to 60 carbon atoms or a substituted or unsubstituted heteroaryl group having 1 to 60 carbon atoms.

In General Formula (G1) or General Formula (G2) above, n is 1.

One embodiment of the present invention is an organic compound represented by General Formula (G3).

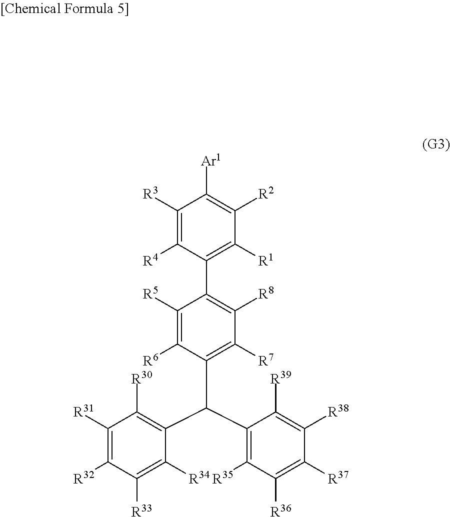

In General Formula (G3), Ar¹ represents any one of General Formulae (g1-1) to (g1-3) above, and each of R¹ to R⁸ and R³⁰ to R³⁹ independently represents hydrogen (including deuterium), an alkyl group having 1 to 6 carbon atoms, a cycloalkyl group having 3 to 10 carbon atoms, an alkoxy group having 1 to 6 carbon atoms, or a substituted or unsubstituted aryl group having 6 to 15 carbon atoms. R² represents General Formula (g2-1) or General Formula (g2-3) below. In the case where each of R³⁰ to R³⁹ represents a substituted aryl group having 6 to 15 carbon atoms, the aryl group having 6 to 15 carbon atoms is bonded to an adjacent aromatic ring to form a ring.

In General Formulae (g1-1) to (g1-3) in General Formulae (G3), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹, R¹¹², R¹¹⁴, and R¹¹⁹, any one of R²¹², R²¹⁴, and R²¹⁵, and any one of R³¹², R³¹⁴, and R³¹⁵ each represent a bond with General Formula (G3).

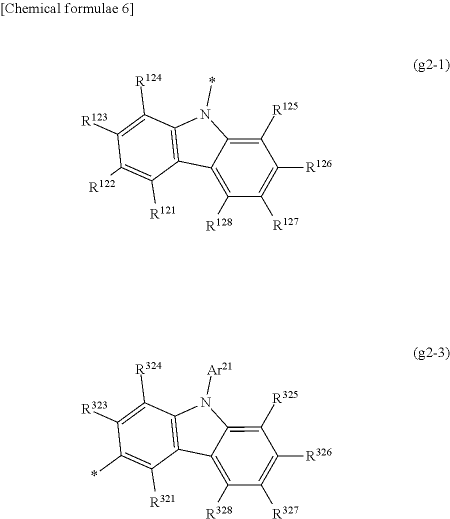

In General Formulae (g2-1) and (g2-3), each of R¹²¹ to R¹²⁸ and R³²¹ to R³²⁸ independently represents hydrogen (including deuterium), an alkyl group having 1 to 6 carbon atoms, an alkoxy group having 1 to 6 carbon atoms, or a substituted or unsubstituted aryl group having 6 to 30 carbon atoms. An asterisk (*) represents a bond with General Formula (G3). A²¹ represents a substituted or unsubstituted aryl group having 6 to 30 carbon atoms or a substituted or unsubstituted heteroaryl group having 1 to 30 carbon atoms.

In the above invention, at least one of R³⁰ to R³⁴ represents a substituted or unsubstituted phenyl group, and at least one of R³⁵ to R³⁹ represents a substituted or unsubstituted phenyl group.

One embodiment of the present invention is an organic compound represented by General Formula (G4).

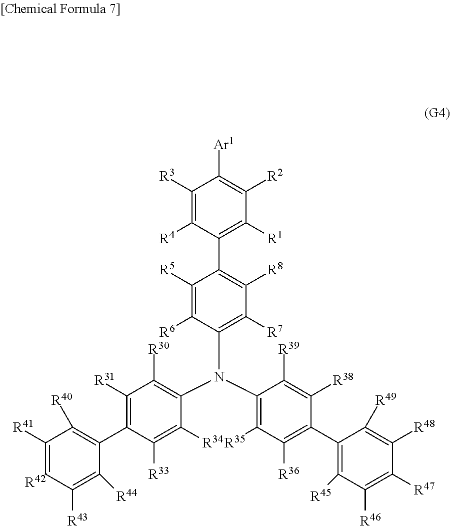

In General Formula (G4), Ar¹ represents any one of General Formulae (g1-1) to (g1-3) below, and each of R¹ to R⁸ and R³⁰ to R⁴⁹ independently represents hydrogen (including deuterium), an alkyl group having 1 to 6 carbon atoms, a cycloalkyl group having 3 to 10 carbon atoms, an alkoxy group having 1 to 6 carbon atoms, or a substituted or unsubstituted aryl group having 6 to 15 carbon atoms. R² represents General Formula (g2-1) or General Formula (g2-3) below.

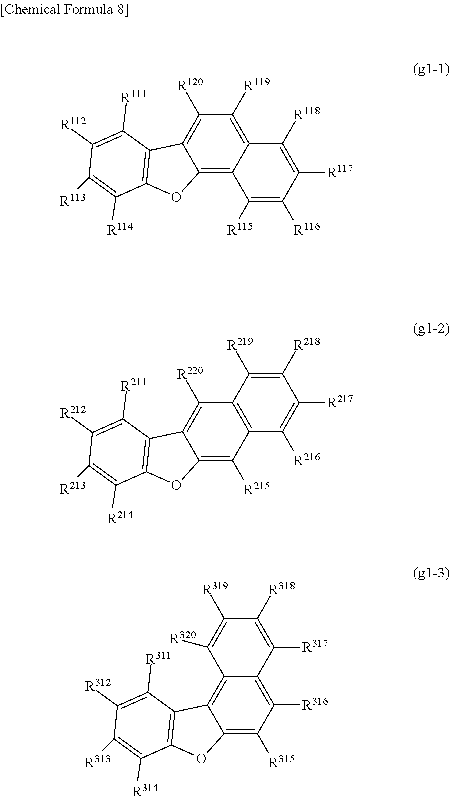

In General Formulae (g1-1) to (g1-3) in General Formulae (G4), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹, R¹¹², R¹¹⁴, and R¹¹⁹, any one of R²¹², R²¹⁴, and R²¹⁵, and any one of R³¹², R³¹⁴, and R³¹⁵ each represent a bond with General Formula (G4).

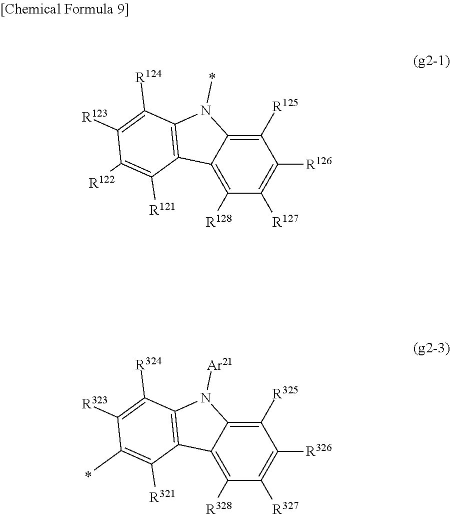

In General Formula (g2-1) and General Formula (g2-3) in General Formula (G4), each of R¹²¹ to R¹²⁸ and R³²¹ to R³²⁸ independently represents hydrogen (including deuterium), an alkyl group having 1 to 6 carbon atoms, an alkoxy group having 1 to 6 carbon atoms, or a substituted or unsubstituted aryl group having 6 to 30 carbon atoms. An asterisk (*) represents a bond with General Formula (G4). Ar²¹ represents a substituted or unsubstituted aryl group having 6 to 30 carbon atoms, or a substituted or unsubstituted heteroaryl group having 1 to 30 carbon atoms.

One embodiment of the present invention is an organic compound represented by General Formula (G5).

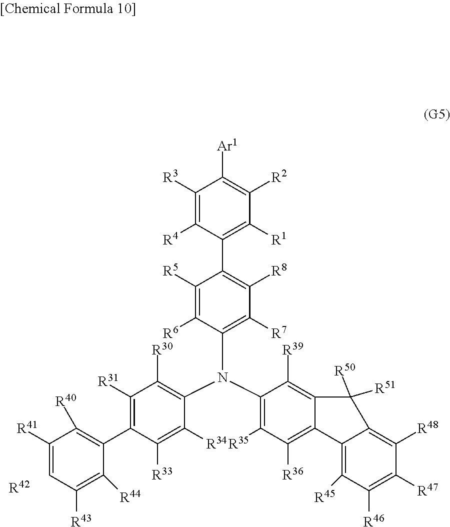

In General Formula (G5), Ar¹ represents any one of General Formulae (g1-1) to (g1-3) below, and each of R¹ to R⁸ and R³⁰ to R⁵¹ independently represents hydrogen (including deuterium), an alkyl group having 1 to 6 carbon atoms, a cycloalkyl group having 3 to 10 carbon atoms, an alkoxy group having 1 to 6 carbon atoms, or a substituted or unsubstituted aryl group having 6 to 15 carbon atoms. In the case where each of R⁵⁰ and R⁵¹ is a substituted or unsubstituted aryl group having 6 to 15 carbon atoms, R⁵⁰ and R⁵¹ may be bonded to each other to form a ring. R² represents General Formula (g2-1) or General Formula (g2-3) below.

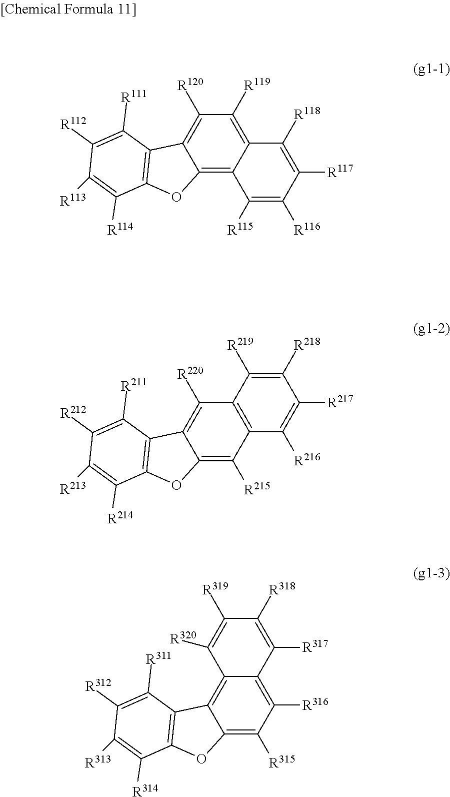

In General Formulae (g1-1) to (g1-3) in General Formulae (G5), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹, R¹¹², R¹¹⁴, and R¹¹⁹, any one of R²¹², R²¹⁴, and R²¹¹, and any one of R³¹², R³¹⁴, and R³¹⁵ each represent a bond with General Formula (G5).

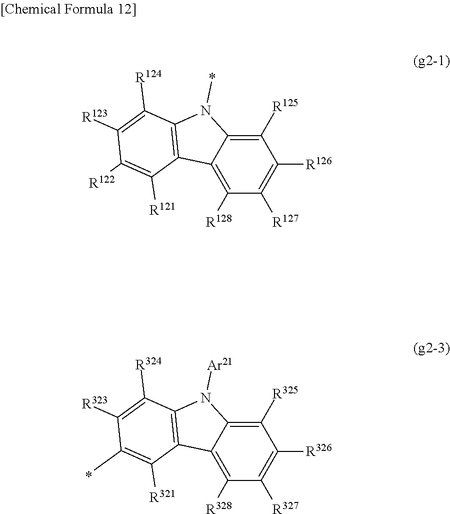

In General Formulae (g2-1) and (g2-3) in General Formula (G5), each of R¹²¹ to R¹²⁸ and R³²¹ to R³²⁸ independently represents hydrogen (including deuterium), an alkyl group having 1 to 6 carbon atoms, an alkoxy group having 1 to 6 carbon atoms, or a substituted or unsubstituted aryl group having 6 to 30 carbon atoms. An asterisk (*) represents a bond with General Formula (G5). Ar²¹ represents a substituted or unsubstituted aryl group having 6 to 30 carbon atoms or a substituted or unsubstituted heteroaryl group having 1 to 30 carbon atoms.

In the above invention, each of R¹¹⁴, R²¹⁴, and R³¹⁴ in General Formulae (g1-1) to (g1-3) represents a bond with General Formula (G5).

One embodiment of the present invention is an organic compound represented by Structural Formula (100) or Structural Formula (101).

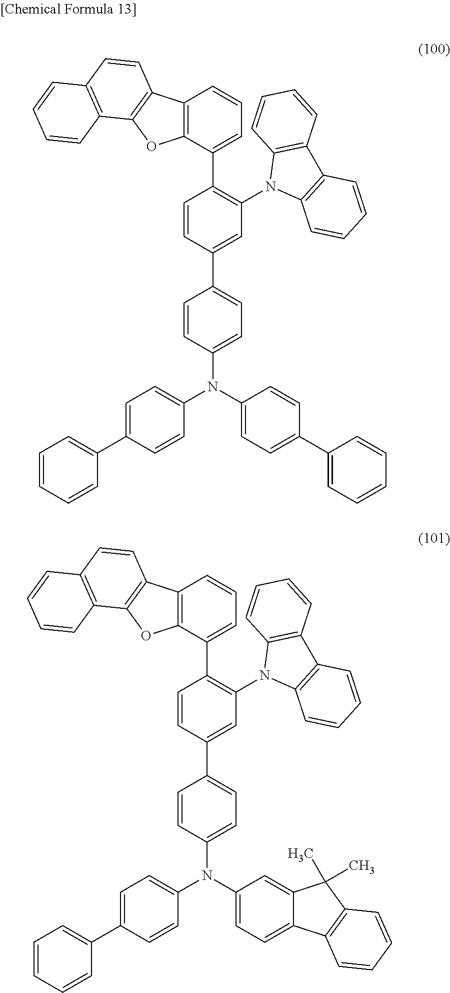 [SMILES: c1ccc(-c2ccc(N(c3ccc(-c4ccccc4)cc3)c3ccc(-c4ccc(-c5cccc6c5oc5c7ccccc7ccc65)c(-n5c6ccccc6c6ccccc65)c4)cc3)cc2)cc1 ; CC1(C)c2ccccc2-c2ccc(N(c3ccc(-c4ccccc4)cc3)c3ccc(-c4ccc(-c5cccc6c5oc5c7ccccc7ccc65)c(-n5c6ccccc6c6ccccc65)c4)cc3)cc21]

Another embodiment of the present invention is a light-emitting device or a light-receiving device including any one of the above-described organic compounds.

Another embodiment of the present invention is a light-emitting device including any of the above-described organic compounds. Another embodiment of the present invention is a display apparatus including any of the above-described light-emitting devices.

Another embodiment of the present invention is an electronic appliance including any of the above-described light-emitting devices, and any of a sensor, an operation button, a speaker, and a microphone.

Another embodiment of the present invention is a lighting device including any of the above-described light-emitting devices and a housing.

One embodiment of the present invention can provide a novel organic compound. Another embodiment of the present invention can provide a method for synthesizing a novel organic compound. Another embodiment of the present invention can provide an organic compound that can be used for a light-emitting device. Another embodiment of the present invention can provide an organic compound that can be used for a light-receiving device. Another embodiment of the present invention can provide a light-emitting device with high emission efficiency. Another embodiment of the present invention can provide a light-emitting device with high color purity. Another embodiment of the present invention can provide a light-emitting device with high reliability. Another embodiment of the present invention can provide any of a low-power-consumption display apparatus, a low-power-consumption electronic appliance, and a low-power-consumption lighting device. Another embodiment of the present invention can provide any of a highly reliable display apparatus, a highly reliable electronic appliance, and a highly reliable lighting device. Another embodiment of the present invention can provide any of a high-color-purity display apparatus, a high-color-purity electronic appliance, and a high-color-purity lighting device.

The description of these effects does not preclude the existence of other effects. One embodiment of the present invention does not necessarily have all these effects. Other effects will be apparent from and can be derived from the description of the specification, the drawings, the claims, and the like.

### BRIEF DESCRIPTION OF THE DRAWINGS

FIGS. 1A and 1B are schematic diagrams of a light-emitting device.

FIGS. 2A to 2E illustrate structures of a light-emitting device.

FIGS. 3A and 3B are a top view and a cross-sectional view, respectively, of a light-emitting apparatus.

FIGS. 4A to 4C each illustrate a light-receiving device of one embodiment of the present invention.

FIGS. 5A to 5C each illustrate a light-emitting and light-receiving apparatus of one embodiment of the present invention.

FIGS. 6A and 6B each illustrate a light-emitting and light-receiving apparatus of one embodiment of the present invention.

FIGS. 7A to 7D illustrate a light-emitting and light-receiving apparatus according to an embodiment.

FIGS. 8A to 8F illustrate a light-emitting and light-receiving apparatus and pixel arrangements according to an embodiment.

FIGS. 9A to 9C illustrate pixel circuits according to an embodiment.

FIG. 10 illustrates a light-emitting and light-receiving apparatus according to an embodiment.

FIGS. 11A to 11E illustrate electronic appliances according to an embodiment.

FIGS. 12A to 12E illustrate electronic appliances according to an embodiment.

FIGS. 13A and 13B illustrate electronic appliances according to an embodiment.

FIG. 14 shows a measurement result of a ¹H NMR spectrum of an organic compound formed in an example.

FIG. 15 shows absorption and emission spectra of a toluene solution of an organic compound.

FIG. 16 shows absorption and emission spectra of a thin film of an organic compound.

FIG. 17 shows a measurement result of a ¹H NMR spectrum of an organic compound formed in an example.

FIG. 18 shows absorption and emission spectra of a toluene solution of an organic compound.

FIG. 19 shows absorption and emission spectra of a thin film of an organic compound.

FIG. 20 illustrates a structure of a light-emitting device.

FIG. 21 shows luminance-current density characteristics of light-emitting devices.

FIG. 22 shows luminance-voltage characteristics of light-emitting devices.

FIG. 23 shows current efficiency-luminance characteristics of light-emitting devices.

FIG. 24 shows current density-voltage characteristics of light-emitting devices.

FIG. 25 shows electroluminescence spectra of light-emitting devices.

FIG. 26 shows luminance changes over driving time of light-emitting devices.

FIG. 27 shows luminance-current density characteristics of light-emitting devices.

FIG. 28 shows luminance-voltage characteristics of light-emitting devices.

FIG. 29 shows current efficiency-luminance characteristics of light-emitting devices.

FIG. 30 shows current density-voltage characteristics of light-emitting devices.

FIG. 31 shows electroluminescence spectra of light-emitting devices.

FIG. 32 shows luminance changes over driving time of light-emitting devices.

FIG. 33 shows luminance-current density characteristics of light-emitting devices.

FIG. 34 shows luminance-voltage characteristics of light-emitting devices.

FIG. 35 shows current efficiency-luminance characteristics of light-emitting devices.

FIG. 36 shows current density-voltage characteristics of light-emitting devices.

FIG. 37 shows electroluminescence spectra of light-emitting devices.

FIG. 38 shows luminance changes over driving time of light-emitting devices.

FIG. 39 shows luminance-current density characteristics of light-emitting devices.

FIG. 40 shows luminance-voltage characteristics of light-emitting devices.

FIG. 41 shows current efficiency-luminance characteristics of light-emitting devices.

FIG. 42 shows current density-voltage characteristics of light-emitting devices.

FIG. 43 shows electroluminescence spectra of light-emitting devices.

FIG. 44 shows luminance changes over driving time of light-emitting devices.

FIG. 45 shows luminance-current density characteristics of light-emitting devices.

FIG. 46 shows luminance-voltage characteristics of light-emitting devices.

FIG. 47 shows current efficiency-luminance characteristics of light-emitting devices.

FIG. 48 shows current density-voltage characteristics of light-emitting devices.

FIG. 49 shows electroluminescence spectra of light-emitting devices.

FIG. 50 shows luminance changes over driving time of light-emitting devices.

FIG. 51 shows luminance-current density characteristics of light-emitting devices.

FIG. 52 shows luminance-voltage characteristics of light-emitting devices.

FIG. 53 shows current efficiency-luminance characteristics of light-emitting devices.

FIG. 54 shows current density-voltage characteristics of light-emitting devices.

FIG. 55 shows electroluminescence spectra of light-emitting devices.

FIG. 56 shows luminance changes over driving time of light-emitting devices.

### DETAILED DESCRIPTION OF THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

In this embodiment, organic compounds of one embodiment of the present invention will be described.

The organic compound of one embodiment of the present invention can be used for a functional layer of alight-emitting device and alight-receiving device. For example, the organic compound of one embodiment of the present invention can be suitably used for a carrier-transport layer of a light-emitting device and a light-receiving device.

### <Examples of Organic Compound>

The organic compound of one embodiment of the present invention can be represented by General Formulae (G1) to (G5) below.

### <<Example 1 of Organic Compound>>

One embodiment of the present invention is an organic compound represented by General Formula (G1).

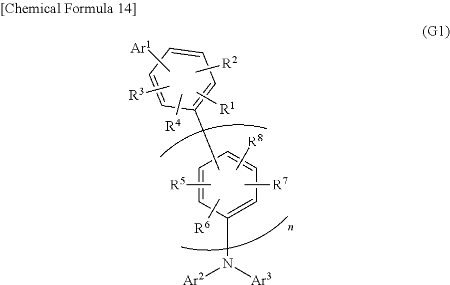

In General Formula (G1), Ar¹ represents any one of General Formulae (g1-1) to (g1-3) below, and each of R¹ to R⁸ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, or a substituted or unsubstituted heteroaryl group having 1 to 30 carbon atoms. Any one of R¹ to R⁴ represents General Formula (g2-1) or General Formula (g2-2) below. Furthermore, n is greater than or equal to 0 and less than or equal to 3. In the case where n is greater than or equal to 2, R⁵s may be the same or different from each other, and the same applies to R⁶s, R⁷s, and R⁸s. Each of Ar² and Ar³ independently represents a substituted or unsubstituted aryl group having 6 to 60 carbon atoms or a substituted or unsubstituted heteroaryl group having 1 to 60 carbon atoms.

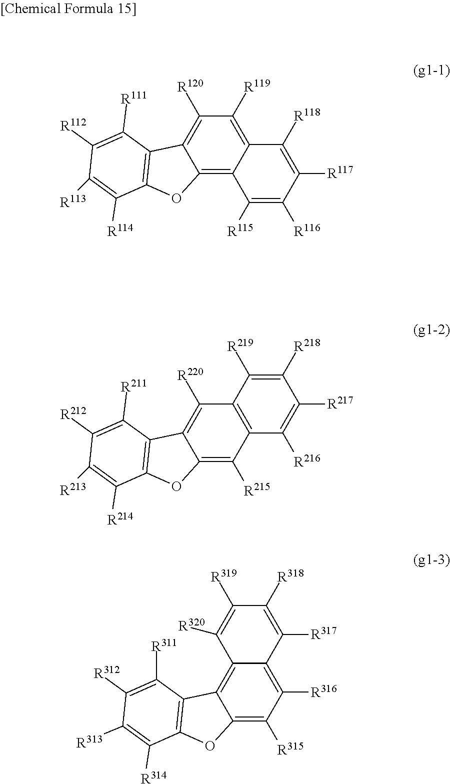

In General Formulae (g1-1) to (g1-3), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹ to R¹²⁰, any one of R²¹¹ to R²²⁰, and any one of R³¹¹ to R³²⁰ each represent a bond with General Formula (G1).

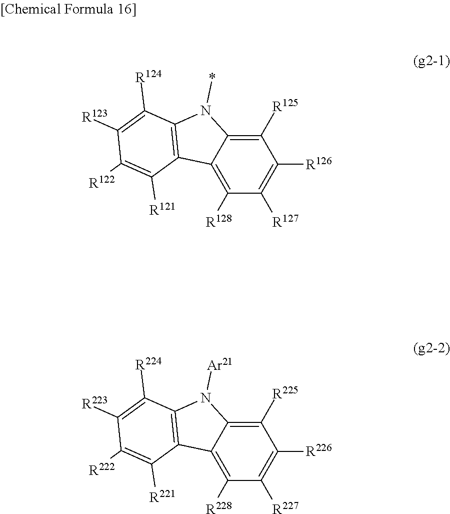

In General Formulae (g2-1) and (g2-2), each of R¹²¹ to R¹²⁸ and R²²¹ to R²²⁸ independently represents hydrogen (including deuterium), an alkyl group having 1 to 6 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. An asterisk (*) and any one of R²²¹ to R²²⁸ represent a bond with General Formula (G1). Ar²¹ represents a substituted or unsubstituted aryl group having 6 to 30 carbon atoms or a substituted or unsubstituted heteroaryl group having 1 to 60 carbon atoms.

The organic compound represented by General Formula (G1) is an organic EL material having a high hole-transport property, high heat resistance, and high reliability when used in a device. With the use of the organic compound of one embodiment of the present invention, a light-emitting device or a light-receiving device with high reliability and low power consumption can be provided.

### <<Example 2 of Organic Compound>>

Another embodiment of the present invention is an organic compound represented by General Formula (G2).

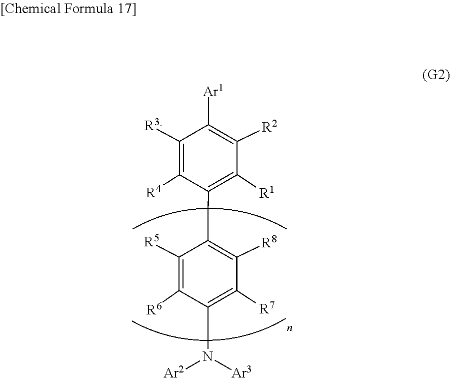

In General Formula (G2), Ar¹ represents any one of General Formulae (g1-1) to (g1-3) below, and each of R¹ to R⁸ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, or a heteroaryl group having 1 to 30 carbon atoms. Any one of R¹ to R⁴ represents General Formula (g2-1) or General Formula (g2-2) above. Furthermore, n is greater than or equal to 0 and less than or equal to 3. Each of Ar² and Ar³ independently represents a substituted or unsubstituted aryl group having 6 to 60 carbon atoms or a substituted or unsubstituted heteroaryl group having 1 to 60 carbon atoms.

In the organic compound represented by General Formula (G2), a phenyl group with Ar¹ bonded at the para-position can cap a portion considered to easily deteriorate, thereby improving the reliability. The easiness of deterioration is due to the para-directing effect of an amino group, which increases the reactivity of hydrogen (or deuterium) at the para-position and makes a hydrogen-carbon bond be easily cut (makes the reactivity of hydrogen (or deuterium) highest). When benzonaphthofuran (Ar¹) having a stable structure is bonded to the amino group at the para-position with high reactivity, the stability of the organic compound in a carrier-receiving state or an excited state can be improved. Furthermore, the bonding at the para-position achieves a rigid structure, thereby improving the heat resistance and the carrier mobility.

With a substituent represented by General Formula (g2-1) or General Formula (g2-2) above, which is a carbazole skeleton, the compound can have high hole mobility. In particular, the substituent represented by General Formula (g2-1) or General Formula (g2-2) above is preferably R² (or R³). Furthermore, when the substituent represented by General Formula (g2-1) or General Formula (g2-2) above is bonded to carbon adjacent to carbon to which Ar¹ is bonded, the molecular structure becomes sterically bulky, which can inhibit crystallization and enables formation of a stable film. Thus, the organic compound of one embodiment of the present invention can be suitably used in a thin-film state for a light-emitting device and a light-receiving device. In addition, a device that is resistant to high-temperature driving and high-temperature environments can be provided.

Note that in the case where n is less than or equal to 2 in the organic compounds represented by General Formulae (G1) and (G2), the sublimation temperature is not too high and decomposition at the time of evaporation can be inhibited. In the case where n is greater than or equal to 2, the HOMO level is not too high and high hole mobility can be achieved. In the case where n is 1, the organic compound has stable film quality and a sublimation property, and thus can have high reliability. Furthermore, the HOMO level of the case where n is 1 is suitable for the use in a light-emitting device, which is preferable.

### <<Organic Compound Example 3>>

Another embodiment of the present invention is an organic compound represented by General Formula (G3).

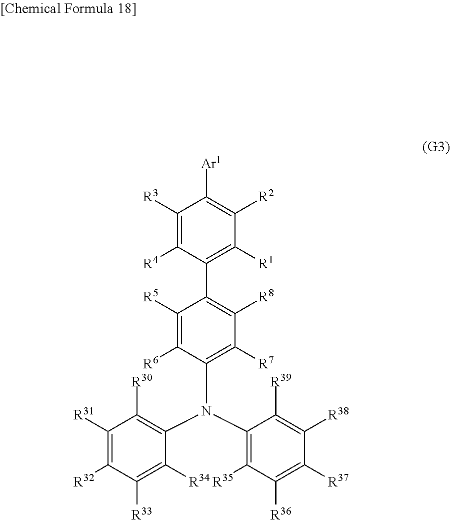

Ar¹ represents any one of General Formulae (g1-1) to (g1-3) below, and each of R¹ to R⁸ and R³⁰ to R³⁹ independently represents hydrogen (including deuterium), an alkyl group having 1 to 6 carbon atoms, a cycloalkyl group having 3 to 10 carbon atoms, an alkoxy group having 1 to 6 carbon atoms, or a substituted or unsubstituted aryl group having 6 to 15 carbon atoms. R² represents General Formula (g2-1) or General Formula (g2-3) below. In the case where each of R³⁰ to R³⁹ represents a substituted aryl group having 6 to 15 carbon atoms, the aryl group having 6 to 15 carbon atoms may be bonded to an adjacent aromatic ring to form a ring, and the formed ring may have a substituent.

The organic compound represented by General Formula (G3) includes an aryl group as each of Ar² and Ar³ of the organic compound represented by General Formula (G2), and thus easily has a desired HOMO level suitable for device design. This enables provision of a hole-transport material suitable for a light-emitting device.

In particular, at least one of R³⁰ to R³⁴ and at least one of R³⁵ to R³⁹ are each preferably a substituted or unsubstituted phenyl group. With this structure, the organic compound has a high hole-transport property and high stability in an excited state and in a hole-receiving state. Thus, the organic compound can have high reliability.

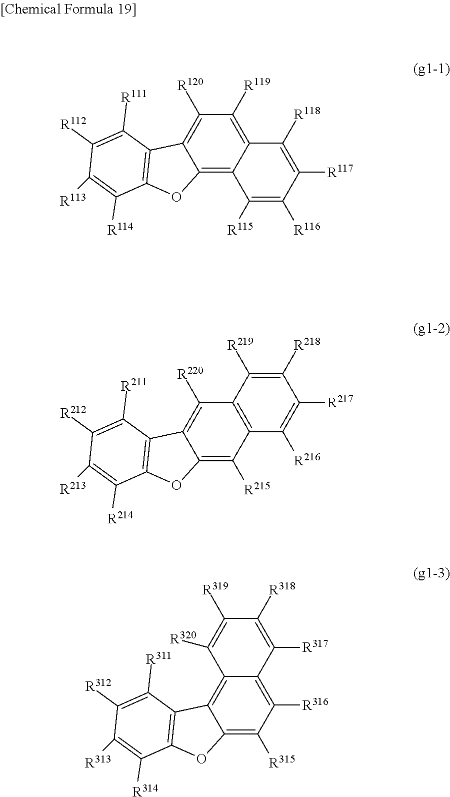

In General Formulae (g1-1) to (g1-3), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹, R¹¹², R¹¹⁴, and R¹¹⁹, any one of R²¹², R²¹⁴, and R²¹⁵, and any one of R³¹², R³¹⁴, and R³¹⁵ each represent a bond with General Formula (G3).

When any one of R¹¹¹, R¹¹², R¹¹⁴, and R¹¹⁹ in General Formula (g1-1) is bonded to the organic compound represented by General Formula (G3), a compound having a desired LUMO level can be obtained; thus, a hole-transport material having an electron-blocking property suitable for device design can be provided. The organic compound in which the bond with General Formula (G3) is made at any one of R¹¹¹, R¹¹², R¹¹⁴, and R¹¹⁹ in General Formula (g1-1) can be synthesized through a smaller number of reactions (a smaller number of synthesis steps) at lower cost than an organic compound in which the bond with General Formula (G3) is made at another position. The kinds and the amounts of impurities increase with the number of synthesis steps. Accordingly, a smaller number of synthesis steps enables a higher-purity compound and higher-yield manufacturing of a highly reliable device.

Similarly, when any one of R²¹², R²¹⁴, and R²¹⁵ in General Formula (g1-2) and any one of R³¹², R³¹⁴, and R³¹⁵ in General Formula (g1-3) are bonded to the organic compound represented by General Formula (G3), a compound having a desired LUMO level can be obtained. The organic compound in which the bonds with General Formula (G3) are made at any one of R²¹², R²¹⁴ and R²¹⁵ in General Formula (g1-2) and any one of R³¹², R³¹⁴ and R³¹⁵ in General Formula (g1-3) can be synthesized through a smaller number of reactions (a smaller number of synthesis steps) than an organic compound in which the bonds with General Formula (G3) are made at other positions. Accordingly, a highly purified organic compound can be provided, so that a highly reliable device can be manufactured with high yield.

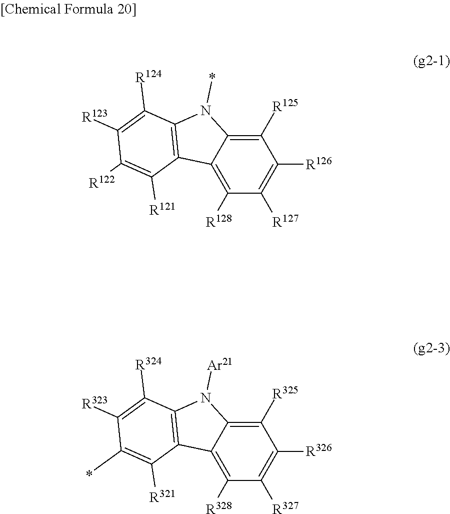

In General Formulae (g2-1) and (g2-3), each of R¹²¹ to R¹²⁸ and R³²¹ to R³²⁸ independently represents hydrogen (including deuterium), an alkyl group having 1 to 6 carbon atoms, an alkoxy group having 1 to 6 carbon atoms, or a substituted or unsubstituted aryl group having 6 to 30 carbon atoms. An asterisk (*) represents a bond with General Formula (G3). Ar²¹ represents a substituted or unsubstituted aryl group having 6 to 30 carbon atoms or a substituted or unsubstituted heteroaryl group having 1 to 30 carbon atoms.

### <<Organic Compound Example 4>>

Another embodiment of the present invention is an organic compound represented by General Formula (G4).

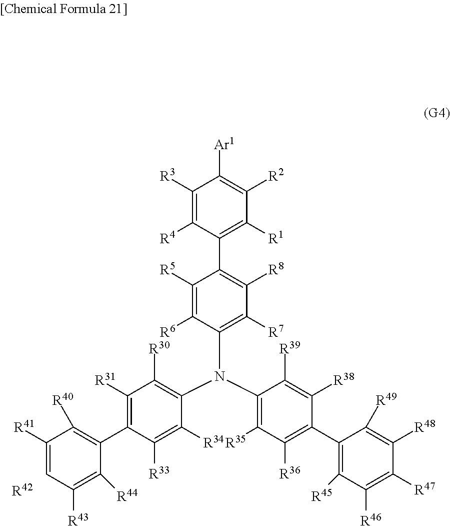

In General Formula (G4), Ar¹ represents any one of General Formulae (g1-1) to (g1-3), and each of R¹ to R⁸ and R³⁰ to R⁴⁹ independently represents hydrogen (including deuterium), an alkyl group having 1 to 6 carbon atoms, a cycloalkyl group having 3 to 10 carbon atoms, an alkoxy group having 1 to 6 carbon atoms, or a substituted or unsubstituted aryl group having 6 to 15 carbon atoms. R² represents General Formula (g2-1) or General Formula (g2-3) below.

The organic compound represented by General Formula (G4) includes a biphenyl group as each of Ar² and Ar³ of the organic compound represented by General Formula (G2), and thus has a high hole-transport property and high heat resistance. In addition, the organic compound represented by General Formula (G4) has a molecular structure where the para-position of an aniline structure (the para-position of an amino group) is substituted by a phenyl group, and thus has high stability in an excited state and a hole-receiving state.

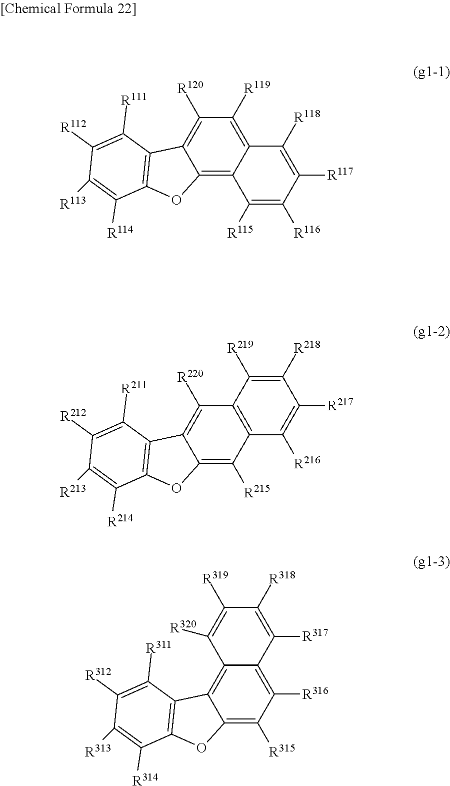

In General Formulae (g1-1) to (g1-3), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹, R¹¹², R¹¹⁴, and R¹¹⁹ any one of R²¹², R²¹⁴, and R²¹⁵, and any one of R³¹², R³¹⁴, and R³¹⁵ each represent a bond with General Formula (G4).

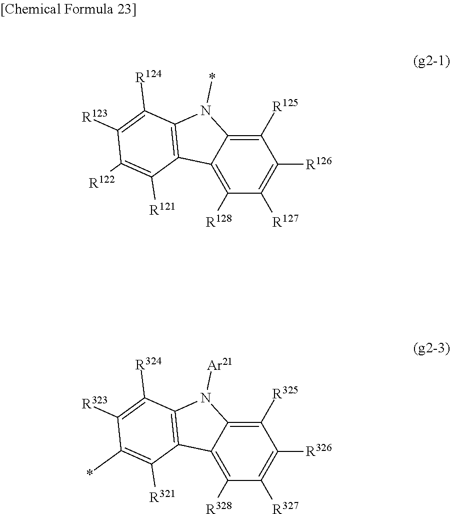

In General Formulae (g2-1) and (g2-3), each of R¹²¹ to R¹²⁸ and R³²¹ to R³²⁸ independently represents hydrogen (including deuterium), an alkyl group having 1 to 6 carbon atoms, an alkoxy group having 1 to 6 carbon atoms, or a substituted or unsubstituted aryl group having 6 to 30 carbon atoms. The asterisk (*) represents a bond with General Formula (G4). Ar²¹ represents a substituted or unsubstituted aryl group having 6 to 30 carbon atoms or a substituted or unsubstituted heteroaryl group having 1 to 30 carbon atoms.

### <<Organic Compound Example 5>>

Another embodiment of the present invention is an organic compound represented by General Formula (G5).

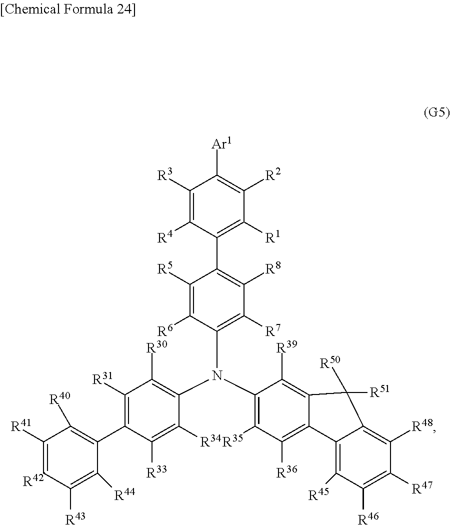

In General Formula (G5), Ar¹ represents any one of General Formulae (g1-1) to (g1-3), and each of R¹ to R⁸ and R³⁰ to R⁵¹ independently represents hydrogen (including deuterium), an alkyl group having 1 to 6 carbon atoms, a cycloalkyl group having 3 to 10 carbon atoms, an alkoxy group having 1 to 6 carbon atoms, or a substituted or unsubstituted aryl group having 6 to 15 carbon atoms. In the case where each of R⁵⁰ and R⁵¹ is a substituted or unsubstituted aryl group having 6 to 15 carbon atoms, R⁵⁰ and R⁵¹ may be bonded to each other to form a ring. R² represents General Formula (g2-1) or General Formula (g2-3) below.

In the organic compound represented by General Formula (G5), a ring is formed by a carbon atom bonded to R⁵⁰ and R⁵¹ at the positions of R³⁸ and R⁴⁹ of the organic compound represented by General Formula (G4), whereby a fluorene structure is formed. Thus, the organic compound represented by General Formula (G5) has a fluorenylamine structure, which has a higher HOMO level than a biphenylamine structure, and thus can have a high hole-transport property. This enables provision of a hole-transport material suitable for device design.

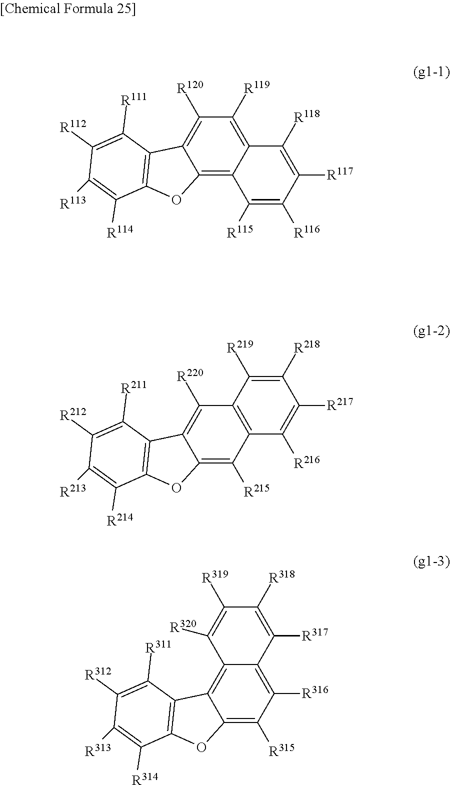

In General Formulae (g1-1) to (g1-3), each of R¹¹¹ to R¹²⁰, R²¹¹ to R²²⁰, and R³¹¹ to R³²⁰ independently represents hydrogen (including deuterium), an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, or a substituted or unsubstituted heteroaryl group having 1 to 30 carbon atoms. Any one of R¹¹¹, R¹¹², R¹¹⁴, and R¹¹⁹ any one of R²¹², R²¹⁴, and R²¹⁵, and any one of R³¹², R³¹⁴, and R³¹⁵ each represent a bond with General Formula (G5).

In particular, each of R¹¹⁴, R²¹⁴, and R³¹⁴ in General Formulae (g1-1) to (g1-3) above is preferably a bond with General Formula (G5). With this structure, an organic compound having a desired LUMO level can be designed, so that a hole-transport material having an electron-blocking property suitable for device design can be provided. The organic compound in which the bonds with General Formula (G5) are made at any one of R¹¹¹, R¹¹², R¹¹⁴, and R¹¹⁹ in General Formula (g1-1), any one of R²¹², R²¹⁴ and R²¹⁵ in General Formula (g1-2), and any one of R³¹², R³¹⁴ and R³¹⁵ in General Formula (g1-3) can be synthesized through a smaller number of reactions (a smaller number of synthesis steps) than an organic compound in which the bonds with General Formula (G5) are made at other positions. Accordingly, a highly purified organic compound can be provided, so that a highly reliable device can be manufactured at low cost with high yield.

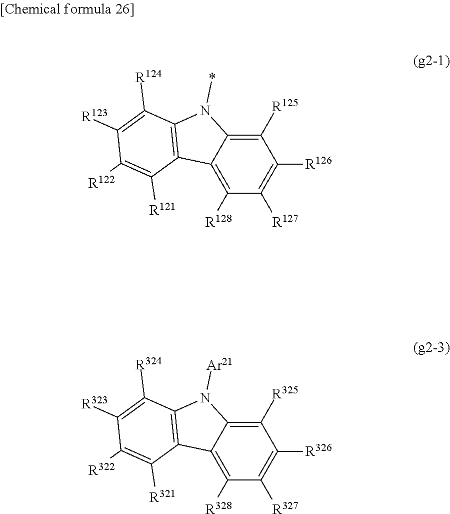

In General Formulae (g2-1) and (g2-3), each of R¹²¹ to R¹²⁸ and R³²¹ to R³²⁸ independently represents hydrogen (including deuterium), an alkyl group having 1 to 6 carbon atoms, an alkoxy group having 1 to 6 carbon atoms, or a substituted or unsubstituted aryl group having 6 to 30 carbon atoms. The asterisk (*) represents a bond with General Formula (G5). Ar²¹ represents a substituted or unsubstituted aryl group having 6 to 30 carbon atoms or a substituted or unsubstituted heteroaryl group having 1 to 30 carbon atoms.

Specific examples of substituents represented by Ar² and Ar³ in General Formulae (G1) and (G2) and a substituent represented by Ar²¹ in General Formula (g2-3) are shown below.

The substituted or unsubstituted aryl group having 6 to 30 carbon atoms, the substituted or unsubstituted aryl group having 6 to 60 carbon atoms, the substituted or unsubstituted heteroaryl group having 1 to 30 carbon atoms, or the substituted or unsubstituted heteroaryl group having 1 to 60 carbon atoms is preferably represented by any of (Ar-1) to (Ar-123). Note that * in (Ar-1) to (Ar-123) represents a bonding position.

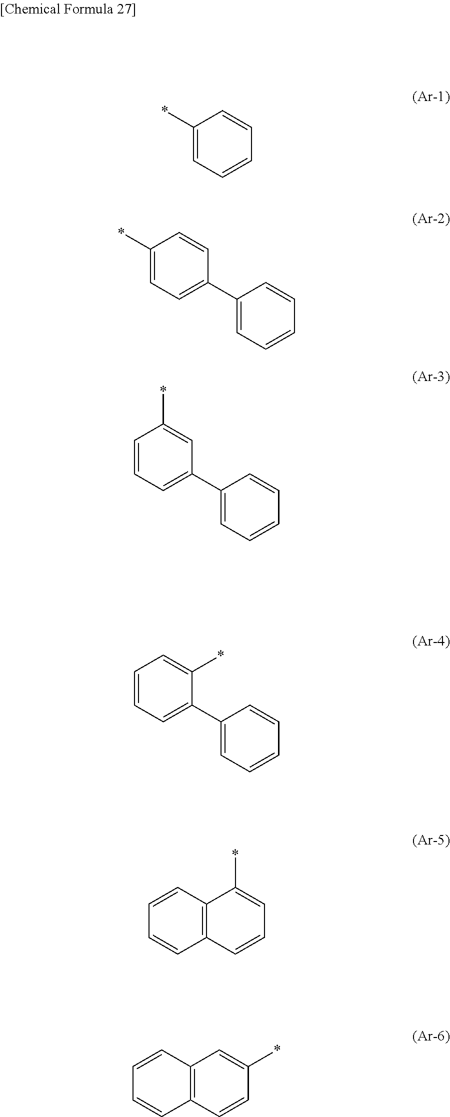 [SMILES: *c1ccccc1 ; *c1ccc(-c2ccccc2)cc1 ; *c1cccc(-c2ccccc2)c1 ; *c1ccccc1-c1ccccc1 ; *c1cccc2ccccc12 ; *c1ccc2ccccc2c1]

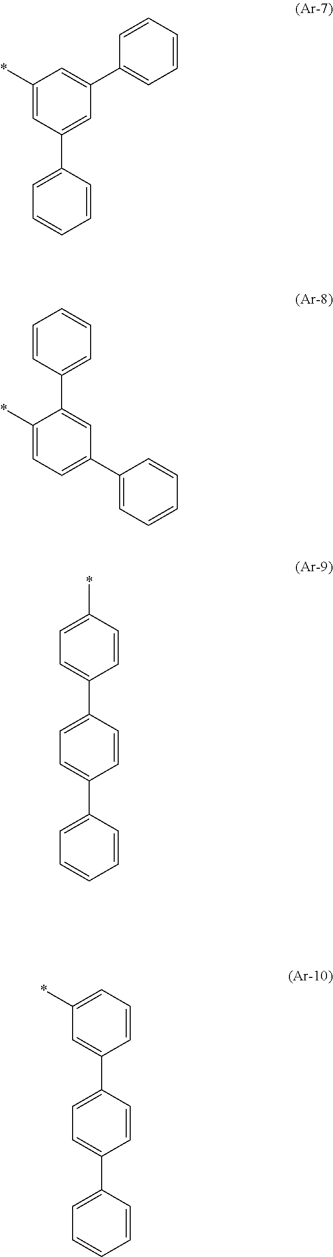 [SMILES: *c1cc(-c2ccccc2)cc(-c2ccccc2)c1 ; *c1ccc(-c2ccccc2)cc1-c1ccccc1 ; *c1ccc(-c2ccc(-c3ccccc3)cc2)cc1 ; *c1cccc(-c2ccc(-c3ccccc3)cc2)c1]

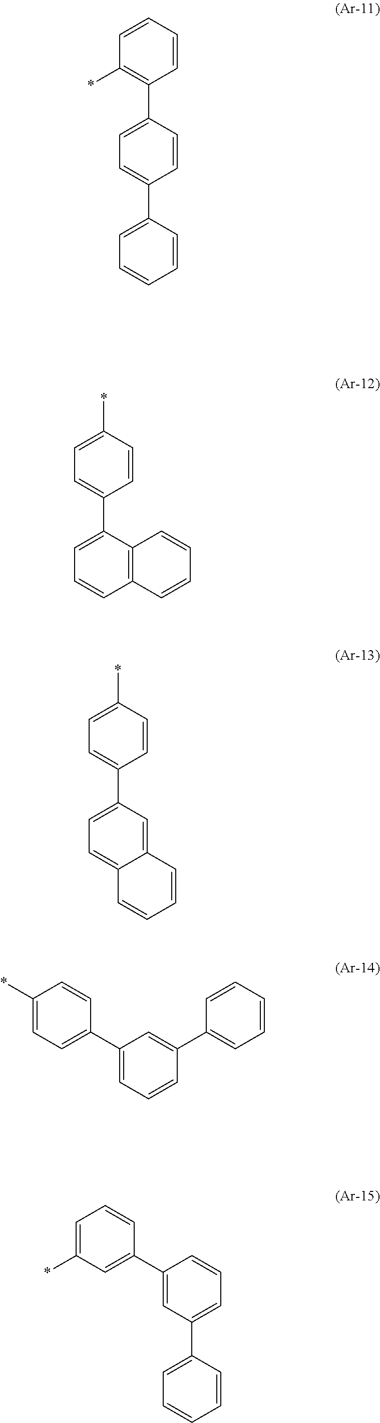 [SMILES: *c1ccccc1-c1ccc(-c2ccccc2)cc1 ; *c1ccc(-c2cccc3ccccc23)cc1 ; *c1ccc(-c2ccc3ccccc3c2)cc1 ; *c1ccc(-c2cccc(-c3ccccc3)c2)cc1 ; *c1cccc(-c2cccc(-c3ccccc3)c2)c1]

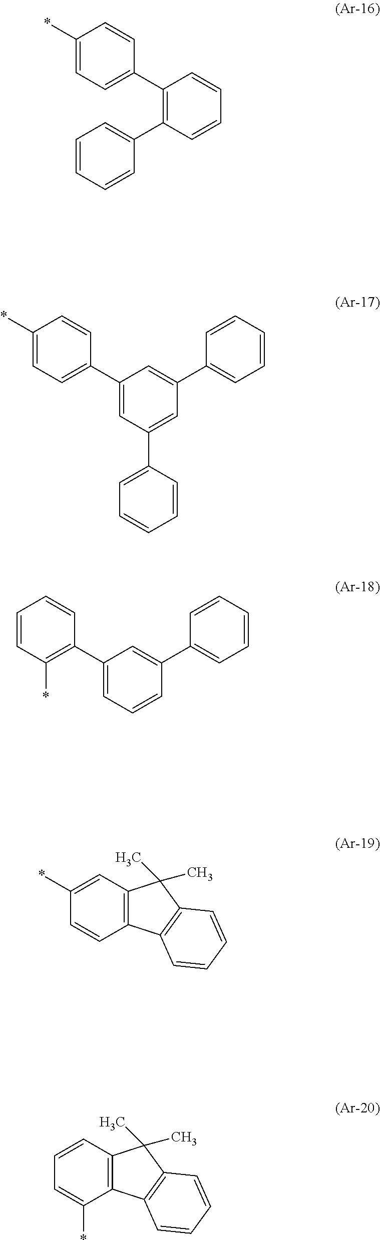 [SMILES: *c1ccc(-c2ccccc2-c2ccccc2)cc1 ; *c1ccc(-c2cc(-c3ccccc3)cc(-c3ccccc3)c2)cc1 ; *c1ccccc1-c1cccc(-c2ccccc2)c1 ; *c1ccc2c(c1)C(C)(C)c1ccccc1-2 ; *c1cccc2c1-c1ccccc1C2(C)C]

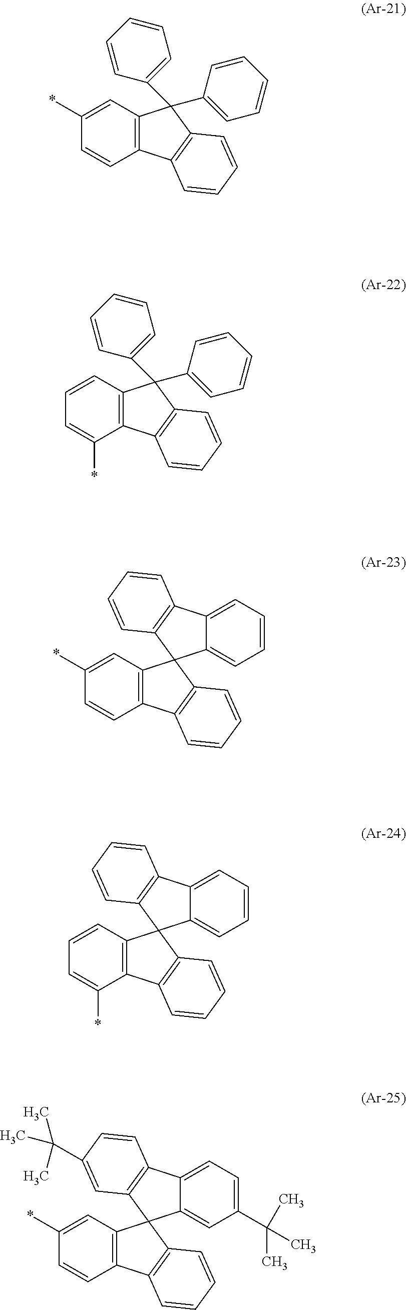 [SMILES: *c1ccc2c(c1)C(c1ccccc1)(c1ccccc1)c1ccccc1-2 ; *c1cccc2c1-c1ccccc1C2(c1ccccc1)c1ccccc1 ; *c1ccc2c(c1)C1(c3ccccc3-c3ccccc31)c1ccccc1-2 ; *c1cccc2c1-c1ccccc1C21c2ccccc2-c2ccccc21 ; *c1ccc2c(c1)C1(c3ccccc3-2)c2cc(C(C)(C)C)ccc2-c2ccc(C(C)(C)C)cc21]

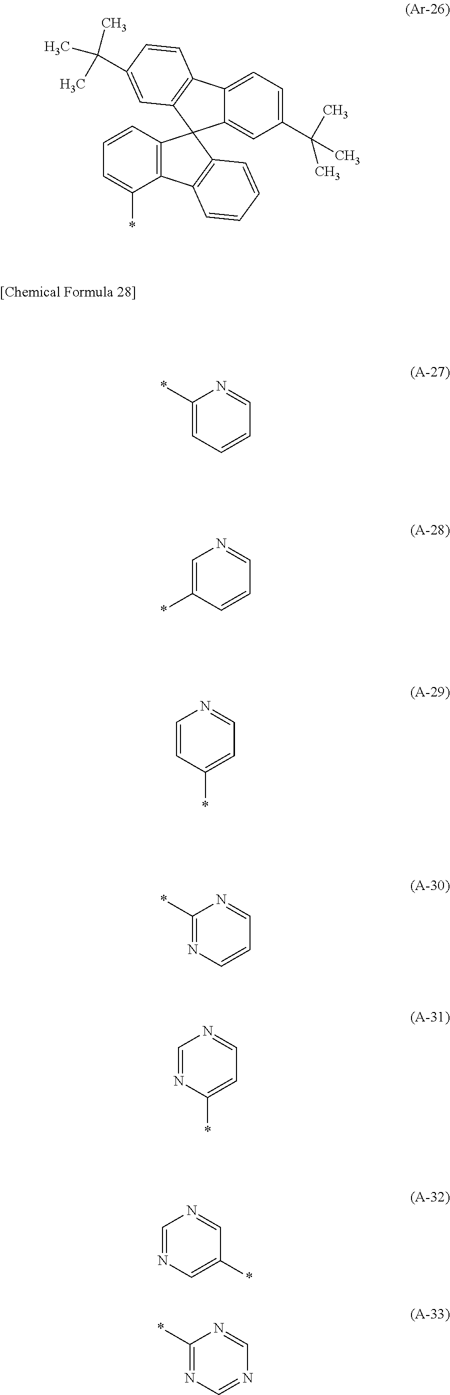

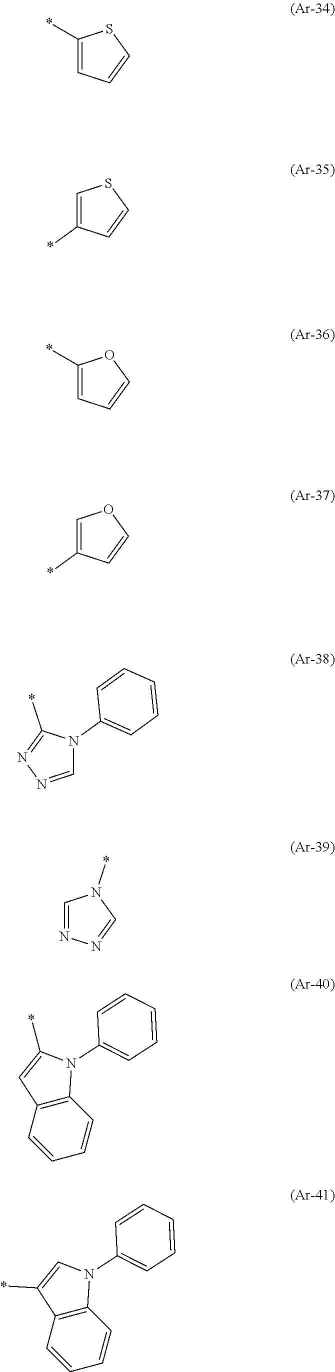

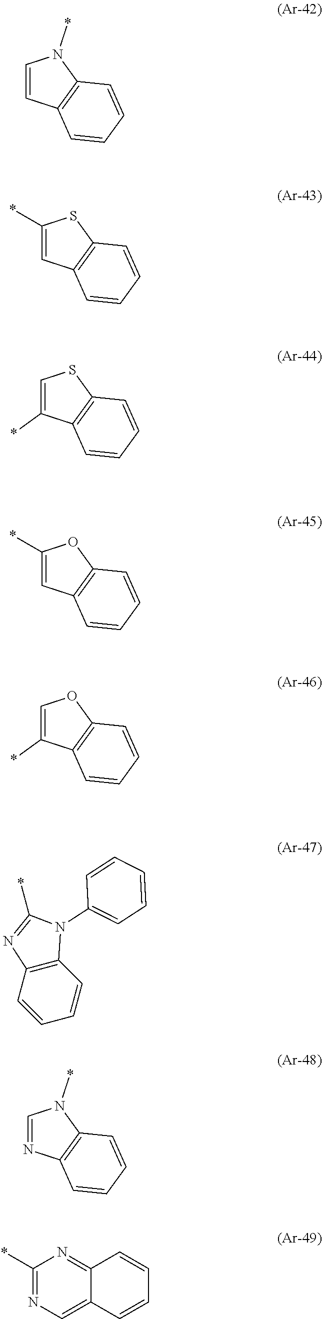

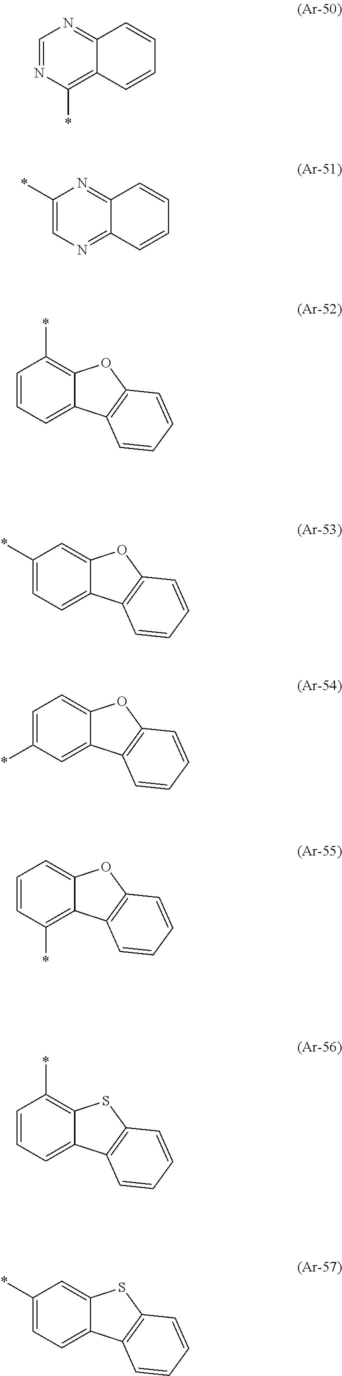

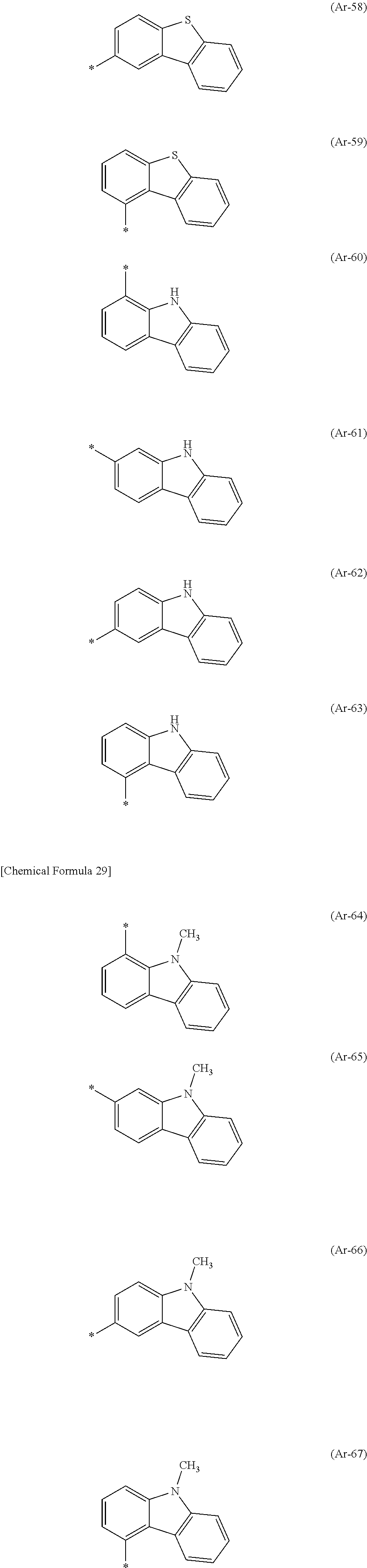

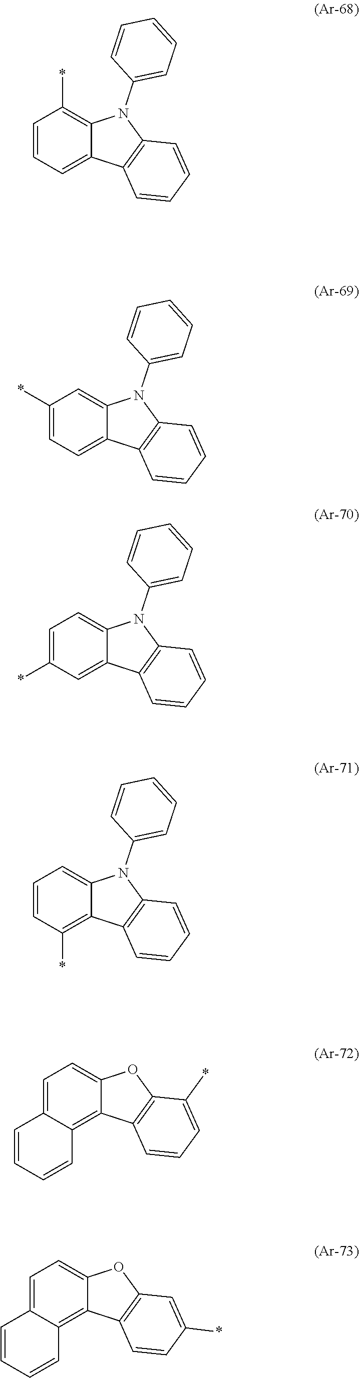

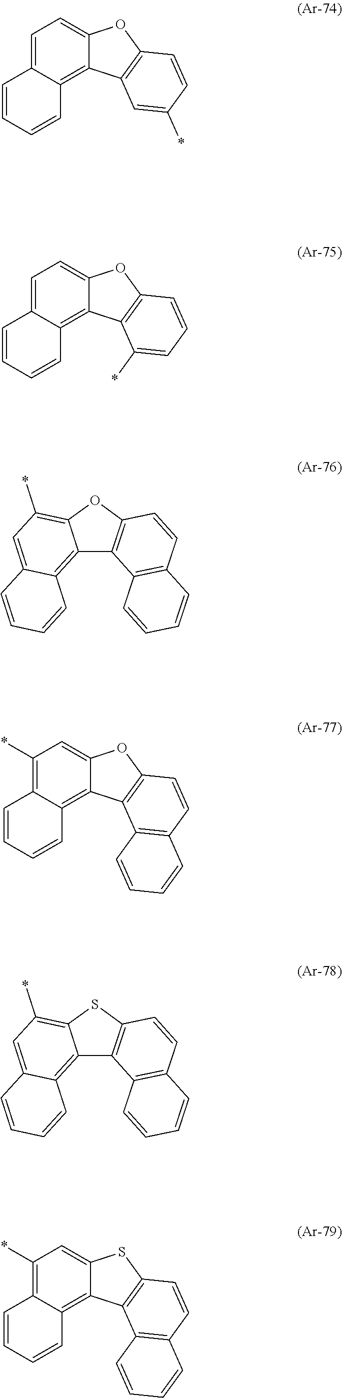 [SMILES: *c1ccc2oc3ccc4ccccc4c3c2c1 ; *c1cccc2oc3ccc4ccccc4c3c12 ; *c1cc2ccccc2c2c1oc1ccc3ccccc3c12 ; *c1cc2oc3ccc4ccccc4c3c2c2ccccc12 ; *c1cc2ccccc2c2c1sc1ccc3ccccc3c12 ; *c1cc2sc3ccc4ccccc4c3c2c2ccccc12]

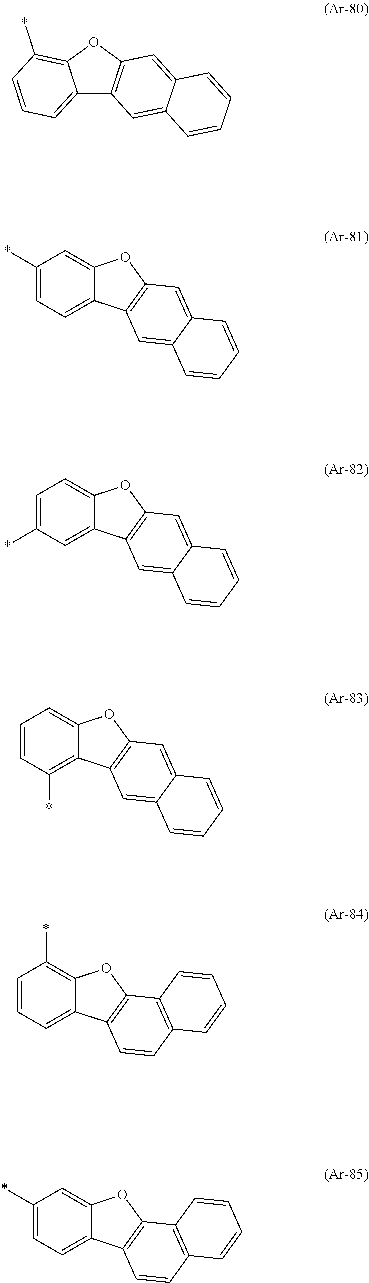 [SMILES: *c1cccc2c1oc1cc3ccccc3cc12 ; *c1ccc2c(c1)oc1cc3ccccc3cc12 ; *c1ccc2oc3cc4ccccc4cc3c2c1 ; *c1cccc2oc3cc4ccccc4cc3c12 ; *c1cccc2c1oc1c3ccccc3ccc21 ; *c1ccc2c(c1)oc1c3ccccc3ccc21]

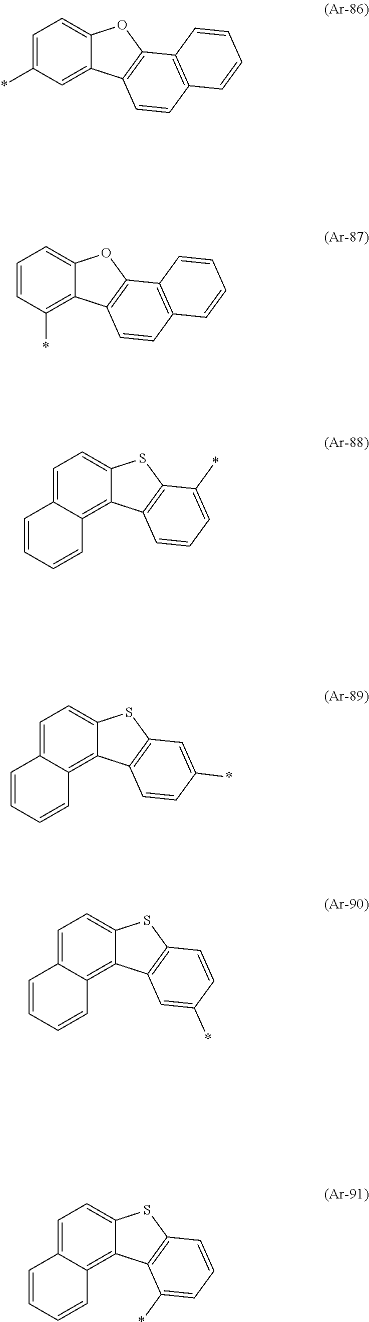 [SMILES: *c1ccc2oc3c4ccccc4ccc3c2c1 ; *c1cccc2oc3c4ccccc4ccc3c12 ; *c1cccc2c1sc1ccc3ccccc3c12 ; *c1ccc2c(c1)sc1ccc3ccccc3c12 ; *c1ccc2sc3ccc4ccccc4c3c2c1 ; *c1cccc2sc3ccc4ccccc4c3c12]

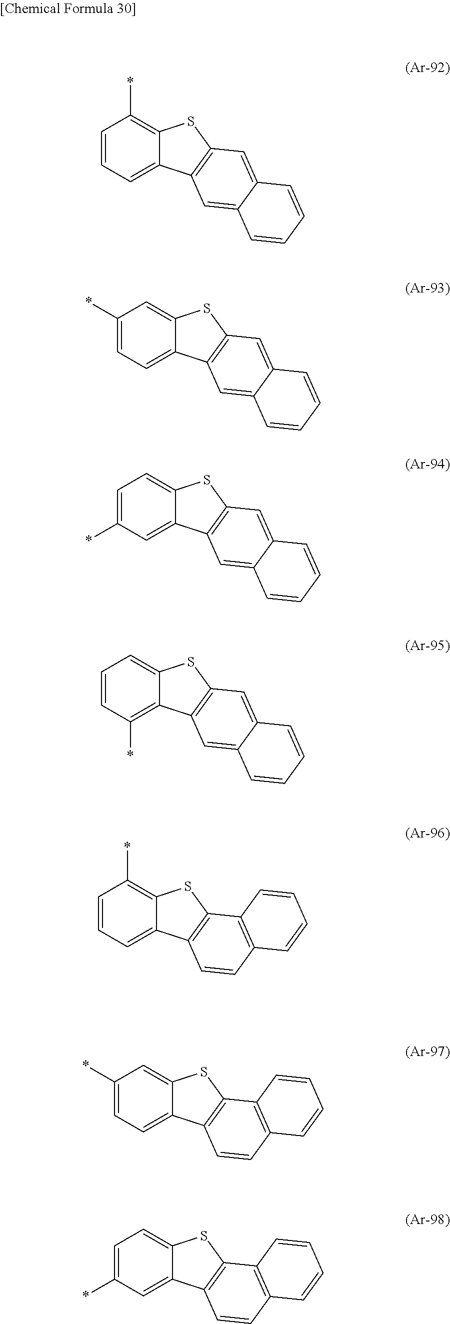

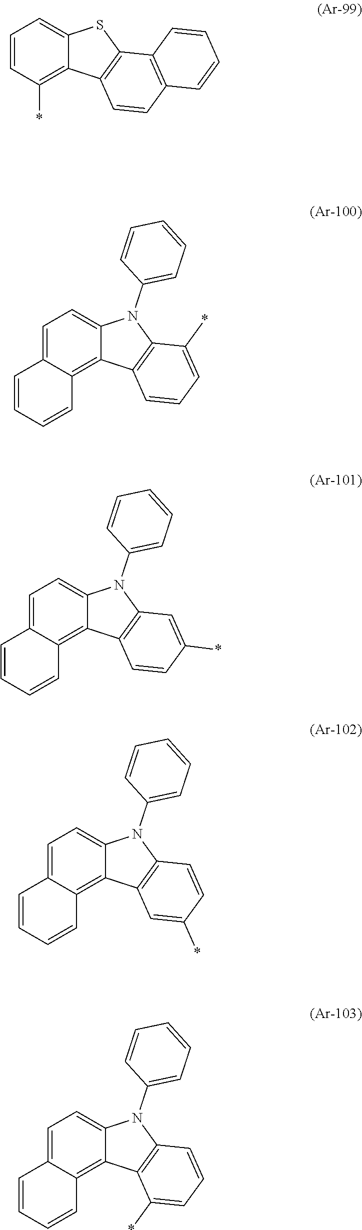 [SMILES: *c1cccc2sc3c4ccccc4ccc3c12 ; *c1cccc2c3c4ccccc4ccc3n(-c3ccccc3)c12 ; *c1ccc2c3c4ccccc4ccc3n(-c3ccccc3)c2c1 ; *c1ccc2c(c1)c1c3ccccc3ccc1n2-c1ccccc1 ; *c1cccc2c1c1c3ccccc3ccc1n2-c1ccccc1]

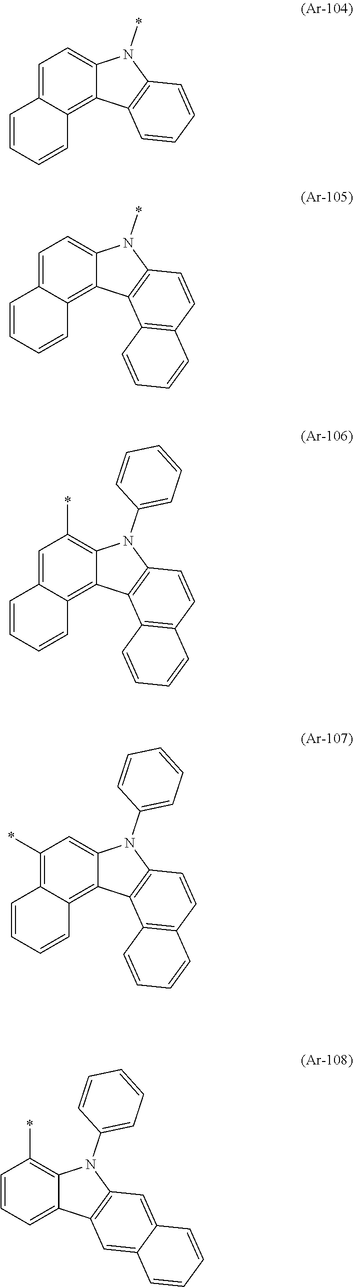 [SMILES: *n1c2ccccc2c2c3ccccc3ccc21 ; *n1c2ccc3ccccc3c2c2c3ccccc3ccc21 ; *c1cc2ccccc2c2c3c4ccccc4ccc3n(-c3ccccc3)c12 ; *c1cc2c(c3ccccc13)c1c3ccccc3ccc1n2-c1ccccc1 ; *c1cccc2c3cc4ccccc4cc3n(-c3ccccc3)c12]

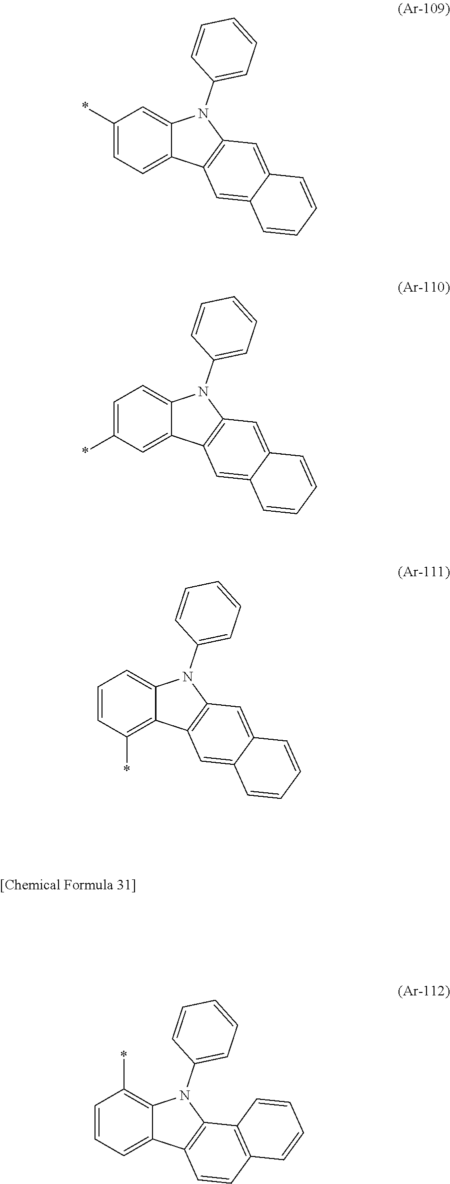 [SMILES: *c1ccc2c3cc4ccccc4cc3n(-c3ccccc3)c2c1 ; *c1ccc2c(c1)c1cc3ccccc3cc1n2-c1ccccc1 ; *c1cccc2c1c1cc3ccccc3cc1n2-c1ccccc1 ; *c1cccc2c3ccc4ccccc4c3n(-c3ccccc3)c12]

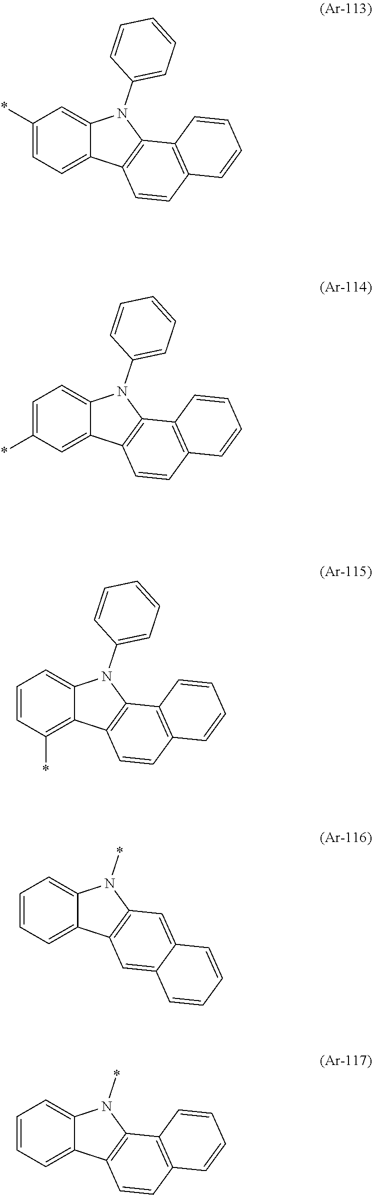 [SMILES: *c1ccc2c3ccc4ccccc4c3n(-c3ccccc3)c2c1 ; *c1ccc2c(c1)c1ccc3ccccc3c1n2-c1ccccc1 ; *c1cccc2c1c1ccc3ccccc3c1n2-c1ccccc1 ; *n1c2ccccc2c2cc3ccccc3cc21 ; *n1c2ccccc2c2ccc3ccccc3c21]

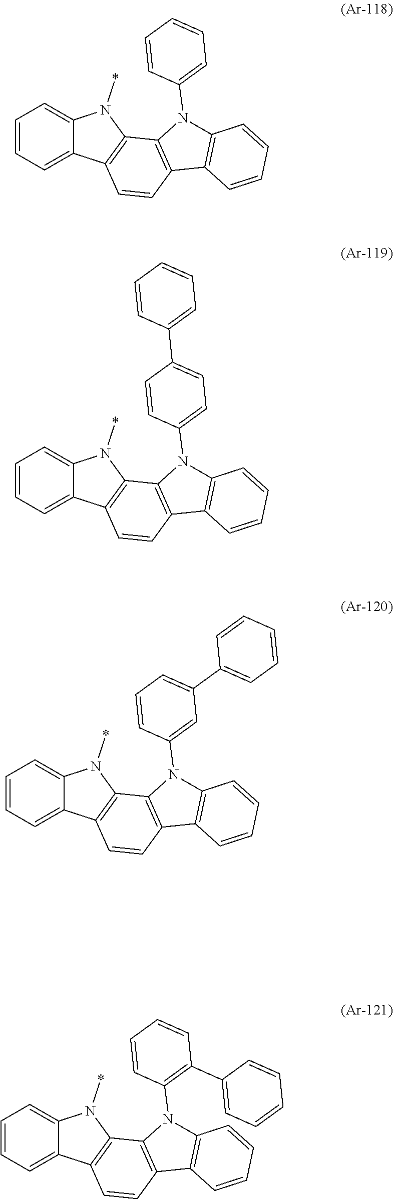 [SMILES: *n1c2ccccc2c2ccc3c4ccccc4n(-c4ccccc4)c3c21 ; *n1c2ccccc2c2ccc3c4ccccc4n(-c4ccc(-c5ccccc5)cc4)c3c21 ; *n1c2ccccc2c2ccc3c4ccccc4n(-c4cccc(-c5ccccc5)c4)c3c21 ; *n1c2ccccc2c2ccc3c4ccccc4n(-c4ccccc4-c4ccccc4)c3c21]

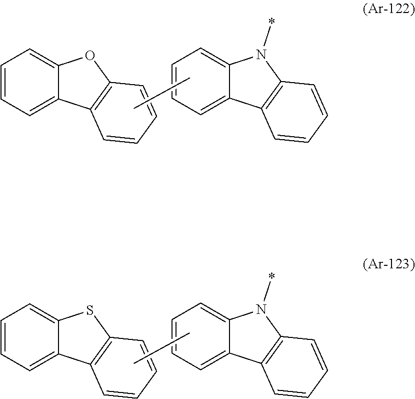

Specific examples of substituents represented by R^{m} (m is a given integer) in General Formulae (G1) to (G5), General Formulae (g1-1) to (g1-3), and General Formulae (g2-1) to (g2-3) are shown below.

Examples of the alkyl group having 1 to 6 carbon atoms include a methyl group, an ethyl group, a propyl group, an isopropyl group, a butyl group, an isobutyl group, a sec-butyl group, a tert-butyl group, a pentyl group, and a hexyl group.

Examples of the cycloalkyl group having 3 to 10 carbon atoms include a cyclopropyl group, a cyclobutyl group, a methylcyclobutyl group, a cyclopentyl group, a methylcyclopentyl group, an isopropylcyclopentyl group, a tert-butylcyclopropyl group, a cyclohexyl group, a methylcyclohexyl group, an isopropylcyclohexyl group, a tert-butylcyclohexyl group, a cycloheptyl group, a methylcycloheptyl group, an isopropylcycloheptyl group, a cyclooctyl group, a methylcyclooctyl group, a cyclononyl group, a methylcyclononyl group, a cyclodecyl group, and an adamantyl group.

Examples of the alkoxy group having 1 to 6 carbon atoms include a methoxy group, an ethoxy group, a propoxy group, an isopropoxy group, a tert-butoxy group, a sec-butoxy group, an isobutoxy group, a pentyloxy group, an octyloxy group, an allyloxy group, a cyclohexyloxy group, a phenoxy group, a benzyloxy group, a vinyloxy group, a propenyloxy group, a butenyloxy group, a pentenyloxy group, and a hexenyloxy group.

Examples of the aryl group having 6 to 15 carbon atoms include a phenyl group, a biphenyl group, a naphthyl group, a fluorenyl group, and a 9,9-dimethylfluorenyl group.

Examples of the heteroaryl group having 2 to 8 carbon atoms include a pyridinyl group, a pyrazinyl group, a pyrimidinyl group, a pyridazinyl group, a triazinyl group, a quinolinyl group, a quinazolinyl group, an isoquinolinyl group, a pyrrolyl group, a naphthrydinyl group, a phenanthrolinyl group, a quinoxalinyl group, an imidazolyl group, a benzimidazolyl group, an oxazolyl group, an isoxazolyl group, a thiazolyl group, an isothiazolyl group, and a benzofuranyl group.

In the case where the substituted or unsubstituted aryl group having 6 to 60 carbon atoms, the substituted or unsubstituted aryl group having 6 to 30 carbon atoms, the substituted or unsubstituted aryl group having 6 to 15 carbon atoms, the substituted or unsubstituted heteroaryl group having 1 to 60 carbon atoms, the substituted or unsubstituted heteroaryl group having 1 to 30 carbon atoms, and the substituted or unsubstituted heteroaryl group having 2 to 8 carbon atoms has a substituent, an alkyl group having 1 to 6 carbon atoms, a cycloalkyl group having 3 to 10 carbon atoms, a cyano group, or a hydroxyl group can be selected as the substituent.

As for the substituent bonded to the aryl group or the heteroaryl group, specific examples of the alkyl group having 1 to 6 carbon atoms include a methyl group, an ethyl group, a propyl group, an isopropyl group, a butyl group, an isobutyl group, a tert-butyl group, and an n-hexyl group. Specific examples of the cycloalkyl group having 3 to 10 carbon atoms include a cyclopropyl group, a cyclobutyl group, a cyclopentyl group, a cyclohexyl group, and an adamantyl group.

In General Formulae (G1) to (G5), (g1-1) to (g1-3), and (g2-1) to (g2-3), hydrogen may be replaced with deuterium as appropriate.

### SPECIFIC EXAMPLES

The following are specific examples of the organic compound of one embodiment of the present invention having the structure represented by any of General Formulae (G1) to (G5).

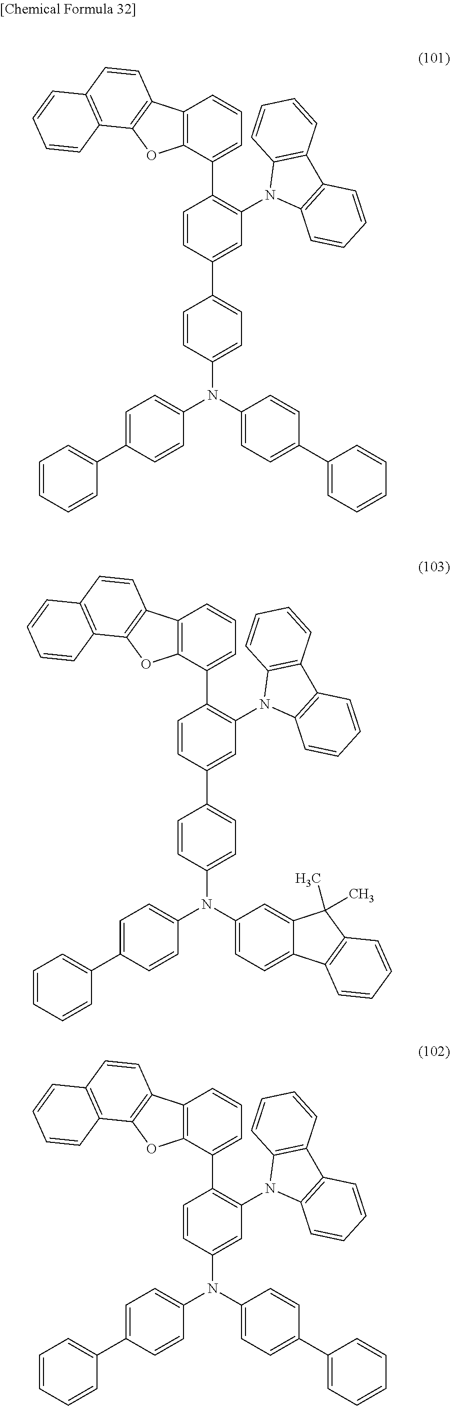

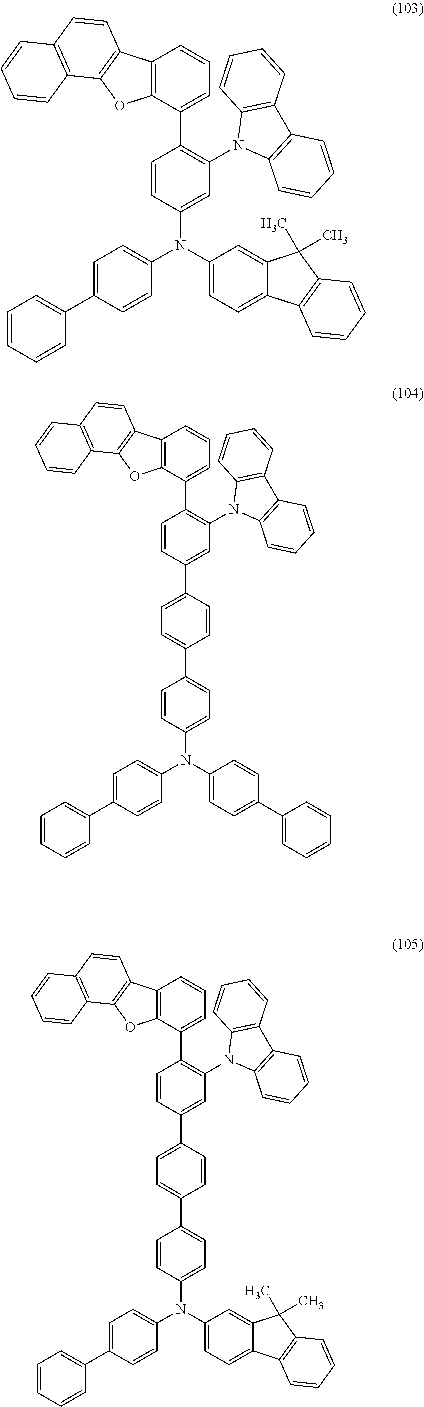

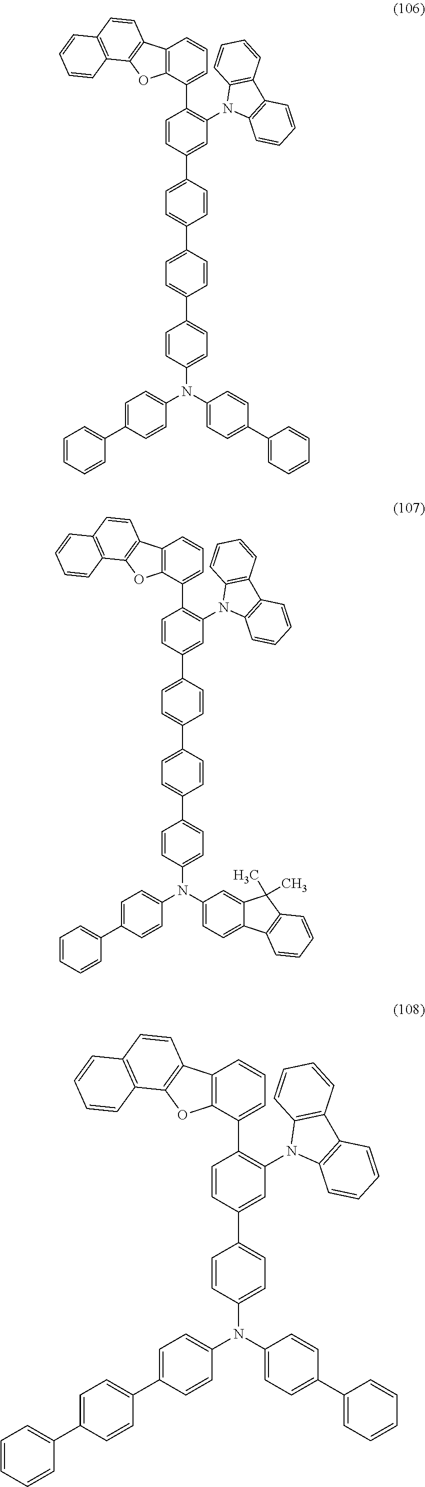

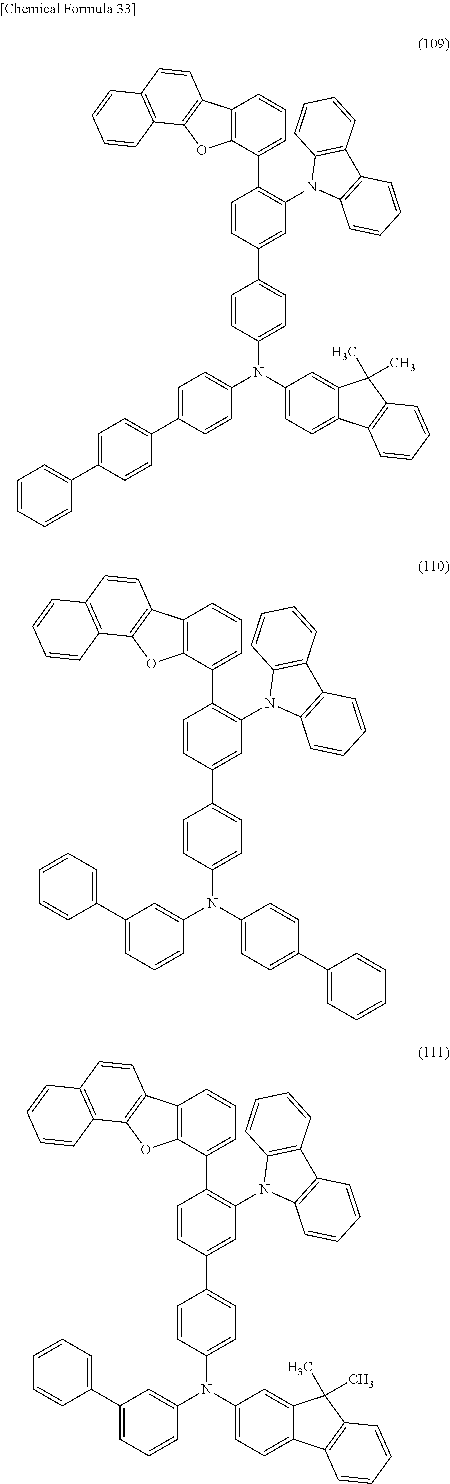

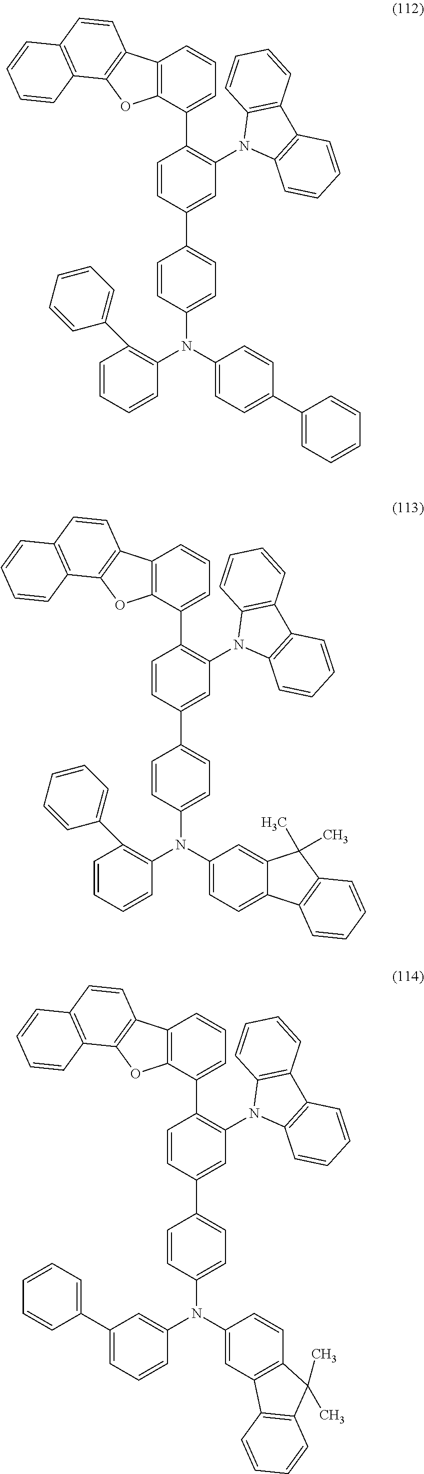

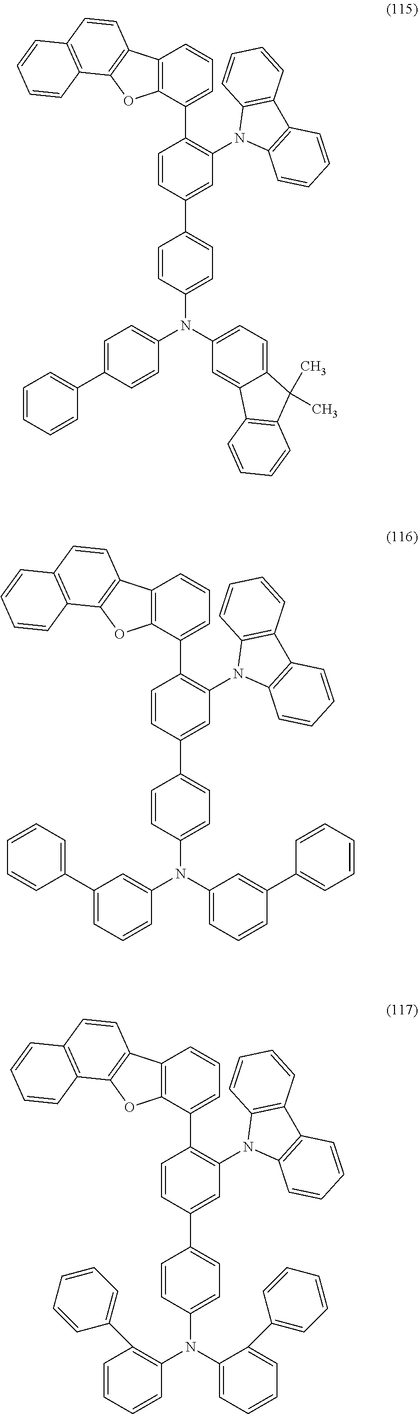

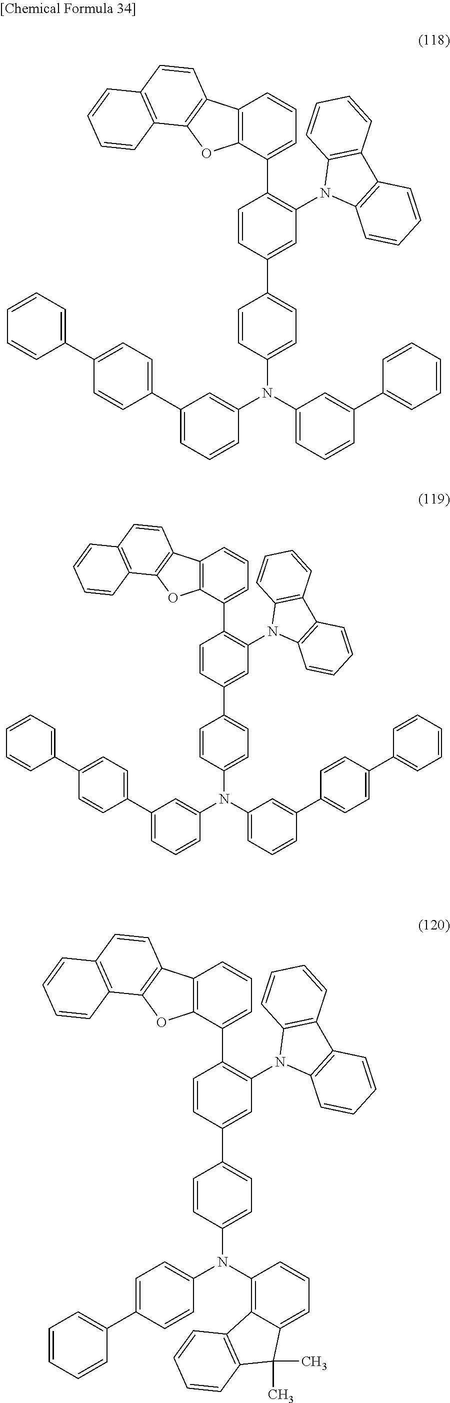

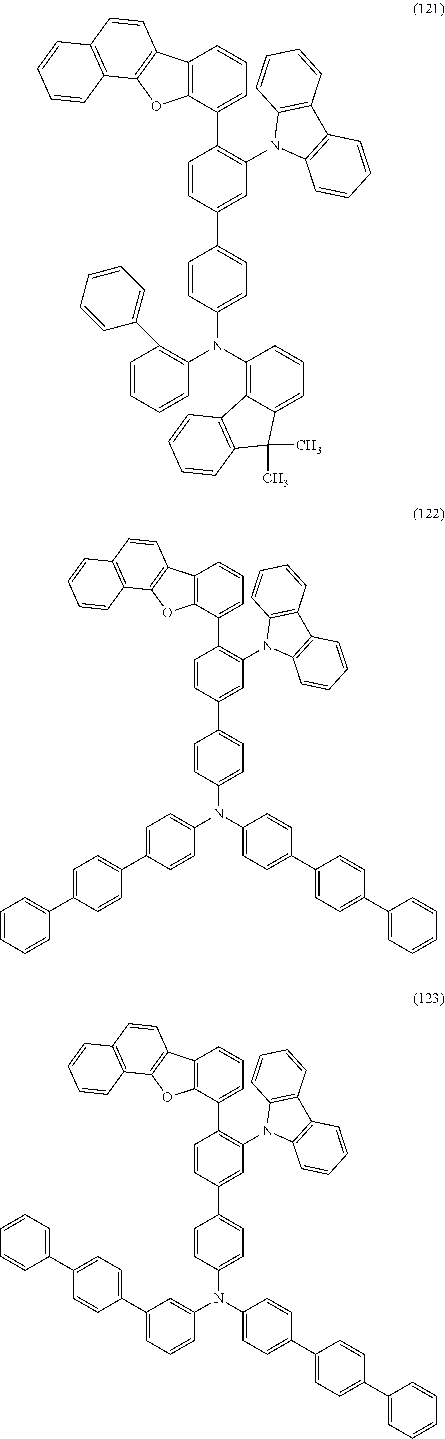

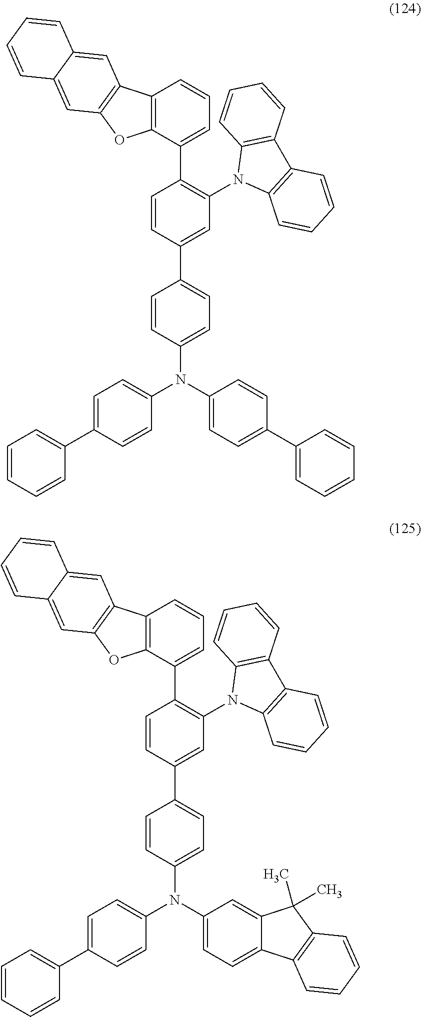

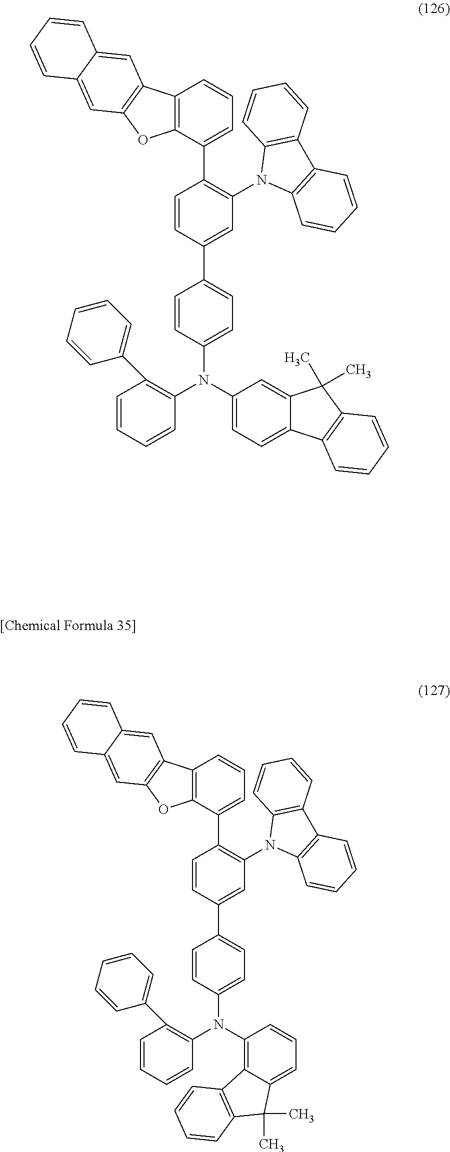

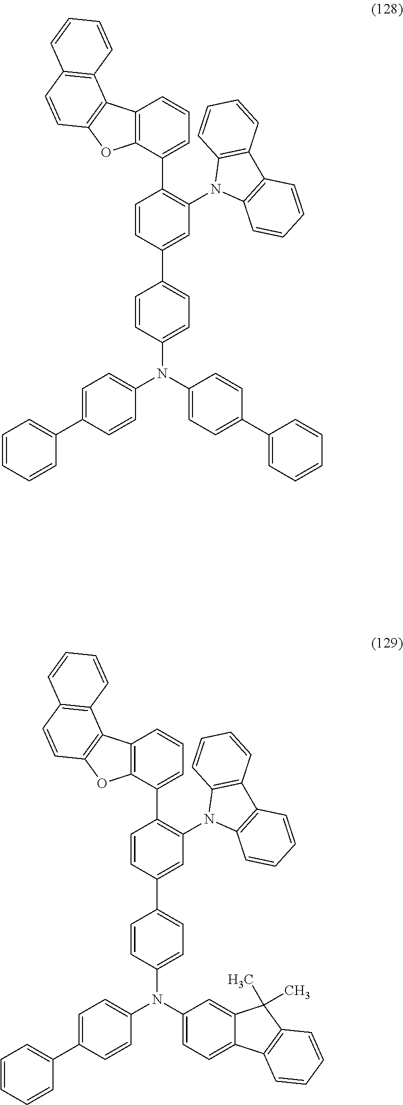

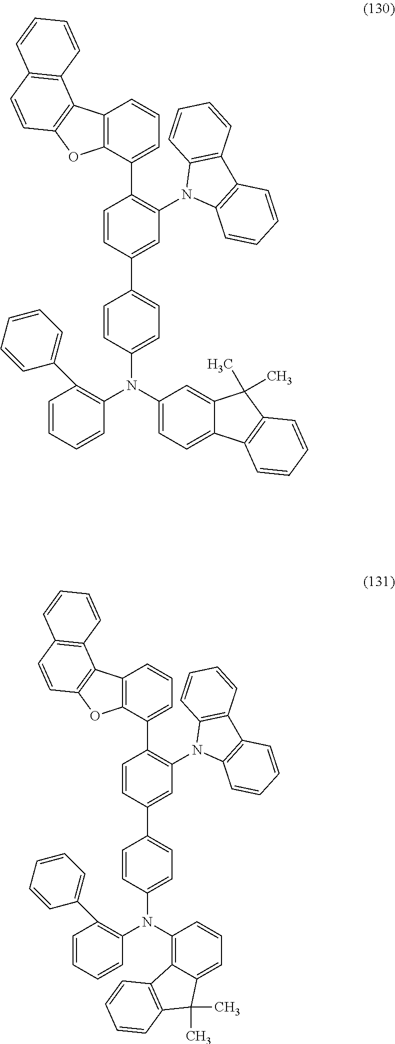

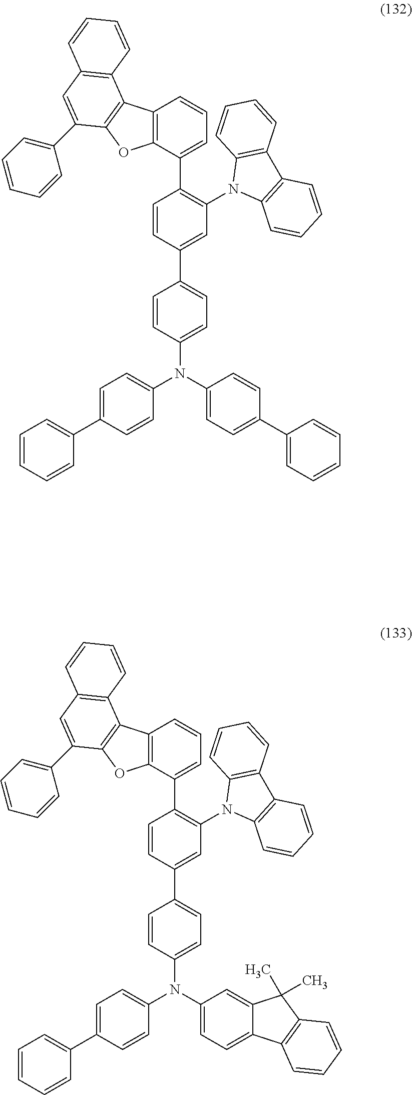

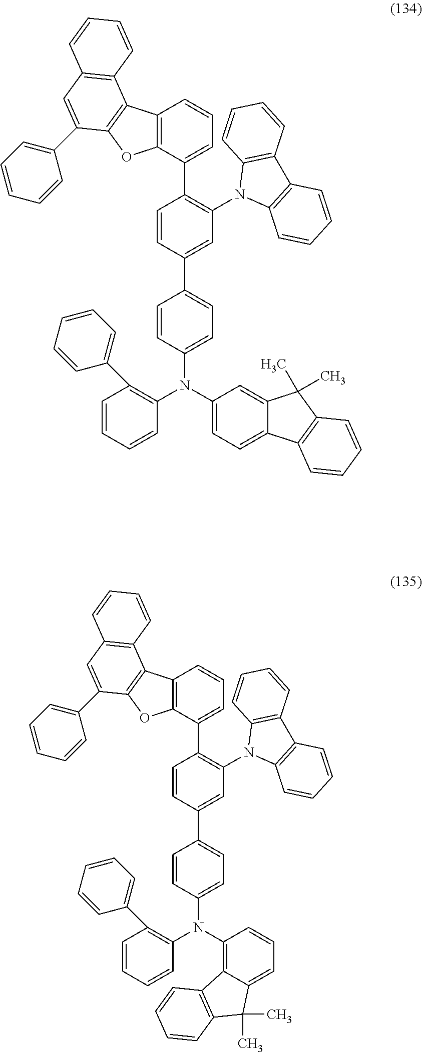

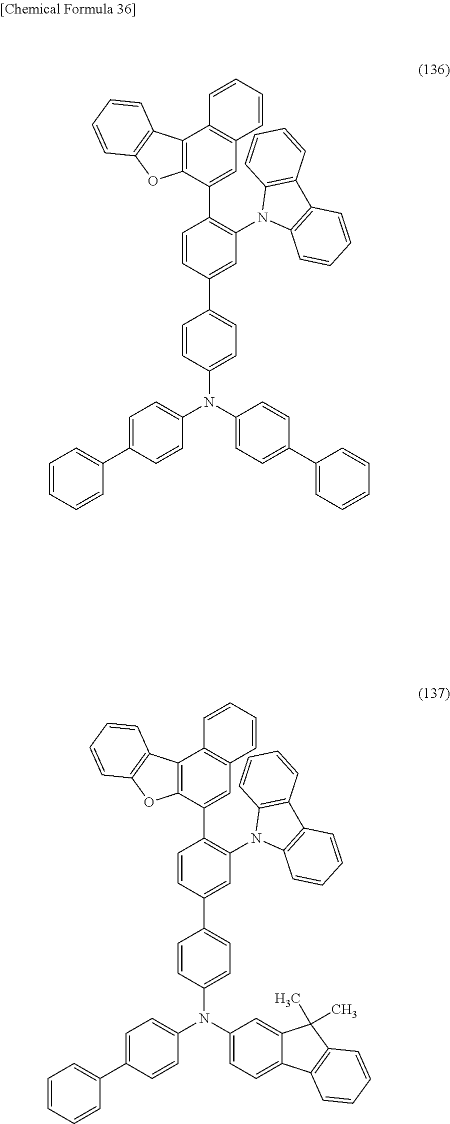

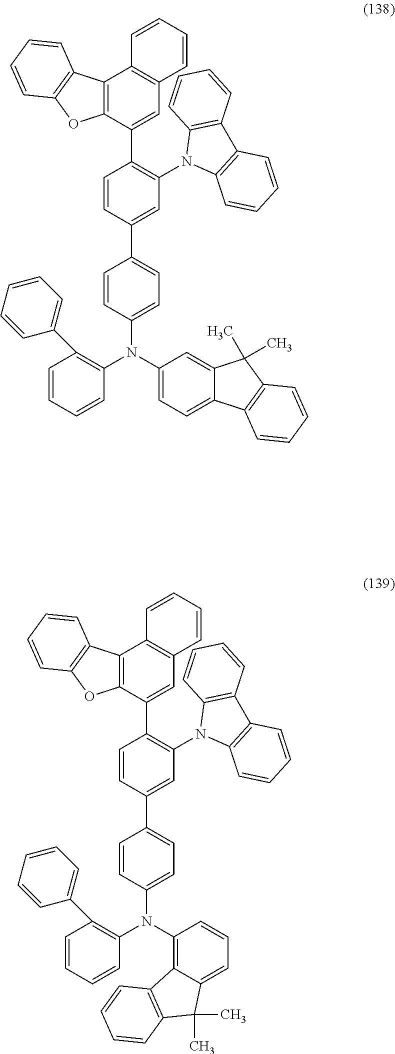

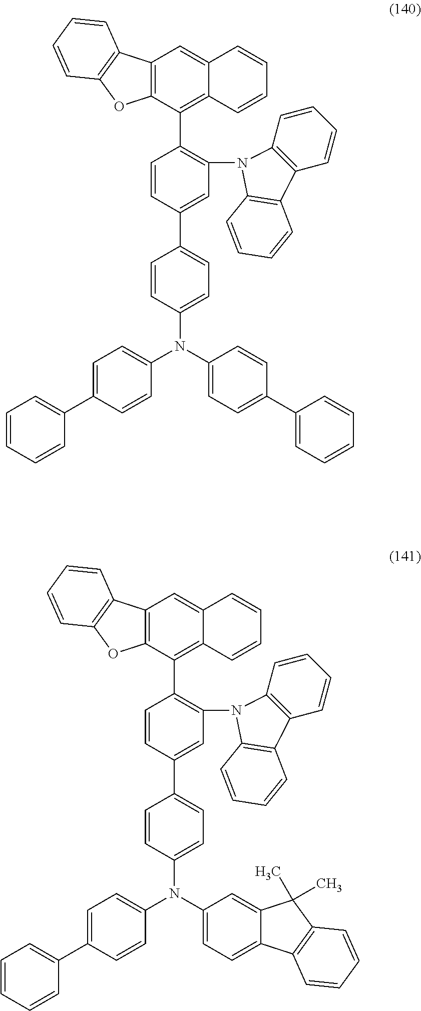

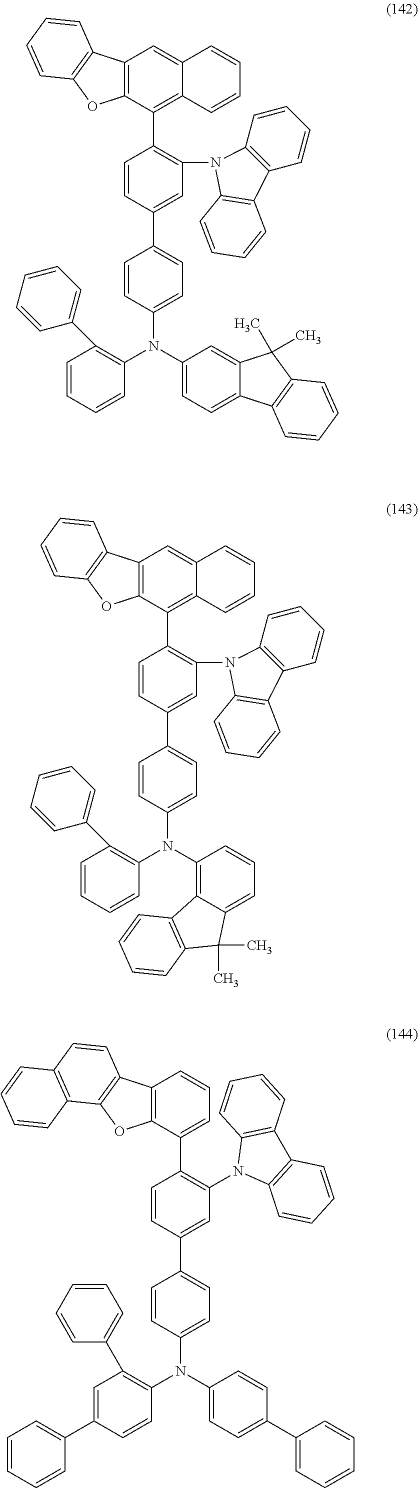

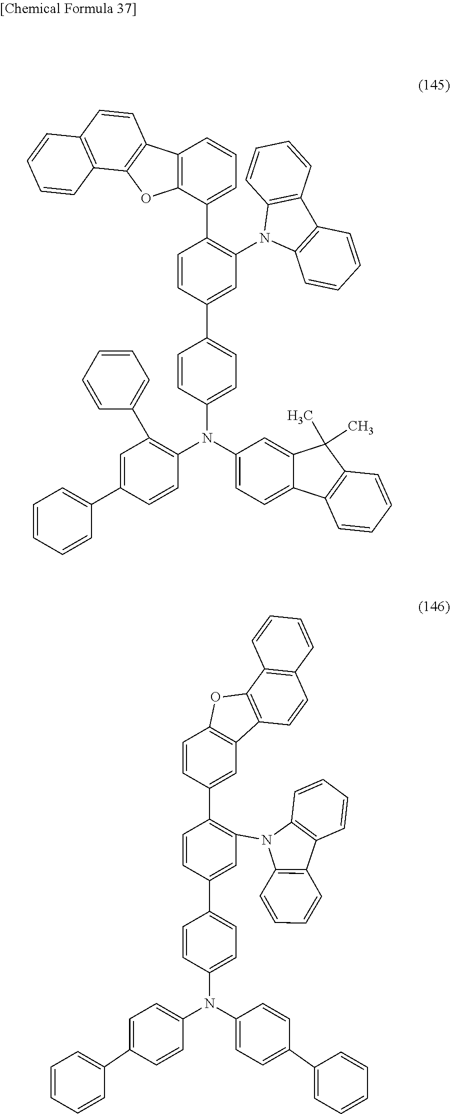

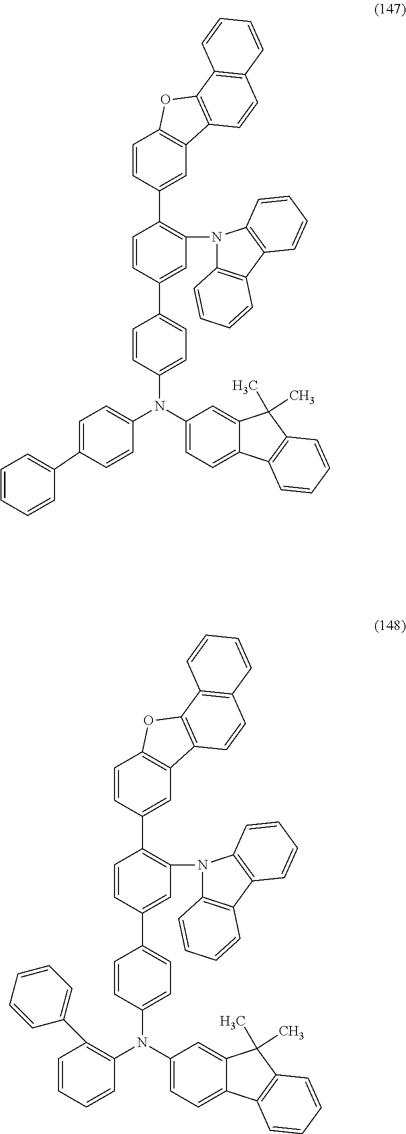

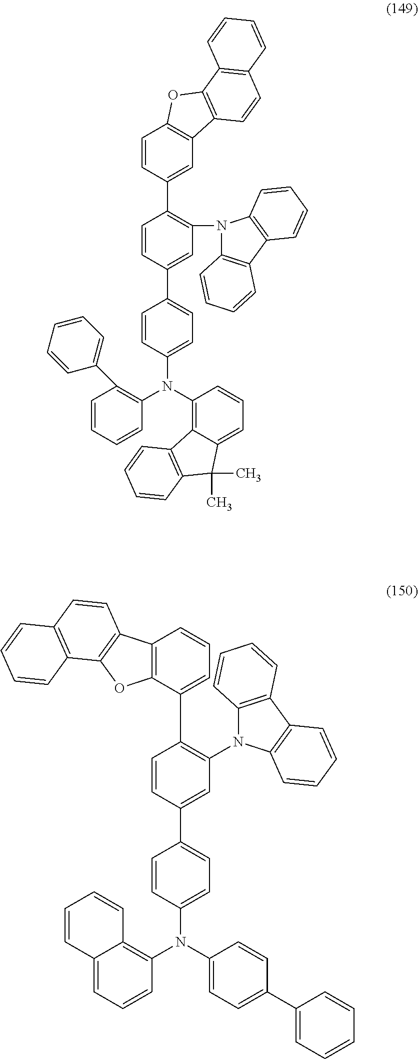

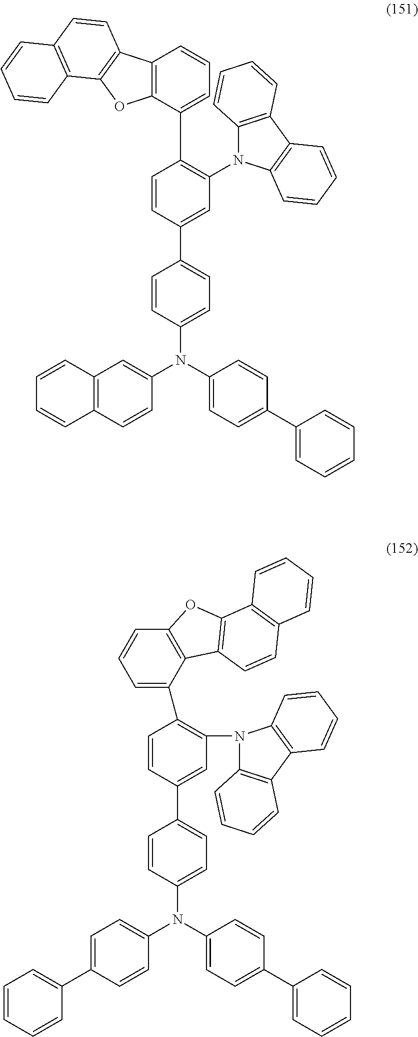

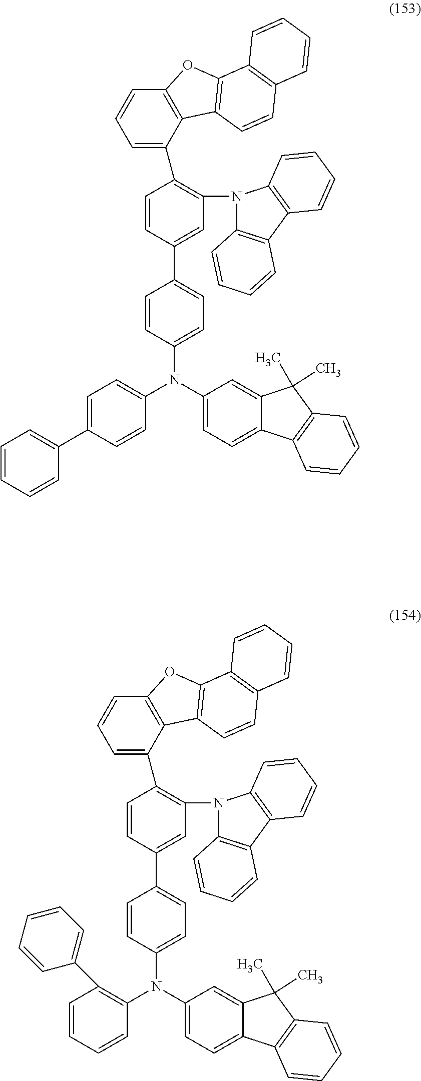

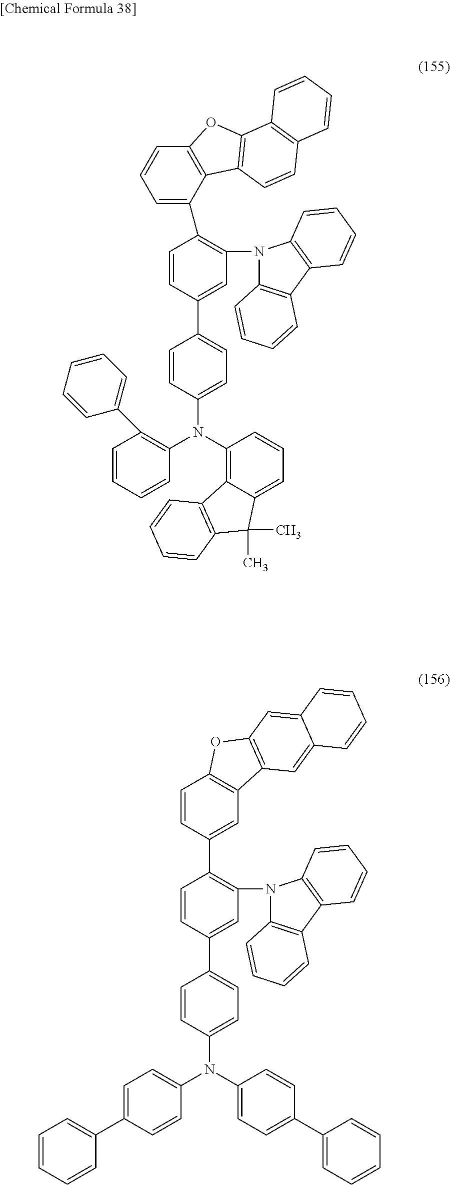

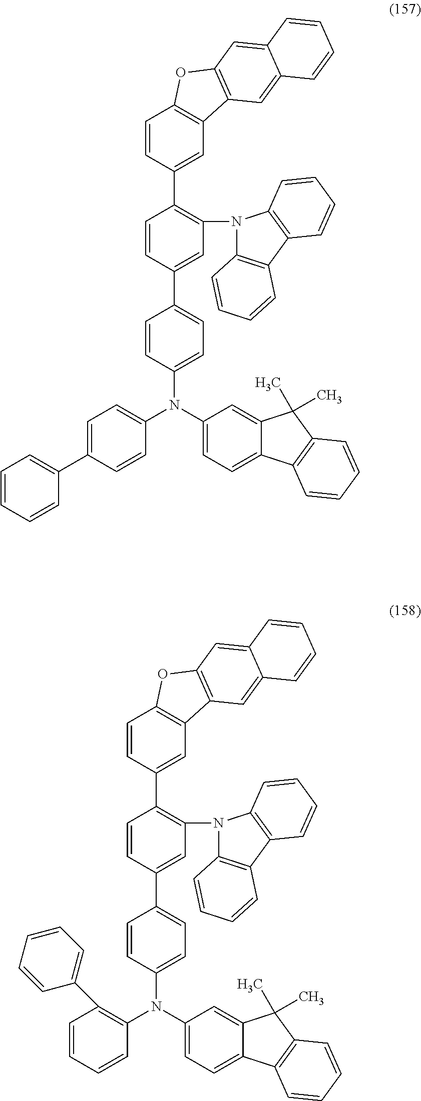

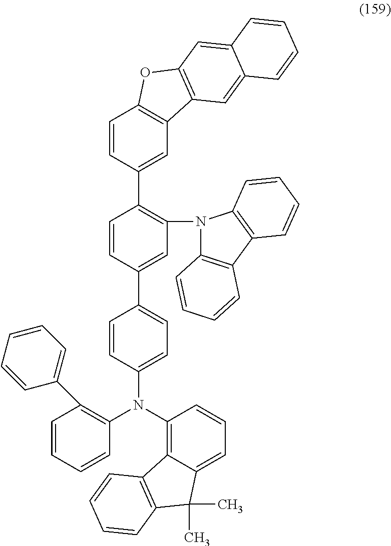

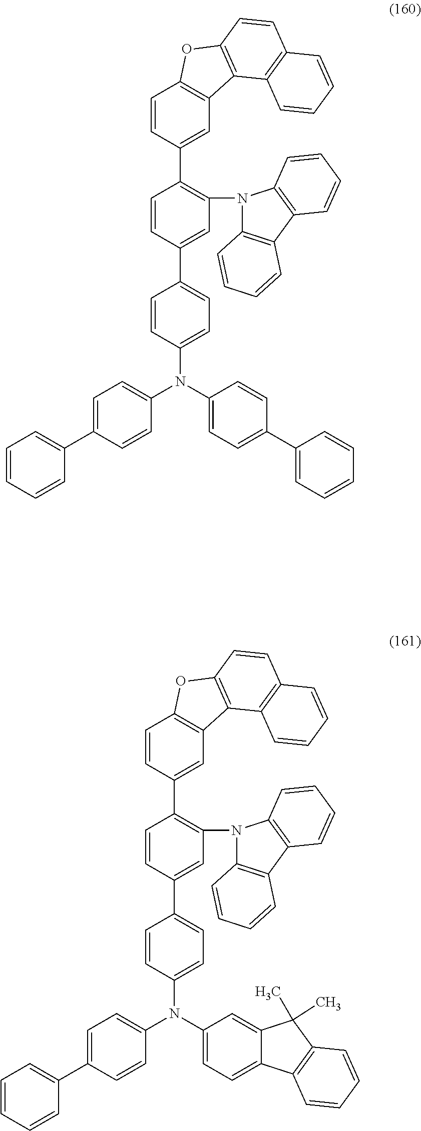

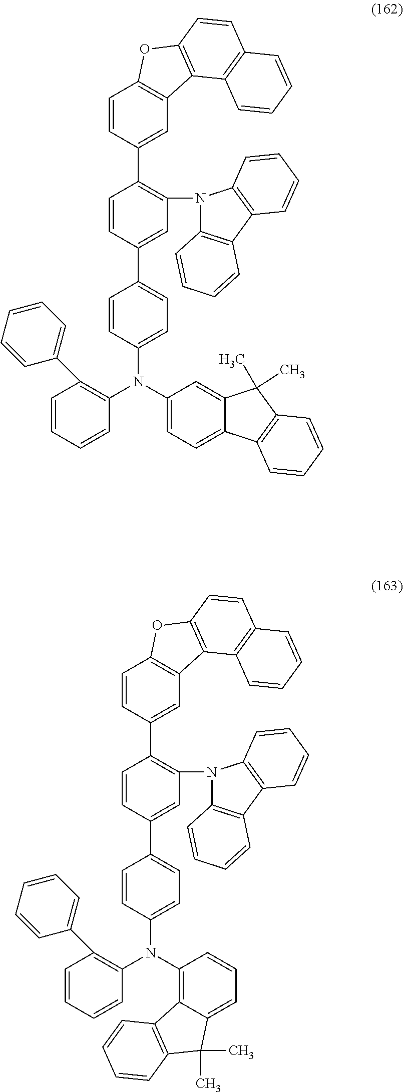

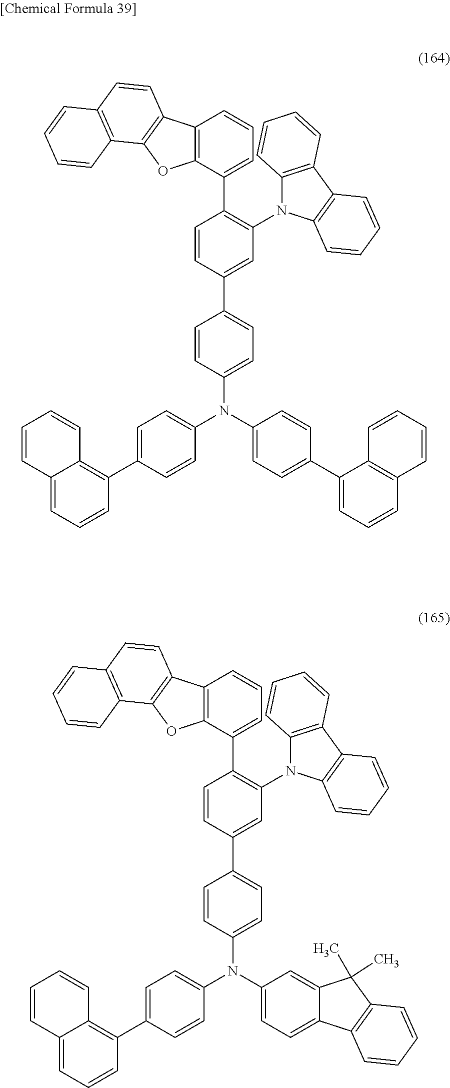

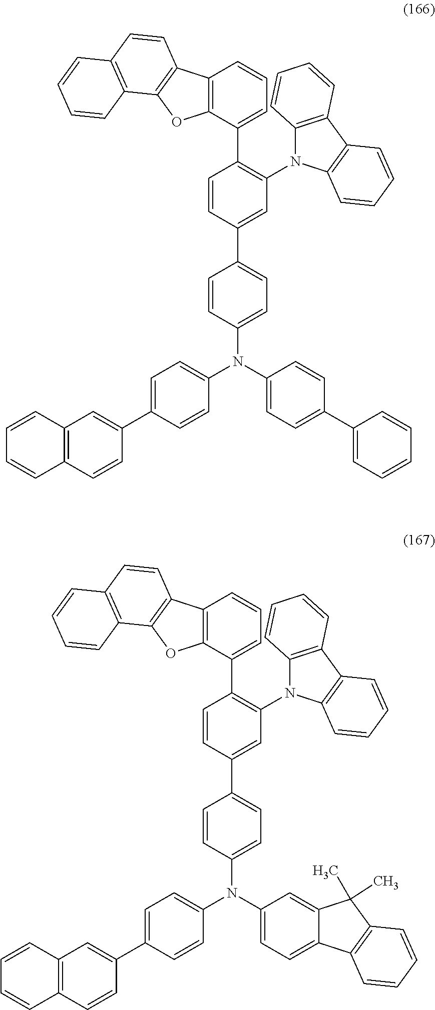

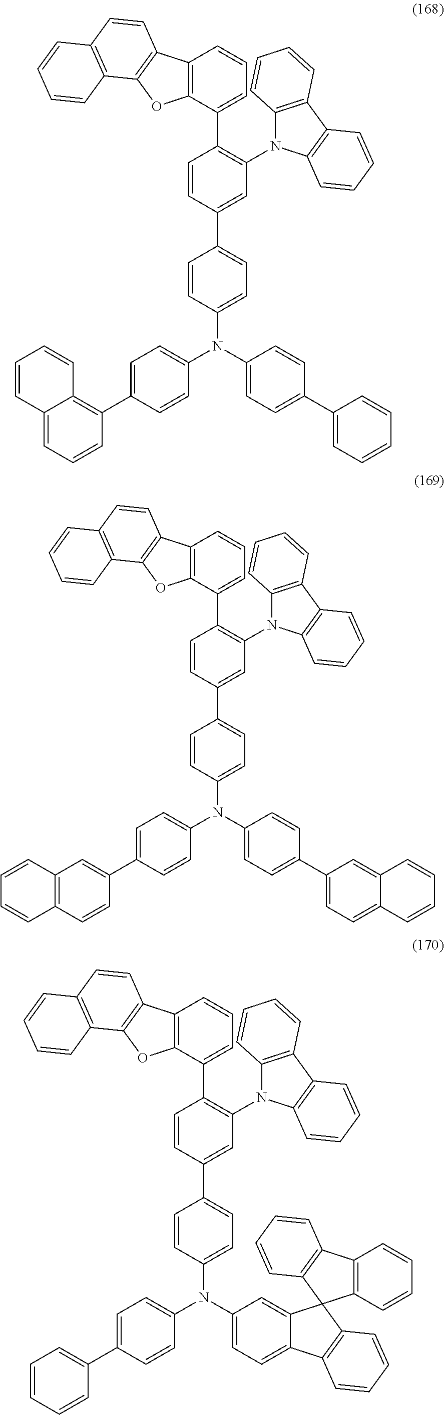

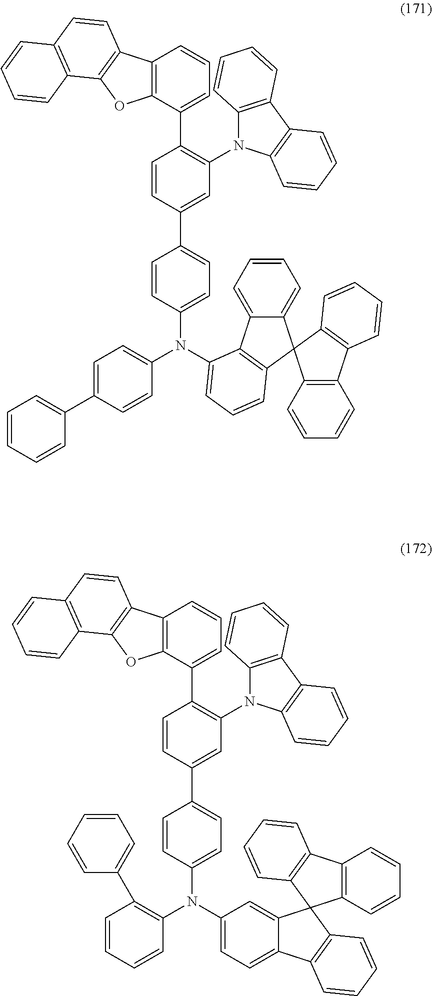

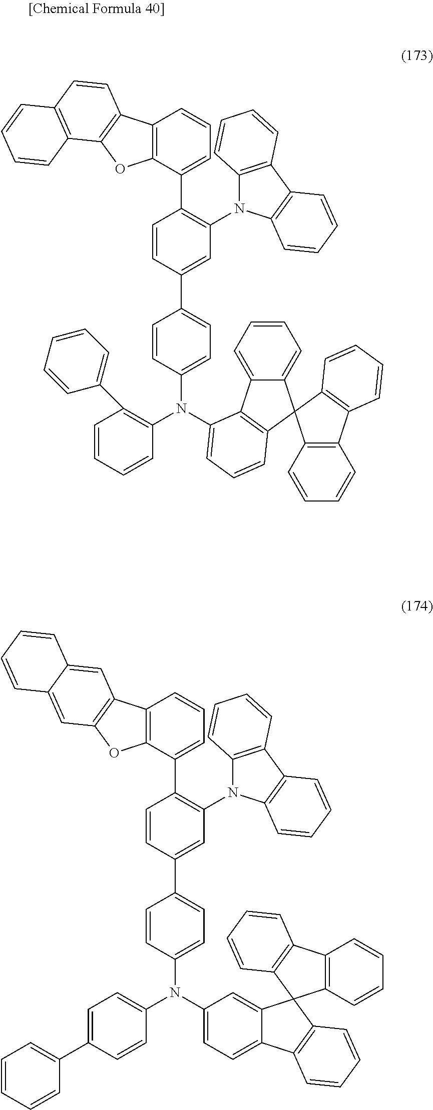

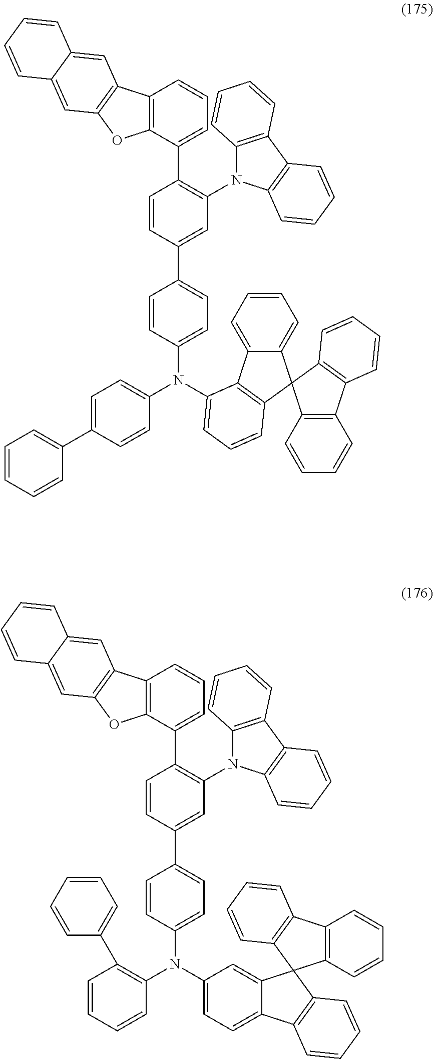

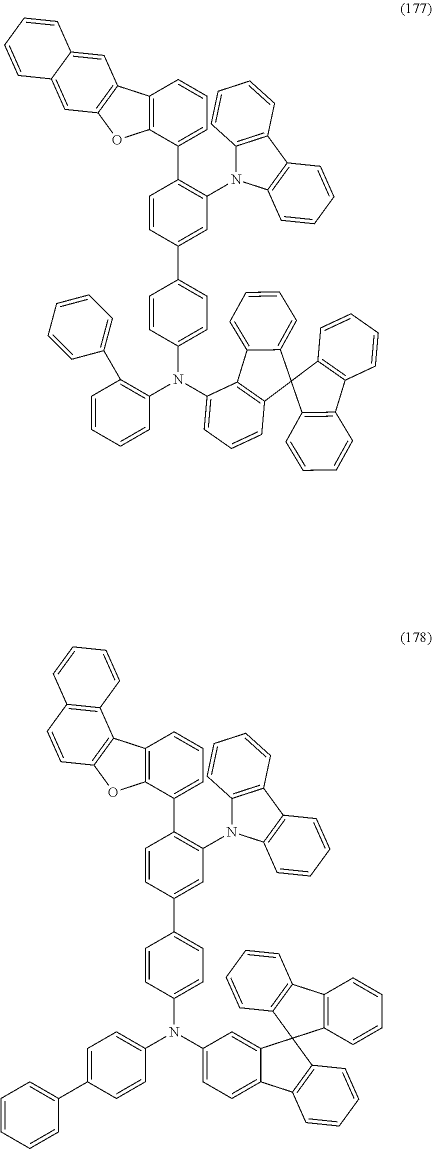

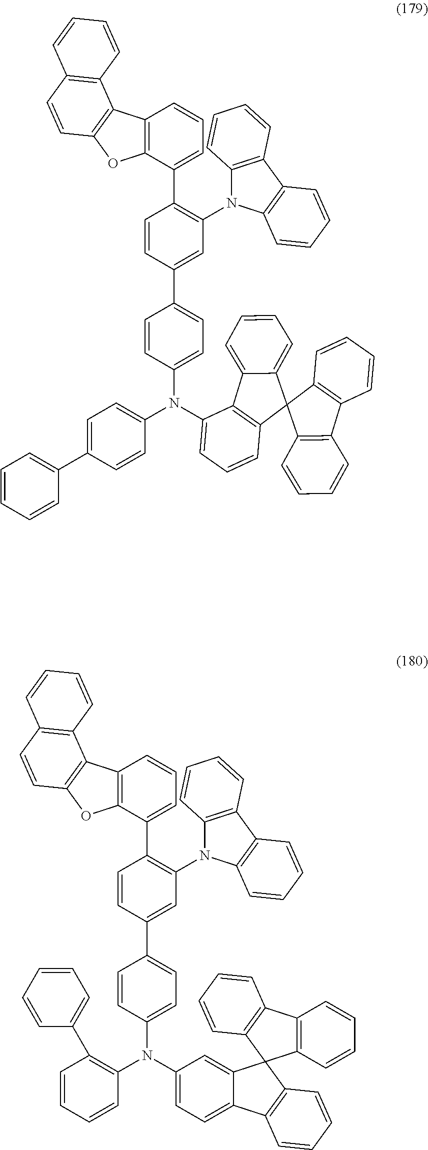

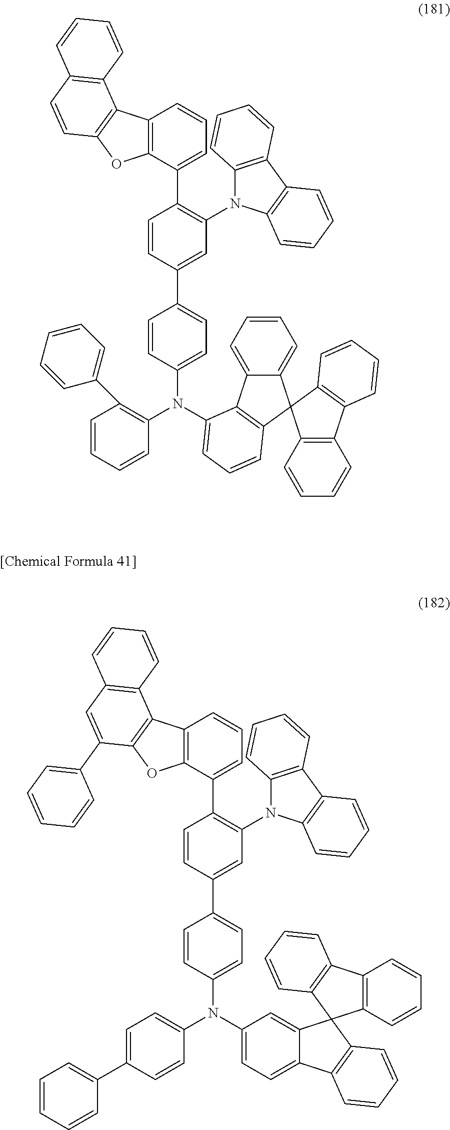

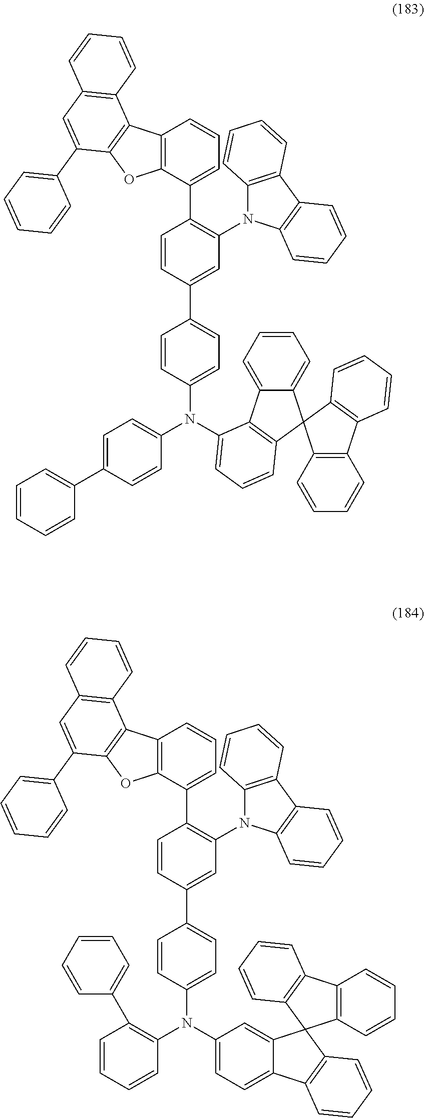

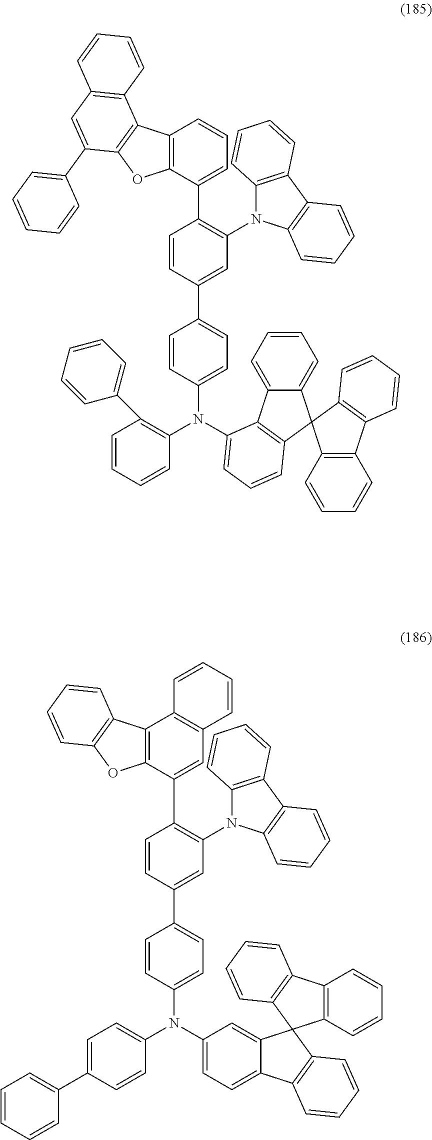

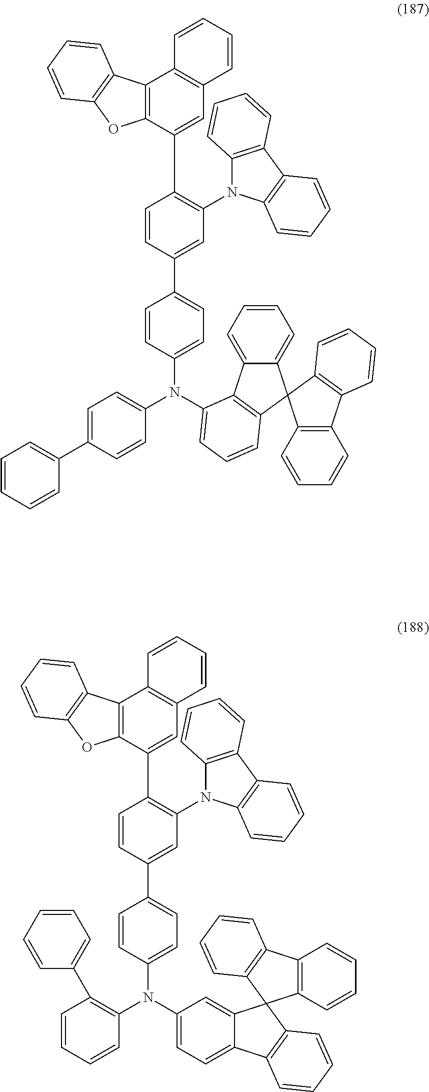

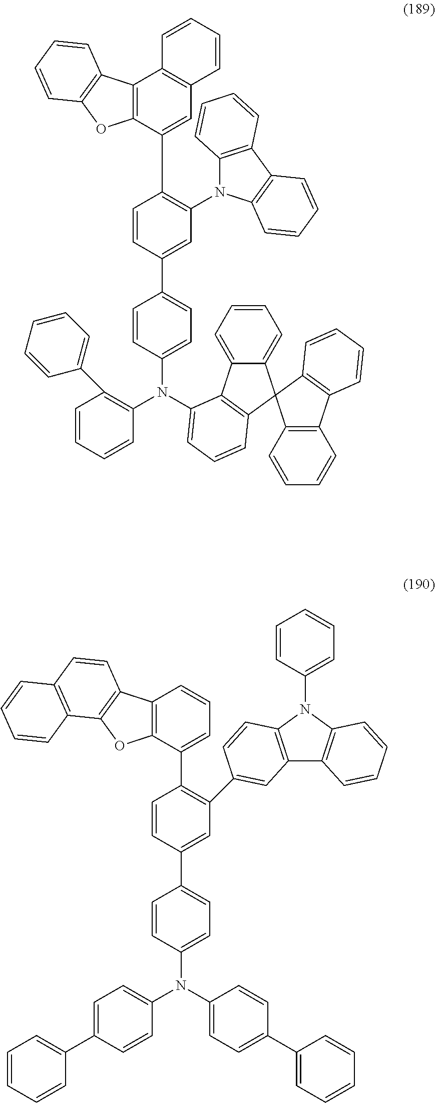

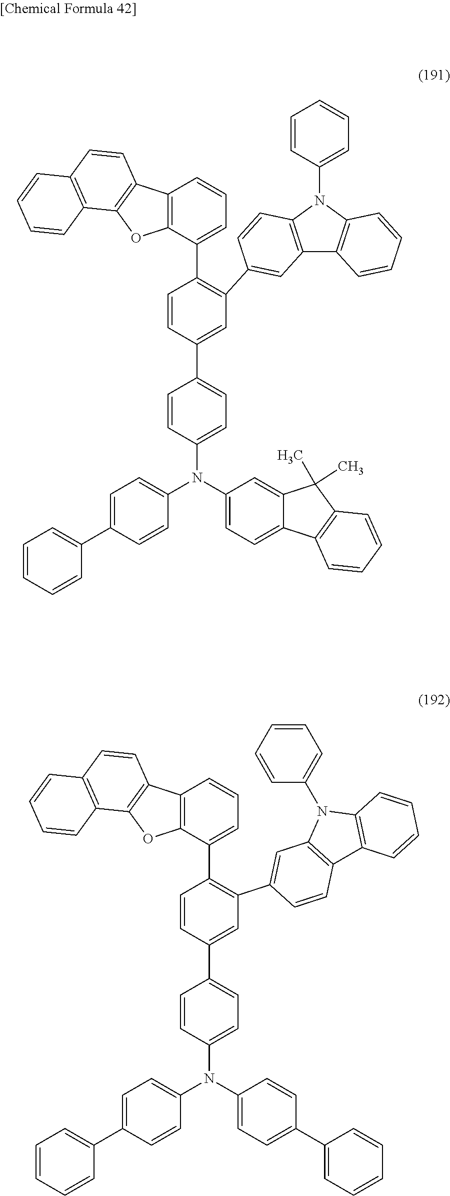

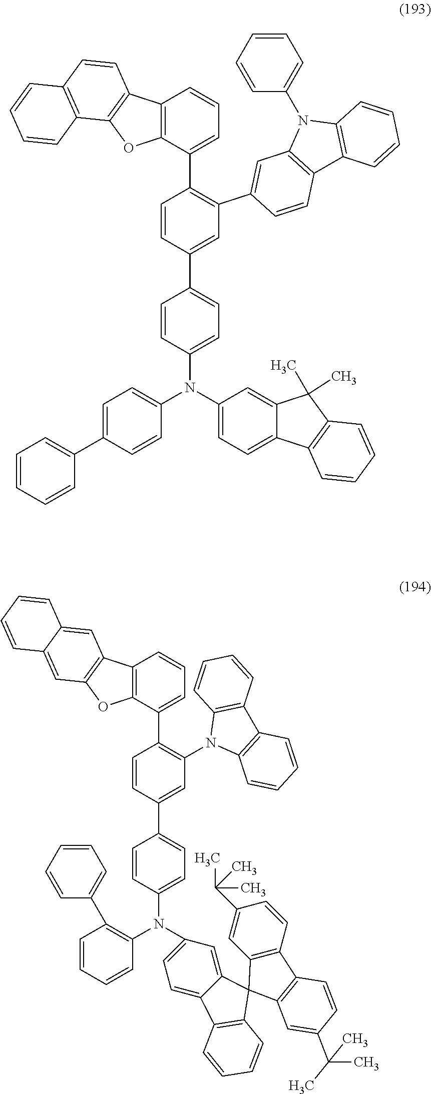

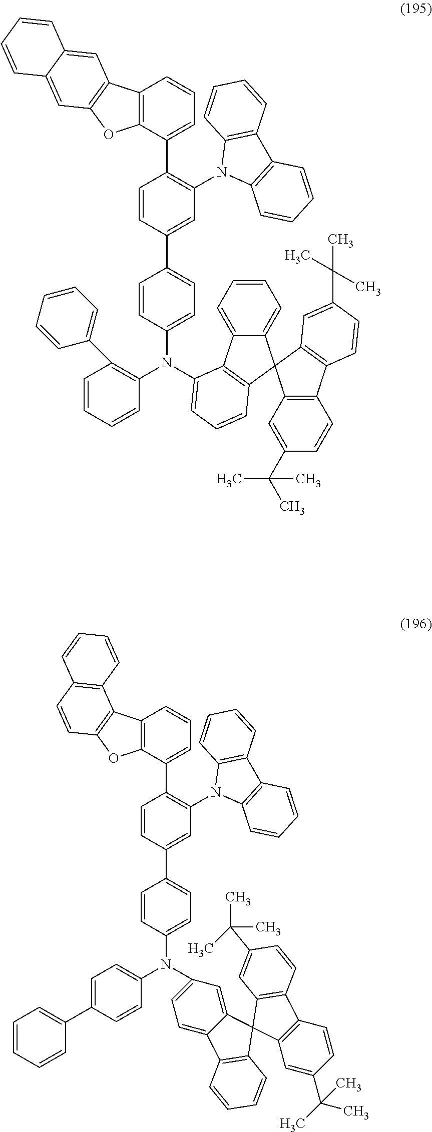

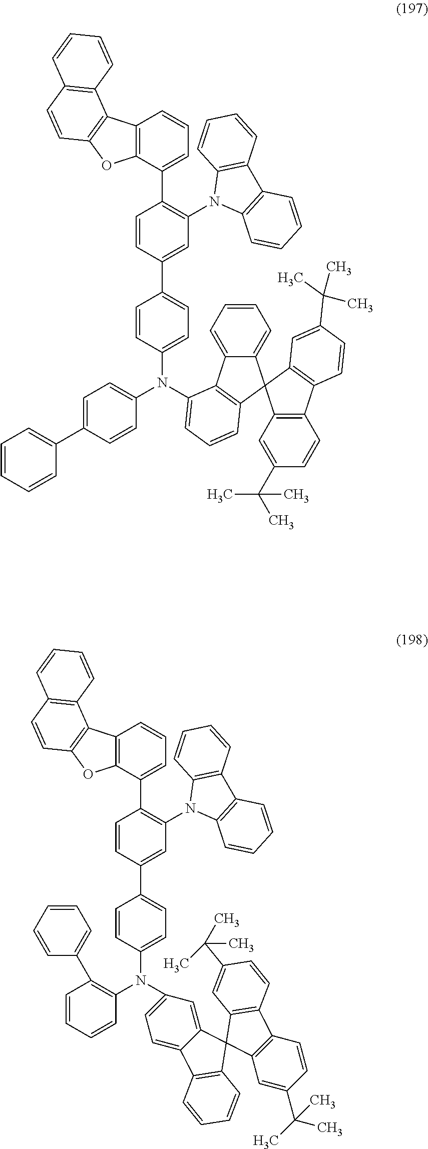

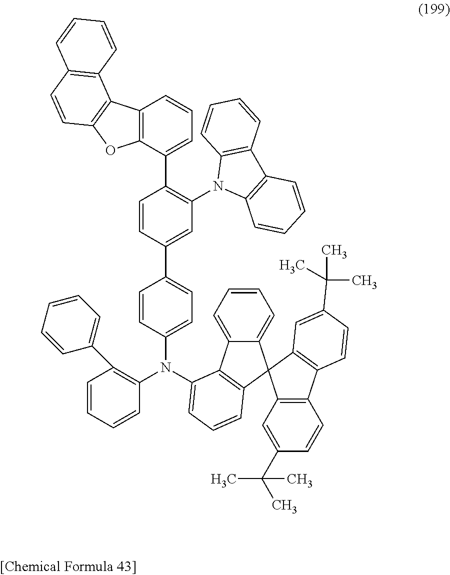

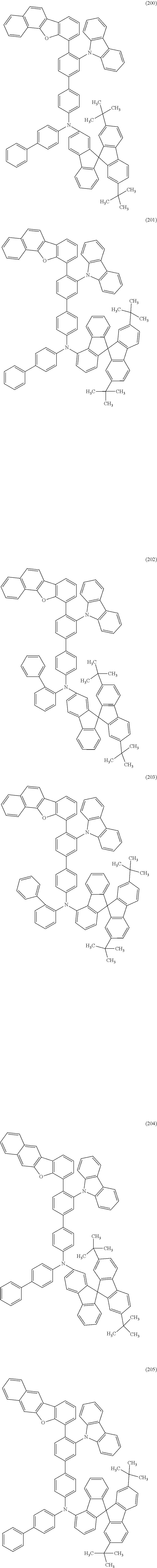

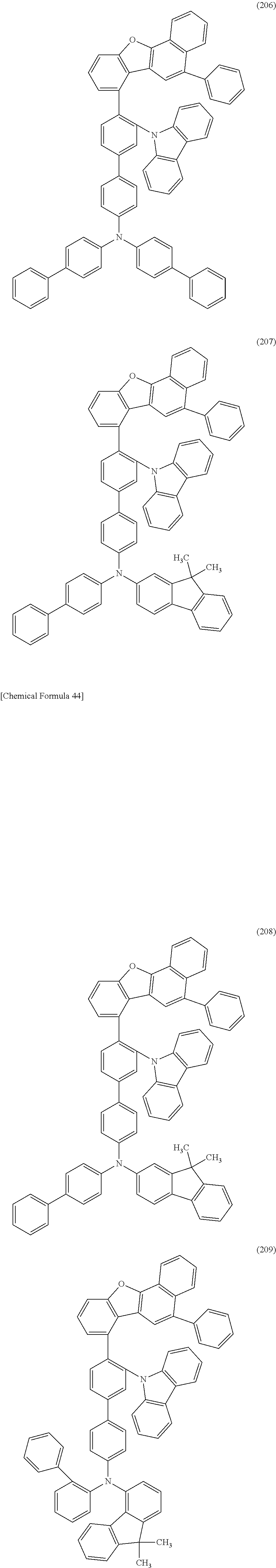

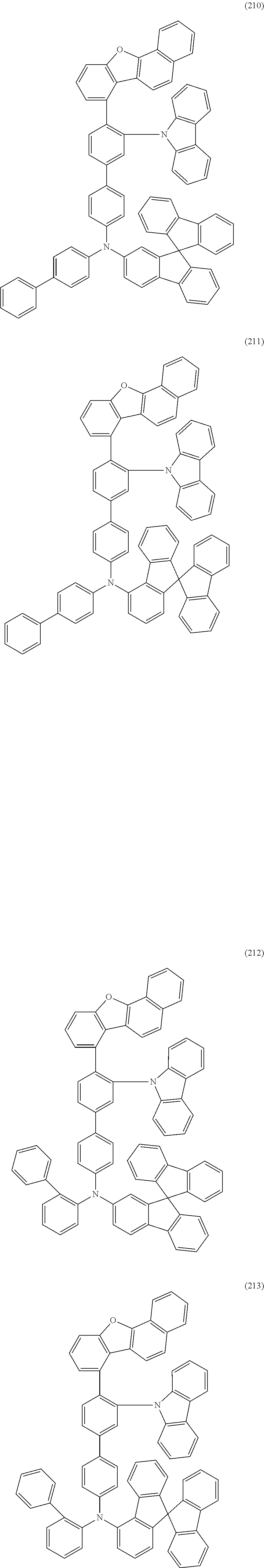

The organic compounds represented by Structural Formulae (100) to (213) above are examples of the organic compound represented by any of General Formulae (G1) to (G5). The organic compound of one embodiment of the present invention is not limited to these examples.

### <Synthesis Methods of Organic Compound>

Synthesis methods of the organic compound of one embodiment of the present invention are described using synthesis schemes shown below. Note that the synthesis methods of the organic compound of one embodiment of the present invention can employ a variety of reactions and are not limited to the following synthesis methods.

Here, a synthesis method of an organic compound represented by General Formula (G2-1) below, which is an example of the organic compound of one embodiment of the present invention, is described. In General Formula (G2-1), n in General Formula (G2) represents 1. In this synthesis example, an organic compound of another embodiment of the present invention which has any of a variety of substituents at Ar¹, R¹ to R⁸, Ar², and Ar³ can also be synthesized by the same method when a raw material having substituents corresponding to Ar¹, R¹ to R⁸, Ar², and Ar³ at the respective substitution sites is used.

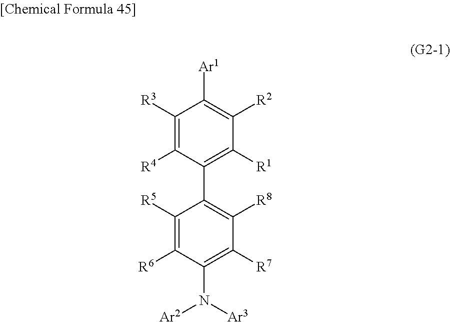

For Ar¹ to Ar³ and R¹ to R⁸ in General Formula (G2-1) and Synthesis Schemes (s1-1) to (s1-5), (s2-1), (s2-2), and (s3-1), the descriptions in <<Example 1 of organic compound>> and <<Example 2 of organic compound>> can be referred to. Note that in the following synthesis methods, the substituent R² represents General Formula (g2-1) or General Formula (g2-2) described in <<Example 1 of organic compound>>.

The synthesis method of the organic compound represented by General Formula (G2-1) can employ a variety of reactions. For example, synthesis reactions described below enable the synthesis of the organic compound represented by General Formula (G2-1).

<Synthesis method 1 of organic compound represented by General Formula (G2-1)>

The organic compound of the present invention represented by General Formula (G2-1) can be synthesized by Synthesis Schemes (s1-1) to (s1-5).

First, Synthesis Scheme (s1-1) is described. Specifically, an aryl compound (Compound 1) and a carbazole compound (Compound 2) are coupled, whereby an aryl compound (Compound 3) can be obtained. Synthesis Scheme (s1-1) is shown below.

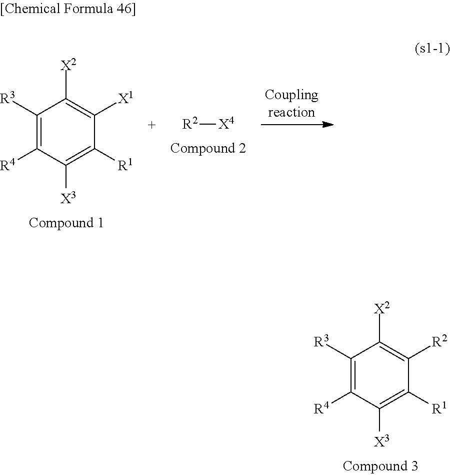

Next, Synthesis Scheme (s1-2) is described. Specifically, the aryl compound (Compound 3) and a benzonaphthofuran compound (Compound 4) are coupled, whereby an arylamine compound (Compound 5) can be obtained. Synthesis Scheme (s1-2) is shown below.

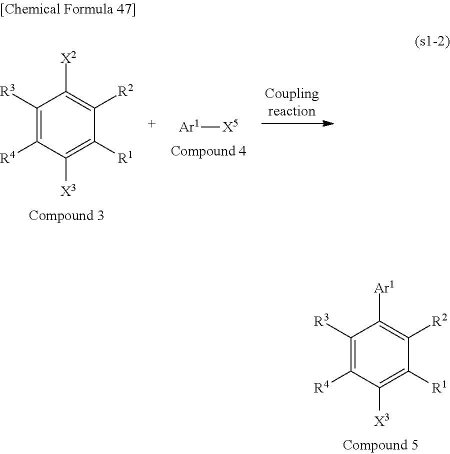

Next, Synthesis Scheme (s1-3) is described. Specifically, the arylamine compound (Compound 5) and an aryl compound (Compound 6) are coupled, whereby an aryl compound (Compound 7) can be obtained. Synthesis Scheme (s1-3) is shown below.

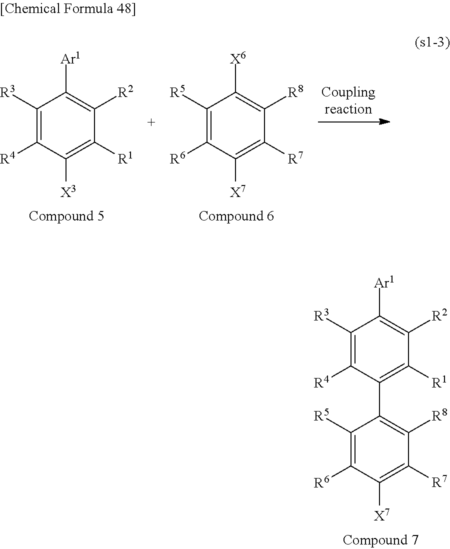

Next, Synthesis Scheme (s1-4) is described. Specifically, the arylamine compound (Compound 7) and an aryl compound (Compound 8) are coupled, whereby an arylamine compound (Compound 9) can be obtained. Synthesis Scheme (s1-4) is shown below.

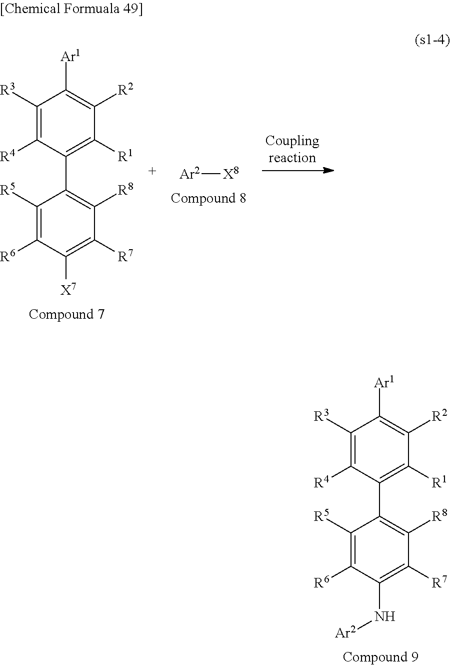

Next, Synthesis Scheme (s1-5) is described. Specifically, the arylamine compound (Compound 9) and an aryl compound (Compound 10) are coupled, whereby the target organic compound represented by General Formula (G2-1) can be obtained. Synthesis Scheme (s1-5) is shown below.

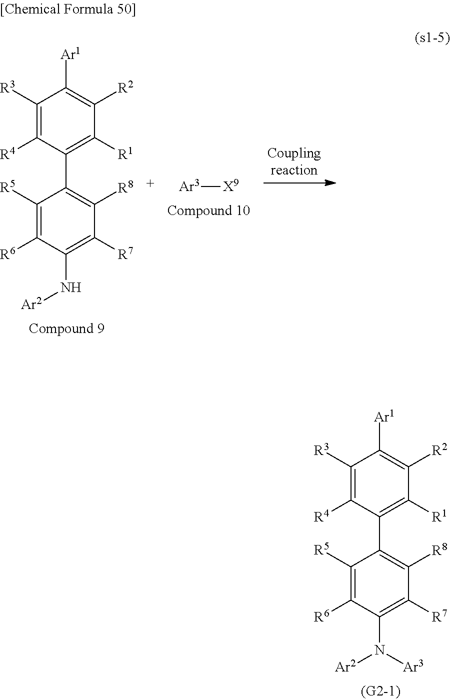

### <Synthesis Method 2 of Organic Compound Represented by General Formula (G2-1)>

The organic compound of the present invention represented by General Formula (G2-1) can be synthesized by Synthesis Schemes (s2-1) and (s2-2) below.

First, Synthesis Scheme (s2-1) is described. Specifically, the arylamine compound (Compound 7) and the aryl compound (Compound 10) are coupled, whereby an arylamine compound (Compound 11) can be obtained. Synthesis Scheme (s2-1) is shown below.

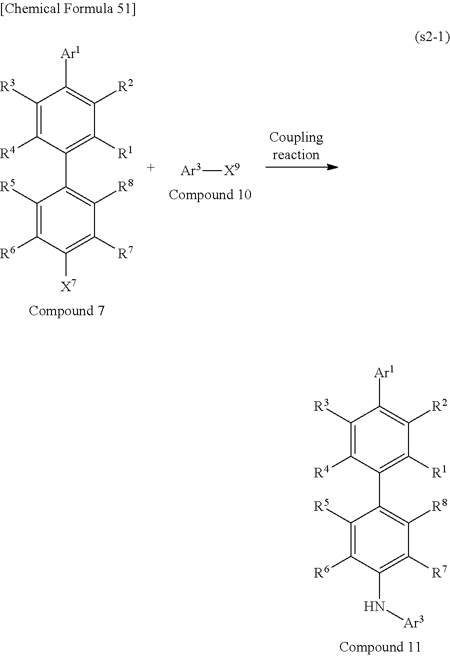

Next, Synthesis Scheme (s2-2) is described. Specifically, the arylamine compound (compound 11) and the aryl compound (compound 8) are coupled, whereby the target organic compound represented by General Formula (G2-1) can be obtained. Synthesis Scheme (s2-2) is shown below.

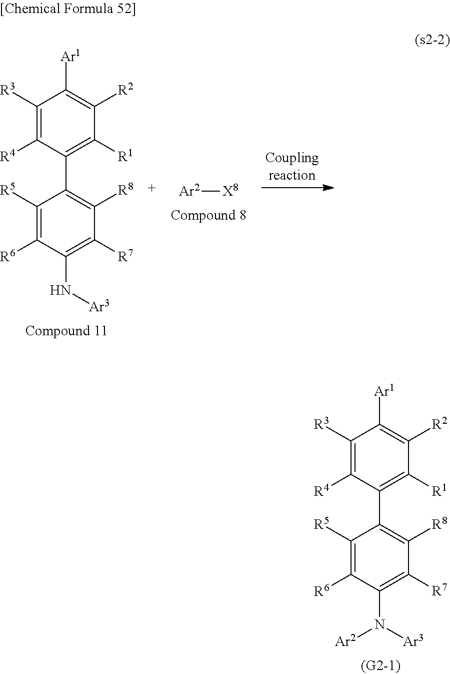

### <Synthesis Method 3 of Organic Compound Represented by General Formula (G2-1)>

The organic compound of the present invention represented by General Formula (G2-1) can be synthesized by Synthesis Scheme (s3-1) below.

Synthesis Scheme (s3-1) is described. Specifically, the arylamine compound (Compound 5) and an aryl compound (Compound 12) are coupled, whereby the target organic compound represented by General Formula (G2-1) can be obtained. Synthesis Scheme (s3-1) is shown below.

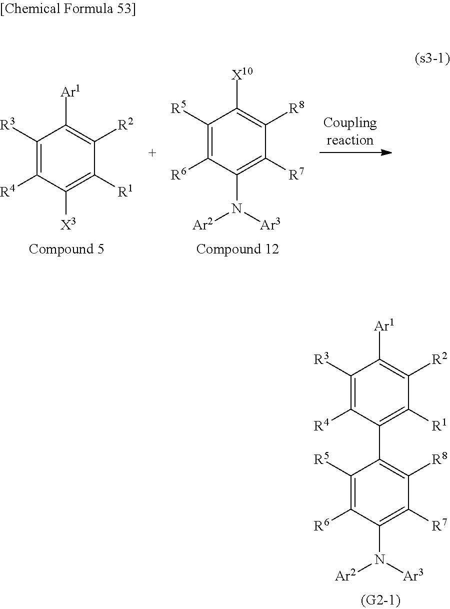

In Synthesis Schemes (s1-1) to (s1-5), (s2-1), (s2-2), and (s3-1) above, each of X¹ to X¹⁰ independently represents hydrogen (including deuterium), a halogen, a boronic acid group, an organoboron group, a triflate group (trifluoromethanesulfonic acid group, hereinafter referred to as triflate group), an organotin group, an organozinc group, an amino group, a magnesium halide group, or the like.

In the case where amination is performed by a nucleophilic substitution reaction in Synthesis Scheme (s1-1), X¹ represents any one of a halogen such as fluorine, chlorine, bromine, or iodine and a triflate group, and X⁴ represents hydrogen (including deuterium).

In the case where X¹ is a halogen in the reaction, fluorine or chlorine is preferable, and fluorine is particularly preferable because of its higher reaction rate.

In the reaction, toluene, xylene, benzene, tetrahydrofuran, dimethylformamide, dimethylacetoamide, diethylacetoamide, dioxane, dimethyl sulfoxide, acetonitrile, diethylene glycol dimethyl ether, ethylene glycol monomethyl ether, or the like can be used as a solvent. Reagents that can be used for the reaction are not limited thereto.

In the reaction, an inorganic base such as potassium carbonate, cesium carbonate, or sodium carbonate, or the like can be used.

In the case where a coupling reaction for forming a carbon-carbon bond is performed in Synthesis Scheme (s1-1), one of X¹ and X⁴ represents a boronic acid group, an organoboron group, an organotin group, an organozinc group, or a magnesium halide group, and the other of X¹ and X⁴ represents chlorine, bromine, iodine, or a triflate group. Regarding the substituents represented by X¹ and X⁴, the same applies to the combinations of X² and X⁵, X³ and X⁶, X³ and X¹⁰, X⁷ and X⁸, and X⁷ and X⁹.

In the reaction, the halogen is preferably chlorine, bromine, or iodine; bromine or iodine is preferable in terms of reactivity, and chlorine or bromine is preferable in terms of cost.

In Synthesis Schemes (s1-1) to (s1-3) and (s3-1), when a Suzuki-Miyaura coupling reaction using a palladium catalyst is performed, each of X¹ to X⁶ and X¹⁰ represents a halogen group, a boronic acid group, an organoboron group, or a triflate group, and the halogen is preferably iodine, bromine, or chlorine. In the reaction, a palladium compound such as bis(dibenzylideneacetone)palladium(0), palladium(II) acetate, [1,1-bis(diphenylphosphino)ferrocene]palladium(II) dichloride, or tetrakis(triphenylphosphine)palladium(0) and a ligand such as tri(t-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, or tri(ortho-tolyl)phosphine can be used. In the reaction, an organic base such as sodium t-butoxide, an inorganic base such as potassium carbonate, cesium carbonate, or sodium carbonate, or the like can be used.

In the reaction, toluene, xylene, mesitylene, benzene, tetrahydrofuran, dioxane, ethanol, methanol, water, diethylene glycol dimethyl ether, ethylene glycol monomethyl ether, or the like can be used as a solvent. Reagents that can be used for the reaction are not limited thereto.

For the reaction represented by Synthesis Schemes (s1-1) to (s1-3) and (s3-1), a Migita-Kosugi-Stille coupling reaction using an organotin compound, a Kumada-Tamao-Corriu coupling reaction using a Grignard reagent, a Negishi coupling reaction using an organozinc compound, a reaction using copper or a copper compound, or the like can also be employed.

In Synthesis Schemes (s1-4), (s1-5), (s2-1), and (s2-2), each of X⁷ to X⁹ represents a halogen, an amino group, or the like, and the halogen is preferably iodine, bromine, or chlorine. In the case where the Buchwald-Hartwig reaction using a palladium catalyst is performed in the reaction, a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride (dimer) and a ligand such as tri(t-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, or di-t-butyl(1-methyl-2,2-diphenylcyclopropyl)phosphine (abbreviation: cBRIDP (registered trademark)), can be used. In the reaction, an organic base such as sodium tert-butoxide, an inorganic base such as potassium carbonate, cesium carbonate, or sodium carbonate, or the like can be used. In the reaction, toluene, xylene, benzene, tetrahydrofuran, dioxane, or the like can be used as a solvent.

In Synthesis Schemes (s1-4), (s1-5), (s2-1), and (s2-2), the Ullmann reaction using copper or a copper compound can be employed. Examples of the base to be used include an inorganic base such as potassium carbonate. Examples of the solvent that can be used in the reaction include 1,3-dimethyl-3,4,5,6-tetrahydro-2(1H)-pyrimidinone (DMPU), toluene, xylene, and benzene. In the Ullmann reaction, when the reaction temperature is higher than or equal to 100° C., the target substance can be obtained in a shorter time in a higher yield; thus, it is preferable to use DMPU or xylene having a high boiling point. A reaction temperature higher than or equal to 150° C. is further preferable, and accordingly, DMPU is further preferably used.

The synthesis method of the organic compound of the present invention represented by General Formula (G2-1) is not limited to Synthesis Schemes (s1-1) to (s1-5), (s2-1), (s2-2), and (s3-1).

This embodiment can be freely combined with any of the other embodiments and the examples.

### Embodiment 2

In this embodiment, structures of a light-emitting device using the organic compound described in Embodiment 1 will be described.

It is a long time since displays (organic EL displays) using organic EL elements (hereinafter also referred to as light-emitting devices) as display elements were put into practical use. These displays are usually provided with pixels emitting light with at least three colors of red, green, and blue to achieve full-color display.

The pixels are provided with light-emitting devices for the respective emission colors. In a display fabricated by a side-by-side method, or what is called a separate coloring method, light-emitting devices contain light-emitting substances corresponding to the respective emission colors of the pixels.

The organic compound described in Embodiment 1, which has a favorable carrier-transport property, particularly an excellent hole-transport property, can be suitably used for a host material or a carrier-transport layer, particularly a hole-transport layer or a hole-injection layer, in a light-emitting device described in this embodiment and a light-receiving device described later.

One embodiment of the present invention provides a light-emitting device using the organic compound described in Embodiment 1 as a hole-transport material.

### <Structure Examples of Light-Emitting Device>

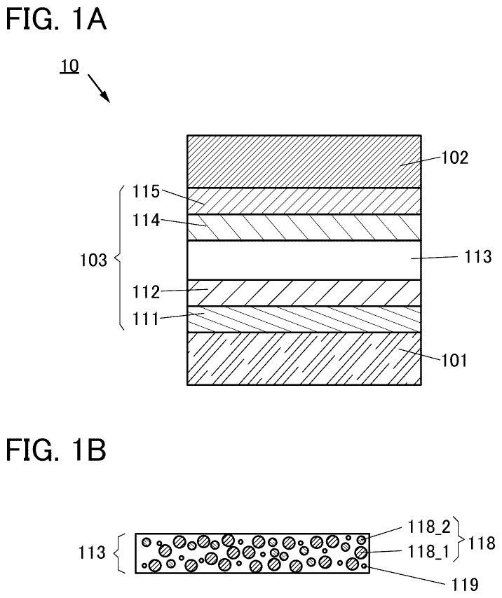

FIG. 1A is a schematic cross-sectional view of a light-emitting device 10 of one embodiment of the present invention. The light-emitting device 10 includes a pair of electrodes (a first electrode 101 and a second electrode 102) and an organic compound layer 103 between the pair of electrodes. The organic compound layer 103 includes at least a light-emitting layer 113.

The organic compound layer 103 illustrated in FIG. TA includes functional layers such as a hole-injection layer 111, a hole-transport layer 112, an electron-transport layer 114, and an electron-injection layer 115, in addition to the light-emitting layer 113.

Although description is made in this embodiment assuming that the first electrode 101 and the second electrode 102 of the pair of electrodes serve as an anode and a cathode, respectively, the structure of the light-emitting device 10 is not limited thereto. That is, the first electrode 101 may be a cathode, the second electrode 102 may be an anode, and the stacking order of the layers between the electrodes may be reversed. In other words, the hole-injection layer 111, the hole-transport layer 112, the light-emitting layer 113, the electron-transport layer 114, and the electron-injection layer 115 may be stacked in this order from the anode side.

The structure of the organic compound layer 103 is not limited to the structure illustrated in FIG. TA, and a structure including at least one layer selected from the hole-injection layer 111, the hole-transport layer 112, the electron-transport layer 114, and the electron-injection layer 115 may be employed. Alternatively, the organic compound layer 103 may include a functional layer which has a function of lowering a hole- or electron-injection barrier, improving a hole- or electron-transport property, inhibiting a hole- or electron-transport property, or reducing quenching by an electrode, for example. Note that the functional layer may be either a single layer or stacked layers.

FIG. 1B is a schematic cross-sectional view illustrating an example of the light-emitting layer 113 illustrated in FIG. TA. The light-emitting layer 113 illustrated in FIG. 1B contains host materials 118 (an organic compound 118_1 and an organic compound 118_2) and a guest material 119 (a light-emitting substance).

The guest material 119 may be a light-emitting organic compound, and the light-emitting organic compound is preferably a substance capable of emitting phosphorescent light (hereinafter also referred to as a phosphorescent compound).

In the light-emitting layer 113, the host material 118 is present in the largest proportion by weight, and the guest material 119 is dispersed in the host material 118. The lowest triplet excitation energy levels (Ti levels) of the host materials 118 (the organic compounds 118_1 and 118_2) in the light-emitting layer 113 are preferably higher than the T₁ level of the guest material 119 in the light-emitting layer 113.

The host materials 118 (the organic compounds 118_1 and 118_2) in the light-emitting layer 113 preferably form an exciplex. Note that an exciplex is an excited state formed by two or more kinds of substances. In photoexcitation, the exciplex is formed by interaction between one substance in an excited state and another substance in a ground state.

### <Basic Structure of Light-Emitting Device>

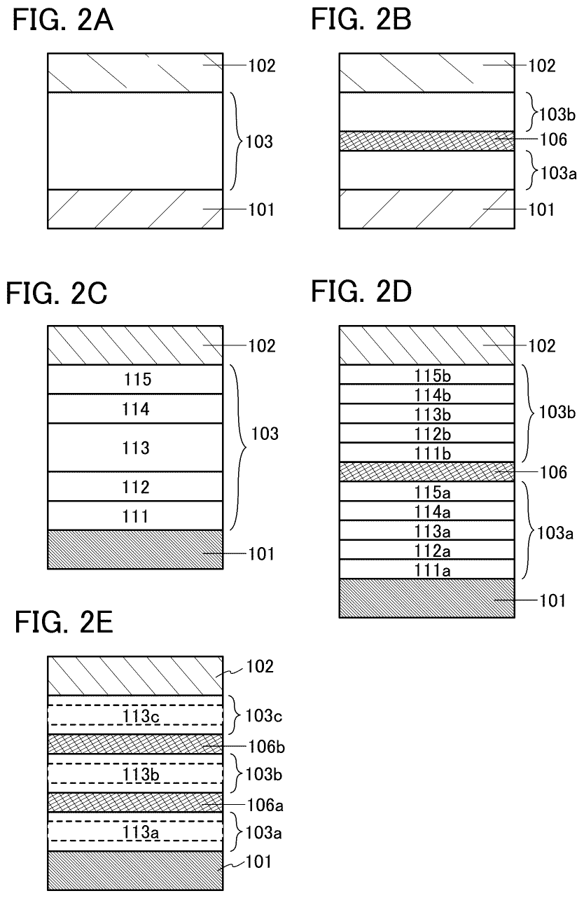

Basic structures of the light-emitting device will be specifically described below with reference to FIGS. 2A to 2E. FIG. 2A illustrates a light-emitting device having a structure (single structure) in which an organic compound layer (also referred to as an EL layer) including a light-emitting layer is provided between a pair of electrodes. Specifically, the organic compound layer 103 is interposed between the first electrode 101 and the second electrode 102.

FIG. 2B illustrates a light-emitting device having a stacked-layer structure (tandem structure) in which a plurality of organic compound layers (two organic compound layers 103a and 103b in FIG. 2B) are provided between a pair of electrodes and a charge-generation layer 106 is provided between the organic compound layers. A light-emitting device having the tandem structure enables fabrication of a light-emitting apparatus that has high efficiency without changing the amount of current.

The charge-generation layer 106 has a function of injecting electrons into one of the organic compound layers 103a and 103b and injecting holes into the other of the organic compound layers 103a and 103b when a potential difference is caused between the first electrode 101 and the second electrode 102. Thus, when a voltage is applied in FIG. 2B such that the potential of the first electrode 101 is higher than that of the second electrode 102, the charge-generation layer 106 injects electrons into the organic compound layer 103a and injects holes into the organic compound layer 103b.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

FIG. 2C illustrates a stacked-layer structure of the organic compound layer 103 in the light-emitting device of one embodiment of the present invention. In this case, the first electrode 101 is regarded as functioning as an anode, and the second electrode 102 is regarded as functioning as a cathode. The organic compound layer 103 has a structure in which the hole-injection layer 111, the hole-transport layer 112, the light-emitting layer 113, the electron-transport layer 114, and the electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can sometimes achieve higher reliability than a single-layer structure. In the case where a plurality of light-emitting layers are provided as in the tandem structure illustrated in FIG. 2B, the layers in each light-emitting layer are sequentially stacked from the anode side as described above. When the first electrode 101 is the cathode and the second electrode 102 is the anode, the stacking order of the layers in the organic compound layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the organic compound layers (103, 103a, and 103b) contains an appropriate combination of a light-emitting substance and a plurality of substances, so that fluorescent light of a desired color or phosphorescent light of a desired color can be obtained. The light-emitting layer 113 may have a stacked-layer structure having different emission colors. In that case, the light-emitting substance and other substances are different between the stacked light-emitting layers. Alternatively, the plurality of organic compound layers (103a and 103b) in FIG. 2B may exhibit their respective emission colors. Also in that case, the light-emitting substance and other substances are different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 2C. Thus, light from the light-emitting layer 113 in the organic compound layer 103 can be resonated between the electrodes and light emitted through the second electrode 102 can be intensified. Thus, high resolution can be easily achieved. In addition, emission intensity at a predetermined wavelength in the front direction can be increased, whereby power consumption can be reduced.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is k, the optical path length between the first electrode 101 and the second electrode 102 (the product of the thickness and the refractive index) is preferably adjusted to be mλ/2 (m is an integer greater than or equal to 1) or close to mλ/2.

To amplify light with a desired wavelength (wavelength: λ) obtained from the light-emitting layer 113, each of the optical path length from the first electrode 101 to a region where the light with the desired wavelength is obtained in the light-emitting layer 113 (light-emitting region) and the optical path length from the second electrode 102 to the region where the light with the desired wavelength is obtained in the light-emitting layer 113 (light-emitting region) is preferably adjusted to be (2m′+1)λ/4 (m′ is an integer greater than or equal to 1) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the light with a desired wavelength is, to be exact, the optical path length between the reflective region in the first electrode 101 and the light-emitting region in the light-emitting layer that emits the light with the desired wavelength. However, it is difficult to precisely determine the reflective region in the first electrode 101 and the light-emitting region in the light-emitting layer that emits the light with the desired wavelength; thus, it is assumed that the above effect can be sufficiently obtained wherever the reflective region and the light-emitting region may be set in the first electrode 101 and the light-emitting layer that emits the light with the desired wavelength, respectively.

The light-emitting device illustrated in FIG. 2D is a light-emitting device having the tandem structure. The tandem structure enables a light-emitting device to emit light with high luminance. Furthermore, the amount of current needed for obtaining a predetermined luminance can be smaller in the tandem structure than in the single structure; thus, the tandem structure enables higher reliability. In addition, power consumption can be reduced.

The light-emitting device illustrated in FIG. 2E is an example of the light-emitting device having the tandem structure illustrated in FIG. 2B, and includes three organic compound layers (103a, 103b, and 103c) stacked with charge-generation layers (106a and 106b) interposed therebetween, as illustrated in FIG. 2E. The three organic compound layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity lower than or equal to 1×10⁻² Ωcm.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity lower than or equal to 1×10⁻² Ωcm.

### <Specific Structure of Light-Emitting Device>

Next, a specific structure of the light-emitting device of one embodiment of the present invention will be described. Here, the description is made using FIG. 2D illustrating the tandem structure. Note that the structure of the organic compound layer applies also to the structure of the light-emitting devices having the single structure illustrated in FIGS. 2A and 2C. When the light-emitting device in FIG. 2D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the organic compound layer 103b, with the use of a material selected as appropriate.

### <Materials of Light-Emitting Device>

### <<Light-Emitting Layer>>

The light-emitting layers (113, 113a, and 113b) contain a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables exhibiting different emission colors (e.g., white light emission obtained by a combination of complementary emission colors). Furthermore, a stacked-layer structure in which one light-emitting layer contains two or more kinds of light-emitting substances may be employed.

The light-emitting layers (113, 113a, and 113b) may each include one or more kinds of organic compounds (e.g., a host material) in addition to a light-emitting substance (a guest material).

Specifically, the light-emitting layer 113 can have the structure described with reference to FIG. 1B. In the light-emitting layer 113, the host materials 118 are present in the largest proportion by weight, and the guest material 119 (phosphorescent compound) is dispersed in the host materials 118. The Ti levels of the host materials 118 (the organic compounds 118_1 and 118_2) in the light-emitting layer 113 are preferably higher than the T₁ level of the guest material (the guest material 119) in the light-emitting layer 113.

The lowest triplet excitation energy level (Ti level) can be calculated, using a thin film of a sample, from an emission edge obtained by measurement of an emission spectrum (phosphorescence spectrum) at a low temperature (e.g., 10 K). Note that the emission spectrum of an emission center substance may be measured using a sample in the form of a thin film or a solution; however, a sample in the form of a solution is preferably used for examination of the state of an isolated molecule. As a solvent of the solution, a solvent with relatively low polarity, such as toluene or chloroform, is preferably used. In the case where the emission center substance is a phosphorescent compound, the temperature at which the lowest triplet excitation energy level (Ti level) is measured may be either low temperature (e.g., 10 K) or room temperature (e.g., 298 K), and the lowest triplet excitation energy level is calculated from an emission edge obtained by measurement of an emission spectrum (phosphorescence spectrum). Note that the emission edge can be determined as the intersection of a tangent and the horizontal axis (representing wavelength) or the baseline. The tangent is drawn at a point at which the slope on a shorter wavelength side of the shortest-wavelength peak (or the shortest-wavelength shoulder peak) of the emission spectrum (phosphorescence spectrum) has the maximum absolute value.

Examples of the light-emitting substance that can be used as the guest material include a substance emitting red light. In addition, the substance emitting red light is preferably a substance emitting phosphorescent light, particularly preferably an organometallic complex. Examples of the light-emitting substances include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(dlnpm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), (3,7-diethyl-4,6-nonanedionato-κO⁴,κO⁶)bis[2,4-dimethyl-6-[7-(1-methylethyl)-1-isoquinolinyl-κN]phenyl-κC]iridium(III), and (3,7-diethyl-4,6-nonanedionato-κO⁴,κO⁶)bis[2,4-dimethyl-6-[5-(1-methylethyl)-2-quinolinyl-κN]phenyl-κC]iridium(III); a platinum complex such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds have an emission peak in the wavelength range from 600 nm to 700 nm. Furthermore, the organometallic iridium complexes having a pyrazine skeleton can provide red light emission with favorable chromaticity. Note that other known red phosphorescent substances can also be used.

In the case where a light-emitting apparatus does not use a red-light-emitting substance as the light-emitting substance or includes light-emitting devices with different structures, the light-emitting substance may be a fluorescent substance, a phosphorescent substance, a substance exhibiting thermally activated delayed fluorescence (TADF), or any other light-emitting substance.

Examples of the material that can be used as a light-emitting substance that emits fluorescent light in the light-emitting layer 113 are as follows. Any other fluorescent substance can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPm), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene- 3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[i/]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPm-03), N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPm, 1,6mMemFLPAPrn, and 1,6BnfAPrn-03 are particularly preferable because of their high hole-trapping properties, high emission efficiency, and high reliability.

Examples of the material that can be used when a phosphorescent substance is used as the light-emitting substance in the light-emitting layer 113 are as follows.

The examples include organometallic iridium complexes having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]) and tris(5-methyl-3,4-diphenyl-4H-1,2,4-triazolato)iridium(III) (abbreviation: [Ir(Mptz)₃]); organometallic iridium complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes having an imidazole skeleton, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN³}-4-cyanophenyl-κC)iridium(III) (abbreviation: CNImIr); organometallic iridium complexes having a benzimidazolidene skeleton, such as tris[(6-tert-butyl-3-phenyl-2H-imidazo[4,5-b]pyrazin-1-yl-κC²)phenyl-κC]iridium(III) (abbreviation: [Ir(cb)₃]); and organometallic iridium complexes in which a phenylpyridine derivative having an electron-withdrawing group is a ligand, such as bis[2-(4′,6′ -difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIracac). These compounds exhibit blue phosphorescence and have an emission peak in the wavelength range from 440 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: [Ir(5mppy-d3)₂(mbfpypy-d3)]), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: [Ir(5mtpy-d₆)₂(mbfpypy-iPr-d₄)]), [2-(methyl-d₃)-8-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy-d₃)]), [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), and [2-methyl-8-(2-pyridinyl-κN)benzofuro[2,3-b]pyridine-κC]bis[2-(2 -pyridinyl-N)phenyl-κC]iridium(III) (abbreviation: [Ir(ppy)₂(mbfpypy)]); and rare earth metal complexes such as tris(acetylacetonato) (monophenanthroline)terbium(III) (abbreviation: [Tb(acac)₃(Phen)]). These are mainly compounds that exhibit green phosphorescence and have an emission peak in the wavelength range from 500 nm to 600 nm. Note that organometallic iridium complexes having a pyrimidine skeleton have distinctively high reliability or emission efficiency and thus are particularly preferable.

Note that any of the aforementioned red phosphorescent materials can also be used. Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

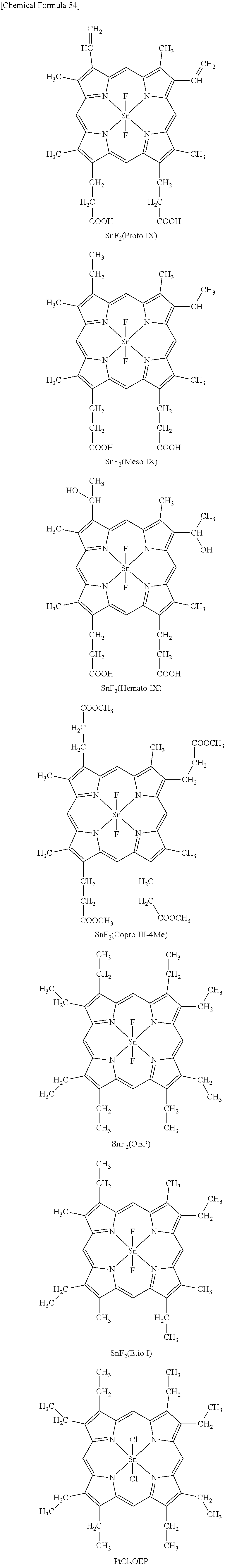

Alternatively, it is possible to use a heterocyclic compound having one or both of a π-electron rich heteroaromatic ring and a n-electron deficient heteroaromatic ring that is represented by any of the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 9-[4-(4,6-diphenyl-1,3,5-triazin-2-yl)phenyl]-9′-phenyl-3,3′-bi-9H-carbazole (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4 -(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of having high electron-transport and hole-transport properties owing to a π-electron rich heteroaromatic ring and a π-electron deficient heteroaromatic ring. Among skeletons having a π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor properties and high reliability. Among skeletons having a π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which a π-electron rich heteroaromatic ring is directly bonded to a π-electron deficient heteroaromatic ring is particularly preferable because the electron-donating property of the π-electron rich heteroaromatic ring and the electron-acceptor property of the π-electron deficient heteroaromatic ring are both improved, the energy difference between the Si level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

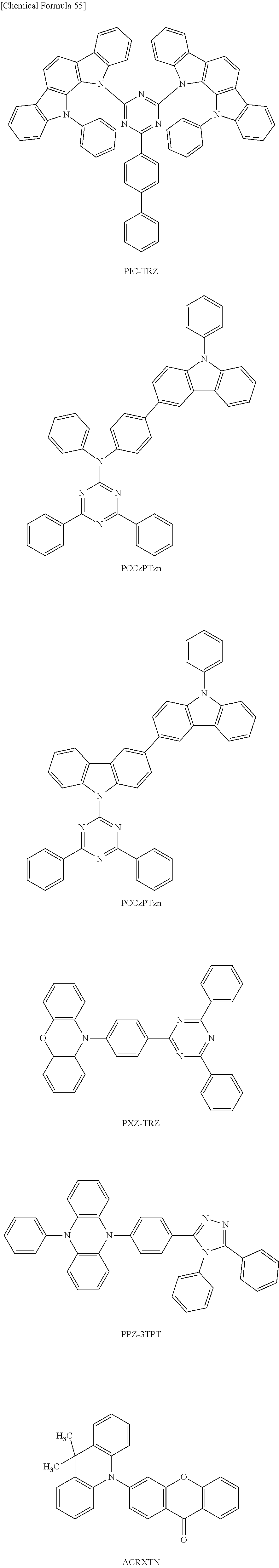 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; CC1(C)c2ccccc2N(c2ccc3c(=O)c4ccccc4oc3c2)c2ccccc21]

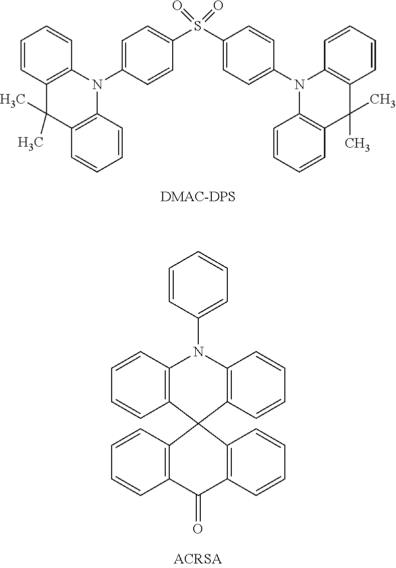 [SMILES: CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

It is also possible to use a TADF material that enables reversible intersystem crossing at extremely high speed and emits light in accordance with a thermal equilibrium model between a singlet excited state and a triplet excited state. Since such a TADF material has an extremely short emission lifetime (excitation lifetime), an efficiency decrease of a light-emitting element in a high-luminance region can be inhibited. Specifically, a material having the following molecular structure can be used.

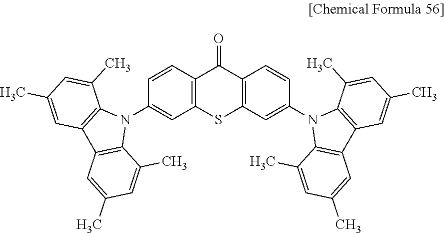

Note that a TADF material is a material having a small difference between the Si level and the T₁ level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light emission.

An exciplex whose excited state is formed by two kinds of substances has an extremely small difference between the Si level and the Ti level and functions as a TADF material capable of converting triplet excitation energy into singlet excitation energy.

A phosphorescence spectrum observed at a low temperature (e.g., 77 K to 10 K) is used for an index of the Ti level. When the level of energy of light with a wavelength of the line obtained by extrapolating a tangent to the fluorescence spectrum at a tail on the short wavelength side is the Si level and the level of energy of light with a wavelength of the line obtained by extrapolating a tangent to the phosphorescence spectrum at a tail on the short wavelength side is the Ti level, the difference between the Si level and the T₁ level of the TADF material is preferably less than or equal to 0.3 eV, further preferably less than or equal to 0.2 eV.

When a TADF material is used as the light-emitting substance, the Si level of the host material is preferably higher than that of the TADF material. In addition, the Ti level of the host material is preferably higher than that of the TADF material.

As an electron-transport material used as the host material, for example, any of metal complexes such as bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ); or an organic compound having a π-electron deficient heteroaromatic ring can be used. Examples of the organic compound having a π-electron deficient heteroaromatic ring include organic compounds having a heteroaromatic ring with an azole skeleton, such as 2-(4-biphenyl)-5-(4-tert-butyl-phenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(4-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), and 2-[3-(dibenzothiophen-4-yl)phenyl]-1-phenyl-1H-benzimidazole (abbreviation: mDBTBIm-II); organic compounds having a heteroaromatic ring with a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 2,4-bis[4-(1-naphthyl)phenyl]-6-[4-(3-pyridyl)phenyl]pyrimidine (abbreviation: 2,4NP-6PyPPm), 6-(biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(biphenyl-4-yl)pyrimidine (abbreviation: 6BP-4Cz2PPm), and 7-[4-(9-phenyl-9H-carbazol-2-yl)quinazolin-2-yl]-7H-dibenzo[c,g]carbazole (abbreviation: PC-cgDBCzQz); organic compounds having a heteroaromatic ring with a pyridine skeleton, such as 3,5-bis[3-(9H-carbazol-9-yl)phenyl]pyridine (abbreviation: 35DCzPPy) and 1,3,5-tri[(3-pyridyl)-phenyl-3-yl]benzene (abbreviation: TmPyPB); and organic compounds having a heteroaromatic ring with a triazine skeleton, such as 2-[3′-(9,9-dimethyl-9H-fluoren-2-yl)biphenyl-3-yl]-4,6-diphenyl-1,3,5-triazine (abbreviation: mFBPTzn), 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), and 2-(biphenyl-3-yl)-4-phenyl-6-[8-([1,1′: 4′,1″-terphenyl]-4-yl)-1-dibenzofuranyl]-1,3,5-triazine (abbreviation: mBP-TPDBfTzn). Among the above materials, the organic compound having a heteroaromatic ring with a diazine skeleton, the organic compound having a heteroaromatic ring with a pyridine skeleton, and the organic compound having a heteroaromatic ring with a triazine skeleton have high reliability and thus are preferable. In particular, the organic compound having a heteroaromatic ring with a diazine (pyrimidine or pyrazine) skeleton and the organic compound having a heteroaromatic ring with a triazine skeleton have a high electron-transport property to contribute to a reduction in driving voltage.

As a hole-transport material used as the host material, an organic compound having an amine skeleton or a π-electron rich heteroaromatic ring can also be used. Examples of the organic compound having an amine skeleton or a π-electron rich heteroaromatic ring include compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 9,9′-diphenyl-9H,9′H-3,3′-bicarbazole (abbreviation: PCCP); compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton or the compound having a carbazole skeleton is preferable because these compounds are highly reliable and have high hole-transport properties to contribute to a reduction in driving voltage. In addition, the organic compounds given as examples of the material having a hole-transport property that can be used for the hole-transport layer 112 can also be used as the hole-transport material that is the host material.

By mixing the electron-transport material having the hole-transport material, the-transport property of the light-emitting layer 113 can be easily adjusted and a recombination region can be easily controlled. A TADF material can be used as the electron-transport material or the hole-transport material.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the Si level of the TADF material is preferably higher than that of the fluorescent substance in order that high emission efficiency can be achieved. Furthermore, the Ti level of the TADF material is preferably higher than the Si level of the fluorescent substance. Therefore, the Ti level of the TADF material is preferably higher than that of the fluorescent substance.

It is also preferable to use a TADF material that emits light whose wavelength overlaps with the wavelength of a lowest-energy absorption band of the fluorescent substance. This enables smooth transfer of excitation energy from the TADF material to the fluorescent substance and accordingly enables efficient light emission, which is preferable.

In order that singlet excitation energy can be efficiently generated from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no 7t bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in cater transport performance, whereby the TADF material and the luminophore of the fluorescent substance can be made away from each other with little influence on carrier transport or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring. Examples of the luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. In particular, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton that are used as the host materials, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further fused to the carbazole skeleton because the HOMO level thereof is shallower than that of the host material having the carbazole skeleton by approximately 0.1 eV and thus holes enter the host material easily. In particular, the host material preferably has a dibenzocarbazole skeleton because the HOMO level thereof is shallower than that of the host material having the carbazole skeleton by approximately 0.1 eV and thus holes enter the host material easily, the hole-transport property is improved, and the heat resistance is increased. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9-(1-naphthyl)-10-(2-naphthyl)anthracene (abbreviation: α,βADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(2-naphthyl)-10-[3-(2-naphthyl)phenyl]anthracene (abbreviation: PN-mPNPAnth), and 1-{4-[10-(biphenyl-4-yl)-9-anthracenyl]phenyl}-2-ethyl-1H-benzimidazole (abbreviation: EtBImPBPhA). In particular, CzPA, cgDBCzPA, 2mBnfPPA, and PCzPA exhibit excellent properties and thus are preferably selected.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of the mixed materials. These mixed materials are preferably selected so as to form an exciplex that exhibits light emission whose wavelength overlaps with the wavelength of a lowest-energy absorption band of the light-emitting substance, in which case energy can be transferred smoothly and light emission can be obtained efficiently. Such a structure is preferably used to reduce the driving voltage.

Note that at least one of the materials forming an exciplex may be a phosphorescent substance. In that case, triplet excitation energy can be efficiently converted into singlet excitation energy by reverse intersystem crossing.

In order to form an exciplex efficiently, a material having an electron-transport property is preferably combined with a material having a hole-transport property and a HOMO level higher than or equal to that of the material having an electron-transport property. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by, for example, comparing the emission spectra of the material having a hole-transport property, the material having an electron-transport property, and the mixed film of these materials, and observing the phenomenon in which the emission spectrum of the mixed film is shifted to the longer wavelength side than the emission spectrum of each of the materials (or has another peak on the longer wavelength side). Alternatively, the formation of an exciplex can be confirmed by a difference in transient response, such as a phenomenon in which the transient photoluminescence (PL) lifetime of the mixed film has longer lifetime components or has a larger proportion of delayed components than that of each of the materials, observed by comparison of transient PL of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

Note that the light-emitting layer 113 can be formed by an evaporation method (including a vacuum evaporation method), an ink-jet method, a coating method, gravure printing, or the like. Besides the above-mentioned materials, an inorganic compound such as a quantum dot or a high molecular compound (e.g., an oligomer, a dendrimer, or a polymer) may be used.

### <<Hole-Injection Layer>>

The hole-injection layers (111, 111a, and 111b) inject holes from the first electrode 101 serving as the anode and the charge-generation layers (106, 106a, and 106b) to the organic compound layers (103, 103a, and 103b) and contain an organic acceptor material and a material having a high hole-injection property. The organic compound described in Embodiment 1 can also be used for the hole-injection layers.

For the hole-injection layers (111, 111a, and 111b), it is possible to use a compound having an electron-withdrawing group (a halogen group or a cyano group); for example, 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene)malononitrile can be used. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group or a halogen group such as a fluoro group) has a very high electron-accepting property and thus is preferable. Specific examples include α,α′,α″-1,2,3-cyclopropanetriylidenetris(4-cyano-2,3,5,6-tetrafluorobenzeneacetonitrile) (abbreviation: Rad), α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a molybdenum oxide, a vanadium oxide, a ruthenium oxide, a tungsten oxide, a manganese oxide, or the like can be used, other than the above-described organic compounds. Alternatively, the hole-injection layers (111, 111a, and 111b) can be formed using a phthalocyanine-based compound such as phthalocyanine (abbreviation: H₂Pc), a phthalocyanine-based complex compound such as copper phthalocyanine (abbreviation: CuPc), an aromatic amine compound such as 4,4′-bis[N-(4-diphenylaminophenyl)-N-phenylamino]biphenyl (abbreviation: DPAB) or N,N′-bis[4-bis(3-methylphenyl)aminophenyl]-N,N′-diphenyl-4,4′-diaminobiphenyl (abbreviation: DNTPD), or a high molecular compound such as poly(3,4-ethylenedioxythiophene)/polystyrenesulfonic acid (abbreviation: PEDOT/PSS). The substance having an acceptor property can extract electrons from an adjacent hole-transport layer (or hole-transport material) by application of an electric field.

Among substances having an acceptor property, an organic compound having an acceptor property, which is easily deposited by evaporation, is easy to use.

Alternatively, a composite material in which a material having a hole-transport property contains any of the aforementioned substances having an acceptor property can be used for the hole-injection layers (111, 111a, and 111b). In the case of using a composite material in which a material having a hole-transport property contains an acceptor substance, a material used to form an electrode can be selected regardless of its work function. In other words, besides a material having a high work function, a material having a low work function can be used for the anode (the first electrode 101).

As the material having a hole-transport property used for the composite material, any of a variety of organic compounds such as aromatic amine compounds, carbazole derivatives, aromatic hydrocarbons, and high molecular compounds (e.g., oligomers, dendrimers, or polymers) can be used. Note that the material having a hole-transport property used for the composite material preferably has a hole mobility higher than or equal to 1×10⁻⁶ cm²/Vs. Organic compounds that can be used as the material having a hole-transport property in the composite material are specifically given below.

Examples of the aromatic amine compound that can be used for the composite material include N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis[4-bis(3-methylphenyl)aminophenyl]-N,N′-diphenyl-4,4′-diaminobiphenyl (abbreviation: DNTPD), and 1,3,5-tris[N-(4-diphenylaminophenyl)-N-phenylamino]benzene (abbreviation: DPA3B). Specific examples of the carbazole derivative include 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), and 1,4-bis[4-(N-carbazolyl)phenyl]-2,3,5,6-tetraphenylbenzene. Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra-tert-butylperylene. Other examples include pentacene and coronene. The aromatic hydrocarbon may have a vinyl skeleton. Examples of the aromatic hydrocarbon having a vinyl group include 4,4′-bis(2,2-diphenylvinyl)biphenyl (abbreviation: DPVBi) and 9,10-bis[4-(2,2-diphenylvinyl)phenyl]anthracene (abbreviation: DPVPA).

Other examples include high molecular compounds such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: poly-TPD).

The material having a hole-transport property used for the composite material further preferably has at least any one of a carbazole skeleton, a dibenzofuran skeleton, a dibenzothiophene skeleton, and an anthracene skeleton. In particular, an aromatic amine having a substituent with a dibenzofuran ring or a dibenzothiophene ring, an aromatic monoamine having a naphthalene ring, or an aromatic monoamine in which a 9-fluorenyl group is bonded to the nitrogen of the amine through an arylene group may be used. Note that the material having a hole-transport property preferably has an N,N-bis(4-biphenyl)amino group to enable fabricating a light-emitting device having a long lifetime. Specific examples of the material having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAPNB), 4-[4-(2-naphthyl)phenyl]-4′,4″-diphenyltriphenylamine (abbreviation: BBAPNBi), 4,4′-diphenyl-4″-([2,1′-binaphthyl]-6-yl)triphenylamine (abbreviation: BBAαNβNB), 4,4′-diphenyl-4″-([2,1′-binaphthyl]-7-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-([2,2′-binaphthyl]-6-yl)triphenylamine (abbreviation: BBA(βN2)B), 4,4′-diphenyl-4″-([2,2′-binaphthyl]-7-yl)triphenylamine (abbreviation: BBA(βN2)B-03), 4,4′-diphenyl-4″-([1,2′-binaphthyl]-4-yl)triphenylamine (abbreviation: BBAβNαNB), 4,4′-diphenyl-4″-([1,2′-binaphthyl]-5-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N′-bis(9,9-dimethyl-9H-fluoren-2-yl)-9,9′-spirobi-9H-fluoren-4-amine, N,N′-bis(9,9-dimethyl-9H-fluoren-2-yl)-9,9′-spirobi-9H-fluoren-3-amine, N,N′-bis(9,9-dimethyl-9H-fluoren-2-yl)-9,9′-spirobi-9H-fluoren-2-amine, and N,N′-bis(9,9-dimethyl-9H-fluoren-2-yl)-9,9′-spirobi-9H-fluoren-1-amine.

It is further preferable that the material having a hole-transport property used in the composite material have a relatively deep HOMO level higher than or equal to −5.7 eV and lower than or equal to −5.4 eV. Using the material having a hole-transport property having a relatively deep HOMO level in the composite material makes it easy to inject holes to the hole-transport layer 112 and to obtain a light-emitting device having a long lifetime. In addition, when the material having a hole-transport property that is used in the composite material has a relatively deep HOMO level, induction of holes can be inhibited properly, so that the light-emitting device can have a longer lifetime.

Note that mixing the above composite material with a fluoride of an alkali metal or an alkaline earth metal (the proportion of fluorine atoms in a layer containing the mixed material is preferably higher than or equal to 20%) can lower the refractive index of the layer. This also enables a layer with a low refractive index to be formed in the organic compound layer 103, leading to higher external quantum efficiency of the light-emitting device.

The formation of the hole-injection layers (111, 111a, and 111b) can improve the hole-injection property, which allows the light-emitting device to be driven at a low voltage.

### <<Hole-Transport Layer>>

The hole-transport layers (112, 112a, and 112b) contain a hole-transport material and can be formed using any of the hole-transport materials given as examples of the material of the hole-injection layers (111, 111a, and 111b). The organic compound described in Embodiment 1 can also be used for the hole-injection layers.

In order that the hole-transport layers (112, 112a, and 112b) can have a function of transporting holes injected into the hole-injection layers (111, 111a, and 111b) to the light-emitting layers (113, 113a, and 113b), the HOMO level of the hole-transport layers (112, 112a, and 112b) is preferably equal or close to the HOMO level of the hole-injection layers (111, 111a, and 111b).

As the hole-transport material, a substance having a hole mobility higher than or equal to 1×10⁻⁶ cm²/Vs is preferably used. Note that other substances may also be used as long as their hole-transport properties are higher than their electron-transport properties. The layer containing a substance having a high hole-transport property is not limited to a single layer and may be a stack of two or more layers each containing any of the above substances.

Examples of the materials that can be used for the hole-transport layers (112, 112a, and 112b) include compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2 -amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 9,9′-diphenyl-9H,9′H-3,3′-bicarbazole (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), and 9-(biphenyl-3-yl)-9′-(biphenyl-4-yl)-9H,9′H-3,3′-bicarbazole (abbreviation: mBPCCBP); compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III) and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton or the compound having a carbazole skeleton is preferable because these compounds are highly reliable and have high hole-transport properties to contribute to a reduction in driving voltage. Note that any of the substances given as examples of the material having a hole-transport property that is used for the composite material for the hole-injection layer 111 can also be suitably used as the material contained in the hole-transport layer 112.

### <<Electron-Transport Layer>>

The electron-transport layers (114, 114a, and 114b) have a function of transporting, to the light-emitting layer 113, electrons injected from the other of the pair of electrodes (the first electrode 101 or the second electrode 102) through the electron-injection layers (115, 115a, and 115b).

As the electron-transport material, it is preferable to use an organic compound having an electron-transport property and an electron mobility higher than or equal to 1×10⁻⁶ cm²/Vs when the square root of the electric field strength [V/cm] is 600. Note that any other substance can also be used as long as the substance has an electron-transport property higher than a hole-transport property. The above organic compound is preferably an organic compound having a R-electron deficient heteroaromatic ring. The organic compound having a π-electron deficient heteroaromatic ring is preferably one or more of an organic compound having a heteroaromatic ring with an azole skeleton, an organic compound having a heteroaromatic ring with a pyridine skeleton, an organic compound having a heteroaromatic ring with a diazine skeleton, and an organic compound having a heteroaromatic ring with a triazine skeleton.

Specific examples of the organic compound having a π-electron deficient heteroaromatic ring and being usable for the above electron-transport layer include organic compounds having an azole skeleton, such as 2-(4-biphenyl)-5-(4-tert-butyl-phenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(4-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs); organic compounds having a heteroaromatic ring with a pyridine skeleton, such as 3,5-bis[3-(9H-carbazol-9-yl)phenyl]pyridine (abbreviation: 35DCzPPy), 1,3,5-tri[(3-pyridyl)-phenyl-3-yl]benzene (abbreviation: TmPyPB), bathophenanthroline (abbreviation: BPhen), bathocuproine (abbreviation: BCP), and 2,9-di(naphthalen-2-yl)-4,7-diphenyl-1,10-phenanthroline (abbreviation: NBPhen); organic compounds having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f;h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 8-[3′-(dibenzothiophen-4-yl)biphenyl-3-yl]naphtho[1′,2′: 4,5]furo[3,2-d]pyrimidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8(PN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis{4-[4-(2-naphthyl)phenyl]-6-phenylpyrimidine}(abbreviation: 2,6(NP-PPm)2Py), 6-(biphenyl-3-yl)-4-[3,5-bis(9H-carbazol-9-yl)phenyl]-2-phenylpyrimidine (abbreviation: 6mBP-4Cz2PPm), 2,4-bis[4-(1-naphthyl)phenyl]-6-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), and 7-[4-(9-phenyl-9H-carbazol-2-yl)quinazolin-2-yl]-7H-dibenzo[c,g]carbazole (abbreviation: PC-cgDBCzQz); and organic compounds having a triazine skeleton, such as 2-[3′-(9,9-dimethyl-9H-fluoren-2-yl)biphenyl-3-yl]-4,6-diphenyl-1,3,5-triazine (abbreviation: mFBPTzn), 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 9-[4-(4,6-diphenyl-1,3,5-triazin-2-yl)phenyl]-9′-phenyl-3,3′-bi-9H-carbazole (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2-[3-(2,6-dimethyl-3-pyridinyl)-5-(9-phenanthryl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), and 2-(biphenyl-3-yl)-4-phenyl-6-[8-[(1,1′: 4′,1″-terphenyl)-4-yl]-1-dibenzofuranyl]-1,3,5-triazine (abbreviation: mBP-TPDBfTzn); and complexes such as 8-quinolinato-lithium (abbreviation: Liq) and tris(8-quinolinolato)aluminum (abbreviation: Alq₃). Among the above materials, the organic compound having a heteroaromatic ring with a diazine skeleton, the organic compound having a heteroaromatic ring with a pyridine skeleton, and the organic compound having a heteroaromatic ring with a triazine skeleton have high reliability and thus are preferable. In particular, the organic compound having a heteroaromatic ring with a diazine (pyrimidine or pyrazine) skeleton and the organic compound having a heteroaromatic ring with a triazine skeleton have a high electron-transport property to contribute to a reduction in driving voltage.

Each of the electron-transport layers (114, 114a, and 114b) is not limited to a single layer and may be a stack of two or more layers each containing any of the above substances.

Between the electron-transport layer (114, 114a, or 114b) and the light-emitting layer (113, 113a, or 113b), a layer that controls transfer of electron carriers may be provided. This is a layer formed by addition of a small amount of a substance having a high electron-trapping property to a material having a high electron-transport property as described above, and the layer is capable of adjusting carrier balance by suppressing transport of electron carriers. Such a structure is very effective in inhibiting a problem (such as a reduction in element lifetime) caused when electrons pass through the light-emitting layer.

### <<Electron-Injection Layer>>

The electron-injection layers (115, 115a, and 115b) have a function of reducing a barrier to electron injection from the second electrode 102 to promote electron injection.

For the electron-injection layers, a Group 1 metal, a Group 2 metal, an oxide of these metals, a halide of these metals, a carbonate of these metals, or the like can be used. Alternatively, a composite material containing any of the electron-transport materials described above and a material having a property of donating electrons to the electron-transport material can also be used. As examples of the material having an electron-donating property, a Group 1 metal, a Group 2 metal, an oxide of any of these metals, and the like can be given. Specifically, an alkali metal, an alkaline earth metal, or a compound thereof, such as lithium fluoride (LiF), sodium fluoride (NaF), cesium fluoride (CsF), calcium fluoride (CaF₂), or lithium oxide (LiOₓ), can be used. Alternatively, a rare earth metal compound like erbium fluoride (ErF₃) can be used. Electride may also be used for the electron-injection layer 115. Examples of the electride include a substance in which electrons are added at high concentration to calcium oxide-aluminum oxide. The electron-injection layers (115, 115a, and 115b) can be formed using the substance that can be used for the electron-transport layers (114, 114a, and 114b).

A composite material in which an organic compound and an electron donor (donor) are mixed may also be used for the electron-injection layers (115, 115a, and 115b). Such a composite material is excellent in an electron-injection property and an electron-transport property because electrons are generated in the organic compound by the electron donor. In this case, the organic compound is preferably a material that is excellent in transporting the generated electrons. Specifically, any of the above-described substances for forming the electron-transport layer 114 (e.g., a metal complex or a heteroaromatic compound) can be used, for example. As the electron donor, a substance having an electron-donating property with respect to the organic compound can be used. Specifically, it is preferable to use an alkali metal, an alkaline earth metal, or a rare earth metal, such as lithium, sodium, cesium, magnesium, calcium, erbium, or ytterbium. It is also preferable to use an alkali metal oxide or an alkaline earth metal oxide, such as a lithium oxide, a calcium oxide, or a barium oxide. Alternatively, a Lewis base such as magnesium oxide can be used. Further alternatively, an organic compound such as tetrathiafulvalene (abbreviation: TTF) can be used.

Note that the light-emitting layer, the hole-injection layer, the hole-transport layer, the electron-transport layer, and the electron-injection layer described above can each be formed by an evaporation method (including a vacuum evaporation method), an ink-jet method, a coating method, a gravure printing method, or the like. Besides the above-mentioned materials, an inorganic compound such as a quantum dot or a high molecular compound (e.g., an oligomer, a dendrimer, or a polymer) may be used for the light-emitting layer, the hole-injection layer, the hole-transport layer, the electron-transport layer, and the electron-injection layer.

The quantum dot may be a colloidal quantum dot, an alloyed quantum dot, a core-shell quantum dot, or a core quantum dot, for example. The quantum dot containing elements belonging to Groups 2 and 16, elements belonging to Groups 13 and 15, elements belonging to Groups 13 and 17, elements belonging to Groups 11 and 17, or elements belonging to Groups 14 and 15 may be used. Alternatively, the quantum dot containing an element such as cadmium (Cd), selenium (Se), zinc (Zn), sulfur (S), phosphorus (P), indium (In), tellurium (Te), lead (Pb), gallium (Ga), arsenic (As), or aluminum (Al) may be used.

### <<Pair of Electrodes>>

The first electrode 101 and the second electrode 102 function as the anode and the cathode of the light-emitting device. The first electrode 101 and the second electrode 102 can be formed using a metal, an alloy, or a conductive compound, a mixture or a stack thereof, or the like.

One of the first electrode 101 and the second electrode 102 is preferably formed using a conductive material having a function of reflecting light. Examples of the conductive material include aluminum (Al), an alloy containing Al, and the like. Examples of the alloy containing Al include an alloy containing Al andL (L represents one or more of titanium (Ti), neodymium (Nd), nickel (Ni), and lanthanum (La)), such as an alloy containing Al and Ti or an alloy containing Al, Ni, and La. Aluminum has low resistance and high light reflectivity. Aluminum is contained in earth's crust in large amount and is inexpensive; thus, it is possible to reduce costs for manufacturing a light-emitting device with aluminum. Alternatively, silver (Ag), an alloy of Ag and N (N represents one or more of yttrium (Y), Nd, magnesium (Mg), ytterbium (Yb), Al, Ti, gallium (Ga), zinc (Zn), indium (In), tungsten (W), manganese (Mn), tin (Sn), iron (Fe), Ni, copper (Cu), palladium (Pd), iridium (Ir), and gold (Au)), or the like may be used. Examples of the alloy containing silver include an alloy containing silver, palladium, and copper, an alloy containing silver and copper, an alloy containing silver and magnesium, an alloy containing silver and nickel, an alloy containing silver and gold, an alloy containing silver and ytterbium, and the like. Besides, a transition metal such as tungsten, chromium (Cr), molybdenum (Mo), copper, or titanium can be used.

Light emitted from the light-emitting layer is extracted through the first electrode 101 and/or the second electrode 102. Thus, at least one of the first electrode 101 and the second electrode 102 is preferably formed using a conductive material having a function of transmitting light. As the conductive material, a conductive material having a visible light transmittance higher than or equal to 40% and lower than or equal to 100%, preferably higher than or equal to 60% and lower than or equal to 100%, and a resistivity lower than or equal to 1×10⁻² Ω·cm can be used.

The first electrode 101 and the second electrode 102 may each be formed using a conductive material having functions of transmitting light and reflecting light. As the conductive material, a conductive material having a visible light reflectivity higher than or equal to 20% and lower than or equal to 80%, preferably higher than or equal to 40% and lower than or equal to 70%, and a resistivity lower than or equal to 1×10⁻² Ω·cm can be used. For example, one or more kinds of conductive metals and alloys, conductive compounds, and the like can be used. Specifically, a metal oxide such as an indium tin oxide (hereinafter, referred to as ITO), an indium tin oxide containing silicon or silicon oxide (ITSO), an indium oxide-zinc oxide (indium zinc oxide), an indium oxide-tin oxide containing titanium, an indium titanium oxide, or an indium oxide containing tungsten oxide and zinc oxide can be used. A metal thin film having a thickness that allows transmission of light (preferably, a thickness greater than or equal to 1 nm and less than or equal to 30 nm) can also be used. As the metal, Ag can be used, for example. As the alloy, an alloy of Ag and Al, an alloy of Ag and Mg, an alloy of Ag and Au, an alloy of Ag and Yb, or the like can be used.

In this specification and the like, as the material having a function of transmitting light, a material having a function of transmitting visible light and having conductivity is used. Examples of the material include, in addition to the above-described oxide conductor typified by ITO, an oxide semiconductor and an organic conductor containing an organic substance. Examples of the organic conductor containing an organic substance include a composite material in which an organic compound and an electron donor (donor) are mixed and a composite material in which an organic compound and an electron acceptor (acceptor) are mixed. Alternatively, an inorganic carbon-based material such as graphene may be used. The resistivity of the material is preferably lower than or equal to 1×10⁵ Ω·cm, further preferably lower than or equal to 1×10⁴ Ω·cm.

The first electrode 101 and/or the second electrode 102 may be formed by stacking two or more of the materials described above.

In order to improve the light extraction efficiency, a material whose refractive index is higher than that of an electrode having a function of transmitting light may be formed in contact with the electrode. The material may be electrically conductive or non-conductive as long as it has a function of transmitting visible light. In addition to the oxide conductors described above, an oxide semiconductor and an organic substance are given as the examples of the material. Examples of the organic substance include the materials for the light-emitting layer, the hole-injection layer, the hole-transport layer, the electron-transport layer, and the electron-injection layer. Alternatively, an inorganic carbon-based material or a metal film thin enough to transmit light can be used. Further alternatively, a plurality of layers each having a thickness of several nanometers to several tens of nanometers may be stacked.

In the case where the first electrode 101 or the second electrode 102 functions as the cathode, the electrode preferably contains a material having a low work function (lower than or equal to 3.8 eV). For example, it is possible to use an element belonging to Group 1 or Group 2 of the periodic table (e.g., an alkali metal such as lithium, sodium, or cesium, an alkaline earth metal such as calcium or strontium, or magnesium), an alloy containing any of these elements (e.g., Ag—Mg or Al—Li), a rare earth metal such as europium (Eu) or Yb, an alloy containing any of these rare earth metals, an alloy containing aluminum or silver, or the like.

When the first electrode 101 or the second electrode 102 is used as the anode, a material with a high work function (higher than or equal to 4.0 eV) is preferably used.

The first electrode 101 and the second electrode 102 may be a stack of a conductive material having a function of reflecting light and a conductive material having a function of transmitting light. This structure is preferably employed, in which case the first electrode 101 and the second electrode 102 can have a function of adjusting the optical path length so that light with a desired wavelength emitted from each light-emitting layer resonates and is intensified.

As the method for forming the first electrode 101 and the second electrode 102, a sputtering method, an evaporation method, a printing method, a coating method, a molecular beam epitaxy (MBE) method, a CVD method, a pulsed laser deposition method, an atomic layer deposition (ALD) method, or the like can be used as appropriate.

### <<Charge-Generation Layer (Intermediate Layer)>>

The charge-generation layer 106 has a function of injecting electrons into the organic compound layer 103a and injecting holes into the organic compound layer 103b when a voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these layers may be stacked. Furthermore, an electron-relay layer may be provided between the p-type layer and the electron-injection buffer layer.

In the case where the charge-generation layer 106 is a p-type layer in which an electron acceptor (a substance having an acceptor property) is added to a hole-transport material, which is an organic compound, any of the hole-transport materials described in this embodiment can be used as the hole-transport material. Examples of the electron acceptor (the substance having an acceptor property) include organic compounds having an electron-withdrawing group (e.g., a halogen group or a cyano group), such as 7,7,8,8-tetracyano-2,3,5,6-tetrafluoroquinodimethane (abbreviation: F4-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylene-1,3,4,5,6,8,9,10-octafluoro-7H-pyren-2-ylidene)malononitrile. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group or a halogen group such as a fluoro group) has a very high electron-accepting property and thus is preferable. Specific examples include α,α′,α″-1,2,3-cyclopropanetriylidenetris(4-cyano-2,3,5,6-tetrafluorobenzeneacetonitrile) (abbreviation: Rad), α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as a molybdenum oxide, a vanadium oxide, a ruthenium oxide, a tungsten oxide, or a manganese oxide can be used, other than the above-described organic compounds. Alternatively, the p-type layer of the charge-generation layer 106 can be formed using a phthalocyanine-based compound such as phthalocyanine (abbreviation: H₂Pc), a phthalocyanine-based complex compound such as copper phthalocyanine (abbreviation: CuPc), or an aromatic skeleton such as 4,4′-bis[N-(4-diphenylaminophenyl)-N-phenylamino]biphenyl (abbreviation: DPAB) or N,N′-bis[4-bis(3-methylphenyl)aminophenyl]-N,N′-diphenyl-4,4′-diaminobiphenyl (abbreviation: DNTPD). The substance having an acceptor property can extract electrons from an adjacent hole-transport layer (or hole-transport material) by application of an electric field. Among substances having an acceptor property, an organic compound having an acceptor property, which is easily deposited by evaporation, is easy to use.

The hole-injection layer 111 is preferably formed using a composite material containing any of the aforementioned materials having an acceptor property and an organic compound having a hole-transport property.

In the case where the charge-generation layer 106 is an electron-injection buffer layer in which an electron donor is added to an electron-transport material, any of the electron-transport materials described in this embodiment may be used as the electron-transport material.

As the electron donor, it is possible to use an alkali metal, an alkaline earth metal, a rare earth metal, a metal belonging to Group 2 or Group 13 of the periodic table, or an oxide or a carbonate thereof. Specific examples include lithium (Li), sodium (Na), cesium (Cs), magnesium (Mg), calcium (Ca), erbium (Er), ytterbium (Yb), indium (In), and silver (Ag). It is also preferable to use an alkali metal oxide or an alkaline earth metal oxide, such as a lithium oxide (Li₂O), a calcium oxide, or a barium oxide. Alternatively, a Lewis base such as indium oxide (In₂O₃), cesium carbonate, or magnesium oxide can be used. Further alternatively, an organic compound such as tetrathiafulvalene (abbreviation: TTF) can be used as the electron donor.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

Although FIG. 2D illustrates the structure in which two of the organic compound layers 103 are stacked, three or more organic compound layers may be stacked with charge-generation layers each provided between different light-emitting layers.

### <<Cap Layer>>

Although not illustrated in FIGS. 2A to 2E, a cap layer may be provided over the second electrode 102 of the light-emitting device. For example, a material with a high refractive index can be used for the cap layer. When the cap layer is provided over the second electrode 102, extraction efficiency of light emitted through the second electrode 102 can be improved.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II).

### <<Substrate>>

A light-emitting device of one embodiment of the present invention may be formed over a substrate of glass, plastic, or the like. As the way of stacking layers over the substrate, layers may be sequentially stacked from the first electrode 101 side or sequentially stacked from the second electrode 102 side.

For the substrate over which the light-emitting device of one embodiment of the present invention can be formed, glass, quartz, plastic, or the like can be used, for example. Alternatively, a flexible substrate may be used. The flexible substrate means a substrate that can be bent, such as a plastic substrate made of polycarbonate or polyarylate, for example. Alternatively, a film, an inorganic vapor deposition film, or the like can be used. Another material may be used as long as the substrate functions as a support in a manufacturing process of the light-emitting devices or the optical elements. Another material having a function of protecting the light-emitting devices or the optical elements may be used.

In this specification and the like, a light-emitting device can be formed using any of a variety of substrates, for example. There is no particular limitation on the type of the substrate. Examples of the substrate include a semiconductor substrate (e.g., a single crystal substrate such as a silicon substrate); an SOI substrate; a glass substrate; a quartz substrate; a plastic substrate; a metal substrate; a stainless steel substrate; a substrate including stainless steel foil; a tungsten substrate; a substrate including tungsten foil; a flexible substrate; an attachment film; and cellulose nanofiber (CNF), paper, and a base material film that include a fibrous material. Examples of a glass substrate include a barium borosilicate glass substrate, an aluminoborosilicate glass substrate, and a soda lime glass substrate. Examples of the flexible substrate, the attachment film, the base material film, and the like are substrates of plastics typified by polyethylene terephthalate (PET), polyethylene naphthalate (PEN), polyether sulfone (PES), and polytetrafluoroethylene (PTFE). Another example is an acrylic resin. Furthermore, polypropylene, polyester, polyvinyl fluoride, and polyvinyl chloride can be given as examples. Other examples include a resin such as a polyamide resin, a polyimide resin, an aramid resin, or an epoxy resin, an inorganic vapor deposition film, and paper.

Alternatively, a flexible substrate may be used as the substrate, and a light-emitting device may be provided directly on the flexible substrate. Further alternatively, a separation layer may be provided between the substrate and the light-emitting device. The separation layer can be used to separate part or the whole of the light-emitting device, which is formed over the separation layer, from the substrate and transfer the separated component onto another substrate. In that case, the light-emitting device can be transferred to a substrate having low heat resistance or a flexible substrate as well. For the above separation layer, a stack including inorganic films, which are a tungsten film and a silicon oxide film, or a structure in which a resin film of polyimide or the like is formed over a substrate can be used, for example.

In other words, after the light-emitting device is formed using a substrate, the light-emitting device may be transferred to another substrate. Examples of the substrate to which the light-emitting device is transferred are, in addition to the above substrates, a cellophane substrate, a stone substrate, a wood substrate, a cloth substrate (including a natural fiber (e.g., silk, cotton, or hemp), a synthetic fiber (e.g., nylon, polyurethane, or polyester), a regenerated fiber (e.g., acetate, cupro, rayon, or regenerated polyester), and the like), a leather substrate, a rubber substrate, and the like. When such a substrate is used, a light-emitting device with high durability, high heat resistance, reduced weight, or reduced thickness can be formed.

The light-emitting device may be formed over an electrode electrically connected to a field-effect transistor (FET), for example, that is formed over any of the above-described substrates. In that case, an active matrix display apparatus in which the FET controls the driving of the light-emitting device can be manufactured.

In this embodiment, one embodiment of the present invention has been described. Other embodiments of the present invention are described in other embodiments. Note that one embodiment of the present invention is not limited thereto. In other words, various embodiments of the invention are described in this embodiment and the other embodiments, and one embodiment of the present invention is not limited to a particular embodiment. For example, although the example in which one embodiment of the present invention is applied to a light-emitting device is described, one embodiment of the present invention is not limited thereto. For example, depending on circumstances or conditions, one embodiment of the present invention is not necessarily used in a light-emitting device. One embodiment of the present invention describes, but is not limited to, an example of including a first organic compound, a second organic compound, and a guest material capable of converting triplet excitation energy into light emission, in which the LUMO level of the first organic compound is lower than that of the second organic compound and the HOMO level of the first organic compound is lower than that of the second organic compound. Depending on circumstances or conditions, in one embodiment of the present invention, for example, the LUMO level of the first organic compound is not necessarily lower than that of the second organic compound. Alternatively, the HOMO level of the first organic compound is not necessarily lower than that of the second organic compound. One embodiment of the present invention describes, but is not limited to, an example where the first organic compound and the second organic compound form an exciplex. Depending on circumstances or conditions, in one embodiment of the present invention, for example, the first organic compound and the second organic compound do not necessarily form an exciplex. One embodiment of the present invention describes, but is not limited to, an example where the LUMO level of the guest material is higher than that of the first organic compound and the HOMO level of the guest material is lower than that of the second organic compound. Depending on circumstances or conditions, in one embodiment of the present invention, for example, the LUMO level of the guest material is not necessarily higher than that of the first organic compound. Alternatively, the HOMO level of the guest material is not necessarily lower than that of the second organic compound.

The structure described above in this embodiment can be used in combination with any of the structures described in the other embodiments as appropriate.

### Embodiment 3

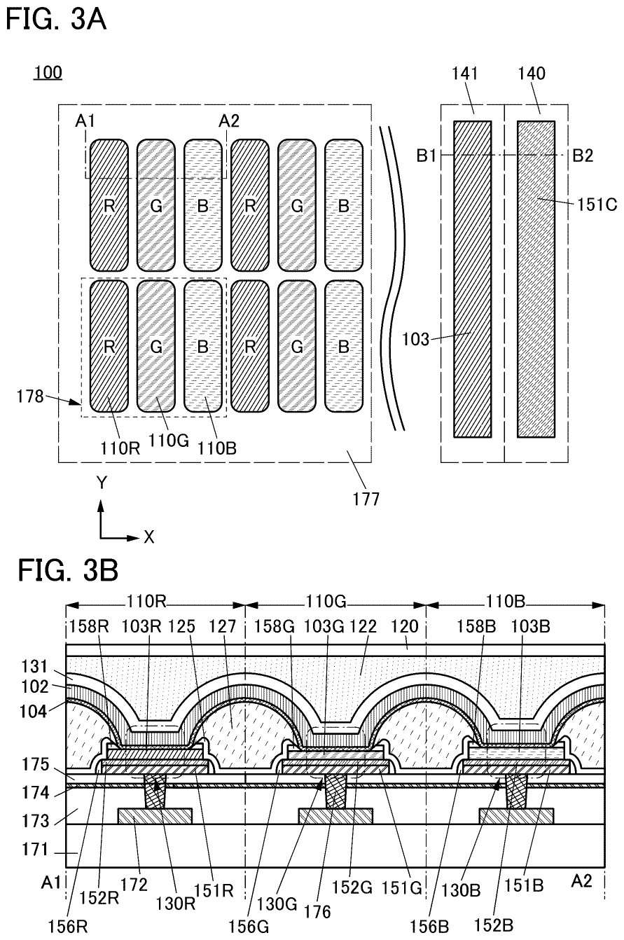

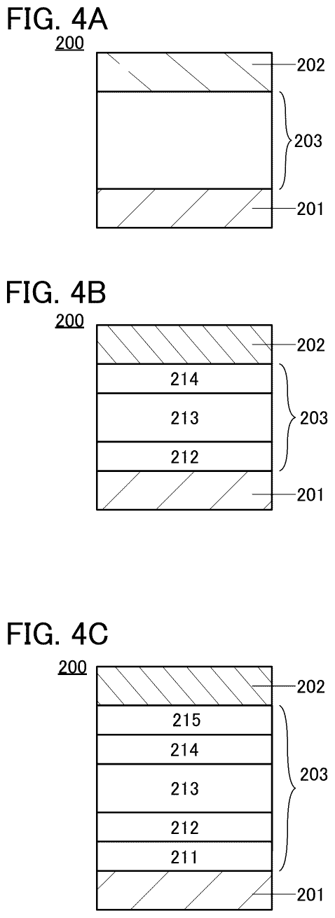

As illustrated in FIGS. 3A and 3B, a plurality of light-emitting devices 130 are formed over an insulating layer 175 to constitute a display apparatus. In this embodiment, a display apparatus of one embodiment of the present invention is described in detail.

A display apparatus 100 includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 1101B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 3A illustrates an example where the region 141 and the connection portion 140 are positioned on the right side of the pixel portion 177, there is no particular limitation on the positions of the region 141 and the connection portion 140. The number of the regions 141 and the number of the connection portions 140 can each be one or more.

FIG. 3B is an example of a cross-sectional view along the dashed-dotted line A1-A2 in FIG. 3A. As illustrated in FIG. 3B, the display apparatus 100 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is attached to the protective layer 131 with a resin layer 122. An inorganic insulating layer 125 and an insulating layer 127 over the inorganic insulating layer 125 are preferably provided between the adjacent light-emitting devices 130.

Although FIG. 3B illustrates cross sections of a plurality of the inorganic insulating layers 125 and a plurality of the insulating layers 127, the inorganic insulating layers 125 are preferably connected to each other and the insulating layers 127 are preferably connected to each other when the display apparatus 100 is seen from above. That is, the insulating layer 127 preferably includes opening portions over first electrodes.

In FIG. 3B, a light-emitting device 130R, a light-emitting device 130G, and a light-emitting device 130B are illustrated as the light-emitting devices 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, the light-emitting device 130G, or the light-emitting device 130B may emit visible light of another color or infrared light.

The display apparatus of one embodiment of the present invention can be, for example, a top-emission display apparatus where light is emitted in the direction opposite to a substrate over which light-emitting devices are formed. Note that the display apparatus of one embodiment of the present invention may be of a bottom-emission type.

Examples of a light-emitting substance included in the light-emitting device 130 include organic compounds or organometallic complexes such as a substance emitting fluorescent light (a fluorescent material), a substance emitting phosphorescent light (a phosphorescent material), and a substance exhibiting thermally activated delayed fluorescence (a thermally activated delayed fluorescent (TADF) material). Other examples include inorganic compounds (e.g., a quantum dot material).

The light-emitting device 130R has a structure illustrated in FIG. TA. The light-emitting device 130R includes the first electrode (pixel electrode) including a conductive layer 151R and a conductive layer 152R, an organic compound layer 103R over the first electrode, a common layer 104 over the organic compound layer 103R, and a second electrode (common electrode) 102 over the common layer 104. Although the common layer 104 is not necessarily provided, it is preferable to provide the common layer 104 to reduce damage to the organic compound layer 103R during processing. In the case where the common layer 104 is provided, the common layer 104 is preferably an electron-injection layer. In the case where the common layer 104 is provided, a stack of the organic compound layer 103R and the common layer 104 corresponds to the organic compound layer 103 described in Embodiment 2.

The light-emitting device 130G has a structure illustrated in FIG. 1A. The light-emitting device 130G includes the first electrode (pixel electrode) including a conductive layer 151G and a conductive layer 152G, an organic compound layer 103G over the first electrode, the common layer 104 over the organic compound layer 103G, and the second electrode (common electrode) 102 over the common layer 104. Although the common layer 104 is not necessarily provided, it is preferable to provide the common layer 104 to reduce damage to the organic compound layer 103G during processing. In the case where the common layer 104 is provided, the common layer 104 is preferably an electron-injection layer. In the case where the common layer 104 is provided, a stack of the organic compound layer 103G and the common layer 104 corresponds to the organic compound layer 103 described in Embodiment 2.

The light-emitting device 130B has a structure illustrated in FIG. 1A. The light-emitting device 130B includes the first electrode (pixel electrode) including a conductive layer 151B and a conductive layer 152B, an organic compound layer 103B over the first electrode, the common layer 104 over the organic compound layer 103B, and the second electrode (common electrode) 102 over the common layer 104. Although the common layer 104 is not necessarily provided, it is preferable to provide the common layer 104 to reduce damage to the organic compound layer 103B during processing. In the case where the common layer 104 is provided, the common layer 104 is preferably an electron-injection layer. Furthermore, in the case where the common layer 104 is provided, a stack of the organic compound layer 103B and the common layer 104 corresponds to the organic compound layer 103 described in Embodiment 2.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, 103G, and 103B are island-shaped layers that are independent of each other on a light-emitting device basis or on an emission color basis. Providing the island-shaped organic compound layer 103 in each of the light-emitting devices 130 can inhibit leakage current between the adjacent light-emitting devices 130 even in a high-resolution display apparatus. This can prevent crosstalk, so that a display apparatus with extremely high contrast can be obtained. Specifically, a display apparatus having high current efficiency at low luminance can be obtained.

The island-shaped organic compound layer 103 is formed by forming an EL film and processing the EL film by a lithography method.

In the display apparatus of one embodiment of the present invention, the first electrode (pixel electrode) of the light-emitting device preferably has a stacked-layer structure. For example, in the example illustrated in FIG. 3B, the first electrode of the light-emitting device 130 is a stack of the conductive layer 151 and the conductive layer 152. In the case where the display apparatus 100 is of a top-emission type and the pixel electrode of the light-emitting device 130 functions as the anode, for example, the conductive layer 151 preferably has high visible light reflectance, and the conductive layer 152 preferably has a visible-light-transmitting property and a high work function. In the case where the display apparatus 100 is of a top-emission type, the higher the visible light reflectance of the pixel electrode is, the higher the efficiency of extraction of the light emitted by the organic compound layer 103 is. In the case where the pixel electrode functions as the anode, the higher the work function of the pixel electrode is, the easier hole injection into the organic compound layer 103 is. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage.

In the case where the conductive layer 151 has high visible light reflectance, the visible light reflectance of the conductive layer 151 is preferably higher than or equal to 40% and lower than or equal to 100%, or higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

In view of the above, an insulating layer 156 is formed on the side surfaces of the conductive layers 151 and 152 in the display apparatus 100 of this embodiment. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display apparatus 100 to be manufactured by a high-yield method and to be accordingly inexpensive. In addition, generation of a defect in the display apparatus 100 can be inhibited, which makes the display apparatus 100 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, an indium tin oxide, an indium zinc oxide, zinc oxide, zinc oxide containing gallium, titanium oxide, an indium zinc oxide containing gallium, an indium zinc oxide containing aluminum, an indium tin oxide containing silicon, an indium zinc oxide containing silicon, and the like. In particular, an indium tin oxide containing silicon can be suitably used for the conductive layer 152 because of having a work function higher than or equal to 4.0 eV, for example.

The conductive layer 151 and the conductive layer 152 may each be a stack of a plurality of layers that contain different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

Note that an end portion of the insulating layer 156 may have a tapered shape. Specifically, when the end portion of the insulating layer 156 has a tapered shape with a taper angle less than 90°, coverage with a component provided along the side surface of the insulating layer 156 can be improved.

The structure described in this embodiment can be used in combination with any of the structures described in other embodiments as appropriate.

### Embodiment 4

In this embodiment, a light-receiving device of one embodiment of the present invention will be described.

The light-receiving device of one embodiment of the present invention has a function of sensing light (hereinafter, also referred to as alight-receiving function). FIGS. 4A to 4C are each a schematic cross-sectional view of a light-receiving device 200 of one embodiment of the present invention.

### <<Basic Structure of Light-Receiving Device>>

Basic structures of the light-receiving device will be described. FIG. 4A illustrates the light-receiving device 200 including at least a light-receiving layer 203 including an active layer and a carrier-transport layer between a pair of electrodes. Specifically, an EL layer 203 is interposed between the first electrode 201 and the second electrode 202.

FIG. 4B illustrates a stacked-layer structure of the light-receiving layer 203 in the light-receiving device 200 of one embodiment of the present invention. The light-receiving layer 203 has a structure in which a first carrier-transport layer 212, an active layer 213, and a second carrier-transport layer 214 are sequentially stacked over the first electrode 201.

FIG. 4C illustrates a stacked-layer structure of the light-receiving layer 203 in the light-receiving device 200 of one embodiment of the present invention. The light-receiving layer 203 has a structure in which a first carrier-injection layer 211, the first carrier-transport layer 212, the active layer 213, the second carrier-transport layer 214, and a second carrier-injection layer 215 are sequentially stacked over the first electrode 201.

The organic compound described in Embodiment 1, which has a favorable carrier-transport property, particularly an excellent hole-transport property, can be suitably used for a carrier-transport layer in a light-receiving device described in this embodiment.

### <<Specific Structure of Light-Receiving Device>>

Next, a specific structure of the light-receiving device 200 of one embodiment of the present invention will be described. Here, description is made with reference to FIG. 4C.

### <First Electrode and Second Electrode>

The first electrode 201 and the second electrode 202 can be formed using materials that can be used for the first electrode 101 and the second electrode 102, which will be described in Embodiment 5.

Note that a microcavity structure can be obtained when the first electrode 201 is a reflective electrode and the second electrode 202 is a semi-transmissive and semi-reflective electrode, for example. The microcavity structure can intensify light with a specific wavelength to be sensed, thereby achieving a light-receiving device with high sensitivity.

### <First Carrier-Injection Layer>

The first carrier-injection layer 211 injects holes from the light-receiving layer 203 to the first electrode 201, and contains a material with a high hole-injection property. Examples of the material with a high hole-injection property include an aromatic amine compound and a composite material containing a hole-transport material and an acceptor material (electron-accepting material).

The first carrier-injection layer 211 can be formed using a material that can be used for the hole-injection layer 111, which will be described in Embodiment 5.

### <First Carrier-Transport Layer>

The first carrier-transport layer 212 transports holes generated in the active layer 213 on the basis of incident light to the first electrode 201, and contains a hole-transport material (also referred to as a first organic compound). The hole-transport material preferably has a hole mobility higher than or equal to 1×10⁻⁶ cm²/Vs. Note that other substances can also be used as long as the substances have a hole-transport property higher than an electron-transport property.

As the hole-transport material (first organic compound), a R-electron rich heteroaromatic compound or an aromatic amine (a compound having an aromatic amine skeleton) can be used.

Alternatively, a carbazole derivative, a thiophene derivative, or a furan derivative can be used as the hole-transport material (first organic compound).

As the hole-transport material (first organic compound), an aromatic monoamine compound or a heteroaromatic monoamine compound having at least one skeleton of biphenylamine, carbazolylamine, dibenzofuranylamine, dibenzothiophenylamine, fluorenylamine, and spirofluorenylamine can be used.

Alternatively, as the hole-transport material (first organic compound), an aromatic monoamine compound or a heteroaromatic monoamine compound having two or more skeletons selected from biphenylamine, carbazolylamine, dibenzofuranylamine, dibenzothiophenylamine, fluorenylamine, and spirofluorenylamine can be used.

In the case where the hole-transport material (first organic compound) is an aromatic monoamine compound or a heteroaromatic monoamine compound having two or more skeletons selected from biphenylamine, carbazolylamine, dibenzofuranylamine, dibenzothiophenylamine, fluorenylamine, and spirofluorenylamine, one nitrogen atom may be shared by two or more skeletons. For example, in the case where fluorene and biphenyl are bonded to a nitrogen atom of a monoamine in an aromatic monoamine compound, the compound can be regarded as an aromatic monoamine compound having a fluorenylamine skeleton and a biphenylamine skeleton.

Note that each of biphenylamine, carbazolylamine, dibenzofuranylamine, dibenzothiophenylamine, fluorenylamine, and spirofluorenylamine listed above as the skeleton included in the hole-transport material (first organic compound) may have a substituent. Examples of the substituent include a substituted or unsubstituted aryl group having 6 to 30 carbon atoms, a substituted or unsubstituted alkyl group having 1 to 20 carbon atoms, a substituted or unsubstituted cycloalkyl group having 1 to 20 carbon atoms, and a substituted or unsubstituted heteroaryl group having 4 to 30 carbon atoms.

The hole-transport material (first organic compound) is preferably an amine compound having a triarylamine skeleton (a heteroaryl group or a carbazolyl group is also included as an aryl group in a triarylamine compound).

The first carrier-transport layer 212 can also be formed using a material that can be used for the hole-transport layer 112, which will be described in Embodiment 5.

The first carrier-transport layer 212 is not limited to a single layer, and may be a stack of two or more layers each containing any of the above substances; each of the layers may be a mixed layer containing two or more kinds of compounds.

In the light-receiving device described in this embodiment, the active layer 213 can be formed using the same organic compound as the first carrier-transport layer 212. The use of the same organic compound for the first carrier-transport layer 212 and the active layer 213 is preferable, in which case carriers can be efficiently transported from the first carrier-transport layer 212 to the active layer 213.

### <Active Layer>

The active layer 213 generates carriers on the basis of incident light and contains a semiconductor. Examples of the semiconductor include an inorganic semiconductor such as silicon and an organic semiconductor including an organic compound. This describes shows an example in which an organic semiconductor is used as the semiconductor contained in the active layer. The use of an organic semiconductor is preferable because the light-emitting layer and the active layer provided in the same device can be formed by the same method (e.g., a coating method or a vacuum evaporation method) and thus the same manufacturing apparatus can be used.

The active layer 213 contains at least a third organic compound and a fourth organic compound.

Examples of the third organic compound include π-electron rich heteroaromatic ring compounds and electron-donating compounds, such as copper(II) phthalocyanine (CuPc), tetraphenyldibenzoperiflanthene (DBP), zinc phthalocyanine (ZnPc), tin phthalocyanine (SnPc), and quinacridone.

Other examples of the third organic compound include a carbazole compound, a thiophene compound, a furan compound, and a compound having an aromatic amine skeleton. Other examples of the third organic compound include a naphthalene compound, an anthracene compound, a pyrene compound, a triphenylene compound, a fluorene compound, a pyrrole compound, a benzofuran compound, a benzothiophene compound, an indole compound, a dibenzofiran compound, a dibenzothiophene compound, an indolocarbazole compound, a porphyrin compound, a phthalocyanine compound, a naphthalocyanine compound, a quinacridone compound, a polyphenylene vinylene compound, a polyparaphenylene compound, a polyfluorene compound, a polyvinylcarbazole compound, and a polythiophene compound.

Examples of the fourth organic compound include π-electron deficient heteroaromatic ring compounds and electron-accepting compounds, such as a perylenetetracarboxylic diimide (PTCDI) compound, an oxadiazole compound, a triazole compound, an imidazole compound, an oxazole compound, a thiazole compound, a phenanthroline compound, a quinoline compound, a benzoquinoline compound, a quinoxaline compound, a dibenzoquinoxaline compound, a pyridine compound, a bipyridine compound, a pyrimidine compound, a naphthalene compound, an anthracene compound, a coumalin compound, a rhodamine compound, a triazine compound, a quinone compound, a metal complex having a quinoline skeleton, a metal complex having a benzoquinoline skeleton, a metal complex having an oxazole skeleton, and a metal complex having a thiazole skeleton.

Examples of the fourth organic compound include electron-accepting organic semiconductor materials such as fullerene (e.g., C₆₀ and C₇₀) and fullerene compounds. Fullerene has a soccer ball-like shape, which is energetically stable. Both the HOMO level and the LUMO level of fullerene are deep (low). Having a deep LUMO level, fullerene has an extremely high electron-accepting property (acceptor property). When π-electron conjugation (resonance) spreads on a plane as in benzene, an electron-donating property (donor property) usually increases; however, fullerene has a spherical shape, and thus has a high electron-accepting property although π-electron conjugation widely spread therein. The high electron-accepting property efficiently causes rapid charge separation and thus is useful for light-receiving devices. Both C₆₀ and C₇₀ have a wide absorption band in the visible light region, and C₇₀ is especially preferable because of having a larger t-electron conjugation system and a wider absorption band in the long wavelength region than C₆₀. Other examples of fullerene compounds include [6,6]-phenyl-C₇₁-butyric acid methyl ester (abbreviation: PC70BM), [6,6]-phenyl-C₆₁-butyric acid methyl ester (abbreviation: PC60BM), and 1′,1″,4′,4″-tetrahydro-di[1,4]methanonaphthaleno[1,2:2′,3′,56,60:2″,3″ ][5,6]fullerene-C₆₀ (abbreviation: ICBA).

The active layer 213 is preferably a stacked film of a first layer containing the third organic compound and a second layer containing the fourth organic compound.

In the light-receiving device having any of the aforementioned structures, the active layer 213 is preferably a mixed film containing the third organic compound and the fourth organic compound.

The HOMO level of the electron-donating organic semiconductor material is preferably shallower (higher) than the HOMO level of the electron-accepting organic semiconductor material. The LUMO level of the electron-donating organic semiconductor material is preferably shallower (higher) than the LUMO level of the electron-accepting organic semiconductor material.

Fullerene having a spherical shape may be used as the electron-accepting organic semiconductor material, and an organic semiconductor material having a substantially planar shape may be used as the electron-donating organic semiconductor material. Molecules of similar shapes tend to aggregate, and aggregated molecules of similar kinds, which have molecular orbital energy levels close to each other, can increase the carrier-transport property.

### <Second Carrier-Transport Layer>

The second carrier-transport layer 214 transports electrons generated in the active layer 213 on the basis of incident light to the second electrode 202, and contains an electron-transport material (also referred to as a second organic compound). The electron-transport material preferably has an electron mobility higher than or equal to 1×10⁻⁶ cm²/Vs. Note that other substances can also be used as long as the substances have an electron-transport property higher than a hole-transport property.

As the electron-transport material (second organic compound), a π-electron deficient heteroaromatic compound can be used.

As the electron-transport material (second organic compound), any of the following materials can be used, for example: a metal complex having a quinoline skeleton, a metal complex having a benzoquinoline skeleton, a metal complex having an oxazole skeleton, a metal complex having a thiazole skeleton, an oxadiazole derivative, a triazole derivative, an imidazole derivative, an oxazole derivative, a thiazole derivative, a phenanthroline derivative, a quinoline derivative having a quinoline ligand, a benzoquinoline derivative, a quinoxaline derivative, a dibenzoquinoxaline derivative, a pyridine derivative, a bipyridine derivative, a pyrimidine derivative, and a π-electron deficient heteroaromatic compound such as a nitrogen-containing heteroaromatic compound.

Alternatively, the electron-transport material (second organic compound) is a compound having a triazine ring.

The second carrier-transport layer 214 can be formed using a material that can be used for the electron-transport layer 114, which will be described in Embodiment 5.

The second carrier-transport layer 214 is not limited to a single layer and may be a stack of two or more layers each containing any of the above substances.

### <Second Carrier-Injection Layer>

The second carrier-injection layer 215 is a layer for increasing the efficiency of electron injection from the light-receiving layer 203 to the second electrode 202, and contains a material with a high electron-injection property. As the material with a high electron-injection property, an alkali metal, an alkaline earth metal, or a compound thereof can be used. As the material with a high electron-injection property, a composite material containing an electron-transport material and a donor material (electron-donating material) can also be used.

The second carrier-injection layer 215 can be formed using a material that can be used for the electron-injection layer 115, which will be described in Embodiment 5.

A structure in which a plurality of light-receiving layers are stacked between a pair of electrodes (the structure is also referred to as a tandem structure) can be obtained by providing a charge-generation layer between two light-receiving layers 203. In addition, three or more light-receiving layers may be stacked with charge-generation layers each provided between adjacent light-receiving layers. The charge-generation layer can be formed using a material that can be used for the charge-generation layer 106, which is described in Embodiment 2.

Materials that can be used for the layers (the first carrier-injection layer 211, the first carrier-transport layer 212, the active layer 213, the second carrier-transport layer 214, and the second carrier-injection layer 215) included in the light-receiving layer 203 of the light-receiving device described in this embodiment are not limited to the materials described in this embodiment, and other materials can be used in combination as long as the functions of the layers are fulfilled.

Note that in this specification and the like, the terms “layer” and “film” can be interchanged with each other as appropriate.

Note that the light-receiving device of one embodiment of the present invention has a function of sensing visible light. The light-receiving device of one embodiment of the present invention has sensitivity to visible light. The light-receiving device of one embodiment of the present invention further preferably has a function of sensing visible light and infrared light. The light-receiving device of one embodiment of the present invention preferably has sensitivity to visible light and infrared light.

In this specification and the like, a blue (B) wavelength range is greater than or equal to 400 nm and less than 490 nm, and blue (B) light has at least one emission spectrum peak in the wavelength range. A green (G) wavelength range is greater than or equal to 490 nm and less than 580 nm, and green (G) light has at least one emission spectrum peak in the wavelength range. A red (R) wavelength range is greater than or equal to 580 nm and less than 700 nm, and red (R) light has at least one emission spectrum peak in the wavelength range. In this specification and the like, a visible light wavelength range is greater than or equal to 400 nm and less than 700 nm, and visible light has at least one emission spectrum peak in the wavelength range. An infrared (IR) wavelength range is greater than or equal to 700 nm and less than 900 nm, and infrared (IR) light has at least one emission spectrum peak in the wavelength range.

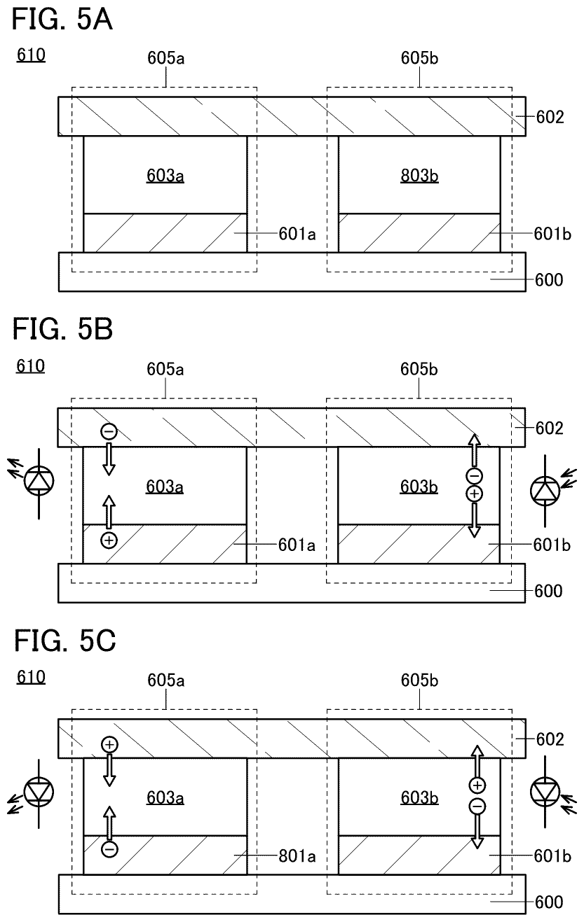

The above-described light-receiving device of one embodiment of the present invention can be used for a display apparatus including an organic EL device. In other words, the light-receiving device of one embodiment of the present invention can be incorporated into a display apparatus including an organic EL device. As an example, FIG. 5A illustrates a schematic cross-sectional view of a light-emitting and light-receiving apparatus 610 used as a display apparatus in which a light-emitting device 605a and a light-receiving device 605b are formed over the same substrate.

The light-emitting and light-receiving apparatus 610 includes the light-emitting device 605a and the light-receiving device 605b, and thus has one or both of an imaging function and a sensing function in addition to an image displaying function.

The light-emitting device 605a has a function of emitting light (hereinafter, also referred to as a light-emitting function). The light-emitting device 605a includes an electrode 601a, an EL layer 603a, and an electrode 602. Thus, the EL layer 603a interposed between the electrode 601a and the electrode 602 at least includes a light-emitting layer. The light-emitting layer contains a light-emitting substance. The EL layer 603a emits light when a voltage is applied between the electrode 601a and the electrode 602. The EL layer 603a may include any of a variety of layers such as a hole-injection layer, a hole-transport layer, an electron-transport layer, an electron-injection layer, a carrier-blocking (hole-blocking or electron-blocking) layer, and a charge-generation layer, in addition to the light-emitting layer. For the light-emitting device 605a, a structure of the light-emitting device, which is an organic EL device to be described in Embodiment 5, can be employed.

The light-receiving device 605b has a function of sensing light (hereinafter, also referred to as a light-receiving function). The light-emitting device 605b includes an electrode 601b, a light-receiving layer 603b, and the electrode 602. The light-receiving layer 603b interposed between the electrode 601b and the electrode 602 at least includes an active layer. The light-receiving device 605b functions as a photoelectric conversion device; when light is incident on the light-receiving layer 603b, electric charge can be generated and extracted as a current. At this time, a voltage may be applied between the electrode 601b and the electrode 602. The amount of generated electric charge depends on the amount of the light incident on the light-receiving layer 603b. For the light-receiving device 605b, the structure of the above-described light-receiving device 200 can be employed.

The light-receiving device 605b, which is easily made thin, lightweight, and large in area and has a high degree of freedom for shape and design, can be used in a variety of display apparatuses. In addition, the EL layer 603a included in the light-emitting device 605a and the light-receiving layer 603b included in the light-receiving device 605b can be formed by the same method (e.g., a vacuum evaporation method) with the same manufacturing apparatus, which is preferable.

The electrode 601a and the electrode 601b are provided on the same plane. In FIG. 5A, the electrodes 601a and 601b are provided over a substrate 600. The electrodes 601a and 601b can be formed by processing a conductive film formed over the substrate 600 into an island shape, for example. In other words, the electrodes 601a and 601b can be formed through the same process.

As the substrate 600, a substrate having heat resistance high enough to withstand the formation of the light-emitting device 605a and the light-receiving device 605b can be used. When an insulating substrate is used as the substrate 600, a glass substrate, a quartz substrate, a sapphire substrate, a ceramics substrate, an organic resin substrate, or the like can be used. Alternatively, a semiconductor substrate can be used. For example, a single crystal semiconductor substrate or a polycrystalline semiconductor substrate of silicon, silicon carbide, or the like; a compound semiconductor substrate of silicon germanium or the like; an SOI substrate; or the like can be used.

As the substrate 600, it is particularly preferable to use the insulating substrate or the semiconductor substrate where a semiconductor circuit including a semiconductor device such as a transistor is formed. The semiconductor circuit preferably forms a pixel circuit, a gate line driver circuit (a gate driver), a source line driver circuit (a source driver), or the like. In addition to the above, an arithmetic circuit, a memory circuit, or the like may be formed.

The electrode 602 is formed of a layer shared by the light-emitting device 605a and the light-receiving device 605b. As the electrode through which light enters or exits, a conductive film that transmits visible light and infrared light is used. As the electrode through which light neither enters nor exits, a conductive film that reflects visible light and infrared light is preferably used.

The electrode 602 in the display apparatus of one embodiment of the present invention functions as one of the electrodes in each of the light-emitting device 605a and the light-receiving device 605b.

In FIG. 5B, the electrode 601a of the light-emitting device 605a has a potential higher than that of the electrode 602. In this case, the electrode 601a functions as an anode and the electrode 602 functions as a cathode in the light-emitting device 605a. The electrode 601b of the light-receiving device 605b has a potential lower than that of the electrode 602. For easy understanding of the direction of current flow, FIG. 5B illustrates a circuit symbol of a light-emitting diode on the left of the light-emitting device 605a and a circuit symbol of a photodiode on the right of the light-receiving device 605b. The flow directions of carriers (electrons and holes) in each device are also schematically indicated by arrows.

In the structure illustrated in FIG. 5B, when a first potential is supplied to the electrode 601a through a first wiring, a second potential is supplied to the electrode 602 through a second wiring, and a third potential is supplied to the electrode 601b through a third wiring, the following relationship is satisfied: the first potential>the second potential>the third potential.

In FIG. 5C, the electrode 601a of the light-emitting device 605a has a potential lower than that of the electrode 602. In this case, the electrode 601a functions as a cathode and the electrode 602 functions as an anode in the light-emitting device 605a. The electrode 601b of the light-receiving device 605b has a potential lower than that of the electrode 602 and a potential higher than that of the electrode 601a. For easy understanding of the direction of current flow, FIG. 5C illustrates a circuit symbol of a light-emitting diode on the left of the light-emitting device 605a and a circuit symbol of a photodiode on the right of the light-receiving device 605b. The flow directions of carriers (electrons and holes) in each device are also schematically indicated by arrows.

In the structure illustrated in FIG. 5C, when a first potential is supplied to the electrode 601a through a first wiring, a second potential is supplied to the electrode 602 through a second wiring, and a third potential is supplied to the electrode 601b through a third wiring, the following relationship is satisfied: the second potential>the third potential>the first potential.

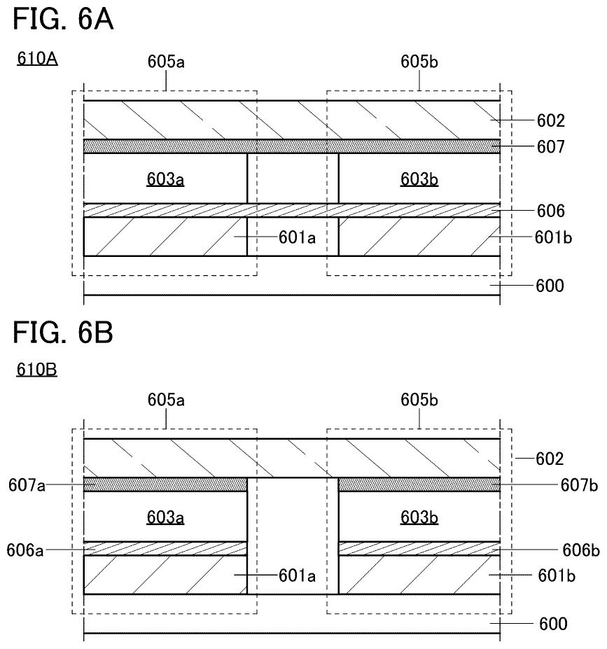

FIG. 6A illustrates a light-emitting and light-receiving apparatus 610A that is a variation example of the light-emitting and light-receiving apparatus 610. The light-emitting and light-receiving apparatus 610A is different from the light-emitting and light-receiving apparatus 610 in including a common layer 606 and a common layer 607. In the light-emitting device 605a, the common layers 606 and 607 function as part of the EL layer 603a. The common layer 606 includes a hole-injection layer and a hole-transport layer, for example. The common layer 607 includes an electron-transport layer and an electron-injection layer, for example.

With the common layers 606 and 607, a light-receiving device can be incorporated without a significant increase in the number of times of separate coloring, whereby the light-emitting and light-receiving apparatus 610A can be manufactured with a high throughput.

FIG. 6B illustrates a light-emitting and light-receiving apparatus 610B that is a variation example of the light-emitting and light-receiving apparatus 610. The light-emitting and light-receiving apparatus 610B is different from the light-emitting and light-receiving apparatus 610A in that the EL layer 603a includes a layer 606a and a layer 607a and the light-receiving layer 603b includes a layer 606b and a layer 607b. The layers 606a and 606b are formed using different materials, and each include a hole-injection layer and a hole-transport layer, for example. Note that the layers 606a and 606b may be formed using the same material. The layers 607a and 607b are formed using different materials, and each include an electron-transport layer and an electron-injection layer, for example. Note that the layers 607a and 607b may be formed using the same material.

An optimum material for forming the light-emitting device 605a is selected for the layers 606a and 607a and an optimum material for forming the light-receiving device 605b is selected for the layers 606b and 607b, whereby the light-emitting device 605a and the light-receiving device 605b can have higher performance in the light-emitting and light-receiving apparatus 610B.

The resolution of pixels including the light-receiving device 605b can be higher than or equal to 100 ppi, preferably higher than or equal to 200 ppi, further preferably higher than or equal to 300 ppi, still further preferably higher than or equal to 400 ppi, and yet further preferably higher than or equal to 500 ppi, and lower than or equal to 2000 ppi, lower than or equal to 1000 ppi, or lower than or equal to 600 ppi, for example. In particular, when the light-receiving devices 605b are arranged at a resolution higher than or equal to 200 ppi and lower than or equal to 600 ppi, preferably higher than or equal to 300 ppi and lower than or equal to 600 ppi, the light-emitting and light-receiving apparatus of one embodiment of the present invention can be suitably used for image capturing of a fingerprint. In fingerprint authentication with the light-emitting and light-receiving apparatus 610, arranging the light-receiving devices 605b at a high resolution enables, for example, highly accurate extraction of the minutiae of fingerprints; thus, the accuracy of the fingerprint authentication can be increased. The resolution is preferably higher than or equal to 500 ppi, in which case the authentication conforms to the standard by the National Institute of Standards and Technology (NIST) or the like. On the assumption that the light-receiving devices are arranged at a resolution of 500 ppi, the size of each pixel is 50.8 μm, which is adequate for image capturing of a fingerprint ridge distance (typically, greater than or equal to 300 μm and less than or equal to 500 μm).

The structures described in this embodiment can be used in appropriate combination with any of the structures described in the other embodiments.

### Embodiment 5

In this embodiment, specific structure examples of a light-emitting and light-receiving apparatus of one embodiment of the present invention will be described.

### <Structure Example of Light-Emitting and Light-Receiving Apparatus 700>

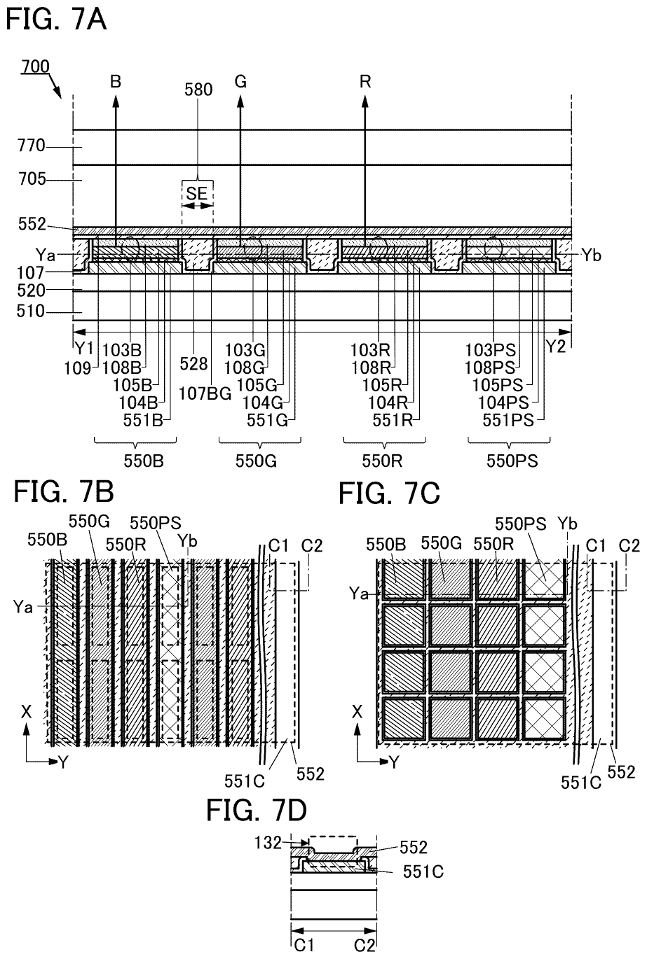

A light-emitting and light-receiving apparatus 700 illustrated in FIG. 7A includes a light-emitting device 550B, a light-emitting device 550G, a light-emitting device 550R, and a light-receiving device 550PS. The light-emitting device 550B, the light-emitting device 550G, the light-emitting device 550R, and the light-receiving device 550PS are formed over a functional layer 520 provided over a first substrate 510. The functional layer 520 includes, for example, circuits such as a circuit GD that are composed of a plurality of transistors, and wirings that electrically connect these circuits. Note that these driver circuits are electrically connected to the light-emitting device 550B, the light-emitting device 550G, the light-emitting device 550R, and the light-receiving device 550PS, for example, to drive them. The light-emitting and light-receiving apparatus 700 includes an insulating layer 705 over the functional layer 520 and the devices (the light-emitting devices and the light-receiving device), and the insulating layer 705 has a function of attaching a second substrate 770 and the functional layer 520.

The light-emitting device 550B, the light-emitting device 550G, the light-emitting device 550R, and the light-receiving device 550PS each have any of the device structures described in Embodiments 2 to 4. Described here is the case where the light-emitting devices have any of the structures illustrated in FIGS. 2A to 2E and the light-receiving device has the structure illustrated in FIG. 4B. Note that the light-emitting and light-receiving apparatus illustrated in FIG. 4B has a structure in which parts of the EL layer (the hole-injection layer, the hole-transport layer, and the electron-transport layer) of the light-emitting device and parts of the active layer (the first transport layer and the second transport layer) of the light-receiving device are concurrently formed using the same material in a manufacturing process; meanwhile, this embodiment describes a case where separation can be made not only between the light-emitting device and the light-receiving device, but also between all the devices (the light-emitting devices and the light-receiving device).

In this specification and the like, a structure in which light-emitting layers in light-emitting devices of different colors (for example, blue (B), green (G), and red (R)) and a light-receiving layer in a light-receiving device are separately formed or separately patterned is sometimes referred to as a side-by-side (SBS) structure. Although the light-emitting device 550B, the light-emitting device 550G, the light-emitting device 550R, and the light-receiving device 550PS are arranged in this order in the light-emitting and light-receiving apparatus 700 illustrated in FIG. 7A, one embodiment of the present invention is not limited to this structure. For example, in the light-emitting and light-receiving apparatus 700, these devices may be arranged in the order of the light-emitting device 550R, the light-emitting device 550G, the light-emitting device 550B, and the light-receiving device 550PS.

In FIG. 7A, the light-emitting device 550B includes an electrode 551B, an electrode 552, and the organic compound layer 103B. The light-emitting device 550G includes an electrode 551G, the electrode 552, and the organic compound layer 103G. The light-emitting device 550R includes an electrode 551R, the electrode 552, and the organic compound layer 103R. The light-receiving device 550PS includes an electrode 551PS, the electrode 552, and alight-receiving layer 103PS. Note that a specific structure of each layer of the light-receiving device is as described in Embodiment 4. In addition, a specific structure of each layer of the light-emitting device is as described in Embodiments 2 and 3. The organic compound layer 103B, the organic compound layer 103G, and the organic compound layer 103R each have a stacked-layer structure of layers having different functions including their respective light-emitting layers (105B, 105G, and 105R). The light-receiving layer 103PS has a stacked-layer structure of layers having different functions including an active layer 105PS. FIG. 7A illustrates a case where the organic compound layer 103B includes a hole-injection/transport layer 104B, a light-emitting layer 105B, an electron-transport layer 108B, and an electron-injection layer 109; the organic compound layer 103G includes a hole-injection/transport layer 104G, a light-emitting layer 105G, an electron-transport layer 108G, and the electron-injection layer 109; the organic compound layer 103R includes a hole-injection/transport layer 104R, a light-emitting layer 105R, an electron-transport layer 108R, and the electron-injection layer 109; and the light-receiving layer 103PS includes a first transport layer 104PS, the active layer 105PS, a second transport layer 108PS, and the electron-injection layer 109. However, the present invention is not limited thereto. Note that each of the hole-injection/transport layers (104B, 104G, and 104R) represents a layer having the functions of the hole-injection layer and the hole-transport layer described in Embodiment 2, and may have a stacked-layer structure.

Note that the electron-transport layers (108B, 108G, and 108R) and the second transport layer 108PS may have a function of blocking holes moving from the anode side to the cathode side through the EL layers (103B, 103G, and 103R) and the light-receiving layer 103PS. The electron-injection layer 109 may have a stacked-layer structure in which some or all of layers are formed using different materials.

As illustrated in FIG. 7A, an insulating layer 107 may be formed on the side surfaces (or end portions) of the hole-injection/transport layers (104B, 104G, and 104R), the light-emitting layers (105B, 105G, and 105R), and the electron-transport layers (108B, 108G, and 108R) included in the EL layers (103B, 103G, and 103R), and the side surfaces (or end portions) of the first transport layer 104PS, the active layer 105PS, and the second transport layer 108PS included in the light-receiving layer 103PS. The insulating layer 107 is formed in contact with the side surfaces (or end portions) of the EL layers (103B, 103G, and 103R) and the light-receiving layer 103PS. This can inhibit entry of oxygen, moisture, or constituent elements thereof into the inside through the side surfaces of the EL layers (103B, 103G, and 103R) and the light-receiving layer 103PS. For the insulating layer 107, aluminum oxide, magnesium oxide, hafnium oxide, gallium oxide, an indium gallium zinc oxide, silicon nitride, or silicon nitride oxide can be used, for example. Some of the above-described materials may be stacked to form the insulating layer 107. The insulating layer 107 can be formed by a sputtering method, a CVD method, an MBE method, a PLD method, an ALD method, or the like and is formed preferably by an ALD method, which achieves favorable coverage. Note that the insulating layer 107 continuously covers the side surfaces (or end portions) of part of the EL layers (103B, 103G, and 103R) and part of the light-receiving layer 103PS of adjacent devices. For example, in FIG. 7A, the side surfaces of the organic compound layer 103B of the light-emitting device 550B and the organic compound layer 103G of the light-emitting device 550G are covered with an insulating layer 107BG. In a region covered with the insulating layer 107BG, a partition wall 528 formed using an insulating material is preferably formed, as illustrated in FIG. 7A.

In addition, the electron-injection layer 109 is formed over the electron-transport layers (108B, 108G, and 108R) that are parts of the EL layers (103B, 103G, and 103R), the second transport layer 108PS that is part of the light-receiving layer 103PS, and the insulating layer 107. Note that the electron-injection layer 109 may have a stacked-layer structure of two or more layers (for example, stacked layers having different electric resistances).

The electrode 552 is formed over the electron-injection layer 109. Note that the electrodes (551B, 551G, and 551R) and the electrode 552 include overlap regions. The light-emitting layer 105B is provided between the electrode 551B and the electrode 552, the light-emitting layer 105G is provided between the electrode 551G and the electrode 552, the light-emitting layer 105R is provided between the electrode 551R and the electrode 552, and the light-receiving layer 103PS is provided between the electrode 551PS and the electrode 552.

The EL layers (103B, 103G, and 103R) illustrated in FIG. 7A each have a structure similar to that of the organic compound layer 103 described in Embodiment 2. The light-receiving layer 103PS has a structure similar to that of the light-receiving layer 203 described in Embodiment 4. The light-emitting layer 105B can emit blue light, the light-emitting layer 105G can emit green light, and the light-emitting layer 105R can emit red light, for example.

The partition walls 528 and the insulating layer 107 are provided between part of the light-emitting device 550B, part of the light-emitting device 550G, part of the light-emitting device 550R, and part of the light-receiving device 550PS. As illustrated in FIG. 7A, the partition walls 528 are in contact with the side surfaces (or end portions) of the electrodes (551B, 551G, 551R, and 551PS), parts of the EL layers (103B, 103G, and 103R), and part of the light-receiving layer 103PS with the insulating layer 107 therebetween.

In each of the EL layers and the light-receiving layer, particularly the hole-injection layer, which is included in the hole-transport region between the anode and the light-emitting layer and between the anode and the active layer, often has high conductivity; thus, a hole-injection layer formed as a layer shared by adjacent devices (the light-receiving device and the light-emitting device, the light-emitting devices, or the light-receiving devices) might cause crosstalk. Thus, as described in this structure example, the partition walls 528 formed using an insulating material are provided between adjacent layers among the EL layers and the light-receiving layers, which can inhibit occurrence of crosstalk between adjacent devices (between the light-receiving device and the light-emitting device, between the light-emitting devices, or between the light-receiving devices).

In the case where the manufacturing process includes a patterning step, the side surfaces (or end portions) of the EL layers and the light-receiving layer are exposed in the middle of the step. This may promote deterioration of the EL layer and the light-receiving layer by allowing the entry of oxygen, water, or the like through the side surfaces (or end portions) of the EL layer and the light-receiving layer. Hence, providing the partition wall 528 can inhibit the deterioration of the EL layer and the light-receiving layer in the manufacturing process.

Providing the partition wall 528 can flatten the surface by reducing a depressed portion formed between adjacent devices (between the light-receiving device and the light-emitting device, between the light-emitting devices, or between the light-receiving devices). When the depressed portion is reduced, disconnection of the electrode 552 formed over the EL layers and the light-receiving layer can be inhibited. Examples of an insulating material used to form the partition wall 528 include organic materials such as an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. Other examples include organic materials such as polyvinyl alcohol (PVA), polyvinyl butyral, polyvinyl pyrrolidone, polyethylene glycol, polyglycerin, pullulan, water-soluble cellulose, and alcohol-soluble polyamide resin. A photosensitive resin such as a photoresist can also be used. Examples of the photosensitive resin include positive-type materials and negative-type materials.

With the use of the photosensitive resin, the partition wall 528 can be fabricated by only light exposure and developing steps. The partition wall 528 may be fabricated using a negative photosensitive resin (e.g., a resist material). In the case where an insulating layer containing an organic material is used as the partition wall 528, a material absorbing visible light is suitably used. When such a material absorbing visible light is used for the partition wall 528, light emission from the EL layer can be absorbed by the partition wall 528, leading to a reduction in light leakage (stray light) to an adjacent EL layer or light-receiving layer. Accordingly, a display panel with high display quality can be provided.

For example, the difference between the top-surface level of the partition wall 528 and the top-surface level of any of the organic compound layer 103B, the organic compound layer 103G, the organic compound layer 103R, and the light-receiving layer 103PS is preferably 0.5 times or less, further preferably 0.3 times or less the thickness of the partition wall 528. The partition wall 528 may be provided such that the top-surface level of any of the organic compound layer 103B, the organic compound layer 103G, the organic compound layer 103R, and the light-receiving layer 103PS is higher than the top-surface level of the partition wall 528, for example. Alternatively, the partition wall 528 may be provided such that the top-surface level of the partition wall 528 is higher than the top-surface level of any of the organic compound layer 103B, the organic compound layer 103G, the organic compound layer 103R, and the light-receiving layer 103PS, for example.

When electrical continuity is established between the organic compound layer 103B, the organic compound layer 103G, the organic compound layer 103R, and the light-receiving layer 103PS in a light-emitting and light-receiving apparatus (display panel) with ahigh resolution more than 1000 ppi, crosstalk occurs, resulting in a narrower color gamut that the light-emitting and light-receiving apparatus is capable of reproducing. Providing the partition wall 528 in a display panel with a high resolution more than 1000 ppi, preferably more than 2000 ppi, or further preferably in a display panel with a ultrahigh resolution more than 5000 ppi allows the display panel to express vivid colors.

FIGS. 7B and 7C are each a schematic top view of the light-emitting and light-receiving apparatus 700 taken along the dashed-dotted line Ya-Yb in the cross-sectional view of FIG. 7A. Specifically, the light-emitting device 550B, the light-emitting device 550G, and the light-emitting device 550R are arranged in a matrix. Note that FIG. 7B illustrates what is called a stripe arrangement, in which the light-emitting devices of the same color are arranged in the X-direction. FIG. 7C illustrates a structure in which the light-emitting devices of the same color are arranged in the X-direction and separated by patterning for each pixel. Note that the arrangement method of the light-emitting devices is not limited thereto; another method such as a delta, zigzag, PenTile, or diamond arrangement may also be employed.

The EL layers (the organic compound layer 103B, the organic compound layer 103G, and the organic compound layer 103R) and the light-receiving layer 103PS are processed to be separated by patterning using a photolithography method; hence, a light-emitting and light-receiving apparatus (display panel) with a high resolution can be manufactured. The end portions (side surfaces) of the EL layer and the light-receiving layer 103PS processed by patterning using a photolithography method have substantially the same surface (or are positioned on substantially the same plane). In this case, the widths (SE) of spaces 580 between adjacent layers among the EL layers and the light-receiving layers are each preferably less than or equal to 5 μm, further preferably less than or equal to 1 μm.

In the EL layer, particularly the hole-injection layer, which is included in the hole-transport region between the anode and the light-emitting layer, often has high conductivity; thus, a hole-injection layer formed as a layer shared by adjacent light-emitting devices might cause crosstalk. Therefore, processing the EL layers to be separated by patterning using a photolithography method as described in this structure example can inhibit occurrence of crosstalk between adjacent light-emitting devices.

FIG. 7D is a schematic cross-sectional view taken along the dashed-dotted line C1-C2 in FIGS. 7B and 7C. FIG. 7D illustrates a connection portion 132 where a connection electrode 551C and the electrode 552 are electrically connected to each other. In the connection portion 132, the electrode 552 is provided over and in contact with the connection electrode 551C. The partition wall 528 is provided to cover an end portion of the connection electrode 551C.

### Embodiment 6

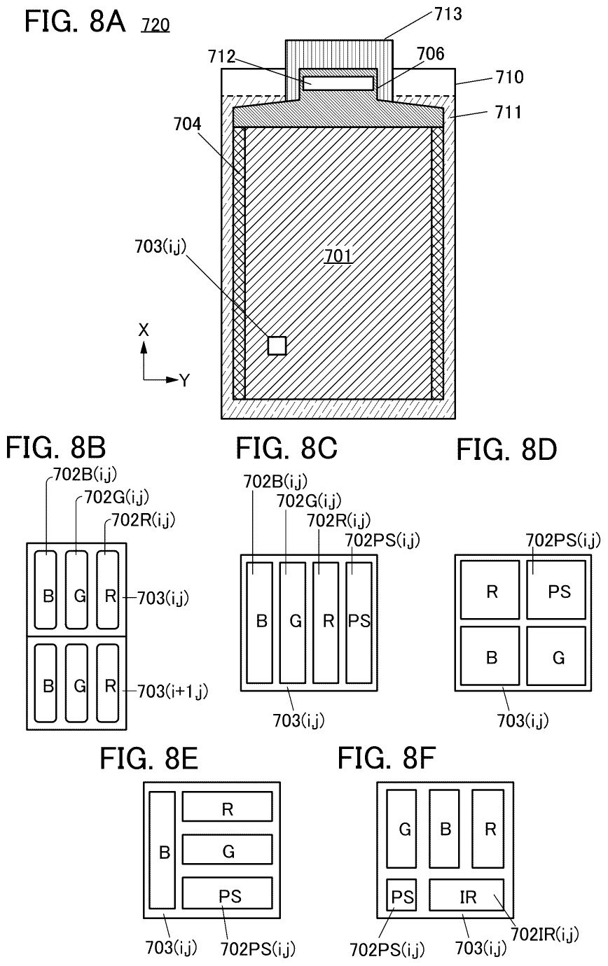

In this embodiment, a light-emitting and light-receiving apparatus 720 is described with reference to FIGS. 8A to 8F, FIGS. 9A to 9C, and FIG. 10. The light-emitting and light-receiving apparatus 720 illustrated in FIGS. 8A to 8F, FIGS. 9A to 9C, and FIG. 10 includes any of the light-receiving devices and the light-emitting devices described in Embodiments 4 and 5 and therefore is a light-emitting and light-receiving apparatus. Furthermore, the light-emitting and light-receiving apparatus 720 described in this embodiment can be used in a display portion of an electronic appliance or the like and therefore can also be referred to as a display panel or a display apparatus. Moreover, the light-emitting and light-receiving apparatus has a structure in which the light-emitting device is used as a light source and the light-receiving device receives light from the light-emitting device.

Furthermore, the light-emitting and light-receiving apparatus of this embodiment can have high definition or large size. Therefore, the light-emitting and light-receiving apparatus of this embodiment can be used, for example, in display portions of electronic appliances such as a digital camera, a digital video camera, a digital photo frame, a mobile phone, a portable game machine, a smart phone, a wristwatch terminal, a tablet terminal, a portable information terminal, and an audio reproducing apparatus, in addition to display portions of electronic appliances with a relatively large screen, such as a television apparatus, a desktop or laptop personal computer, a monitor of a computer or the like, digital signage, and a large game machine such as a pachinko machine.

FIG. 8A is a top view of the light-emitting and light-receiving apparatus 720.

In FIG. 8A, the light-emitting and light-receiving apparatus 720 has a structure in which a substrate 710 and a substrate 711 are attached to each other. In addition, the light-emitting and light-receiving apparatus 720 includes a display region 701, a circuit 704, a wiring 706, and the like. Note that the display region 701 includes a plurality of pixels. As illustrated in FIG. 8B, a pixel 703(i, j) illustrated in FIG. 8A and a pixel 703(i+1, j) are adjacent to each other.

Furthermore, in the example of the light-emitting and light-receiving apparatus 720 illustrated in FIG. 8A, the substrate 710 is provided with an integrated circuit (IC) 712 by a chip on glass (COG) method, a chip on film (COF) method, or the like. As the IC 712, an IC including a scan line driver circuit, a signal line driver circuit, or the like can be used, for example. In the example illustrated in FIG. 8A, an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through a flexible printed circuit (FPC) 713 or to the wiring 706 from the IC 712. Note that the light-emitting and light-receiving apparatus 720 is not necessarily provided with the IC. The IC may be mounted on the FPC by a COF method or the like.

FIG. 8B illustrates the pixel 703(1,j) and the pixel 703(i+1, j) of the display region 701. A plurality of kinds of subpixels including light-emitting devices that emit light of different colors can be included in the pixel 703(i, j). Alternatively, a plurality of subpixels including light-emitting devices that emit light of the same color may be included in addition to those described above. For example, the pixel can include three kinds of subpixels. The three subpixels can be of three colors of red (R), green (G), and blue (B) or of three colors of yellow (Y), cyan (C), and magenta (M), for example. Alternatively, the pixel can include four kinds of subpixels. The four subpixels can be of four colors of R, G, B, and white (W) or of four colors of R, G, B, and yellow (Y), for example. Specifically, the pixel 703(1,j) can consist of a subpixel 702B(j) for blue display, a subpixel 702G(i, j) for green display, and a subpixel 702R(i, j) for red display.

Other than the subpixels including the light-emitting devices, a subpixel including a light-receiving device may also be provided.

FIGS. 8C to 8F illustrate various layout examples of the pixel 703(i, j) including a subpixel 702PS(i, j) including a light-receiving device. The pixel arrangement in FIG. 8C is stripe arrangement, and the pixel arrangement in FIG. 8D is matrix arrangement. The pixel arrangement in FIG. 8E has a structure where three subpixels (the subpixels R, G, and PS) are vertically arranged next to one subpixel (the subpixel B). In the pixel arrangement in FIG. 8F, the vertically oriented three subpixels G, B, and R are arranged laterally, and the subpixel PS and the horizontally oriented subpixel IR are arranged laterally below the three subpixels. Note that the wavelength of light sensed by the subpixel 702PS(i, j) is not particularly limited; however, the light-receiving device included in the subpixel 702PS(i, j) preferably has sensitivity to light emitted by the light-emitting device included in the subpixel 702R(i, j), the subpixel 702G(i, j), the subpixel 702B(i, j), or a subpixel 702IR(i, j). For example, the light-receiving device preferably senses one or more kinds of light in blue, violet, bluish violet, green, yellowish green, yellow, orange, red, and infrared wavelength ranges, and the like.

Furthermore, as illustrated in FIG. 8F, the subpixel 702IR(i, j) that emits infrared rays may be added to any of the above-described sets of subpixels in the pixel 703(1,j). Specifically, the subpixel that emits light including light with a wavelength of higher than or equal to 650 nm and lower than or equal to 1000 nm may be used in the pixel 703(i, j).

Note that the arrangement of subpixels is not limited to the structures illustrated in FIGS. 8B to 8F and a variety of arrangement methods can be employed. The arrangement of subpixels may be stripe arrangement, S-stripe arrangement, matrix arrangement, delta arrangement, Bayer arrangement, or PenTile arrangement, for example.

Furthermore, the top surfaces of the subpixels may have a triangular shape, a quadrangular shape (including a rectangular shape and a square shape), a polygonal shape such as a pentagonal shape, a polygonal shape with rounded corners, an elliptical shape, or a circular shape, for example. The top surface shape of a subpixel herein refers to a top surface shape of a light-emitting region of a light-emitting device.

Furthermore, in the case where not only a light-emitting device but also a light-receiving device is included in a pixel, the pixel has a light-receiving function and thus can detect a contact or approach of an object while displaying an image. For example, an image can be displayed by using all the subpixels included in a light-emitting apparatus; or light can be emitted by some of the subpixels as a light source and an image can be displayed by using the remaining subpixels.

Note that the light-receiving area of the subpixel 702PS(i, j) is preferably smaller than the light-emitting areas of the other subpixels. A smaller light-receiving area leads to a narrower image-capturing range, prevents a blur in a captured image, and improves the definition. Thus, by using the subpixel 702PS(i, j), high-resolution or high-definition image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i, j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably senses infrared light. Thus, touch sensing is possible even in a dark place.

Here, the touch sensor or the near touch sensor can detect an approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the light-emitting and light-receiving apparatus can preferably detect the object when the distance between the light-emitting and light-receiving apparatus and the object is greater than or equal to 0.1 mm and less than or equal to 300 mm, preferably greater than or equal to 3 mm and less than or equal to 50 mm. With this structure, light-emitting and light-receiving apparatus can be controlled without the object directly contacting with the light-emitting and light-receiving apparatus. In other words, the light-emitting and light-receiving apparatus can be controlled in a contactless (touchless) manner. With the above-described structure, the light-emitting and light-receiving apparatus can be operated with a reduced risk of being dirty or damaged, or without direct contact between the object and a dirt (e.g., dust, bacteria, or a virus) attached to the light-emitting and light-receiving apparatus.

For high-resolution image capturing, the subpixel 702PS(i, j) is preferably provided in every pixel included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) is provided in some subpixels in the light-emitting and light-receiving apparatus. When the number of subpixels 702PS(i, j) included in the light-emitting and light-receiving apparatus is smaller than the number of subpixels 702R(i, j) or the like, higher detection speed can be achieved.

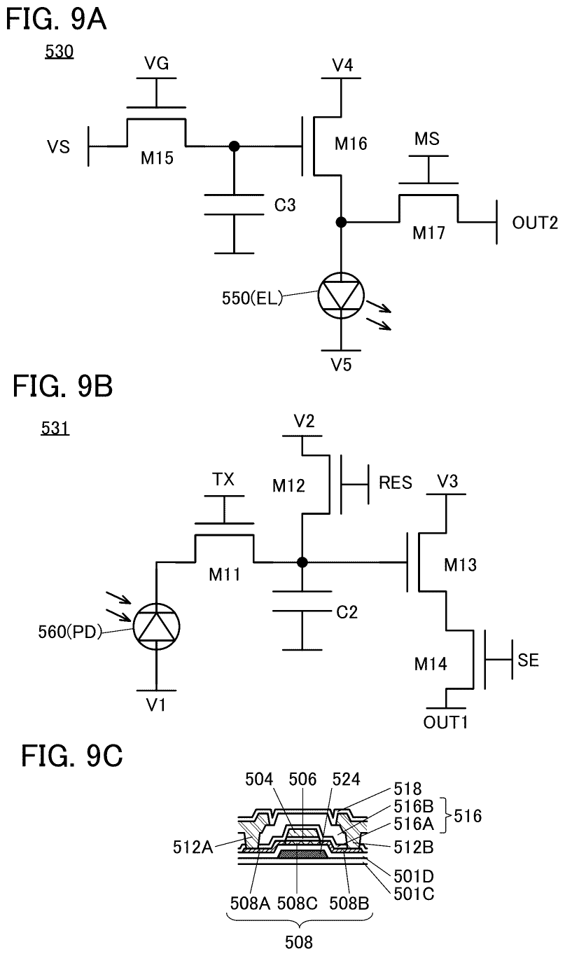

Next, an example of a pixel circuit of a subpixel including the light-emitting device is described with reference to FIG. 9A. A pixel circuit 530 illustrated in FIG. 9A includes a light-emitting device (EL) 550, a transistor M15, a transistor M16, a transistor M17, and a capacitor C3. Note that a light-emitting diode can be used as the light-emitting device 550. In particular, any of the light-emitting devices described in Embodiments 2 and 3 is preferably used as the light-emitting device 550.

In FIG. 9A, a gate of the transistor M15 is electrically connected to a wiring VG, one of a source and a drain of the transistor M15 is electrically connected to a wiring VS, and the other of the source and the drain of the transistor M15 is electrically connected to one electrode of the capacitor C3 and a gate of the transistor M16. One of a source and a drain of the transistor M16 is electrically connected to a wiring V4, and the other is electrically connected to an anode of the light-emitting device 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device 550 is electrically connected to a wiring V5.

A constant potential is supplied to the wiring V4 and the wiring V5. In the light-emitting device 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device 550 in accordance with a potential supplied to the gate of the transistor M16. When the transistor M15 is on, a potential supplied to the wiring VS is supplied to the gate of the transistor M16, and the luminance of the light-emitting device 550 can be controlled in accordance with the potential. The transistor M17 is controlled by a signal supplied to the wiring MS and has a function of outputting a potential between the transistor M16 and the light-emitting device 550 to the outside through the wiring OUT2.

Here, a transistor using a metal oxide (an oxide semiconductor) in a semiconductor layer where a channel is formed is preferably used as each of the transistors M15, M16, and M17 included in the pixel circuit 530 in FIG. 9A and each of transistors M11, M12, M13, and M14 included in a pixel circuit 531 in FIG. 9B.

A transistor using a metal oxide having a wider band gap and a lower carrier density than silicon can achieve an extremely low off-state current. Such a low off-state current enables retention of charges accumulated in a capacitor that is connected in series with the transistor for a long time. Therefore, it is particularly preferable to use a transistor including an oxide semiconductor as the transistors M11, M12, and M15 each of which is connected in series with a capacitor C2 or the capacitor C3. When each of the other transistors also includes an oxide semiconductor, manufacturing cost can be reduced.

Alternatively, transistors containing silicon as a semiconductor in which a channel is formed can be used as each of the transistors M11 to M17. It is particularly preferable to use silicon with high crystallinity such as single crystal silicon or polycrystalline silicon because high field-effect mobility can be achieved and higher-speed operation can be performed.

Alternatively, a transistor including an oxide semiconductor may be used as at least one of the transistors M11 to M17, and transistors containing silicon may be used as the other transistors.

Next, an example of a pixel circuit of a subpixel including a light-receiving device is described with reference to FIG. 9B. The pixel circuit 531 illustrated in FIG. 9B includes alight-receiving device (PD) 560, the transistor M11, the transistor M12, the transistor M13, the transistor M14, and the capacitor C2. In the example illustrated here, a photodiode is used as the light-receiving device (PD) 560.

In FIG. 9B, an anode of the light-receiving device (PD) 560 is electrically connected to a wiring V1, and a cathode of the light-receiving device (PD) 560 is electrically connected to one of a source and a drain of the transistor M11. A gate of the transistor M11 is electrically connected to a wiring TX, and the other of the source and the drain of the transistor M11 is electrically connected to one electrode of the capacitor C2, one of a source and a drain of the transistor M12, and a gate of the transistor M13. A gate of the transistor M12 is electrically connected to a wiring RES, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE, and the other of the source and the drain of the transistor M14 is electrically connected to a wiring OUT1.

A constant potential is supplied to the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring RES and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for outputting a signal corresponding to the potential of the node. The transistor M14 is controlled by a signal supplied to the wiring SE and functions as a selection transistor for reading an output corresponding to the potential of the node by an external circuit connected to the wiring OUT1.

Although n-channel transistors are illustrated in FIGS. 9A and 9B, p-channel transistors can alternatively be used.

The transistors included in the pixel circuit 530 and the transistors included in the pixel circuit 531 are preferably formed side by side over the same substrate. It is particularly preferable that the transistors included in the pixel circuit 530 and the transistors included in the pixel circuit 531 be periodically arranged in one region.

One or more layers including the transistor and/or the capacitor are preferably provided to overlap with the light-receiving device (PD) 560 or the light-emitting device (EL) 550. Thus, the effective area of each pixel circuit can be reduced, and a high-resolution light-receiving portion or display portion can be achieved.

FIG. 9C illustrates an example of a specific structure of a transistor that can be used in the pixel circuit described with reference to FIGS. 9A and 9B. As the transistor, a bottom-gate transistor, a top-gate transistor, or the like can be used as appropriate.

The transistor illustrated in FIG. 9C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other.

A conductive film 524 can be used in the transistor. The semiconductor film 508 is positioned between the conductive film 504 and a region included in the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For the insulating film 518, a material that has a function of inhibiting diffusion of oxygen, hydrogen (including deuterium), water, an alkali metal, an alkaline earth metal, and the like is preferably used. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film having the same composition as the semiconductor film used in the transistor of the pixel circuit can be used in the driver circuit, for example.

For the semiconductor film 508, a semiconductor containing a Group 14 element can be used. Specifically, a semiconductor containing silicon can be used for the semiconductor film 508.

Hydrogenated amorphous silicon can be used for the semiconductor film 508.

Microcrystalline silicon or the like can also be used for the semiconductor film 508. In such cases, it is possible to provide an apparatus having less display unevenness than an apparatus (including a light-emitting apparatus, a display panel, a display apparatus, and a light-emitting and light-receiving apparatus) using polysilicon for the semiconductor film 508, for example. Moreover, it is easy to increase the size of the apparatus.

Polysilicon can be used for the semiconductor film 508. In this case, for example, the field-effect mobility of the transistor can be higher than that of a transistor using hydrogenated amorphous silicon for the semiconductor film 508. For another example, the driving capability can be higher than that of a transistor using hydrogenated amorphous silicon for the semiconductor film 508. For another example, the aperture ratio of the pixel can be higher than that of the case of employing a transistor using hydrogenated amorphous silicon for the semiconductor film 508.

For another example, the reliability of the transistor can be higher than that of a transistor using hydrogenated amorphous silicon for the semiconductor film 508.

The temperature required for manufacturing the transistor can be lower than that required for a transistor using single crystal silicon, for example.

The semiconductor film used in the transistor of the driver circuit can be formed in the same step as the semiconductor film used in the transistor of the pixel circuit. The driver circuit can be formed over a substrate where the pixel circuit is formed. The number of components of an electronic appliance can be reduced.

Single crystal silicon can be used for the semiconductor film 508. In this case, for example, the resolution can be higher than that of a light-emitting apparatus (or a display panel) using hydrogenated amorphous silicon for the semiconductor film 508. For another example, it is possible to provide a light-emitting apparatus having less display unevenness than a light-emitting apparatus using polysilicon for the semiconductor film 508. For another example, smart glasses or a head-mounted display can be provided.

A metal oxide can be used for the semiconductor film 508. In this case, the pixel circuit can hold an image signal for a longer time than a pixel circuit including a transistor that uses amorphous silicon for the semiconductor film. Specifically, a selection signal can be supplied at a frequency of lower than 30 Hz, preferably lower than 1 Hz, further preferably less than once per minute while flickering is suppressed. Consequently, fatigue of a user of an electronic appliance can be reduced. Furthermore, power consumption for driving can be reduced.

An oxide semiconductor can be used for the semiconductor film 508. Specifically, an oxide semiconductor containing indium, an oxide semiconductor containing indium, gallium, and zinc, or an oxide semiconductor containing indium, gallium, zinc, and tin can be used for the semiconductor film 508.

The use of an oxide semiconductor for the semiconductor film achieves a transistor having lower leakage current in the off state than a transistor using amorphous silicon for the semiconductor film. Thus, a transistor using an oxide semiconductor for the semiconductor film is preferably used as a switch or the like. Note that a circuit in which a transistor using an oxide semiconductor for the semiconductor film is used as a switch is capable of retaining the potential of a floating node for a longer time than a circuit in which a transistor using amorphous silicon for the semiconductor film is used as a switch.

In the case of using an oxide semiconductor for a semiconductor film, the light-emitting and light-receiving apparatus 720 includes a light-emitting device including an oxide semiconductor in its semiconductor film and having a metal maskless (MML) structure. With this structure, the leakage current that might flow through the transistor and the leakage current that might flow between adjacent light-emitting devices (also referred to as a lateral leakage current, a side leakage current, or the like) can become extremely low. With the structure, a viewer can notice any one or more of the image crispness, the image sharpness, a high chroma, and a high contrast ratio in an image displayed on the display apparatus. When the leakage current that might flow through the transistor and the lateral leakage current that might flow between light-emitting devices are extremely low, display with little leakage of light at the time of black display (so-called black floating) (such display is also referred to as deep black display) can be achieved.

In particular, in the case where a light-emitting device having an MML structure employs the above-described SBS structure, a layer provided between light-emitting devices (for example, also referred to as an organic compound layer or a common layer which is commonly used between the light-emitting devices) is disconnected; accordingly, display with no or extremely small lateral leakage can be achieved.

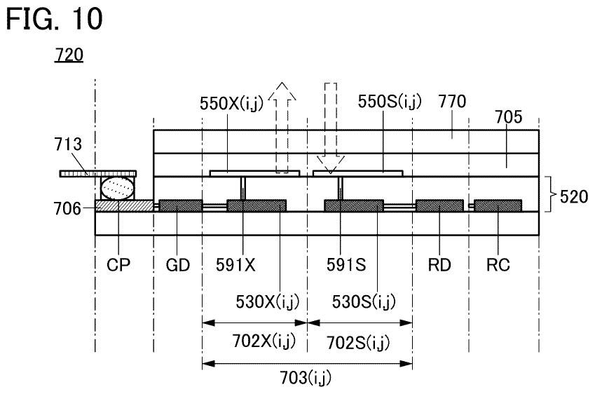

Next, a cross-sectional view of a light-emitting and light-receiving apparatus is shown. FIG. 10 is a cross-sectional view of the light-emitting and light-receiving apparatus illustrated in FIG. 8A.

FIG. 10 is a cross-sectional view of part of the display region 701 including the pixel 703(i, j) and part of a region including the FPC 713 and the wiring 706.

In FIG. 10, the light-emitting and light-receiving apparatus 720 includes the functional layer 520 between the first substrate 510 and the second substrate 770. The functional layer 520 includes, as well as the above-described transistors (M11, M12, M13, M14, M15, M16, and M17), the capacitor (C2 and C3), and the like described with reference to FIGS. 9A to 9C, the wirings (VS, VG, V1, V2, V3, V4, and V5) electrically connected to these components, for example. Although the functional layer 520 includes a pixel circuit 530X(i, j), a pixel circuit 530S(i, j), the circuit GD, a circuit RD, a circuit RC, and a conductor CP, in FIG. 10, one embodiment of the present invention is not limited thereto.

Furthermore, each pixel circuit (e.g., the pixel circuit 530X(i, j) and the pixel circuit 530S(i, j) in FIG. 10) included in the functional layer 520 is electrically connected to a light-emitting device and a light-receiving device (e.g., a light-emitting device 550X(i, j) and a light-receiving device 550S(i, j) in FIG. 10) formed over the functional layer 520. Specifically, the light-emitting device 550X(i, j) is electrically connected to the pixel circuit 530X(i, j) through a wiring 591X, and the light-receiving device 550S(i, j) is electrically connected to the pixel circuit 530S(i, j) through a wiring 591S. The insulating layer 705 is provided over the functional layer 520, the light-emitting devices, and the light-receiving device, and has a function of attaching the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light-receiving apparatus of one embodiment of the present invention can be used as a touch panel.

The structures described in this embodiment can be used in appropriate combination with any of the structures described in the other embodiments.

### Embodiment 7

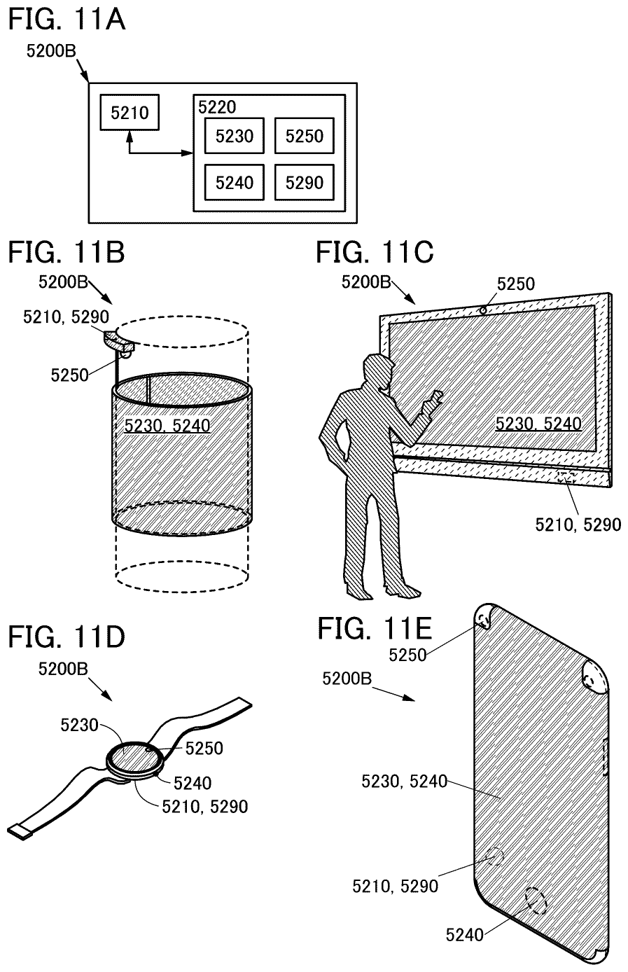

In this embodiment, structures of electronic appliances of one embodiment of the present invention will be described with reference to FIGS. 11 to 11E, FIGS. 12A to 12E, and FIGS. 13A and 13B. Note that the electronic appliances described in this embodiment can each include a light-emitting and light-receiving apparatus of one embodiment of the present invention.

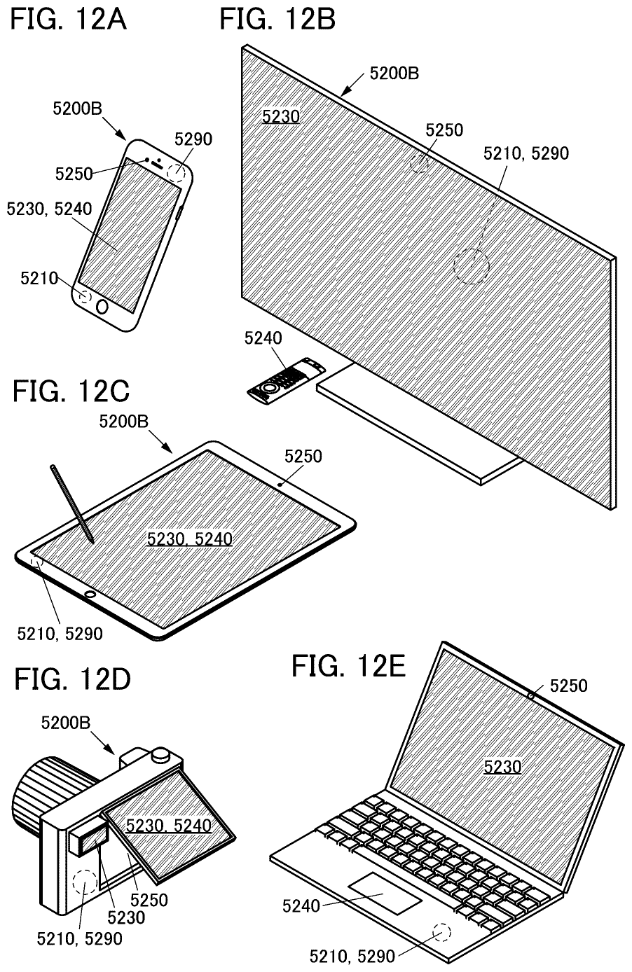

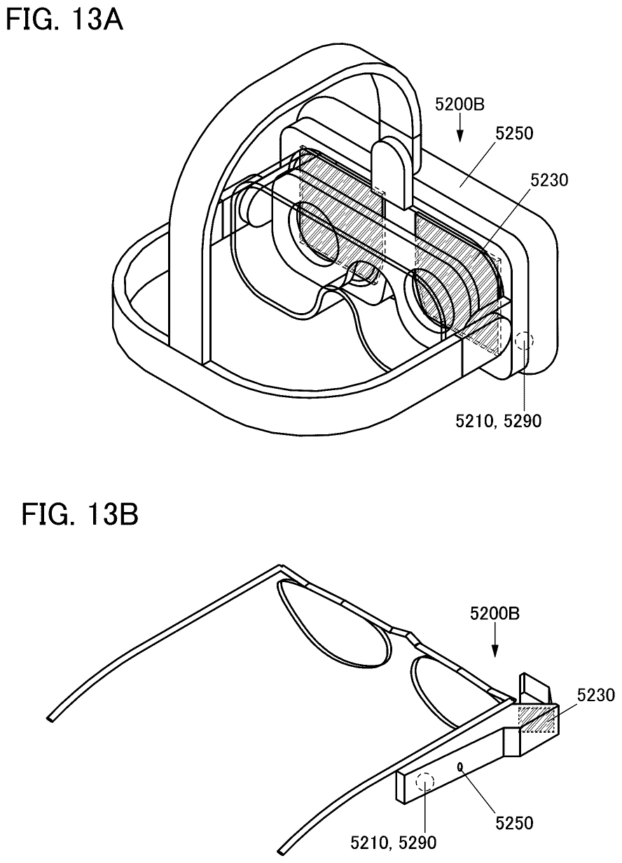

FIGS. 11 to 11E, FIGS. 12A to 12E, and FIGS. 13A and 13B each illustrate a structure of the electronic appliance of one embodiment of the present invention. FIG. 11A is a block diagram of the electronic appliance and FIGS. 11B to 11E are perspective views illustrating structures of the electronic appliance. FIGS. 12A to 12E are perspective views illustrating structures of the electronic appliance. FIGS. 13A and 13B are perspective views illustrating structures of the electronic appliance.

An electronic appliance 5200B described in this embodiment includes an arithmetic device 5210 and an input/output device 5220 (see FIG. 11A).

The arithmetic device 5210 has a function of receiving handling data and a function of supplying image data on the basis of the handling data.

The input/output device 5220 includes a display unit 5230, an input unit 5240, a sensor unit 5250, and a communication unit 5290, and has a function of supplying handling data and a function of receiving image data. The input/output device 5220 also has a function of supplying sensing data, a function of supplying communication data, and a function of receiving communication data.

The input unit 5240 has a function of supplying handling data. For example, the input unit 5240 supplies handling data on the basis of handling by a user of the electronic appliance 5200B. Specifically, a keyboard, a hardware button, a pointing device, a touch sensor, an illuminance sensor, an imaging device, an audio input device, an eye-gaze input device, an attitude sensing device, or the like can be used as the input unit 5240.

The display unit 5230 includes a display panel and has a function of displaying image data. For example, the display panel described in Embodiment 3 can be used for the display unit 5230.

The sensor unit 5250 has a function of supplying sensing data. For example, the sensor unit 5250 has a function of sensing a surrounding environment where the electronic appliance is used and supplying the sensing data.

Specifically, an illuminance sensor, an imaging device, an attitude sensing device, a pressure sensor, a human motion sensor, or the like can be used as the sensor unit 5250.

The communication unit 5290 has a function of receiving and supplying communication data. For example, the communication unit 5290 has a function of being connected to another electronic appliance or a communication network by wireless communication or wired communication. Specifically, the communication unit 5290 has a function of wireless local area network communication, telephone communication, near field communication, or the like.

FIG. 11B illustrates an electronic appliance having an outer shape along a cylindrical column or the like. An example of such an electronic appliance is digital signage. The display panel of one embodiment of the present invention can be used for the display unit 5230. The electronic appliance may have a function of changing its display method in accordance with the illuminance of a usage environment. The electronic appliance has a function of changing the displayed content when sensing the existence of a person. Thus, for example, the electronic appliance can be provided on a column of a building. The electronic appliance can display advertising, guidance, or the like. The electronic appliance can be used for digital signage or the like.

FIG. 11C illustrates an electronic appliance having a function of generating image data on the basis of the path of a pointer used by the user. Examples of such an electronic appliance include an electronic blackboard, an electronic bulletin board, and digital signage. Specifically, a display panel with a diagonal size of 20 inches or longer, preferably 40 inches or longer, further preferably 55 inches or longer can be used. A plurality of display panels can be arranged and used as one display region. Alternatively, a plurality of display panels can be arranged and used as a multiscreen.

FIG. 11D illustrates an electronic appliance that is capable of receiving data from another device and displaying the data on the display unit 5230. An example of such an electronic appliance is a wearable electronic appliance. Specifically, the electronic appliance can display several options, and the user can choose some from the options and send a reply to the data transmitter. As another example, the electronic appliance has a function of changing its display method in accordance with the illuminance of a usage environment. Thus, for example, power consumption of the wearable electronic appliance can be reduced. As another example, the wearable electronic appliance can display an image so as to be suitably used even in an environment under strong external light, e.g., outdoors in fine weather.

FIG. 11E illustrates an electronic appliance including the display unit 5230 having a surface gently curved along a side surface of a housing. An example of such an electronic appliance is a mobile phone. The display unit 5230 includes a display panel having a function of displaying images on the front surface, the side surfaces, the top surface, and the rear surface, for example. Thus, a mobile phone can display data on not only its front surface but also its side surfaces, top surface, and rear surface, for example.

FIG. 12A illustrates an electronic appliance that is capable of receiving data via the Internet and displaying the data on the display unit 5230. An example of such an electronic appliance is a smartphone. For example, the user can check a created message on the display unit 5230 and send the created message to another device. As another example, the electronic appliance has a function of changing its display method in accordance with the illuminance of a usage environment. Thus, power consumption of the smartphone can be reduced. As another example, it is possible to obtain a smartphone which can display an image such that the smartphone can be suitably used in an environment under strong external light, e.g., outdoors in fine weather.

FIG. 12B illustrates an electronic appliance that can use a remote controller as the input unit 5240. An example of such an electronic appliance is a television system. For example, data received from a broadcast station or via the Internet can be displayed on the display unit 5230. An image of a user can be taken using the sensor unit 5250. The image of the user can be transmitted. The electronic appliance can acquire a viewing history of the user and provide it to a cloud service. The electronic appliance can acquire recommendation data from a cloud service and display the data on the display unit 5230. A program or a moving image can be displayed on the basis of the recommendation data. As another example, the electronic appliance has a function of changing its display method in accordance with the illuminance of a usage environment. Accordingly, the television system can display an image so as to be suitably used even under strong external light entering the room from the outside in fine weather.

FIG. 12C illustrates an electronic appliance that is capable of receiving educational materials via the Internet and displaying them on the display unit 5230. An example of such an electronic appliance is a tablet computer. The user can input an assignment with the input unit 5240 and send it via the Internet. The user can obtain a corrected assignment or the evaluation from a cloud service and have it displayed on the display unit 5230. The user can select a suitable educational material on the basis of the evaluation and have it displayed.

For example, an image signal can be received from another electronic appliance and displayed on the display unit 5230. When the electronic appliance is placed on a stand or the like, the display unit 5230 can be used as a sub-display. Thus, for example, the tablet computer can display an image so as to be suitably used even in an environment under strong external light, e.g., outdoors in fine weather.

FIG. 12D illustrates an electronic appliance including a plurality of display units 5230. An example of such an electronic appliance is a digital camera. For example, the display unit 5230 can display an image that the sensor unit 5250 is capturing. A captured image can be displayed on the sensor unit. A captured image can be decorated using the input unit 5240. A message can be attached to a captured image. A captured image can be transmitted via the Internet. The electronic appliance has a function of changing shooting conditions in accordance with the illuminance of a usage environment. Accordingly, for example, the digital camera can display a subject such that an image is suitably viewed even in an environment under strong external light, e.g., outdoors in fine weather.

FIG. 12E illustrates an electronic appliance in which the electronic appliance of this embodiment is used as a master to control another electronic appliance used as a slave. An example of such an electronic appliance is a portable personal computer. For example, part of image data can be displayed on the display unit 5230 and another part of the image data can be displayed on a display unit of another electronic appliance. Image signals can be supplied. Data written from an input unit of another electronic appliance can be obtained with the communication unit 5290. Thus, a large display region can be utilized in the case of using a portable personal computer, for example.

FIG. 13A illustrates an electronic appliance including the sensor unit 5250 that senses an acceleration or a direction. An example of such an electronic appliance is a goggles-type electronic appliance. The sensor unit 5250 can supply data on the position of the user or the direction in which the user faces. The electronic appliance can generate image data for the right eye and image data for the left eye in accordance with the position of the user or the direction in which the user faces. The display unit 5230 includes a display region for the right eye and a display region for the left eye. Thus, a virtual reality image that gives the user a sense of immersion can be displayed on the goggles-type electronic appliance, for example.

FIG. 13B illustrates an electronic appliance including an imaging device and the sensor unit 5250 that senses an acceleration or a direction. An example of such an electronic appliance is a glasses-type electronic appliance. The sensor unit 5250 can supply data on the position of the user or the direction in which the user faces. The electronic appliance can generate image data in accordance with the position of the user or the direction in which the user faces. Accordingly, the data can be shown together with areal-world scene, for example. Alternatively, an augmented reality image can be displayed on the glasses-type electronic appliance.

This embodiment can be combined with any of the other embodiments in this specification as appropriate.

### Example 1

### Synthesis Example 1

In this example, a method for synthesizing N,N-bis(biphenyl-4-yl)-4′-(benzo[b]naphtho[2,1-d]furan-10-yl)-3′-(9H-carbazol-9-yl)biphenyl-4-amine (abbreviation: aBnf-YGBBi1BP), which is represented by Structural Formula (100) in Embodiment 1, is specifically described. The structure of aBnf-YGBBi1BP is shown below.

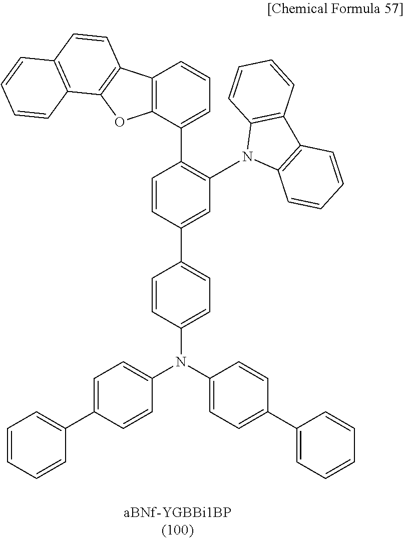 [SMILES: c1ccc(-c2ccc(N(c3ccc(-c4ccccc4)cc3)c3ccc(-c4ccc(-c5cccc6c5oc5c7ccccc7ccc65)c(-n5c6ccccc6c6ccccc65)c4)cc3)cc2)cc1]

### Step 1: Synthesis of 9-[5-chloro-2-(benzo[b]naphtho[2,1-d]furan-10-yl)phenyl]-9H-carbazole

Into a 100-mL three-neck flask equipped with a reflux pipe were put 5.0 g (14 mmol) of 9-(2-bromo-5-chlorophenyl)-9H-carbazole, 5.2 g (15 mmol) of 10-(4,4,5,5-tetramethyl-1,3,2-dioxaborolane-2-yl)benzo[b]naphtho[2,1-d]furan, 0.17 g (0.56 mmol) of tri(o-tolyl)phosphine, 3.9 g (28 mmol) of potassium carbonate, 60 mL of toluene, 12 mL of ethanol, and 14 mL of water, the mixture was degassed under reduced pressure, and then the air in the three-neck flask was replaced with nitrogen. To this mixture heated at 60° C. was added 62 mg (0.28 mmol) of palladium(II) acetate, and the obtained mixture was stirred while being heated at 90° C. for 10 hours. After the stirring, water was added to the three-neck flask, the precipitated solid was collected by suction filtration, and the obtained solid was washed with toluene, ethanol, and water. The obtained solid was dissolved in heated toluene, followed by filtration through Celite (Catalog No. 537-02305, FUJIFILM Wako Pure Chemical Co., Ltd.) and alumina, whereby 1.3 g of a solid 1A was obtained. In addition, water was added to the filtrate obtained by the suction filtration, and an aqueous layer was subjected to extraction with toluene. The solution of the extract was washed with water twice, and further washed with saturated saline. Magnesium sulfate was added to the mixture for drying, and magnesium sulfate was removed by gravity filtration. The filtrate obtained by the gravity filtration was concentrated under reduced pressure, and the obtained solid was recrystallized with toluene and hexane to give 2.9 g of a solid 1B. The solid 1A and the solid 1B were combined and purified by silica gel chromatography to give 3.6 g of a target white solid in a yield of 52%. Note that as a developing solvent of silica gel column chromatography, hexane was used at first and a mixed solvent of hexane:toluene=4:1 was used from the middle. Synthesis scheme (a-1) of Step 1 is shown below.

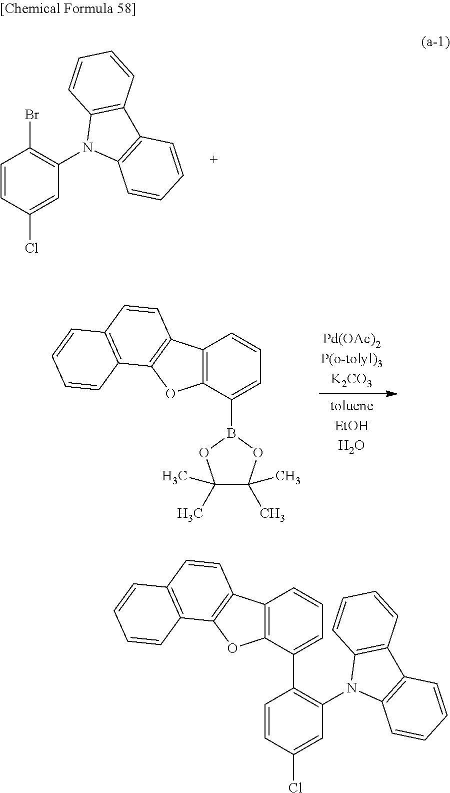 [SMILES: Clc1ccc(Br)c(-n2c3ccccc3c3ccccc32)c1 ; CC1(C)OB(c2cccc3c2oc2c4ccccc4ccc32)OC1(C)C ; Clc1ccc(-c2cccc3c2oc2c4ccccc4ccc32)c(-n2c3ccccc3c3ccccc32)c1]

The molecular weight of the white solid obtained in Step 1 was measured by LC/MS. As a result, m/z 494 (indicating a proton adduct of the target object) was observed while the calculated mass of the target substance was 493. This reveals that 9-[5-chloro-2-(benzo[b]naphtho[2,1-d]furan-10-yl)phenyl]-9H-carbazole was obtained.

Note that the solid obtained in Step 1 was analyzed by proton nuclear magnetic resonance measurements (¹H NMR). Given below are ¹H NMR numerical data of the resulting solid.

¹H NMR (chloroform-d, 500 MHz): δ=7.98-7.96 (m, 2H), 7.93-7.91 (m, 1H), 7.87 (d, J=8.0 Hz, 2H), 7.83 (d, J=8.6 Hz, 1H), 7.72-7.68 (m, 4H), 7.54-7.52 (m, 2H), 7.21-7.18 (m, 4H), 7.09 (td, J=7.5 Hz, 1.2 Hz, 2H), 7.00-6.98 (m, 2H).

### Step 2: Synthesis of N,N′-bis(biphenyl-4-yl)-4′-(benzo[b]naphtho[2,1-d]furan-10-yl)-3′-(9H-carbazol-9-yl)biphenyl-4-amine (abbreviation: aBnf-YGBBi1BP)

Into a 100-mL three-neck flask equipped with a reflux pipe were put 3.5 g (7.1 mmol) of 9-[5-chloro-2-(benzo[b]naphtho[2,1-d]furan-10-yl)phenyl]-9H-carbazole, 3.7 g (7.1 mmol) of N,N-di(4-biphenylyl)-4-(4,4,5,5-tetramethyl-1,3,2-dioxaborolan-2-yl)aniline, 50 mg (0.14 mmol) of di(1-adamantyl)-n-butylphosphine, 4.5 g (21 mmol) of tripotassium phosphate, 40 mL of diethylene glycol dimethyl ether, and 1.6 g (21 mmol) of tert-butyl alcohol, the mixture was degassed under reduced pressure, and then the air in the three-neck flask was replaced with nitrogen. To this mixture heated at 60° C. was added 16 mg (71 μmol) of palladium(II) acetate, and the obtained mixture was stirred while being heated at 90° C. for 10 hours. After the stirring, water was added to the three-neck flask, the precipitated solid was collected by suction filtration, and the obtained solid was washed with toluene, ethanol, and water. The obtained solid was dissolved in heated toluene, followed by filtration through Celite (Catalog No. 537-02305, FUJIFILM Wako Pure Chemical Co., Ltd.) and alumina, whereby 3.4 g of a solid was obtained. The obtained solid was purified by high performance liquid chromatography (mobile phase: chloroform) to give 3.0 g of a target white solid in a yield of 49%. Synthesis Scheme (a-2) of Step 2 is shown below.

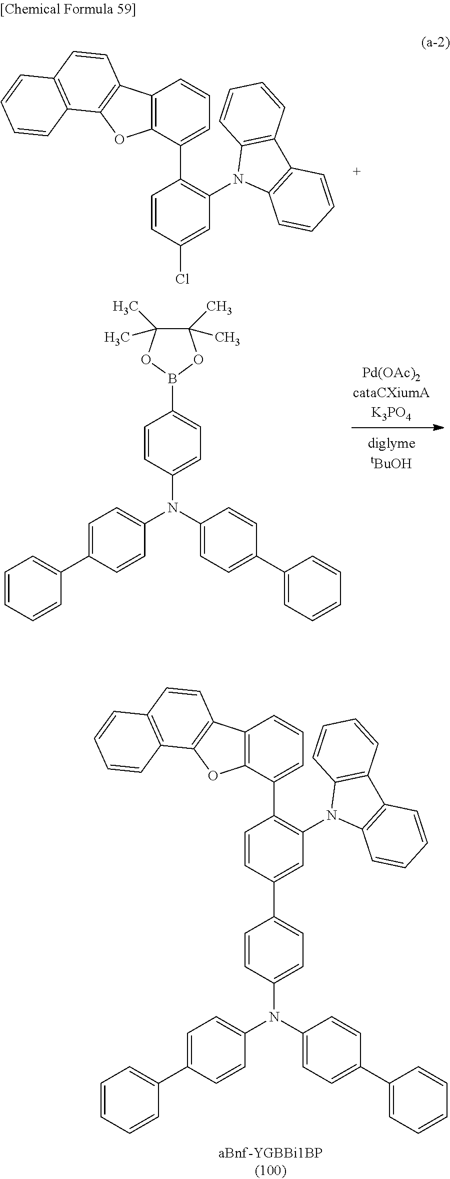

Then, 2.4 g of the obtained solid was purified by sublimation using a train sublimation method. In the purification by sublimation, the solid was heated at 365° C. to 355° C. under a pressure of 2.6 Pa with an argon flow rate of 10 mL/min for 25 hours, and a solid precipitated at 270° C. was collected. As a result, 1.9 g of a pale yellow solid was obtained at a collection rate of 79%.

The molecular weight of the pale yellow solid obtained in Step 2 was measured by LC/MS. As a result, m/z 855 was observed while the calculated mass of the target substance was 855. This reveals that N,N′-bis(biphenyl-4-yl)-4′-(benzo[b]naptho[2,1-d]furan-10-yl)-3′-(9H-carbazol-9-yl)biphenyl-4-amine (abbreviation: aBnf-YGBBi1BP) was obtained.

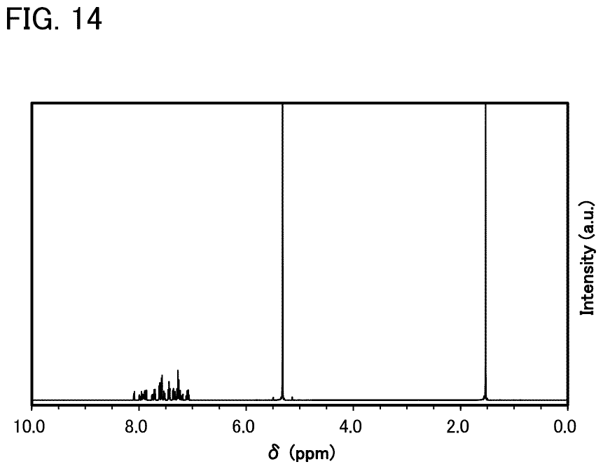

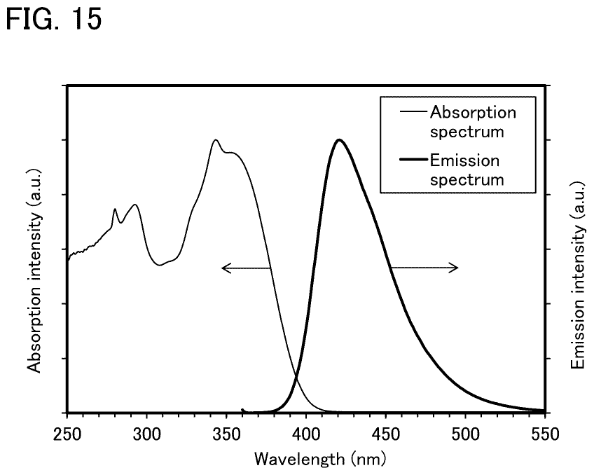

Note that the pale yellow solid obtained in the Step 2 was analyzed by proton nuclear magnetic resonance measurements (¹H NMR). The obtained values are shown below. FIG. 14 is a ¹H NMR chart. Accordingly, it was found that aBnf-YGBBi1BP represented by Structural Formula (100) described above was obtained in this synthesis example.

¹H NMR (dichloromethane-d₂, 500 MHz): δ=8.09 (d, J=8.0 Hz, 1H), 7.99 (dd, J=8.0 Hz, 1.7 Hz, 1H), 7.95-7.86 (m, 6H), 7.75 (dd, J=7.5 Hz, 1.2 Hz, 1H), 7.72-7.70 (m, 3H), 7.63-7.61 (m, 4H), 7.57 (d, J=8.6 Hz, 4H), 7.54-7.52 (m, 2H), 7.44 (t, J=7.5 Hz, 4H), 7.36 (d, J=8.0 Hz, 2H), 7.34-7.31 (m, 2H), 7.29-7.18 (m, 9H), 7.12-7.07 (m, 3H).

### <Measurement of Physical Properties>

The physical property values of aBnf-YGBBi1BP obtained in this synthesis example were measured.

### [Spectrum Measurement]

First, the ultraviolet-visible absorption spectra (hereinafter, simply referred to as “absorption spectra”) and photoluminescence (PL) spectra of a toluene solution and a thin film of aBnf-YGBBi1BP were measured.

The absorption spectrum of the solution was measured with an ultraviolet-visible spectrophotometer (V-770DS, JASCO Corporation), and the absorption spectrum of the thin film was measured with an ultraviolet-visible spectrophotometer (U-4100, Hitachi-High-Tech Corporation). The PL spectrum was measured with a fluorescence spectrophotometer (FP-8600DS, JASCO Corporation).

To calculate the absorption spectrum of aBnf-YGBBi1BP, the absorption spectrum of toluene put in a quartz cell was measured and then subtracted from the absorption spectrum of the toluene solution of aBnf-YGBBi1BP put in a quartz cell.

To obtain the absorption spectrum and the PL spectrum of the thin film, a measurement sample was measured. The measurement sample was fabricated in the following manner: aBnf-YGBBi1BP was formed over a quartz substrate by a vacuum evaporation method and sealed using another quartz substrate as a counter substrate. Note that the PL spectrum was obtained by measuring the sealed sample, and the absorption spectrum was obtained by measuring the sample from which the sealing was removed and the counter substrate was detached. The absorption spectrum was obtained by subtraction of the absorption spectrum of the quartz substrate from the absorption spectrum of aBnf-YGBBi1BP formed over the quartz substrate.

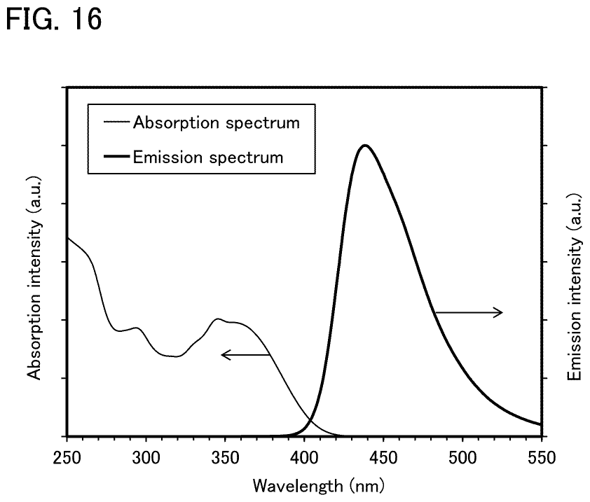

FIG. 15 and FIG. 16 show the measurement results of the toluene solution and the thin film, respectively. The measurement results show that the toluene solution of aBnf-YGBBi1BP has an absorption peak at around 358 nm, the thin film of aBnf-YGBBi1BP has an absorption peak at around 355 nm, and there is no absorption band on a longer wavelength side than 430 nm in both cases of the toluene solution and the thin film. The results suggest that the absorption does not reduce the emission efficiency at the wavelength employed for display, showing the suitability of aBnf-YGBBi1BP for a light-emitting element. In addition, the toluene solution of aBnf-YGBBi1BP has an emission wavelength peak at around 421 nm (excitation wavelength: 355 nm), and the thin film of aBnf-YGBBi1BP has an emission wavelength peak at around 438 nm (excitation wavelength: 345 nm).

### [Cyclic Voltammetry Measurement]

Next, the HOMO level and the LUMO level of aBnf-YGBBi1BP were obtained through a cyclic voltammetry (CV) measurement. The calculation method is described below.

An electrochemical analyzer (ALS model 600A or 600C, BAS Inc.) was used as a measurement apparatus. To prepare a solution for the CV measurement, dehydrated dimethylformamide (DMF) (Sigma-Aldrich Inc., 99.8%, catalog No. 22705-6) was used as a solvent, and tetra-n-butylammonium perchlorate (n-Bu₄NClO₄) (Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, BAS Inc.) was used as a working electrode, another platinum electrode (Pt counter electrode for VC-3 (5 cm), BAS Inc.) was used as an auxiliary electrode, and an Ag/Ag⁺ electrode (RE7 reference electrode for nonaqueous solvent, BAS Inc.) was used as a reference electrode. Note that the measurement was performed at room temperature (higher than or equal to 20° C. and lower than or equal to 25° C.). The scan speed in the CV measurement was fixed to 0.1 V/sec, and an oxidation potential Eₐ [V] and a reduction potential E, [V] with respect to the reference electrode were measured. The potential Eₐ is an intermediate potential of an oxidation-reduction wave, and the potential E, is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Eₐ and LUMO level [eV]=−4.94−E_{c}.

The CV measurement was repeated 100 times, and the oxidation-reduction wave in the 100th cycle was compared with the oxidation-reduction wave in the first cycle to examine the electrical stability of the compound.

As a result, in the measurement of the oxidation potential Eₐ [V] of aBnf-YGBBi1BP, the HOMO level was found to be −5.49 eV. The LUMO level was found to be −2.38 eV in the measurement of the reduction potential E_{c} [V]. Comparison of the waveforms in the first cycle and the 100th cycle in repeated measurements of the oxidation-reduction wave shows that the peak intensity in the 100th cycle of the Eₐ measurement was maintained at 92% of that in the first cycle and the peak intensity in the 100th cycle of the E_{c} measurement was maintained at 90% of that in the first cycle. These results revealed that aBnf-YGBBi1BP is highly resistant to repeated oxidation and reduction.

### [Differential Scanning Calorimetry Measurement]

Differential scanning calorimetry (DSC) measurement of aBnf-YGBBi1BP was performed with DSC8500 produced by PerkinElmer, Inc. The temperature was raised from −10° C. to 340° C. at a temperature rising rate of 40° C./min and held for three minutes; then, the temperature was lowered to −10° C. at a temperature falling rate of 100° C./min and held for three minutes. This operation was performed twice in succession. The DSC measurement results of the second operation shows that the glass transition point of aBnf-YGBBi1BP is 168° C. This indicates that aBnf-YGBBi1BP is a substance having extremely high heat resistance and the film of aBnf-YGBBi1BP can maintain a thermally stable quality.

### [Thermogravimetry-Differential Thermal Analysis]

The thermogravimetry-differential thermal analysis (TG-DTA) was performed on aBnf-YGBBi1BP. For the measurement, a high-sensitivity differential type differential thermogravimeter (STA-2500 Regulus, NETZSCH Japan K.K.) was used. The measurement was performed under an atmospheric pressure at a temperature rising rate of 10° C./min under a nitrogen stream (flow rate: 200 mL/min). In the thermogravimetry-differential thermal analysis, the temperature (decomposition temperature) at which the weight obtained by thermogravimetry was reduced by 5% of the weight at the beginning of the measurement was found to be higher than or equal to 500° C., which shows that aBnf-YGBBi1BP is a substance having extremely high heat resistance. Note that in this example, the weight of aBnf-YGBBi1BP used for the thermogravimetry-differential thermal analysis was 3 mg.

### Example 2

### Synthesis Example 2

In this example, a method for synthesizing N-(biphenyl-4-yl)-4′-(benzo[b]naphtho[2,1-d]furan-10-yl)-3′-(9H-carbazol-9-yl)-N-(9,9-dimethyl-9H-fluoren-2-yl)biphenyl-4-amine (abbreviation: aBnf-YGBBiF), which is represented by Structural Formula (101) in Embodiment 1, is specifically described. The structure of aBnf-YGBBiF is shown below.

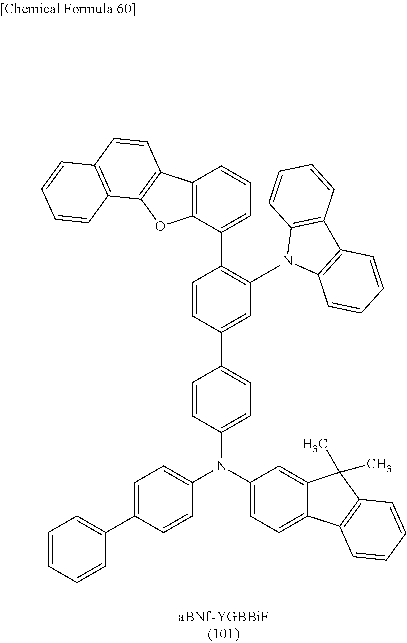 [SMILES: CC1(C)c2ccccc2-c2ccc(N(c3ccc(-c4ccccc4)cc3)c3ccc(-c4ccc(-c5cccc6c5oc5c7ccccc7ccc65)c(-n5c6ccccc6c6ccccc65)c4)cc3)cc21]

### Step 1: Synthesis of N-(biphenyl-4-yl)-4′-(benzo[b]naphtho[2,1-d]furan-10-yl)-3′-(9H-carbazol-9-yl)-N-(9,9-dimethyl-9H-fluoren-2-yl)biphenyl-4-amine (abbreviation: aBnf-YGBBiF)

Into a 200-mL three-neck flask equipped with a reflux pipe were put 2.6 g (5.3 mmol) of 9-[5-chloro-2-(benzo[b]naptho[2,1-d]furan-10-yl)phenyl]-9H-carbazole, 3.0 g (5.3 mmol) of N-(biphenyl- 4-yl)-9,9-dimethyl-N-[4-(4,4,5,5-tetramethyl-1,3,2-dioxaborolane-2-yl)phenyl]-9H-fluoren-2-amine, 88 mg (0.24 mmol) of di(1-adamantyl)-n-butylphosphine, 2.6 g (24 mmol) of tripotassium phosphate, 30 mL of diethylene glycol dimethyl ether, and 0.90 g (12 mmol) of tert-butyl alcohol, the obtained mixture was degassed under reduced pressure, and the air in the three-neck flask was replaced with nitrogen. To this mixture heated at 60° C. was added 27 mg (0.12 mmol) of palladium(II) acetate, and the obtained mixture was stirred while being heated at 130° C. for 14 hours. After the stirring, water was added to the three-neck flask, and an aqueous layer was subjected to extraction with toluene. The solution of the extract was washed with water twice, and further washed with saturated saline. Magnesium sulfate was added to the mixture for drying, and magnesium sulfate was removed by gravity filtration. The filtrate obtained by the gravity filtration was concentrated under reduced pressure, and the obtained solid was purified by silica gel chromatography. Note that as a developing solvent of silica gel column chromatography, hexane was used at first and a mixed solvent of hexane:toluene=3:2 was used from the middle. The obtained solid was recrystallized with toluene, ethanol, and hexane to give 1.7 g of a target white solid in a yield of 30%. Synthesis scheme (b-1) of Step 1 is shown below.

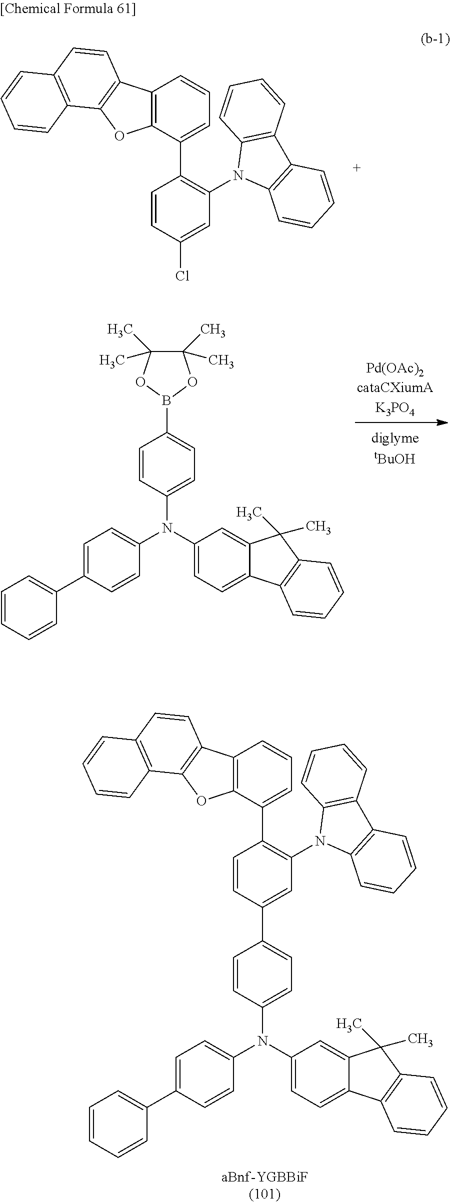 [SMILES: Clc1ccc(-c2cccc3c2oc2c4ccccc4ccc32)c(-n2c3ccccc3c3ccccc32)c1 ; CC1(C)c2ccccc2-c2ccc(N(c3ccc(B4OC(C)(C)C(C)(C)O4)cc3)c3ccc(-c4ccccc4)cc3)cc21 ; CC1(C)c2ccccc2-c2ccc(N(c3ccc(-c4ccccc4)cc3)c3ccc(-c4ccc(-c5cccc6c5oc5c7ccccc7ccc65)c(-n5c6ccccc6c6ccccc65)c4)cc3)cc21]

Then, 1.4 g of the obtained solid was purified by sublimation using a train sublimation method. In the purification by sublimation, the solid was heated at 365° C. to 350° C. under a pressure of 2.7 Pa with an argon flow rate of 7 mL/min for 19 hours, and a solid precipitated at 270° C. was collected. As a result, 1.1 g of a yellow solid was obtained at a collection rate of 80%.

The molecular weight of the yellow solid obtained in Step 1 was measured by LC/MS. As a result, m/z 895 was observed while the calculated mass of the target substance was 895. This reveals that N-(biphenyl-4-yl)-4′-(benzo[b]naphtho[2,1-d]furan-10-yl)-3′-(9H-carbazol-9-yl)-N-(9,9-dimethyl-9H-fluoren-2-yl)biphenyl-4-amine (abbreviation: aBnf-YGBBiF) was obtained.

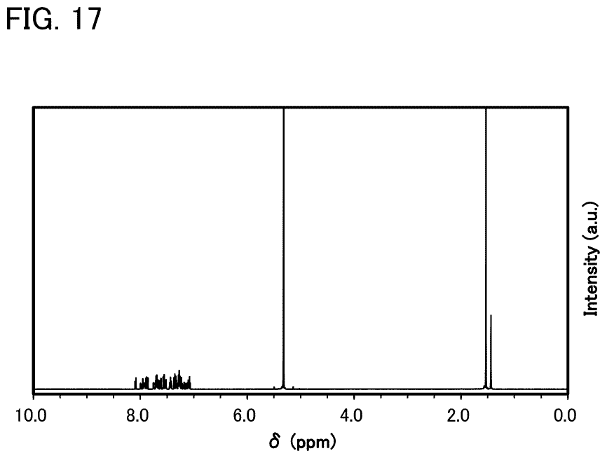

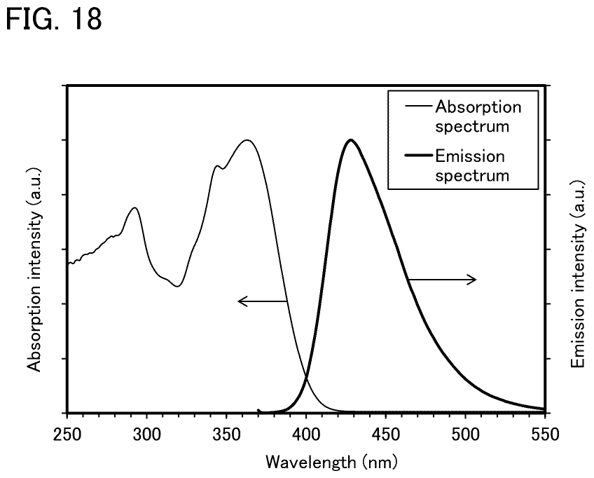

Note that the yellow solid obtained in the Step 1 was analyzed by proton nuclear magnetic resonance measurements (¹H NMR). The obtained values are shown below. FIG. 17 is a ¹H NMR chart. Accordingly, it was found that aBnf-YGBBiF represented by Structural Formula (101) described above was obtained in this synthesis example.

¹H NMR (dichloromethane-d₂, 500 MHz): δ=8.09 (d, J=8.0 Hz, 1H), 7.99 (dd, J=8.0 Hz, 2.3 Hz, 1H), 7.95-7.86 (m, 6H), 7.75 (dd, J=7.5 Hz, 1.2 Hz, 1H), 7.71-7.66 (m, 5H), 7.63-7.61 (m, 2H), 7.56 (d, J=8.6 Hz, 2H), 7.54-7.51 (m, 2H), 7.45-7.41 (m, 3H), 7.37-7.31 (m, 5H), 7.29-7.18 (m, 8H), 7.15-7.13 (m, 1H), 7.12-7.07 (m, 3H), 1.44 (s, 6H).

### <Measurement of Physical Properties>

The physical property values of aBnf-YGBBiF obtained in this synthesis example were measured.

### [Spectrum Measurement]

First, the ultraviolet-visible absorption spectra (hereinafter, simply referred to as “absorption spectra”) and photoluminescence (PL) spectra of a toluene solution and a thin film of aBnf-YGBBiF were measured.

The absorption spectrum of the solution was measured with an ultraviolet-visible spectrophotometer (V-770DS, JASCO Corporation), and the absorption spectrum of the thin film was measured with an ultraviolet-visible spectrophotometer (U-4100, Hitachi-High-Tech Corporation). The PL spectrum was measured with a fluorescence spectrophotometer (FP-8600DS, JASCO Corporation).

To calculate the absorption spectrum of aBnf-YGBBiF, the absorption spectrum of toluene put in a quartz cell was measured and then subtracted from the absorption spectrum of aBnf-YGBBiF put in a quartz cell.

To obtain the absorption spectrum and the PL spectrum of the thin film, a measurement sample was measured. The measurement sample was fabricated in the following manner: aBnf-YGBBiF was formed over a quartz substrate by a vacuum evaporation method and sealed using another quartz substrate as a counter substrate. Note that the PL spectrum was obtained by measuring the sealed sample, and the absorption spectrum was obtained by measuring the sample from which the sealing was removed and the counter substrate was detached. The absorption spectrum was obtained by subtraction of the absorption spectrum of the quartz substrate from the absorption spectrum of aBnf-YGBBiF formed over the quartz substrate.

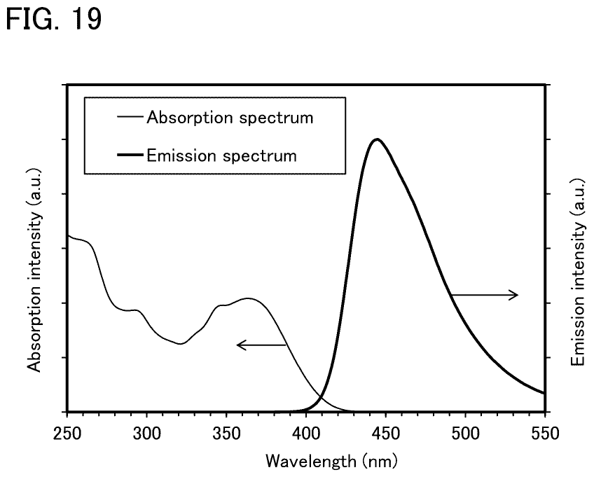

FIG. 18 and FIG. 19 show the measurement results of the toluene solution and the thin film, respectively. The measurement results show that the toluene solution of aBnf-YGBBiF has an absorption peak at around 363 nm, the thin film of aBnf-YGBBiF has an absorption peak at around 364 nm, and there is no absorption band on a longer wavelength side than 430 nm in both cases of the toluene solution and the thin film. The results suggest that the absorption does not reduce the emission efficiency at the wavelength employed for display, showing the suitability of aBnf-YGBBiF for a light-emitting element. In addition, the toluene solution of aBnf-YGBBiF has an emission wavelength peak at around 428 nm (excitation wavelength: 365 nm), and the thin film of aBnf-YGBBiF has an emission wavelength peak at around 445 nm (excitation wavelength: 365 nm).

### [Cyclic Voltammetry Measurement]

Next, the HOMO level and the LUMO level of aBnf-YGBBiF were obtained through a cyclic voltammetry (CV) measurement. The calculation method is described below.

An electrochemical analyzer (ALS model 600A or 600C, BAS Inc.) was used as a measurement apparatus. To prepare a solution for the CV measurement, dehydrated dimethylformamide (DMF) (Sigma-Aldrich Inc., 99.8%, catalog No. 22705-6) was used as a solvent, and tetra-n-butylammonium perchlorate (n-Bu₄NClO₄) (Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, BAS Inc.) was used as a working electrode, another platinum electrode (Pt counter electrode for VC-3 (5 cm), BAS Inc.) was used as an auxiliary electrode, and an Ag/Ag⁺ electrode (RE7 reference electrode for nonaqueous solvent, BAS Inc.) was used as a reference electrode. Note that the measurement was performed at room temperature (higher than or equal to 20° C. and lower than or equal to 25° C.). The scan speed in the CV measurement was fixed to 0.1 V/sec, and an oxidation potential Eₐ [V] and a reduction potential E_{c} [V] with respect to the reference electrode were measured. The potential Eₐ is an intermediate potential of an oxidation-reduction wave, and the potential E_{c} is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Eₐ and LUMO level [eV]=−4.94−E_{c}.

The CV measurement was repeated 100 times, and the oxidation-reduction wave in the 100th cycle was compared with the oxidation-reduction wave in the first cycle to examine the electrical stability of the compound.

As a result, in the measurement of the oxidation potential Eₐ [V] of aBnf-YGBBiF, the HOMO level was found to be −5.42 eV. The LUMO level was found to be −2.38 eV in the measurement of the reduction potential E_{c} [V]. Comparison of the waveforms in the first cycle and the 100th cycle in repeated measurements of the oxidation-reduction wave shows that the peak intensity in the 100th cycle of the Eₐ measurement was maintained at 91% of that in the first cycle and the peak intensity in the 100th cycle of the E_{c} measurement was maintained at 99% of that in the first cycle. These results revealed that aBnf-YGBBiF is highly resistant to repeated oxidation and reduction.

### [Differential Scanning Calorimetry Measurement]

Differential scanning calorimetry (DSC) measurement of aBnf-YGBBiF was performed with DSC8500 produced by PerkinElmer, Inc. The temperature was raised from −10° C. to 340° C. at a temperature rising rate of 40° C./min and held for three minutes; then, the temperature was lowered to −10° C. at a temperature falling rate of 100° C./min and held for three minutes. This operation was performed twice in succession. The DSC measurement results of the second operation shows that the glass transition point of aBnf-YGBBiF is 170° C. This indicates that aBnf-YGBBiF is a substance having extremely high heat resistance and the film of aBnf-YGBBiF can maintain a thermally stable quality.

### [Thermogravimetry-Differential Thermal Analysis]

The thermogravimetry-differential thermal analysis (TG-DTA) was performed on aBnf-YGBBiF. For the measurement, a high-sensitivity differential type differential thermogravimeter (STA-2500 Regulus, NETZSCH Japan K.K.) was used. The measurement was performed under an atmospheric pressure at a temperature rising rate of 10° C./min under a nitrogen stream (flow rate: 200 mL/min). In the thermogravimetry-differential thermal analysis, the temperature (decomposition temperature) at which the weight obtained by thermogravimetry was reduced by 5% of the weight at the beginning of the measurement was found to be higher than or equal to 500° C., which shows that aBnf-YGBBiF is a substance having extremely high heat resistance. Note that in this example, the weight of aBnf-YGBBiF used for the thermogravimetry-differential thermal analysis was 3 mg.

### Example 3

In this example, a light-emitting device 1A of one embodiment of the present invention was fabricated. A light-emitting device 1B for comparison was also fabricated and their characteristics were compared.

The structural formulae of organic compounds used for the light-emitting devices 1A and 1B are shown below.

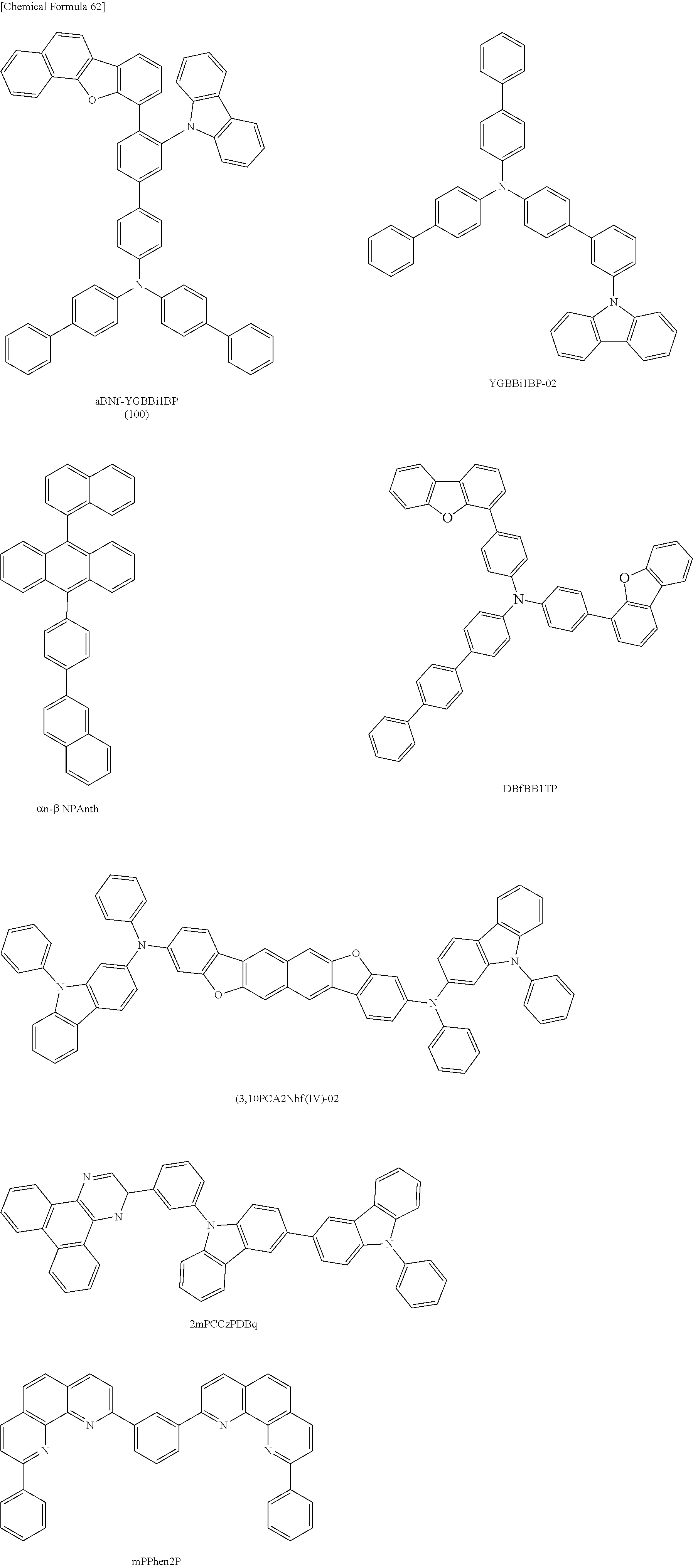

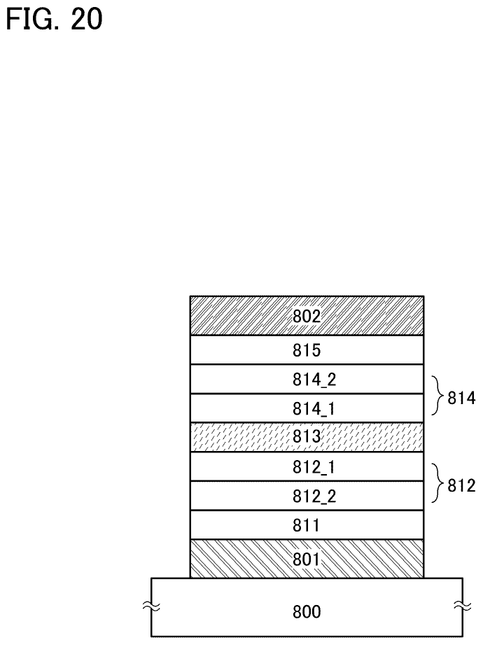

In the devices, as illustrated in FIG. 20, a hole-injection layer 811, a hole-transport layer 812, a light-emitting layer 813, an electron-transport layer 814, and an electron-injection layer 815 are stacked in this order over a first electrode 801 formed over a glass substrate 800, and a second electrode 802 is stacked over the electron-injection layer 815.

### <Method for Fabricating Light-Emitting Device 1A>

As the first electrode 801, a film of indium tin oxide containing silicon oxide (ITSO) was formed to a thickness of 110 nm over the glass substrate 800 by a sputtering method. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for 45 minutes.

Then, the substrate provided with the first electrode 801 was fixed to a substrate holder provided in the vacuum evaporation apparatus such that the surface on which the first electrode 801 was formed faced downward. Over the first electrode 801, N,N′-bis(biphenyl-4-yl)-4′-(benzo[b]naptho[2,1-d]furan-10-yl)-3′-(9H-carbazol-9-yl)biphenyl-4-amine (abbreviation: aBnf-YGBBi1BP) and a fluorine-containing electron acceptor material with a molecular weight of 672 (OCHD-003) were deposited by co-evaporation to a thickness of 10 nm such that the weight ratio of aBnf-YGBBi1BP to OCHD-003 was 1:0.10, whereby the hole-injection layer 811 was formed.

Next, over the hole-injection layer 811, aBnf-YGBBi1BP was deposited by evaporation using resistance heating to a thickness of 90 nm as a hole-transport layer 812_2, and then N,N-bis[4-(dibenzofuran-4-yl)phenyl]-4-amino-p-terphenyl (abbreviation: DBfBB1TP) was deposited by evaporation using resistance heating to a thickness of 10 nm as a hole-transport layer 812_1, whereby the hole-transport layer 812 was formed.

Next, over the hole-transport layer 812, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited by co-evaporation using resistance heating to a thickness of 25 nm such that the weight ratio of αN-βNPAnth to 3,10PCA2Nbf(IV)-02 was 1:0.015, whereby the light-emitting layer 813 was formed.

Next, over the light-emitting layer 813, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm as an electron-transport layer 814_1. Then, 2,2′-(1,3-phenylene)bis(9-phenyl-1,10-phenanthroline) (abbreviation: mPPhen2P) was deposited by evaporation to a thickness of 15 nm as an electron-transport layer 8142, whereby the electron-transport layer 814 was formed.

Next, over the electron-transport layer 814, lithium fluoride (LiF) was deposited by evaporation using resistance heating to a thickness of 1 nm, whereby the electron-injection layer 815 was formed.

Then, over the electron-injection layer 815, aluminum (Al) was deposited by evaporation to a thickness of 150 nm, whereby the second electrode 802 was formed.

### <Method for Fabricating Light-Emitting Device 1B>

A method for fabricating the light-emitting device 1B is described. The light-emitting device 1B is different from the light-emitting device 1A in the structures of the hole-injection layer 811 and the hole-transport layer 812_2. The other components were formed in a manner similar to that for the light-emitting device 1A.

Specifically, in the light-emitting device 1B, over the first electrode 801, N,N-bis(biphenyl-4-yl)-3′-(9H-carbazol-9-yl)biphenyl-4-amine (abbreviation: YGBBi1BP-02) and a fluorine-containing electron acceptor material (OCHD-003) with a molecular weight of 672 were deposited by co-evaporation to a thickness of 10 nm such that the weight ratio of YGBBi1BP-02 to OCHD-003 was 1:0.10, whereby the hole-injection layer 811 was formed.

Next, over the hole-injection layer 811, YGBBi1BP-02 was deposited by evaporation using resistance heating to a thickness of 90 nm as the hole-transport layer 8122, and then DBfBB1TP was deposited by evaporation using resistance heating to a thickness of 10 nm as the hole-transport layer 812_1, whereby the hole-transport layer 812 was formed.

The structures of the light-emitting device 1A and the light-emitting device 1B for comparison are listed in the following table.

TABLE 1

| | Thickness | | Light-emitting device 1B |
|---|---|---|---|
| | [nm] | Light-emitting device 1A | (comparison) |
| Second electrode 802 | 150 | A1 | |
| Electron-injection layer 815 | 1 | LiF | |
| Electron-transport layer 814_2 | 15 | mPPhen2P | |
| Electron-transport layer 814_1 | 10 | 2mPCCzPDBq | |
| Light-emitting layer 813 | 25 | αN-βNP Anth: 3,10PCA2Nbf(IV)-02 (1: 0.015) | |
| Hole-transport layer 812_1 | 10 | DBfBB1TP | |
| Hole-transport layer 812_2 | 90 | aBnf-YGBBi1BP | YGBBi1BP-02 |
| Hole-injection layer 811 | 10 | aBnf-YGBBi1BP: OCHD-003 | YGBBi1BP-02: OCHD-003 |
| | | (1: 0.10) | (1: 0.10) |

| | | |
|---|---|---|
| First electrode 801 | 110 | ITSO |

### <Characteristics of Light-Emitting Devices>

The light-emitting devices 1A and 1B were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

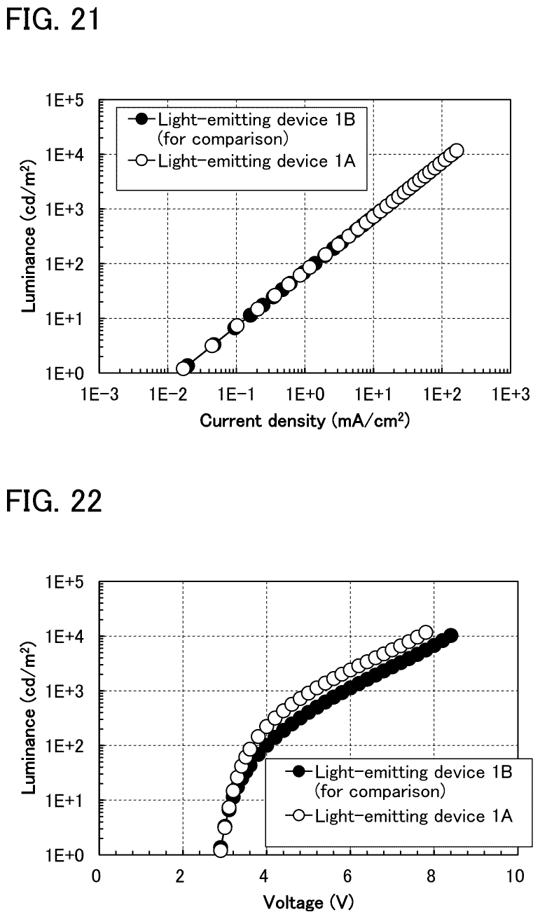

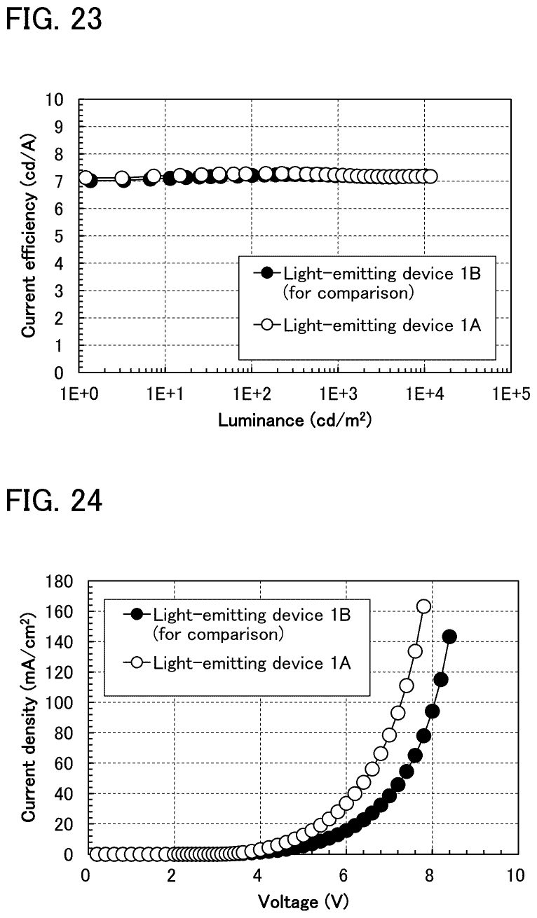

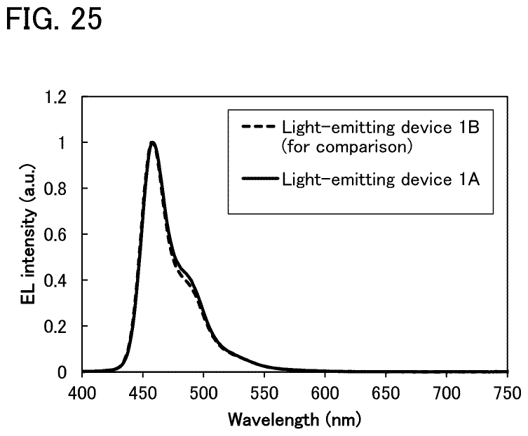

FIG. 21 shows the luminance-current density characteristics of the light-emitting devices 1A and 1B. FIG. 22 shows the luminance-voltage characteristics of the light-emitting devices 1A and 1B. FIG. 23 shows the current efficiency-luminance characteristics of the light-emitting devices 1A and 1B. FIG. 24 shows the current density-voltage characteristics of the light-emitting devices 1A and 1B. FIG. 25 shows the electroluminescence spectra of the light-emitting devices 1A and 1B.

The main characteristics of the devices at a luminance of approximately 1000 cd/m² are shown in the table below. Note that the luminance, CIE chromaticity, and emission spectra were measured with a spectroradiometer (SR-UL1R, TOPCON TECHNOHOUSE CORPORATION).

TABLE 2

| | | | | | | | | | External |
|---|---|---|---|---|---|---|---|---|---|
| | | | Current | | | | Current | Power | quantum |
| | Voltage | Current | density | Chromaticity | Chromaticity | Luminance | efficiency | efficiency | efficiency |
| | [V] | [mA] | [mA/cm²] | x | y | [cd/m²] | [cd/A] | [lm/W] | [%] |
| Light-emitting device 1A | 5.00 | 0.505 | 12.6 | 0.136 | 0.110 | 912 | 7.23 | 4.54 | 7.59 |
| Light-emitting device 1B | 5.60 | 0.424 | 10.6 | 0.137 | 0.107 | 766 | 7.22 | 4.05 | 7.70 |

The above table and FIG. 21 to FIG. 25 show that the light-emitting device 1A is a favorable light-emitting device. Specifically, the light-emitting device 1A is found to be driven at a lower voltage than the light-emitting device 1B. This is probably because benzonaphthofuran included in aBnf-YGBBi1BP used for the light-emitting device 1A has an excellent hole-transport property, which increases the proportion of holes reaching the inside of the light-emitting layer and enables low-voltage driving.

### <Results of Reliability Test>

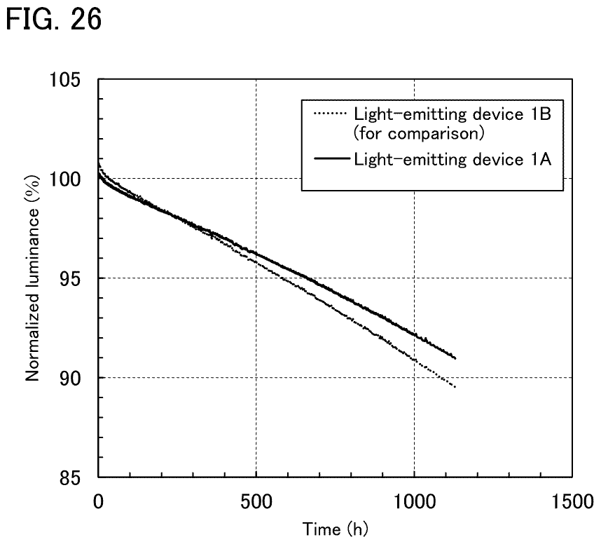

A reliability test was performed on the light-emitting devices 1A and 1B. FIG. 26 shows a time-dependent change in normalized luminance at the time of constant current density driving (50 [mA/cm²]). In FIG. 26, the vertical axis represents the luminance (%) normalized with the luminance at the time of the start of emission as 100%, and the horizontal axis represents time (h).

LT95 (h), which is the time that has elapsed until the measured luminance decreases to 95% of the initial luminance, of the light-emitting device 1A was 660 hours. LT95 (h) of the light-emitting device 1B for comparison was 579 hours. This shows that LT95 of the light-emitting device 1A is more than or equal to 1.13 times that of the light-emitting device 1B for comparison.

Since benzonaphthofuran included in aBnf-YGBBi1BP used for the light-emitting device 1A has an excellent hole-transport property, more holes can be transported to the light-emitting layer and to the electron-transport layer side. This probably leads to less generation of unnecessary excitons at the interface between the hole-transport layer and the light-emitting layer, thereby improving the overall device reliability.

Thus, it was confirmed that the light-emitting device of one embodiment of the present invention has favorable characteristics and improved reliability.

### Example 4

In this example, a light-emitting device 2A of one embodiment of the present invention was fabricated. A light-emitting device 2B for comparison was also fabricated and their characteristics were compared.

Structural formulae of organic compounds used for the light-emitting devices 2A and 2B are shown below.

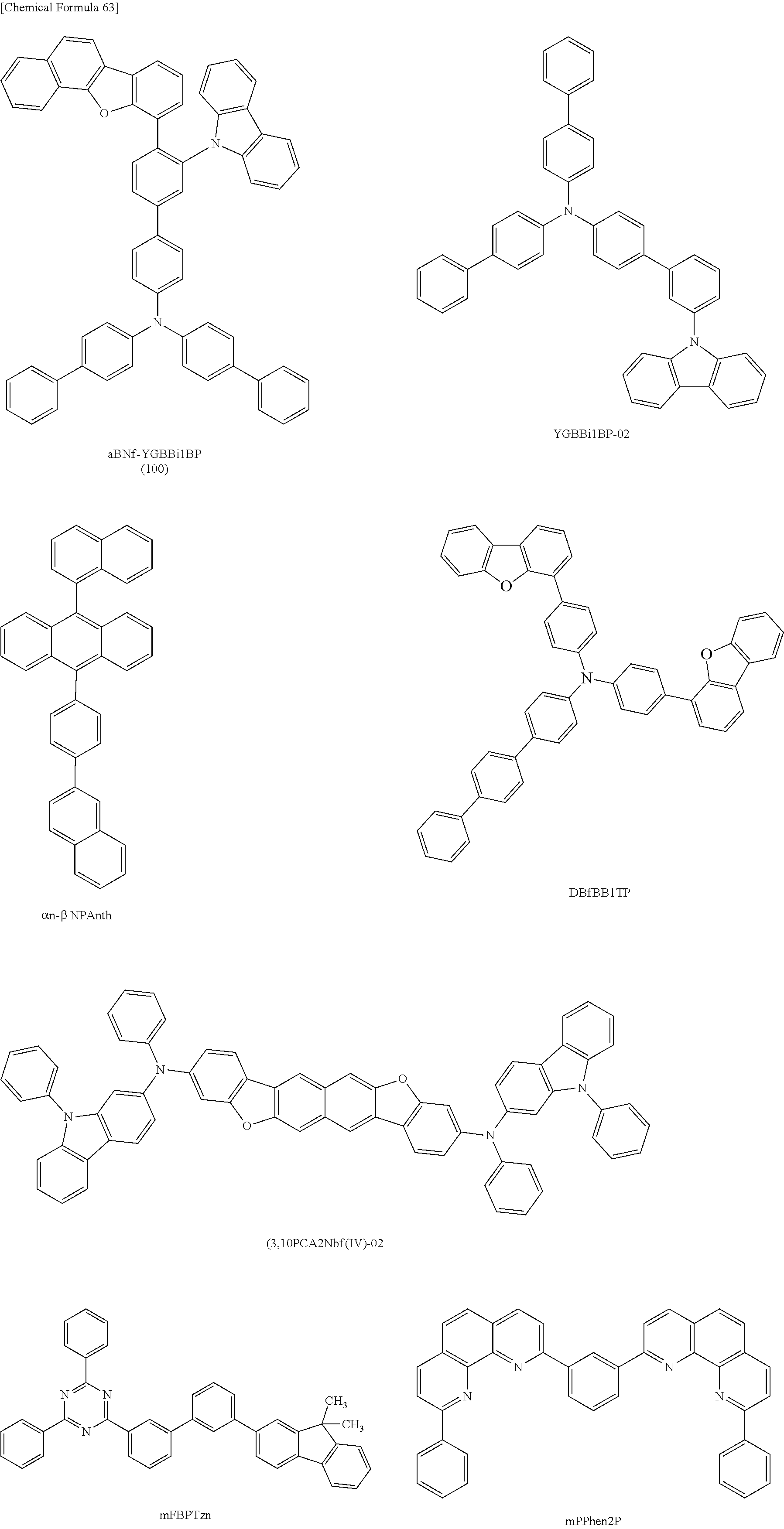

In the devices, as illustrated in FIG. 20, the hole-injection layer 811, the hole-transport layer 812, the light-emitting layer 813, the electron-transport layer 814, and the electron-injection layer 815 are stacked in this order over the first electrode 801 formed over the glass substrate 800, and the second electrode 802 is stacked over the electron-injection layer 815.

### <Method for Fabricating Light-Emitting Device 2A>

As the first electrode 801, a film of indium tin oxide containing silicon oxide (ITSO) was formed to a thickness of 110 nm over the glass substrate 800 by a sputtering method. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for 45 minutes.

Then, the substrate provided with the first electrode 801 was fixed to a substrate holder provided in the vacuum evaporation apparatus such that the surface on which the first electrode 801 was formed faced downward. Over the first electrode 801, N,N-bis(biphenyl-4-yl)-4′-(benzo[b]naptho[2,1-d]furan-10-yl)-3′-(9H-carbazol-9-yl)biphenyl-4-amine (abbreviation: aBnf-YGBBi1BP) and a fluorine-containing electron acceptor material with a molecular weight of 672 (OCHD-003) were deposited by co-evaporation to a thickness of 10 nm such that the weight ratio of aBnf-YGBBi1BP to OCHD-003 was 1:0.10, whereby the hole-injection layer 811 was formed.

Next, over the hole-injection layer 811, aBnf-YGBBi1BP was deposited by evaporation using resistance heating to a thickness of 90 nm as the hole-transport layer 8122, and then N,N-bis[4-(dibenzofuran-4-yl)phenyl]-4-amino-p-terphenyl (abbreviation: DBfBB1TP) was deposited by evaporation using resistance heating to a thickness of 10 nm as the hole-transport layer 812_1, whereby the hole-transport layer 812 was formed.

Next, over the hole-transport layer 812, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited by co-evaporation using resistance heating to a thickness of 25 nm such that the weight ratio of αN-[3NPAnth to 3,10PCA2Nbf(IV)-02 was 1:0.015, whereby the light-emitting layer 813 was formed.

Next, over the light-emitting layer 813, 2-[3′-(9,9-dimethyl-9H-fluoren-2-yl)biphenyl-3-yl]-4,6-diphenyl-1,3,5-triazine (abbreviation: mFBPTzn) was deposited by evaporation to a thickness of 10 nm as the electron-transport layer 814_1. Then, 2,2′-(1,3-phenylene)bis(9-phenyl-1,10-phenanthroline) (abbreviation: mPPhen2P) was deposited by evaporation to a thickness of 15 nm as the electron-transport layer 814_2, whereby the electron-transport layer 814 was formed.

Next, over the electron-transport layer 814, lithium fluoride (LiF) was deposited by evaporation using resistance heating to a thickness of 1 nm, whereby the electron-injection layer 815 was formed.

Then, over the electron-injection layer 815, aluminum (Al) was deposited by evaporation to a thickness of 150 nm, whereby the second electrode 802 was formed.

### <Method for Fabricating Light-Emitting Device 2B>

A method for fabricating the light-emitting device 2B is described. The light-emitting device 2B is different from the light-emitting device 2A in the structures of the hole-injection layer 811 and the hole-transport layer 812_2. The other components were formed in a manner similar to that for the light-emitting device 2A.

Specifically, in the light-emitting device 2B, over the first electrode 801, N,N-bis(biphenyl-4-yl)-3′-(9H-carbazol-9-yl)biphenyl-4-amine (abbreviation: YGBBi1BP-02) and a fluorine-containing electron acceptor material (OCHD-003) with a molecular weight of 672 were deposited by co-evaporation to a thickness of 10 nm such that the weight ratio of YGBBi1BP-02 to OCHD-003 was 1:0.10, whereby the hole-injection layer 811 was formed.

Next, over the hole-injection layer 811, YGBBi1BP-02 was deposited by evaporation using resistance heating to a thickness of 90 nm as the hole-transport layer 8122, and then DBfBB1TP was deposited by evaporation using resistance heating to a thickness of 10 nm as the hole-transport layer 812_1, whereby the hole-transport layer 812 was formed.

The structures of the light-emitting device 2A and the light-emitting device 2B for comparison are listed in the following table.

TABLE 3

| | Thickness | | Light-emitting device 2B |
|---|---|---|---|
| | [nm] | Light-emitting device 2A | (comparison) |
| Second electrode 802 | 150 | A1 | |
| Electron-injection layer 815 | 1 | LiF | |
| Electron-transport layer 814_2 | 15 | mPPhen2P | |
| Electron-transport layer 814_1 | 10 | mFBPTzn | |
| Light-emitting layer 813 | 25 | αN-βNPAnth: 3,10PCA2Nbf(IV)-02 (1: 0.015) | |
| Hole-transport layer 812_1 | 10 | DBfBB1TP | |
| Hole-transport layer 812_2 | 90 | aBnf-YGBBi1BP | YGBBi1BP-02 |
| Hole-injection layer 811 | 10 | aBnf-YGBBi1BP: OCHD-003 | YGBBi1BP-02: OCHD-003 |
| | | (1:0.10) | (1: 0.10) |

| | | |
|---|---|---|
| First electrode 801 | 110 | ITSO |

### <Characteristics of Light-Emitting Devices>

The light-emitting devices 2A and 2B were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

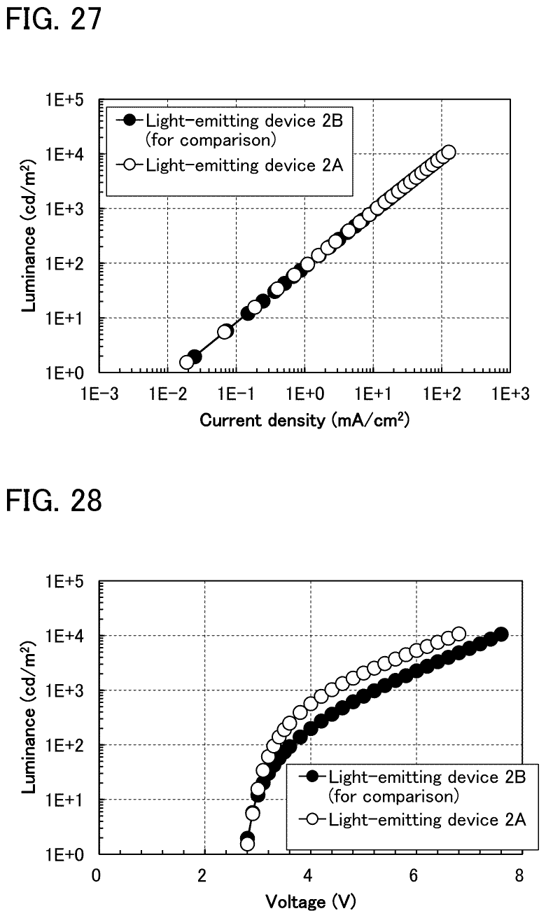

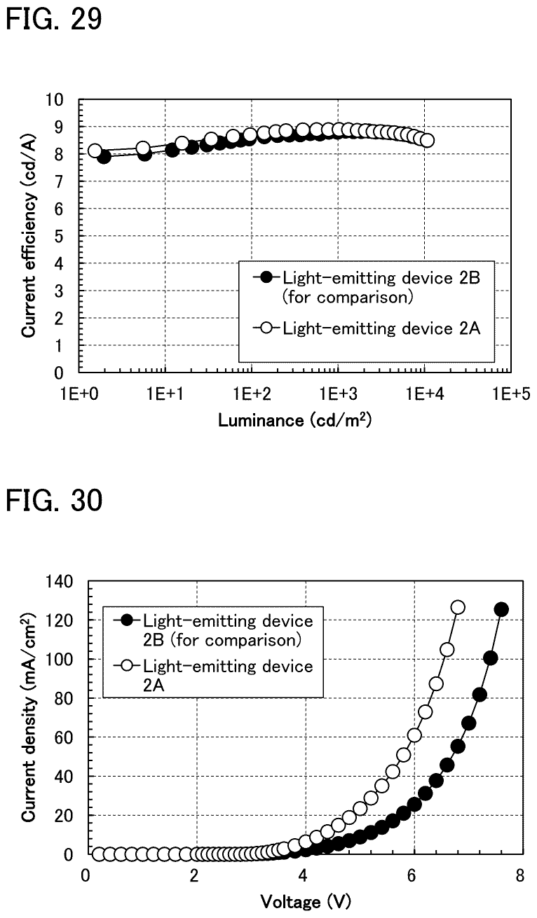

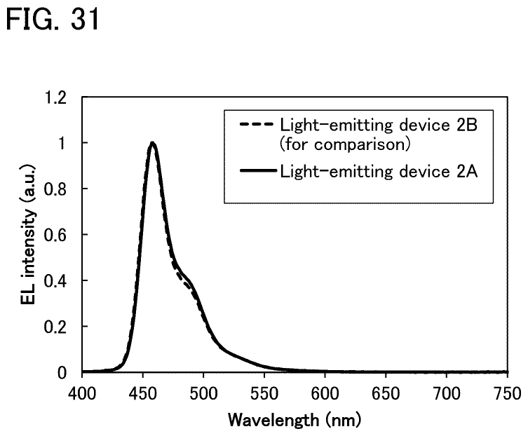

FIG. 27 shows the luminance-current density characteristics of the light-emitting devices 2A and 2B. FIG. 28 shows the luminance-voltage characteristics of the light-emitting devices 2A and 2B. FIG. 29 shows the current efficiency-luminance characteristics of the light-emitting devices 2A and 2B. FIG. 30 shows the current density-voltage characteristics of the light-emitting devices 2A and 2B. FIG. 31 shows the electroluminescence spectra of the light-emitting devices 2A and 2B.

The main characteristics of the devices at a luminance of approximately 1000 cd/m² are shown in the table below. Note that the luminance, CIE chromaticity, and emission spectra were measured with a spectroradiometer (SR-UL1R, TOPCON TECHNOHOUSE CORPORATION).

TABLE 4

| | | | | | | | | | External |
|---|---|---|---|---|---|---|---|---|---|
| | | | Current | | | | Current | Power | quantum |
| | Voltage | Current | density | Chromaticity | Chromaticity | Luminance | efficiency | efficiency | efficiency |
| | [V] | [mA] | [mA/cm²] | x | y | [cd/m²] | [cd/A] | [lm/W] | [%] |
| Light-emitting device 2A | 4.40 | 0.461 | 11.5 | 0.137 | 0.108 | 1025 | 8.89 | 6.35 | 9.46 |
| Light-emitting device 2B | 5.20 | 0.447 | 11.2 | 0.138 | 0.105 | 982 | 8.79 | 5.31 | 9.46 |

The above table and FIG. 27 to FIG. 31 show that the light-emitting device 2A is a favorable light-emitting device. Specifically, the light-emitting device 2A is found to be driven at a lower voltage than the light-emitting device 2B. This is probably because benzonaphthofuran included in aBnf-YGBBi1BP used for the light-emitting device 2A has an excellent hole-transport property, which increases the proportion of holes reaching the inside of the light-emitting layer and enables low-voltage driving.

### <Results of Reliability Test>

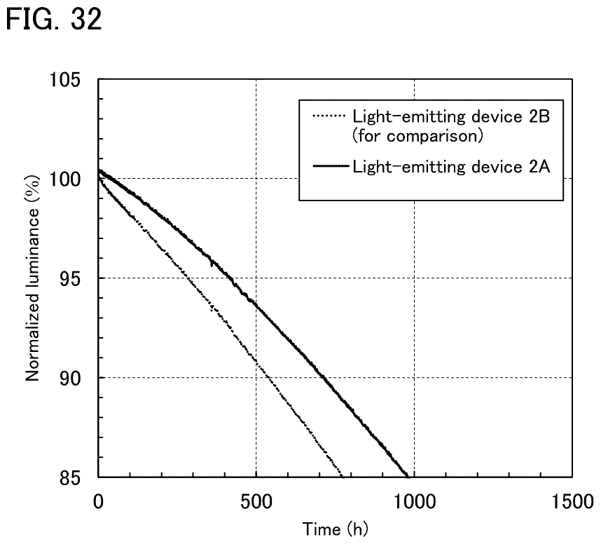

A reliability test was performed on the light-emitting devices 2A and 2B. FIG. 32 shows a time-dependent change in normalized luminance at the time of constant current density driving (50 [mA/cm²]). In FIG. 32, the vertical axis represents the luminance (%) normalized with the luminance at the time of the start of emission as 100%, and the horizontal axis represents time (h).

LT95 (h), which is the time that has elapsed until the measured luminance decreases to 95% of the initial luminance, of the light-emitting device 2A was 416 hours. LT95 (h) of the light-emitting device 2B for comparison was 283 hours. This shows that LT95 of the light-emitting device 2A is more than or equal to 1.4 times that of the light-emitting device 2B for comparison.

Since benzonaphthofuran included in aBnf-YGBBi1BP used for the light-emitting device 2A has an excellent hole-transport property, more holes can be transported to the light-emitting layer and to the electron-transport layer side. This probably leads to less generation of unnecessary excitons at the interface between the hole-transport layer and the light-emitting layer, thereby improving the overall device reliability.

Thus, it was confirmed that the light-emitting device of one embodiment of the present invention has favorable characteristics and improved reliability.

### Example 5

In this example, a light-emitting device 3A of one embodiment of the present invention was fabricated. A light-emitting device 3B for comparison was also fabricated and their characteristics were compared.

The structural formulae of organic compounds used for the light-emitting devices 3A and 3B are shown below.

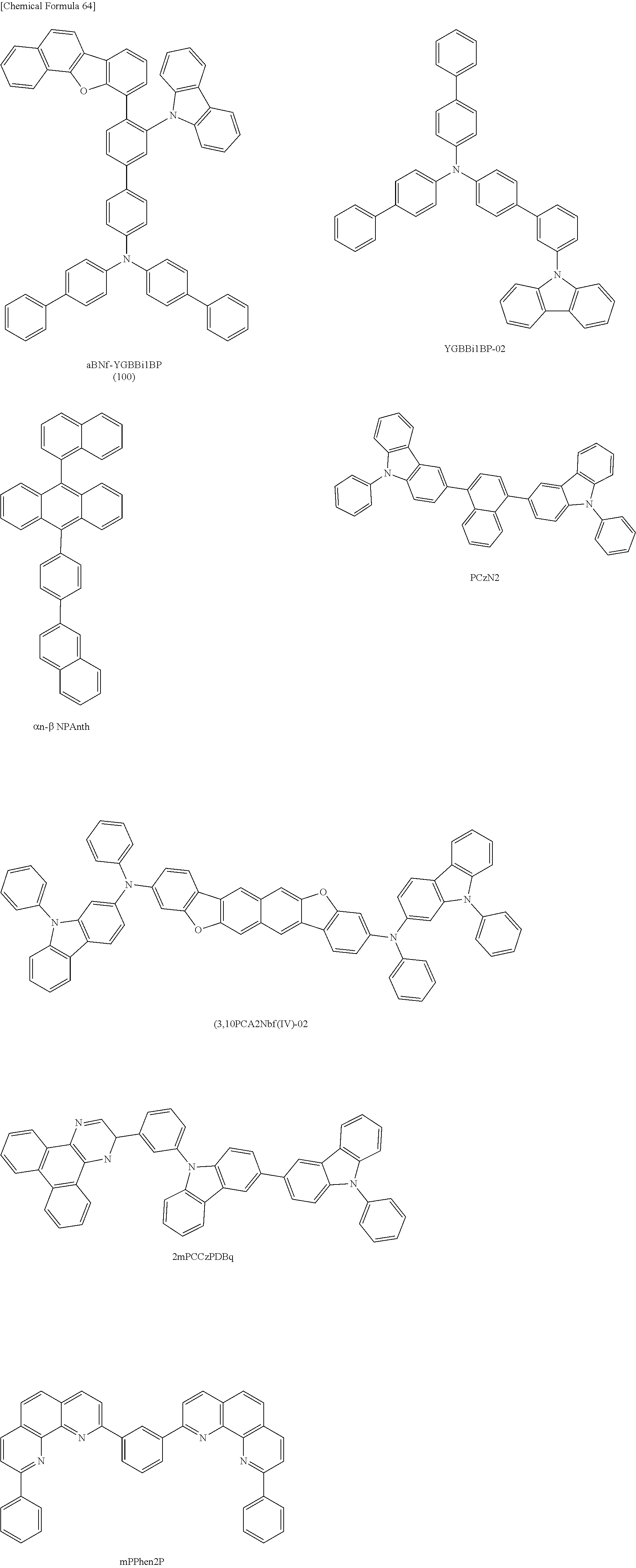

In the devices, as illustrated in FIG. 20, the hole-injection layer 811, the hole-transport layer 812, the light-emitting layer 813, the electron-transport layer 814, and the electron-injection layer 815 are stacked in this order over the first electrode 801 formed over the glass substrate 800, and the second electrode 802 is stacked over the electron-injection layer 815.

### <Method for Fabricating Light-Emitting Device 3A>

As the first electrode 801, a film of indium tin oxide containing silicon oxide (ITSO) was formed to a thickness of 110 nm over the glass substrate 800 by a sputtering method. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for 45 minutes.

Then, the substrate provided with the first electrode 801 was fixed to a substrate holder provided in the vacuum evaporation apparatus such that the surface on which the first electrode 801 was formed faced downward. Over the first electrode 801, N,N′-bis(biphenyl-4-yl)-4′-(benzo[b]naptho[2,1-d]furan-10-yl)-3′-(9H-carbazol-9-yl)biphenyl-4-amine (abbreviation: aBnf-YGBBi1BP) and a fluorine-containing electron acceptor material with a molecular weight of 672 (OCHD-003) were deposited by co-evaporation to a thickness of 10 nm such that the weight ratio of aBnf-YGBBi1BP to OCHD-003 was 1:0.10, whereby the hole-injection layer 811 was formed.

Next, over the hole-injection layer 811, aBnf-YGBBi1BP was deposited by evaporation using resistance heating to a thickness of 90 nm as the hole-transport layer 8122, and then 3,3′-(naphthalene-1,4-diyl)bis(9-phenyl-9H-carbazole) (abbreviation: PCzN2) was deposited by evaporation using resistance heating to a thickness of 10 nm as the hole-transport layer 812_1, whereby the hole-transport layer 812 was formed.

Next, over the hole-transport layer 812, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited by co-evaporation using resistance heating to a thickness of 25 nm such that the weight ratio of αN-βNPAnth to 3,10PCA2Nbf(IV)-02 was 1:0.015, whereby the light-emitting layer 813 was formed.

Next, over the light-emitting layer 813, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm as the electron-transport layer 814_1. Then, 2,2′-(1,3-phenylene)bis(9-phenyl-1,10-phenanthroline) (abbreviation: mPPhen2P) was deposited by evaporation to a thickness of 15 nm as the electron-transport layer 814_2, whereby the electron-transport layer 814 was formed.

Next, over the electron-transport layer 814, lithium fluoride (LiF) was deposited by evaporation using resistance heating to a thickness of 1 nm, whereby the electron-injection layer 815 was formed.

Then, over the electron-injection layer 815, aluminum (Al) was deposited by evaporation to a thickness of 150 nm, whereby the second electrode 802 was formed.

### <Method for Fabricating Light-Emitting Device 3B>

A method for fabricating the light-emitting device 3B is described. The light-emitting device 3B is different from the light-emitting device 3A in the structures of the hole-injection layer 811 and the hole-transport layer 812_2. The other components were formed in a manner similar to that for the light-emitting device 3A.

Specifically, in the light-emitting device 3B, over the first electrode 801, N,N-bis(biphenyl-4-yl)-3′-(9H-carbazol-9-yl)biphenyl-4-amine (abbreviation: YGBBi1BP-02) and a fluorine-containing electron acceptor material (OCHD-003) with a molecular weight of 672 were deposited by co-evaporation to a thickness of 10 nm such that the weight ratio of YGBBi1BP-02 to OCHD-003 was 1:0.10, whereby the hole-injection layer 811 was formed.

Next, over the hole-injection layer 811, YGBBi1BP-02 was deposited by evaporation using resistance heating to a thickness of 90 nm as the hole-transport layer 8122, and then DBfBB1TP was deposited by evaporation using resistance heating to a thickness of 10 nm as the hole-transport layer 812_1, whereby the hole-transport layer 812 was formed.

The structures of the light-emitting device 3A and the light-emitting device 3B for comparison are listed in the following table.

TABLE 5

| | Thickness | | Light-emitting device 3B |
|---|---|---|---|
| | [nm] | Light-emitting device 3A | (comparison) |
| Second electrode 802 | 150 | A1 | |
| Electron-injection layer 815 | 1 | LiF | |
| Electron-transport layer 814_2 | 15 | mPPhen2P | |
| Electron-transport layer 814_1 | 10 | 2mPCCzPDBq | |
| Light-emitting layer 813 | 25 | αN-βNP Anth: 3,10PCA2Nbf(IV)-02 (1: 0.015) | |
| Hole-transport layer 812_1 | 10 | PCzN2 | |
| Hole-transport layer 812_2 | 90 | aBnf-YGBBi1BP | YGBBi1BP-02 |
| Hole-injection layer 811 | 10 | aBnf-YGBBi1BP: OCHD-003 | YGBBi1BP-02: OCHD-003 |
| | | (1: 0.10) | (1: 0.10) |

| | | |
|---|---|---|
| First electrode 801 | 110 | ITSO |

### <Characteristics of Light-Emitting Devices>

The light-emitting devices 3A and 3B were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

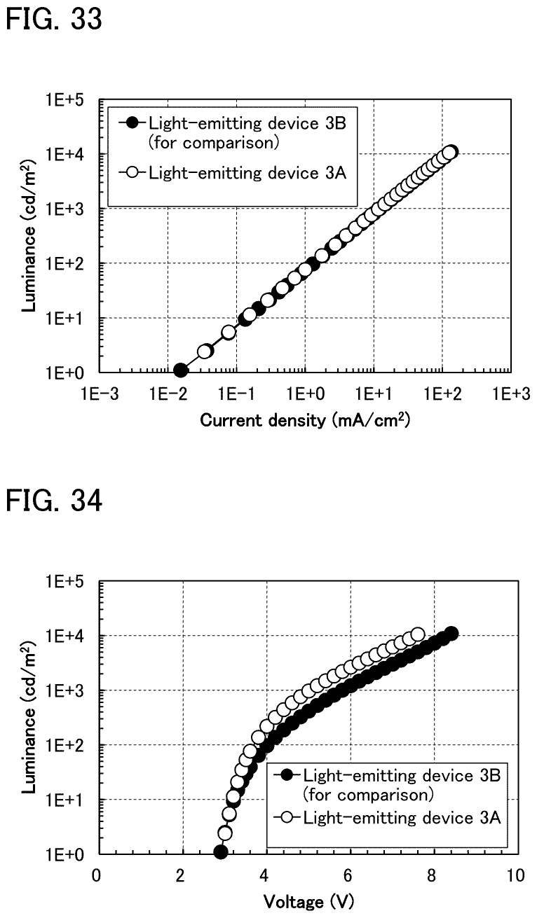

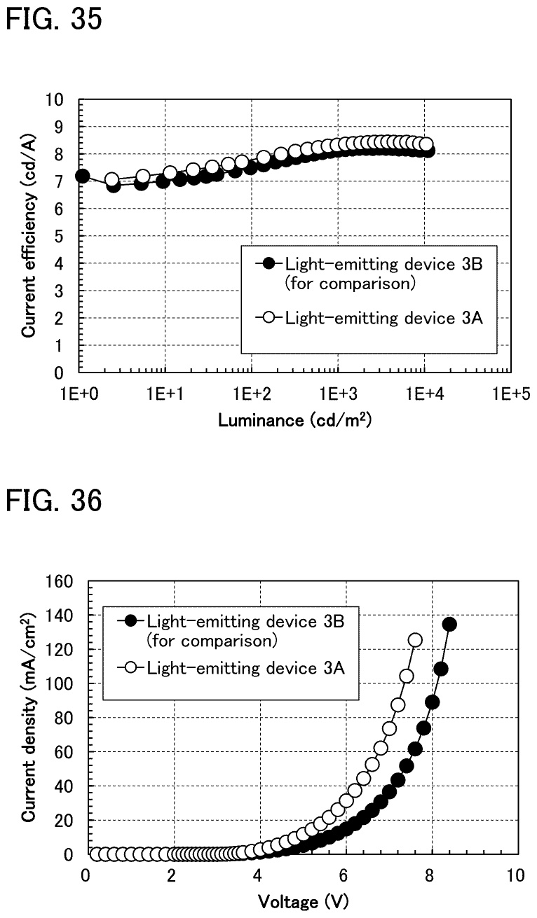

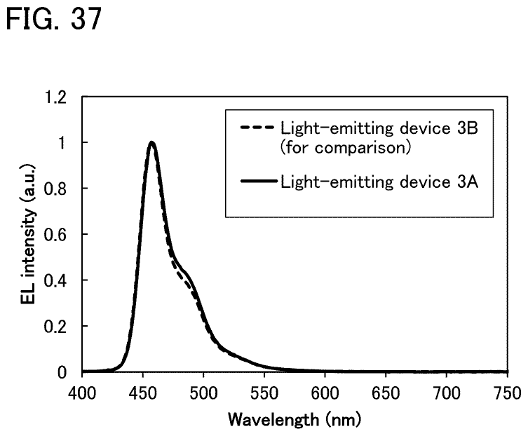

FIG. 33 shows the luminance-current density characteristics of the light-emitting devices 3A and 3B. FIG. 34 shows the luminance-voltage characteristics of the light-emitting devices 3A and 3B. FIG. 35 shows the current efficiency-luminance characteristics of the light-emitting devices 3A and 3B. FIG. 36 shows the current density-voltage characteristics of the light-emitting devices 3A and 3B. FIG. 37 shows the electroluminescence spectra of the light-emitting devices 3A and 3B.

The main characteristics of the devices at a luminance of approximately 1000 cd/m² are shown in the table below. Note that the luminance, CIE chromaticity, and emission spectra were measured with a spectroradiometer (SR-UL1R, TOPCON TECHNOHOUSE CORPORATION).

TABLE 6

| | | | | | | | | | External |
|---|---|---|---|---|---|---|---|---|---|
| | | | Current | | | | Current | Power | quantum |
| | Voltage | Current | density | Chromaticity | Chromaticity | Luminance | efficiency | efficiency | efficiency |
| | [V] | [mA] | [mA/cm²] | x | y | [cd/m²] | [cd/A] | [lm/W] | [%] |
| Light-emitting device 3A | 5.00 | 0.466 | 11.7 | 0.137 | 0.106 | 970 | 8.32 | 5.23 | 8.99 |
| Light-emitting device 3B | 5.80 | 0.491 | 12.3 | 0.138 | 0.102 | 998 | 8.14 | 4.41 | 9.00 |

The above table and FIG. 33 to FIG. 37 show that the light-emitting device 3A is a favorable light-emitting device. Specifically, the light-emitting device 3A is found to be driven at a lower voltage than the light-emitting device 3B. This is probably because benzonaphthofuran included in aBnf-YGBBi1BP used for the light-emitting device 3A has an excellent hole-transport property, which increases the proportion of holes reaching the inside of the light-emitting layer and enables low-voltage driving.

### <Results of Reliability Test>

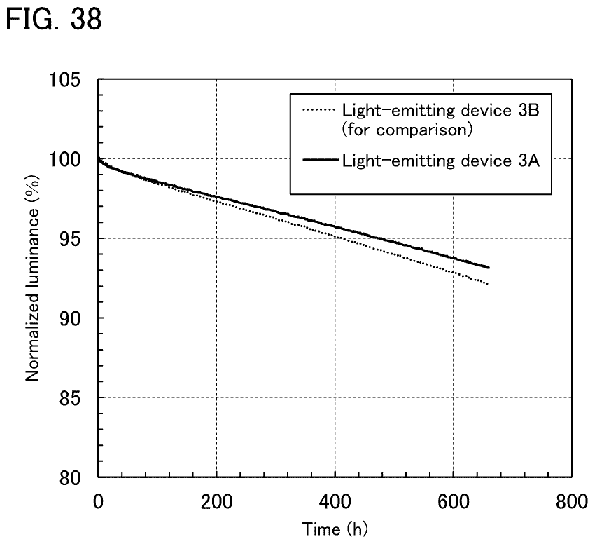

A reliability test was performed on the light-emitting devices 3A and 3B. FIG. 38 shows a time-dependent change in normalized luminance at the time of constant current density driving (50 [mA/cm²]). In FIG. 38, the vertical axis represents the luminance (%) normalized with the luminance at the time of the start of emission as 100%, and the horizontal axis represents time (h). In FIG. 38, the vertical axis represents the luminance (%) normalized with the luminance at the time of the start of emission as 100%, and the horizontal axis represents time (h).

LT95 (h), which is the time that has elapsed until the measured luminance decreases to 95% of the initial luminance, of the light-emitting device 3A was 473 hours. LT95 (h) of the light-emitting device 3B for comparison was 411 hours. This shows that LT95 of the light-emitting device 3A is longer than that of the light-emitting device 3B for comparison.

Since benzonaphthofuran included in aBnf-YGBBi1BP used for the light-emitting device 3A has an excellent hole-transport property, more holes can be transported to the light-emitting layer and to the electron-transport layer side. This probably leads to less generation of unnecessary excitons at the interface between the hole-transport layer and the light-emitting layer, thereby improving the overall device reliability.

Thus, it was confirmed that the light-emitting device of one embodiment of the present invention has favorable characteristics and improved reliability.

### Example 6

In this example, a light-emitting device 4A of one embodiment of the present invention was fabricated. A light-emitting device 4B for comparison was also fabricated and their characteristics were compared.

The structural formulae of organic compounds used for the light-emitting devices 4A and 4B are shown below.

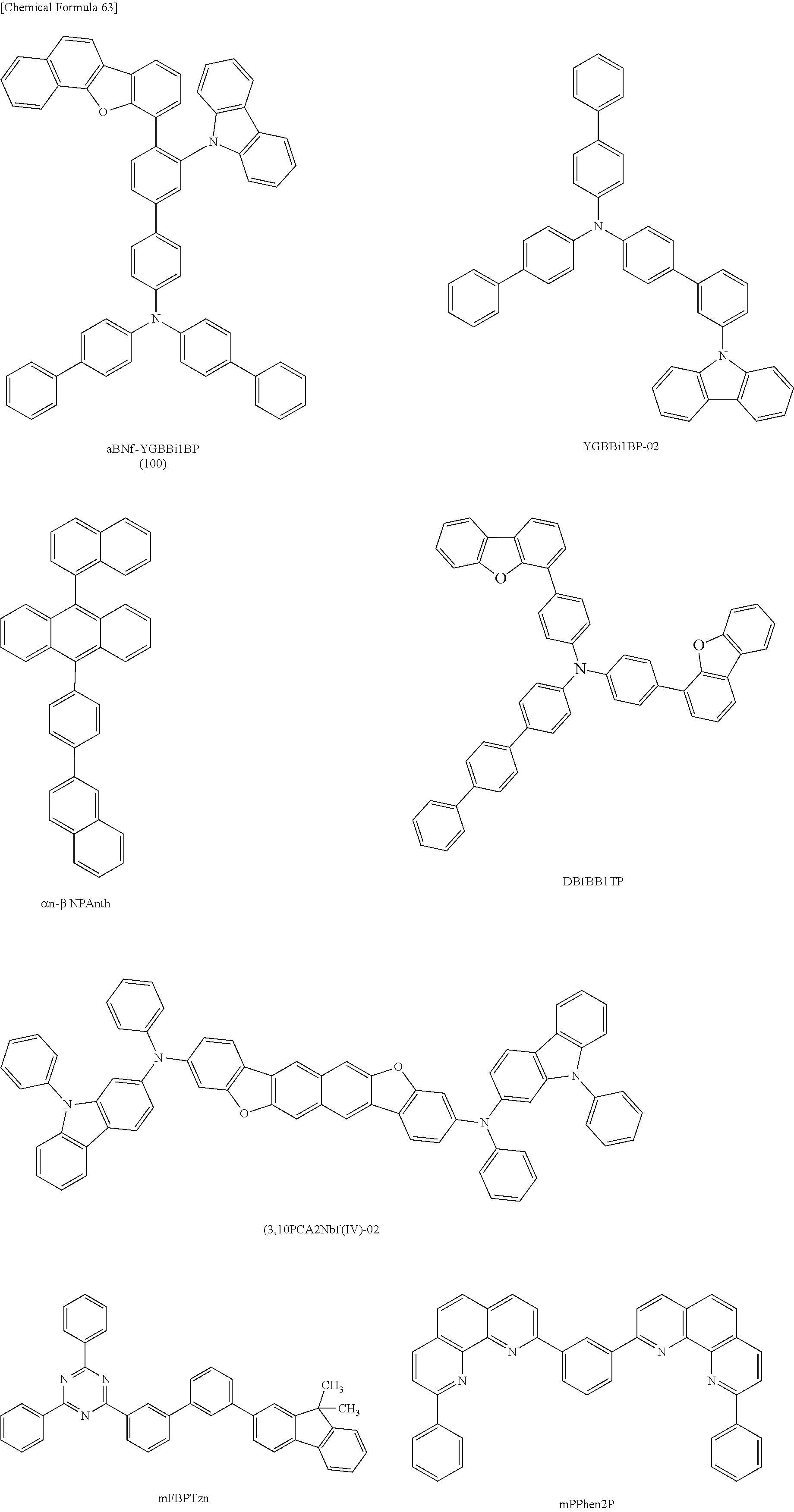

In the devices, as illustrated in FIG. 20, the hole-injection layer 811, the hole-transport layer 812, the light-emitting layer 813, the electron-transport layer 814, and the electron-injection layer 815 are stacked in this order over the first electrode 801 formed over the glass substrate 800, and the second electrode 802 is stacked over the electron-injection layer 815.

### <Method for Fabricating Light-Emitting Device 4A>

As the first electrode 801, a film of indium tin oxide containing silicon oxide (ITSO) was formed to a thickness of 110 nm over the glass substrate 800 by a sputtering method. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for 45 minutes.

Then, the substrate provided with the first electrode 801 was fixed to a substrate holder provided in the vacuum evaporation apparatus such that the surface on which the first electrode 801 was formed faced downward. Over the first electrode 801, N,N′-bis(biphenyl-4-yl)-4′-(benzo[b]naptho[2,1-d]furan-10-yl)-3′-(9H-carbazol-9-yl)biphenyl-4-amine (abbreviation: aBnf-YGBBi1BP) and a fluorine-containing electron acceptor material with a molecular weight of 672 (OCHD-003) were deposited by co-evaporation to a thickness of 10 nm such that the weight ratio of aBnf-YGBBi1BP to OCHD-003 was 1:0.10, whereby the hole-injection layer 811 was formed.

Next, over the hole-injection layer 811, aBnf-YGBBi1BP was deposited by evaporation using resistance heating to a thickness of 90 nm as the hole-transport layer 8122, and then 3,3′-(naphthalene-1,4-diyl)bis(9-phenyl-9H-carbazole) (abbreviation: PCzN2) was deposited by evaporation using resistance heating to a thickness of 10 nm as the hole-transport layer 812_1, whereby the hole-transport layer 812 was formed.

Next, over the hole-transport layer 812, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited by co-evaporation using resistance heating to a thickness of 25 nm such that the weight ratio of αN-βNPAnth to 3,10PCA2Nbf(IV)-02 was 1:0.015, whereby the light-emitting layer 813 was formed.

Next, over the light-emitting layer 813, 2-[3′-(9,9-dimethyl-9H-fluoren-2-yl)biphenyl-3-yl]-4,6-diphenyl-1,3,5-triazine (abbreviation: mFBPTzn) was deposited by evaporation to a thickness of 10 nm as the electron-transport layer 814_1. Then, 2,2′-(1,3-phenylene)bis(9-phenyl-1,10-phenanthroline) (abbreviation: mPPhen2P) was deposited by evaporation to a thickness of 15 nm as the electron-transport layer 814_2, whereby the electron-transport layer 814 was formed.

Next, over the electron-transport layer 814, lithium fluoride (LiF) was deposited by evaporation using resistance heating to a thickness of 1 nm, whereby the electron-injection layer 815 was formed.

Then, over the electron-injection layer 815, aluminum (Al) was deposited by evaporation to a thickness of 150 nm, whereby the second electrode 802 was formed.

### <Method for Fabricating Light-Emitting Device 4B>

A method for fabricating the light-emitting device 4B is described. The light-emitting device 4B is different from the light-emitting device 4A in the structures of the hole-injection layer 811 and the hole-transport layer 812_2. The other components were formed in a manner similar to that for the light-emitting device 4A.

Specifically, in the light-emitting device 4B, over the first electrode 801, N,N-bis(biphenyl-4-yl)-3′-(9H-carbazol-9-yl)biphenyl-4-amine (abbreviation: YGBBi1BP-02) and a fluorine-containing electron acceptor material (OCHD-003) with a molecular weight of 672 were deposited by co-evaporation to a thickness of 10 nm such that the weight ratio of YGBBi1BP-02 to OCHD-003 was 1:0.10, whereby the hole-injection layer 811 was formed.

Next, over the hole-injection layer 811, YGBBi1BP-02 was deposited by evaporation using resistance heating to a thickness of 90 nm as the hole-transport layer 8122, and then DBfBB1TP was deposited by evaporation using resistance heating to a thickness of 10 nm as the hole-transport layer 812_1, whereby the hole-transport layer 812 was formed.

The structures of the light-emitting device 4A and the light-emitting device 4B for comparison are listed in the following table.

TABLE 7

| | Thickness | | Light-emitting device 4B |
|---|---|---|---|
| | [nm] | Light-emitting device 4A | (comparison) |
| Second electrode 802 | 150 | A1 | |
| Electron-injection layer 815 | 1 | LiF | |
| Electron-transport layer 814_2 | 15 | mPPhen2P | |
| Electron-transport layer 814_1 | 10 | mFBPTzn | |
| Light-emitting layer 813 | 25 | αN-βNPAnth: 3,10PCA2Nbf(IV)-02 (1: 0.015) | |
| Hole-transport layer 812_1 | 10 | PCzN2 | |
| Hole-transport layer 812_2 | 90 | aBnf-YGBBi1BP | YGBBi1BP-02 |
| Hole-injection layer 811 | 10 | aBnf-YGBBi1BP: OCHD-003 | YGBBi1BP-02: OCHD-003 |
| | | (1: 0.10) | (1: 0.10) |

| | | |
|---|---|---|
| First electrode 801 | 110 | ITSO |

### <Characteristics of Light-Emitting Devices>

The light-emitting devices 4A and 4B were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

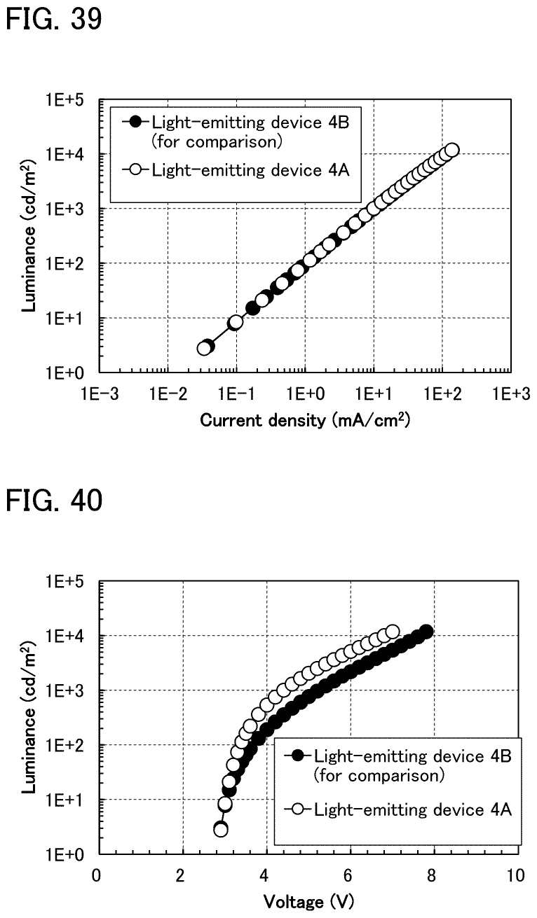

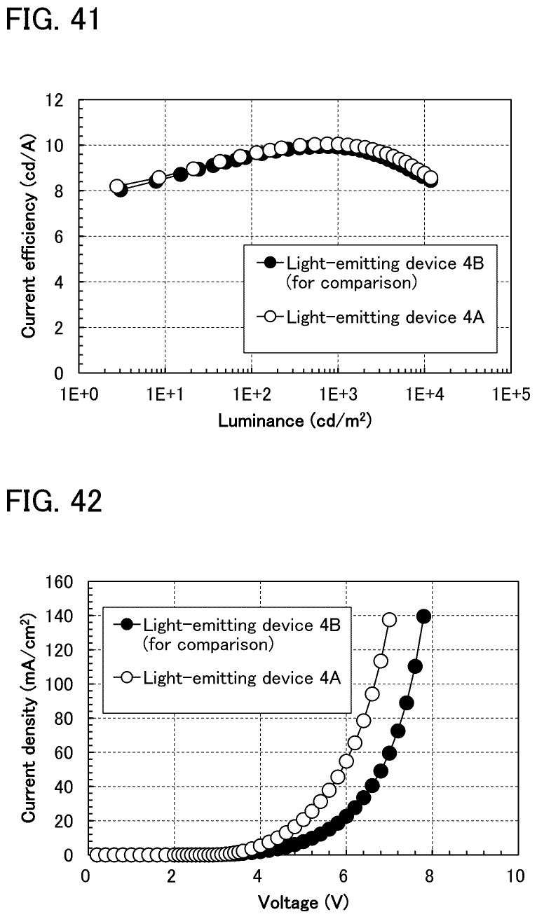

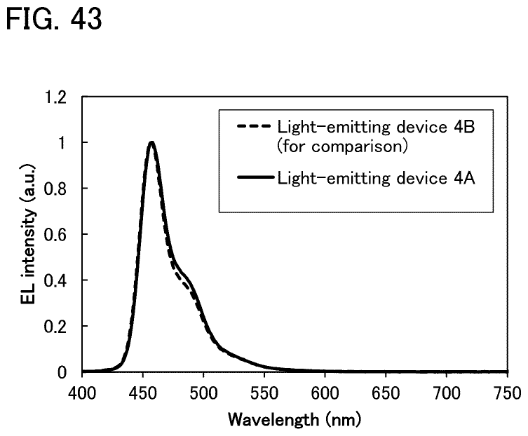

FIG. 39 shows the luminance-current density characteristics of the light-emitting devices 4A and 4B. FIG. 40 shows the luminance-voltage characteristics of the light-emitting devices 4A and 4B. FIG. 41 shows the current efficiency-luminance characteristics of the light-emitting devices 4A and 4B. FIG. 42 shows the current density-voltage characteristics of the light-emitting devices 4A and 4B. FIG. 43 shows the electroluminescence spectra of the light-emitting devices 4A and 4B.

The main characteristics of the devices at a luminance of approximately 1000 cd/m² are shown in the table below. Note that the luminance, CIE chromaticity, and emission spectra were measured with a spectroradiometer (SR-UL1R, TOPCON TECHNOHOUSE CORPORATION).

TABLE 8

| | | | | | | | | | External |
|---|---|---|---|---|---|---|---|---|---|
| | | | Current | | | | Current | Power | quantum |
| | Voltage | Current | density | Chromaticity | Chromaticity | Luminance | efficiency | efficiency | efficiency |
| | [V] | [mA] | [mA/cm²] | x | y | [cd/m²] | [cd/A] | [lm/W] | [%] |
| Light-emitting device 4A | 4.4 | 0.40 | 10.0 | 0.137 | 0.103 | 1002 | 10.0 | 7.17 | 11.1 |
| Light-emitting device 4B | 5.2 | 0.39 | 9.74 | 0.139 | 0.0993 | 967 | 9.92 | 5.99 | 11.1 |

The above table and FIG. 39 to FIG. 43 show that the light-emitting device 4A is a favorable light-emitting device. Specifically, the light-emitting device 4A is found to be driven at a lower voltage than the light-emitting device 4B. This is probably because benzonaphthofuran included in aBnf-YGBBi1BP used for the light-emitting device 4A has an excellent hole-transport property, which increases the proportion of holes reaching the inside of the light-emitting layer and enables low-voltage driving.

### <Results of Reliability Test>

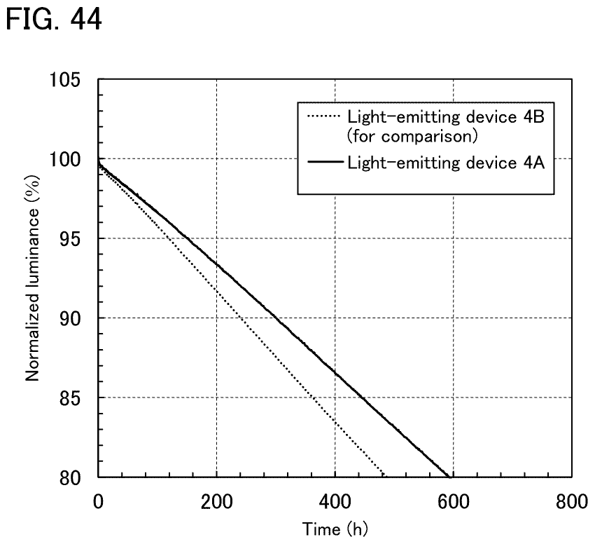

A reliability test was performed on the light-emitting devices 4A and 4B. FIG. 44 shows a time-dependent change in normalized luminance at the time of constant current density driving (50 [mA/cm²]). In FIG. 44, the vertical axis represents the luminance (%) normalized with the luminance at the time of the start of emission as 100%, and the horizontal axis represents time (h).

LT95 (h), which is the time that has elapsed until the measured luminance decreases to 95% of the initial luminance, of the light-emitting device 4A was 151 hours. LT95 (h) of the light-emitting device 4B for comparison was 120 hours. This shows that LT95 of the light-emitting device 4A is longer than that of the light-emitting device 4B for comparison.

Since benzonaphthofuran included in aBnf-YGBBi1BP used for the light-emitting device 4A has an excellent hole-transport property, more holes can be transported to the light-emitting layer and to the electron-transport layer side. This probably leads to less generation of unnecessary excitons at the interface between the hole-transport layer and the light-emitting layer, thereby improving the overall device reliability.

Thus, it was confirmed that the light-emitting device of one embodiment of the present invention has favorable characteristics and improved reliability.

### Example 7

In this example, a light-emitting device 5A of one embodiment of the present invention was fabricated. A light-emitting device 5B for comparison was also fabricated and their characteristics were compared.

The structural formulae of organic compounds used for the light-emitting devices 5A and 5B are shown below.

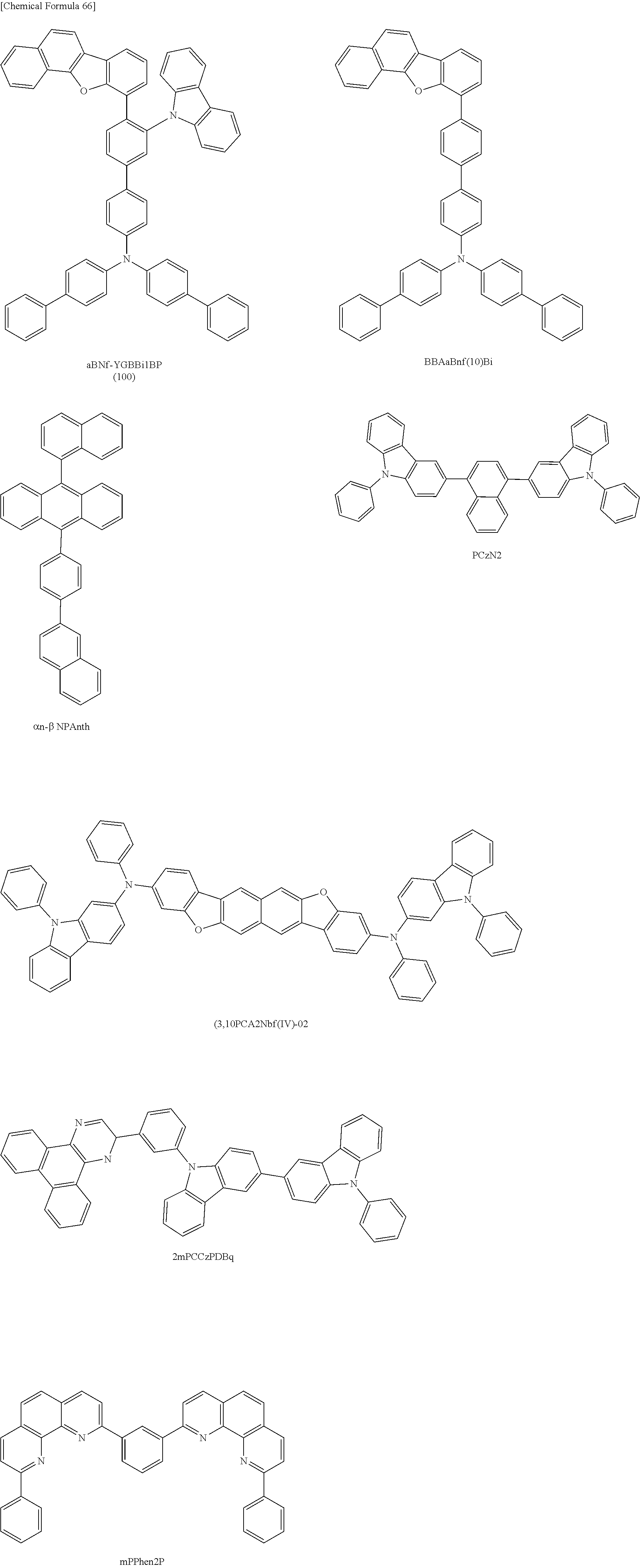

In the devices, as illustrated in FIG. 20, the hole-injection layer 811, the hole-transport layer 812, the light-emitting layer 813, the electron-transport layer 814, and the electron-injection layer 815 are stacked in this order over the first electrode 801 formed over the glass substrate 800, and the second electrode 802 is stacked over the electron-injection layer 815.

### <Method for Fabricating Light-Emitting Device 5A>

As the first electrode 801, a film of indium tin oxide containing silicon oxide (ITSO) was formed to a thickness of 110 nm over the glass substrate 800 by a sputtering method. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for 45 minutes.

Then, the substrate provided with the first electrode 801 was fixed to a substrate holder provided in the vacuum evaporation apparatus such that the surface on which the first electrode 801 was formed faced downward. Over the first electrode 801, N,N-bis(biphenyl-4-yl)-4′-(benzo[b]naptho[2,1-d]furan-10-yl)-3′-(9H-carbazol-9-yl)biphenyl-4-amine (abbreviation: aBnf-YGBBi1BP) and a fluorine-containing electron acceptor material with a molecular weight of 672 (OCHD-003) were deposited by co-evaporation to a thickness of 10 nm such that the weight ratio of aBnf-YGBBi1BP to OCHD-003 was 1:0.10, whereby the hole-injection layer 811 was formed.

Next, over the hole-injection layer 811, aBnf-YGBBi1BP was deposited by evaporation using resistance heating to a thickness of 90 nm as the hole-transport layer 8122, and then 3,3′-(naphthalene-1,4-diyl)bis(9-phenyl-9H-carbazole) (abbreviation: PCzN2) was deposited by evaporation using resistance heating to a thickness of 10 nm as the hole-transport layer 812_1, whereby the hole-transport layer 812 was formed.

Next, over the hole-transport layer 812, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited by co-evaporation using resistance heating to a thickness of 25 nm such that the weight ratio of αN-βNPAnth to 3,10PCA2Nbf(IV)-02 was 1:0.015, whereby the light-emitting layer 813 was formed.

Next, over the light-emitting layer 813, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm as the electron-transport layer 814_1. Then, 2,2′-(1,3-phenylene)bis(9-phenyl-1,10-phenanthroline) (abbreviation: mPPhen2P) was deposited by evaporation to a thickness of 15 nm as the electron-transport layer 814_2, whereby the electron-transport layer 814 was formed.

Next, over the electron-transport layer 814, lithium fluoride (LiF) was deposited by evaporation using resistance heating to a thickness of 1 nm, whereby the electron-injection layer 815 was formed.

Then, over the electron-injection layer 815, aluminum (Al) was deposited by evaporation to a thickness of 150 nm, whereby the second electrode 802 was formed.

### <Method for Fabricating Light-Emitting Device 5B>

A method for fabricating the light-emitting device 5B is described. The light-emitting device 5B is different from the light-emitting device 5A in the structures of the hole-injection layer 811 and the hole-transport layer 812_2. The other components were formed in a manner similar to that for the light-emitting device 5A.

Specifically, in the light-emitting device 5B, over the first electrode 801, N-[4′-(benzo[b]naphtho[2,1-d]furan-10-yl)biphenyl-4-yl]bis(biphenyl-4-yl)amine (abbreviation: BBAaBnf(10)Bi) and a fluorine-containing electron acceptor material (OCHD-003) with a molecular weight of 672 were deposited by co-evaporation to a thickness of 10 nm such that the weight ratio of BBAaBnf(10)Bi to OCHD-003 was 1:0.10, whereby the hole-injection layer 811 was formed.

Next, over the hole-injection layer 811, BBAaBnf(10)Bi was deposited by evaporation using resistance heating to a thickness of 90 nm as the hole-transport layer 812_2, and then PCzN2 was deposited by evaporation using resistance heating to a thickness of 10 nm as the hole-transport layer 812_1, whereby the hole-transport layer 812 was formed.

The structures of the light-emitting device 5A and the light-emitting device 5B for comparison are listed in the following table.

TABLE 9

| | Thickness | | Light-emitting device 5B |
|---|---|---|---|
| | [nm] | Light-emitting device SA | (comparison) |
| Second electrode 802 | 150 | A1 | |
| Electron-injection layer 815 | 1 | LiF | |
| Electron-transport layer 814_2 | 15 | mPPhen2P | |
| Electron-transport layer 814_1 | 10 | 2mPCCzPDBq | |
| Light-emitting layer 813 | 25 | αN-βNPAnth: 3,10PCA2Nbf(IV)-02 (1: 0.015) | |
| Hole-transport layer 812_1 | 10 | PCzN2 | |
| Hole-transport layer 812_2 | 90 | aBnf-YGBBi1BP | BBAaBnf(10)Bi |
| Hole-injection layer 811 | 10 | aBnf-YGBBi1BP: OCHD-003 | BBAaBnf(10)Bi: OCHD-003 |
| | | (1: 0.10) | (1: 0.10) |

| | | |
|---|---|---|
| First electrode 801 | 110 | ITSO |

### <Characteristics of Light-Emitting Devices>

The light-emitting devices 5A and 5B were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

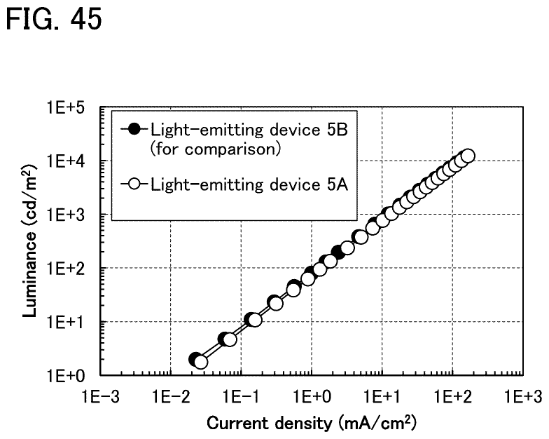

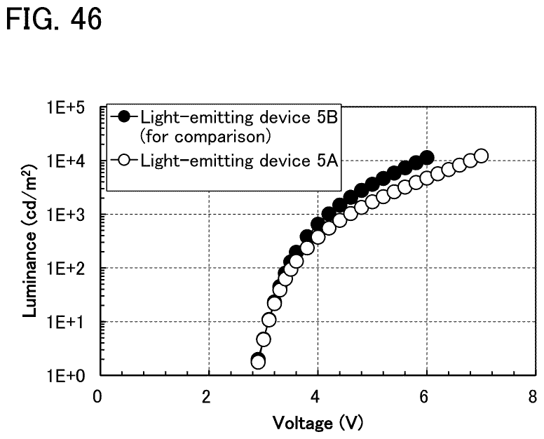

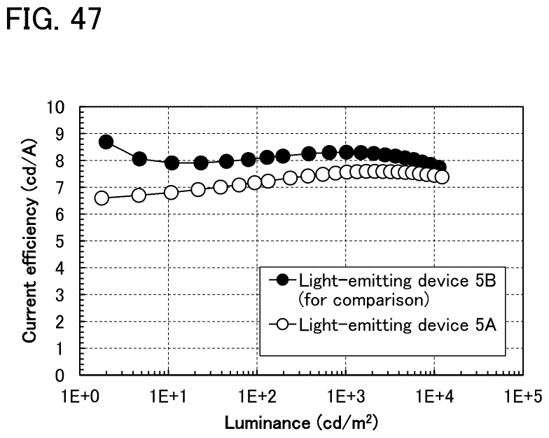

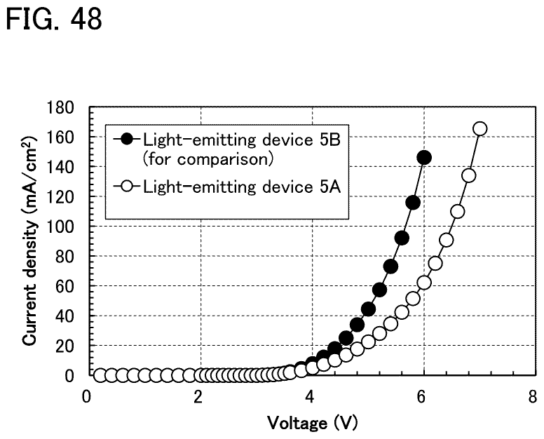

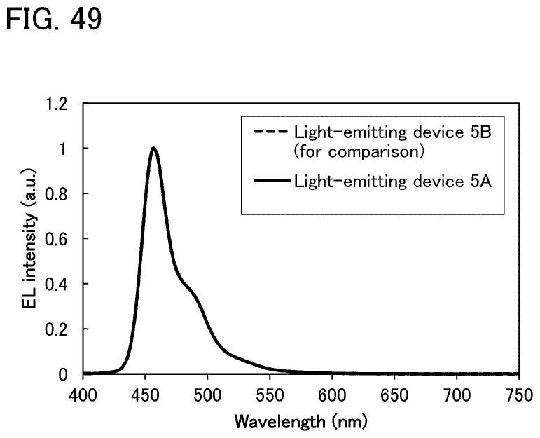

FIG. 45 shows the luminance-current density characteristics of the light-emitting devices 5A and 5B. FIG. 46 shows the luminance-voltage characteristics of the light-emitting devices 5A and 5B. FIG. 47 shows the current efficiency-luminance characteristics of the light-emitting devices 5A and 5B. FIG. 48 shows the current density-voltage characteristics of the light-emitting devices 5A and 5B. FIG. 49 shows the electroluminescence spectra of the light-emitting devices 5A and 5B.

The main characteristics of the devices at a luminance of approximately 1000 cd/m² are shown in the table below. Note that the luminance, CIE chromaticity, and emission spectra were measured with a spectroradiometer (SR-UL1R, TOPCON TECHNOHOUSE CORPORATION).

TABLE 10

| | | | | | | | | | External |
|---|---|---|---|---|---|---|---|---|---|
| | | | Current | | | | Current | Power | quantum |
| | Voltage | Current | density | Chromaticity | Chromaticity | Luminance | efficiency | efficiency | efficiency |
| | [V] | [mA] | [mA/cm²] | x | y | [cd/m²] | [cd/A] | [lm/W] | [%] |
| Light-emitting device 5A | 4.60 | 0.545 | 13.6 | 0.139 | 0.100 | 1030 | 7.56 | 5.17 | 8.41 |
| Light-emitting device 5B | 4.20 | 0.490 | 12.3 | 0.139 | 0.100 | 1018 | 8.31 | 6.21 | 9.27 |

The above table and FIG. 45 to FIG. 49 show that the light-emitting devices 5A and 5B are favorable light-emitting devices.

### <Results of Reliability Test>

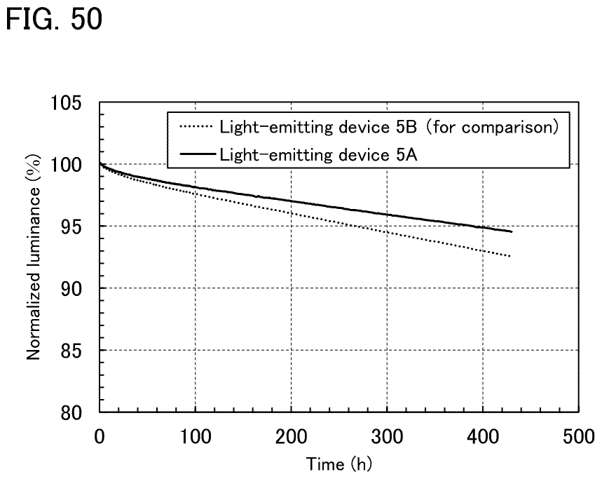

A reliability test was performed on the light-emitting devices 5A and 5B. FIG. 50 shows a time-dependent change in normalized luminance at the time of constant current density driving (50 [mA/cm²]). In FIG. 50, the vertical axis represents the luminance (%) normalized with the luminance at the time of the start of emission as 100%, and the horizontal axis represents time (h).

LT95 (h), which is the time that has elapsed until the measured luminance decreases to 95% of the initial luminance, of the light-emitting device 5A was 389 hours. LT95 (h) of the light-emitting device 5B for comparison was 268 hours. This shows that LT95 of the light-emitting device 5A is longer than that of the light-emitting device 5B for comparison by 100 or more hours.

Since benzonaphthofuran included in aBnf-YGBBi1BP used for the light-emitting device 5A has a carbazole skeleton, degradation of the molecule while holes are held is inhibited, which probably leads to the improvement of reliability. Furthermore, aBnf-YGBBi1BP has extremely high heat resistance with a glass transition point of 168° C., which enables highly stable film quality and improves the overall device reliability.

Thus, it was confirmed that the light-emitting device of one embodiment of the present invention has favorable characteristics and improved reliability.

### Example 8

In this example, a light-emitting device 6A of one embodiment of the present invention was fabricated. A light-emitting device 6B for comparison was also fabricated and their characteristics were compared.

The structural formulae of organic compounds used for the light-emitting devices 6A and 6B are shown below.

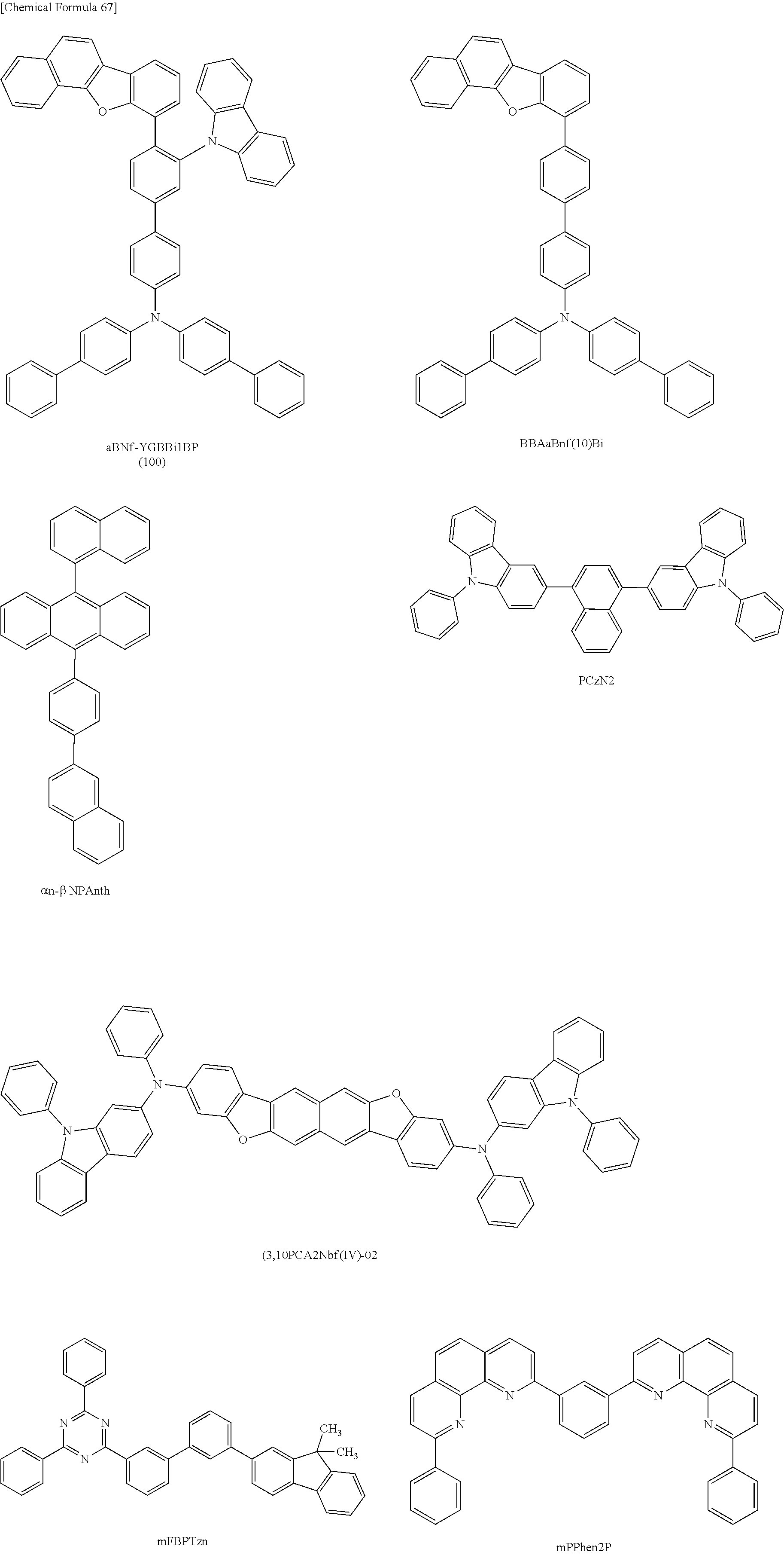

In the devices, as illustrated in FIG. 20, the hole-injection layer 811, the hole-transport layer 812, the light-emitting layer 813, the electron-transport layer 814, and the electron-injection layer 815 are stacked in this order over the first electrode 801 formed over the glass substrate 800, and the second electrode 802 is stacked over the electron-injection layer 815.

### <Method for Fabricating Light-Emitting Device 6A>

As the first electrode 801, a film of indium tin oxide containing silicon oxide (ITSO) was formed to a thickness of 110 nm over the glass substrate 800 by a sputtering method. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for 45 minutes.

Then, the substrate provided with the first electrode 801 was fixed to a substrate holder provided in the vacuum evaporation apparatus such that the surface on which the first electrode 801 was formed faced downward. Over the first electrode 801, N,N′-bis(biphenyl-4-yl)-4′-(benzo[b]naptho[2,1-d]furan-10-yl)-3′-(9H-carbazol-9-yl)biphenyl-4-amine (abbreviation: aBnf-YGBBi1BP) and a fluorine-containing electron acceptor material with a molecular weight of 672 (OCHD-003) were deposited by co-evaporation to a thickness of 10 nm such that the weight ratio of aBnf-YGBBi1BP to OCHD-003 was 1:0.10, whereby the hole-injection layer 811 was formed.

Next, over the hole-injection layer 811, aBnf-YGBBi1BP was deposited by evaporation using resistance heating to a thickness of 90 nm as the hole-transport layer 8122, and then 3,3′-(naphthalene-1,4-diyl)bis(9-phenyl-9H-carbazole) (abbreviation: PCzN2) was deposited by evaporation using resistance heating to a thickness of 10 nm as the hole-transport layer 812_1, whereby the hole-transport layer 812 was formed.

Next, over the hole-transport layer 812, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited by co-evaporation using resistance heating to a thickness of 25 nm such that the weight ratio of αN-βNPAnth to 3,10PCA2Nbf(IV)-02 was 1:0.015, whereby the light-emitting layer 813 was formed.

Next, over the light-emitting layer 813, 2-[3′-(9,9-dimethyl-9H-fluoren-2-yl)biphenyl-3-yl]-4,6-diphenyl-1,3,5-triazine (abbreviation: mFBPTzn) was deposited by evaporation to a thickness of 10 nm as the electron-transport layer 814_1. Then, 2,2′-(1,3-phenylene)bis(9-phenyl-1,10-phenanthroline) (abbreviation: mPPhen2P) was deposited by evaporation to a thickness of 15 nm as the electron-transport layer 814_2, whereby the electron-transport layer 814 was formed.

Next, over the electron-transport layer 814, lithium fluoride (LiF) was deposited by evaporation using resistance heating to a thickness of 1 nm, whereby the electron-injection layer 815 was formed.

Then, over the electron-injection layer 815, aluminum (Al) was deposited by evaporation to a thickness of 150 nm, whereby the second electrode 802 was formed.

### <Method for Fabricating Light-Emitting Device 6B>

A method for fabricating the light-emitting device 6B is described. The light-emitting device 6B is different from the light-emitting device 6A in the structures of the hole-injection layer 811 and the hole-transport layer 812_2. The other components were formed in a manner similar to that for the light-emitting device 6A.

Specifically, in the light-emitting device 6B, over the first electrode 801, N-[4′-(benzo[b]naphtho[2,1-d]furan-10-yl)biphenyl-4-yl]bis(biphenyl-4-yl)amine (abbreviation: BBAaBnf(10)Bi) and a fluorine-containing electron acceptor material (OCHD-003) with a molecular weight of 672 were deposited by co-evaporation to a thickness of 10 nm such that the weight ratio of BBAaBnf(10)Bi to OCHD-003 was 1:0.10, whereby the hole-injection layer 811 was formed.

Next, over the hole-injection layer 811, BBAaBnf(10)Bi was deposited by evaporation using resistance heating to a thickness of 90 nm as the hole-transport layer 812_2, and then PCzN2 was deposited by evaporation using resistance heating to a thickness of 10 nm as the hole-transport layer 812_1, whereby the hole-transport layer 812 was formed.

The structures of the light-emitting device 6A and the light-emitting device 6B for comparison are listed in the following table.

TABLE 11

| | Thickness | | Light-emitting device 6B |
|---|---|---|---|
| | [nm] | Light-emitting device 6A | (comparison) |
| Second electrode 802 | 150 | A1 | |
| Electron-injection layer 815 | 1 | LiF | |
| Electron-transport layer 814_2 | 15 | mPPhen2P | |
| Electron-transport layer 814_1 | 10 | mFBPTzn | |
| Light-emitting layer 813 | 25 | αN-βNPAnth: 3, 10PCA2Nbf(IV)-02 (1: 0.015) | |
| Hole-transport layer 812_1 | 10 | PCzN2 | |
| Hole-transport layer 812_2 | 90 | aBnf-YGBBi1BP | BBAaBnf(10)Bi |
| Hole-injection layer 811 | 10 | aBnf-YGBBilBP: OCHD-003 | BBAaBnf(10)Bi: OCHD-003 |
| | | (1: 0.10) | (1: 0.10) |

| | | |
|---|---|---|
| First electrode 801 | 110 | ITSO |

### <Characteristics of Light-Emitting Devices>

The light-emitting devices 6A and 6B were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

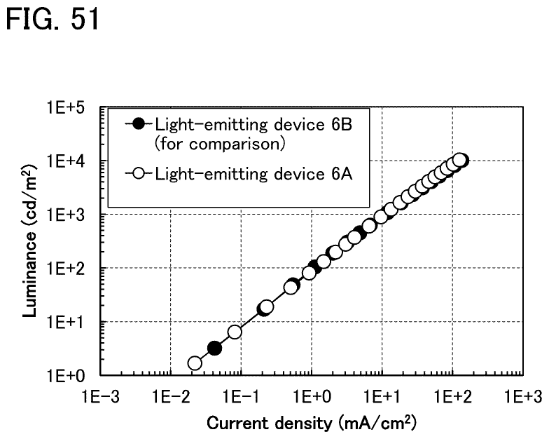

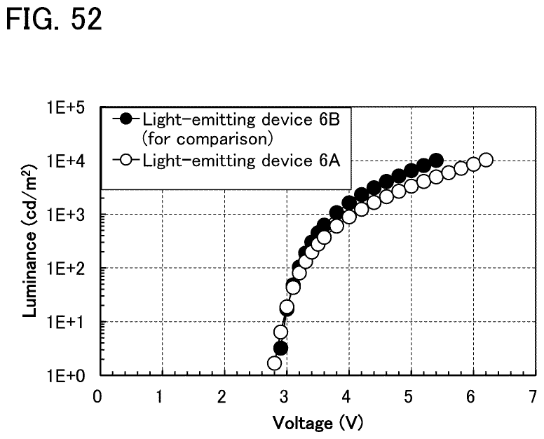

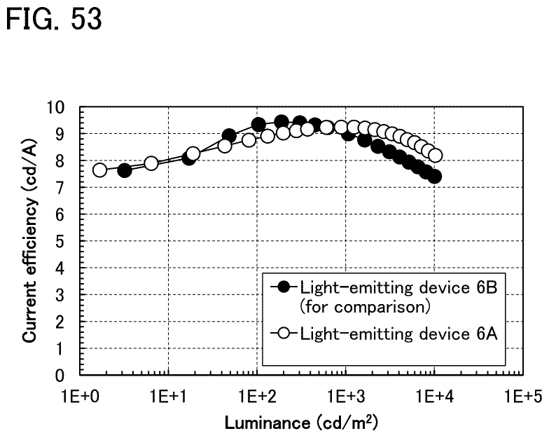

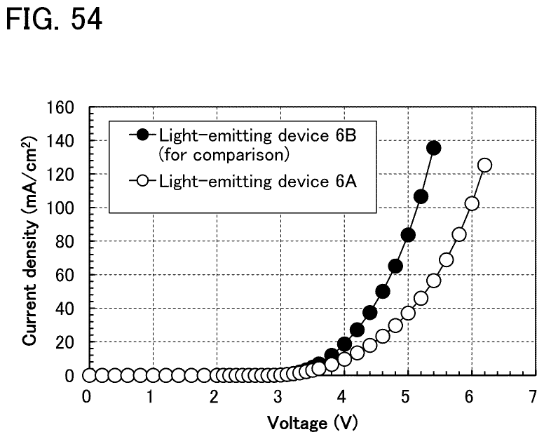

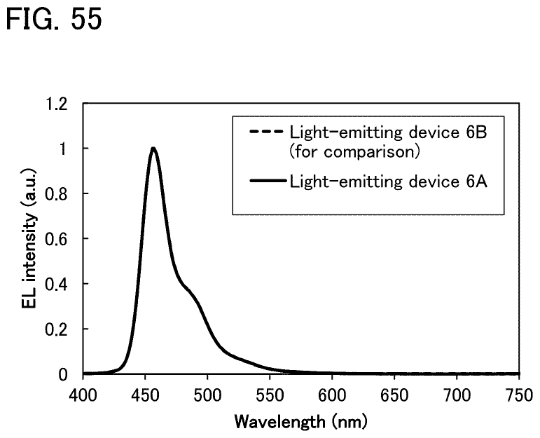

FIG. 51 shows the luminance-current density characteristics of the light-emitting devices 6A and 6B. FIG. 52 shows the luminance-voltage characteristics of the light-emitting devices 6A and 6B. FIG. 53 shows the current efficiency-luminance characteristics of the light-emitting devices 6A and 6B. FIG. 54 shows the current density-voltage characteristics of the light-emitting devices 6A and 6B. FIG. 55 shows the electroluminescence spectra of the light-emitting devices 6A and 6B.

The main characteristics of the devices at a luminance of approximately 1000 cd/m² are shown in the table below. Note that the luminance, CIE chromaticity, and emission spectra were measured with a spectroradiometer (SR-UL1R, TOPCON TECHNOHOUSE CORPORATION).

TABLE 12

| | | | | | | | | | External |
|---|---|---|---|---|---|---|---|---|---|
| | | | Current | | | | Current | Power | quantum |
| | Voltage | Current | density | Chromaticity | Chromaticity | Luminance | efficiency | efficiency | efficiency |
| | [V] | [mA] | [mA/cm²] | x | y | [cd/mm²] | [cd/A] | [lm/W] | [%] |
| Light-emitting device 6A | 4.00 | 0.386 | 9.6 | 0.139 | 0.0991 | 891 | 9.24 | 7.26 | 10.35 |
| Light-emitting device 6B | 3.80 | 0.476 | 11.9 | 0.139 | 0.0985 | 1070 | 8.99 | 7.43 | 10.13 |

The above table and FIG. 51 to FIG. 55 show that the light-emitting devices 6A and 6B are favorable light-emitting devices.

### <Results of Reliability Test>

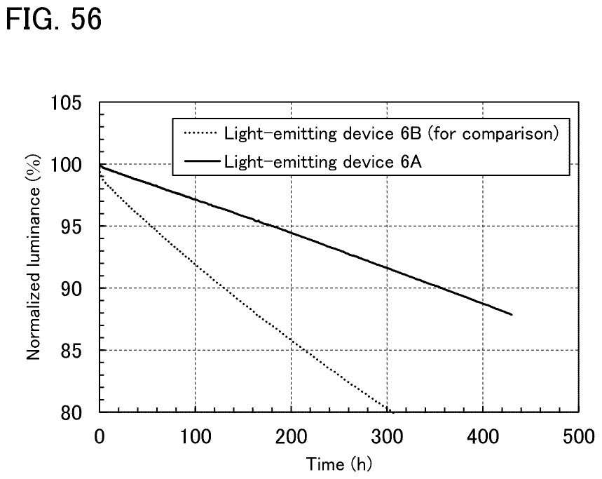

A reliability test was performed on the light-emitting devices 6A and 6B. FIG. 56 shows a time-dependent change in normalized luminance at the time of constant current density driving (50 [mA/cm²]). In FIG. 56, the vertical axis represents the luminance (%) normalized with the luminance at the time of the start of emission as 100%, and the horizontal axis represents time (h).

LT95 (h), which is the time that has elapsed until the measured luminance decreases to 95% of the initial luminance, of the light-emitting device 6A was 180 hours. LT95 (h) of the light-emitting device 6B for comparison was 54 hours. This shows that LT95 of the light-emitting device 6A is more than or equal to 3 times that of the light-emitting device 6B for comparison.

Since benzonaphthofuran included in aBnf-YGBBi1BP used for the light-emitting device 6A has a carbazole skeleton, degradation of the molecule while holes are held is inhibited, which probably leads to the improvement of reliability. Furthermore, aBnf-YGBBi1BP has extremely high heat resistance with a glass transition point of 168° C., which enables highly stable film quality and improves the overall device reliability.

Thus, it was confirmed that the light-emitting device of one embodiment of the present invention has favorable characteristics and improved reliability.

This application is based on Japanese Patent Application Serial No. 2025-027127 filed with Japan Patent Office on Feb. 21, 2025, the entire contents of which are hereby incorporated by reference.

What is claimed is:

1. An organic compound represented by General Formula (G1): 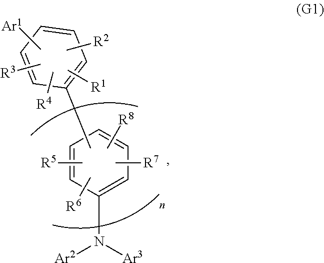
wherein:
Ar¹ represents any one of General Formulae (g1-1) to (g1-3);
each of R¹ to R⁸ independently represents any one of hydrogen, an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, and a substituted or unsubstituted heteroaryl group having 1 to 30 carbon atoms;
any one of R¹ to R⁴ represents General Formula (g2-1) or (g2-2);
n is greater than or equal to 0 and less than or equal to 3;
each of Ar² and Ar³ independently represents a substituted or unsubstituted aryl group having 6 to 60 carbon atoms or a substituted or unsubstituted heteroaryl group having 1 to 60 carbon atoms; 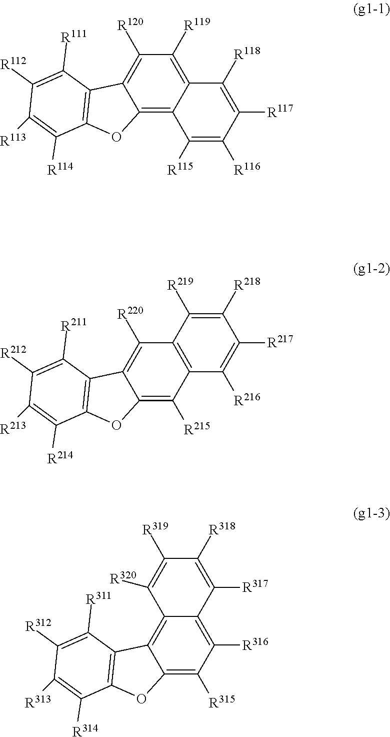
each of R¹¹¹ to R¹²⁰, R²¹¹ to R²²⁰, and R³¹¹ to R³²⁰ independently represents any one of hydrogen, an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, and a substituted or unsubstituted heteroaryl group having 1 to 30 carbon atoms;
any one of R¹¹¹ to R¹²⁰, any one of R²¹¹ to R²²⁰, and any one of R³¹¹ to R³²⁰ each represent a bond; 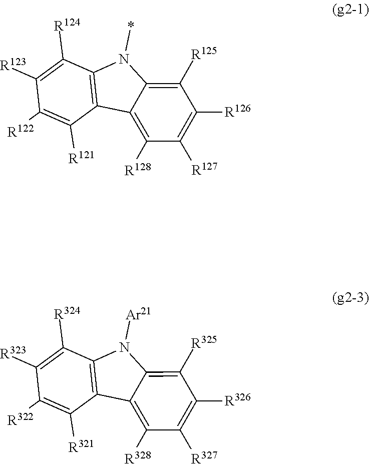
each of R¹²¹ to R¹²⁸ and R²²¹ to R²²⁸ independently represents any one of hydrogen, an alkyl group having 1 to 6 carbon atoms, an alkoxy group having 1 to 6 carbon atoms, a substituted or unsubstituted aryl group having 6 to 30 carbon atoms, and a substituted or unsubstituted heteroaryl group having 1 to 30 carbon atoms;
an asterisk (*) and any one of R²²¹ to R²²⁸ represent a bond; and
Ar²¹ represents a substituted or unsubstituted aryl group having 6 to 60 carbon atoms or a substituted or unsubstituted heteroaryl group having 1 to 60 carbon atoms.

2. The organic compound according to claim 1,
wherein the organic compound is represented by General Formula (G2): 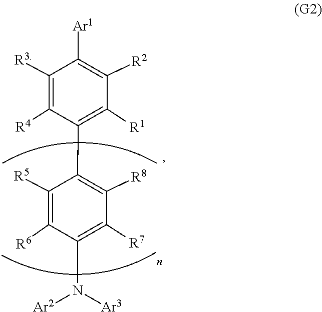
wherein:
each of R¹ to R⁸ independently represents any one of hydrogen, an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, and a heteroaryl group having 1 to 30 carbon atoms; and
Ar²¹ represents a substituted or unsubstituted aryl group having 6 to 30 carbon atoms or a substituted or unsubstituted heteroaryl group having 1 to 60 carbon atoms.

3. The organic compound according to claim 1,
wherein the organic compound is represented by General Formula (G2): 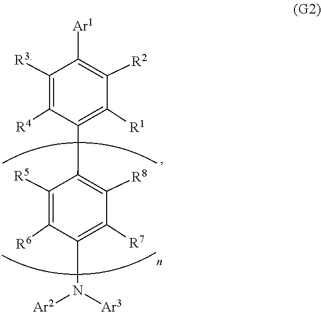
wherein:
each of R¹ and R³ to R⁸ independently represents any one of hydrogen, an alkyl group having 1 to 6 carbon atoms, a cycloalkyl group having 3 to 10 carbon atoms, an alkoxy group having 1 to 6 carbon atoms, a substituted or unsubstituted aryl group having 6 to 15 carbon atoms, and a heteroaryl group having 1 to 30 carbon atoms;
R² represents General Formula (g2-1) or (g2-2);
any one of R²²¹ to R²²⁸ represents a bond; and
Ar²¹ represents a substituted or unsubstituted aryl group having 6 to 30 carbon atoms or a substituted or unsubstituted heteroaryl group having 1 to 60 carbon atoms.

4. The organic compound according to claim 1, wherein n is 1.

5. A light-emitting device comprising the organic compound according to claim 1.

6. A light-receiving device comprising the organic compound according to claim 1.

7. An organic compound represented by General Formula (G3): 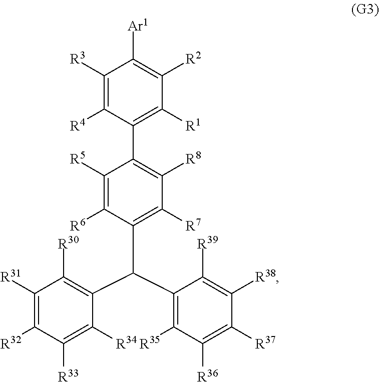
wherein:
Ar¹ represents any one of General Formulae (g1-1) to (g1-3);
each of R¹ to R⁸ and R³⁰ to R³⁹ independently represents any one of hydrogen, an alkyl group having 1 to 6 carbon atoms, a cycloalkyl group having 3 to 10 carbon atoms, an alkoxy group having 1 to 6 carbon atoms, and a substituted or unsubstituted aryl group having 6 to 15 carbon atoms;
R² represents General Formula (g2-1) or (g2-3); and
when each of R³⁰ to R³⁹ represents a substituted aryl group having 6 to 15 carbon atoms, the aryl group having 6 to 15 carbon atoms is bonded to an adjacent aromatic ring to form a ring, 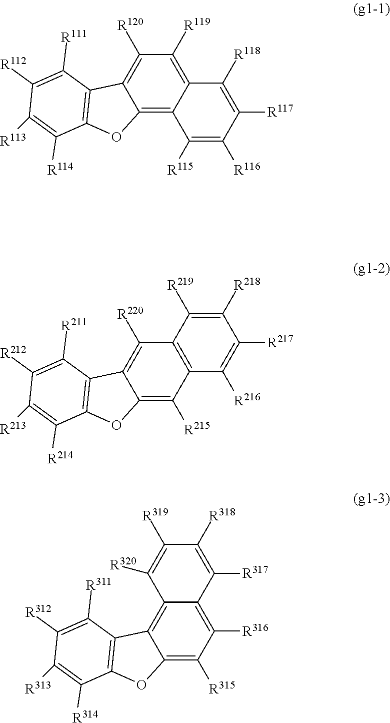
each of R¹¹¹ to R¹²⁰, R²¹¹ to R²²⁰, and R³¹¹ to R³²⁰ independently represents any one of hydrogen, an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, and a substituted or unsubstituted heteroaryl group having 1 to 30 carbon atoms; and
any one of R¹¹¹, R¹¹², R¹¹⁴ and R¹¹⁹ any one of R²¹², R²¹⁴ and R²¹⁵, and any one of R³¹², R³¹⁴ and R³¹⁵ each represent a bond; 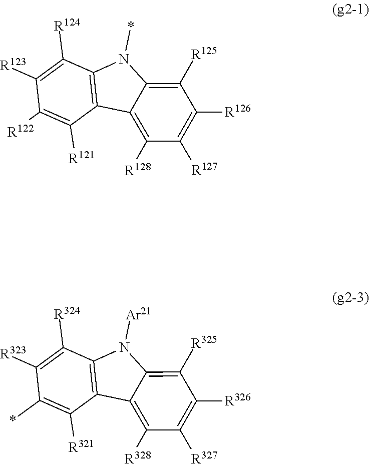
each of R¹²¹ to R¹²⁸ and R³²¹ to R³²⁸ independently represents any one of hydrogen, an alkyl group having 1 to 6 carbon atoms, an alkoxy group having 1 to 6 carbon atoms, and a substituted or unsubstituted aryl group having 6 to 30 carbon atoms;
an asterisk (*) represents a bond; and
Ar²¹ represents a substituted or unsubstituted aryl group having 6 to 30 carbon atoms or a substituted or unsubstituted heteroaryl group having 1 to 30 carbon atoms.

8. The organic compound according to claim 7,
wherein at least one of R³⁰ to R³⁴ represents a substituted or unsubstituted phenyl group, and
wherein at least one of R³⁵ to R³⁹ represents a substituted or unsubstituted phenyl group.

9. The organic compound according to claim 7,
wherein the organic compound is represented by General Formula (G4): 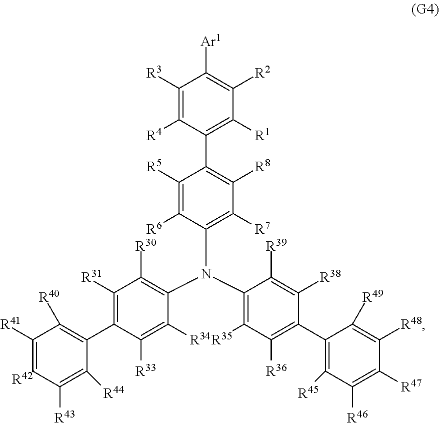
and
wherein each of R⁴⁰ to R⁴⁹ independently represents any one of hydrogen, an alkyl group having 1 to 6 carbon atoms, a cycloalkyl group having 3 to 10 carbon atoms, an alkoxy group having 1 to 6 carbon atoms, and a substituted or unsubstituted aryl group having 6 to 15 carbon atoms.

10. The organic compound according to claim 7, wherein the organic compound is represented by Structural Formula (100):

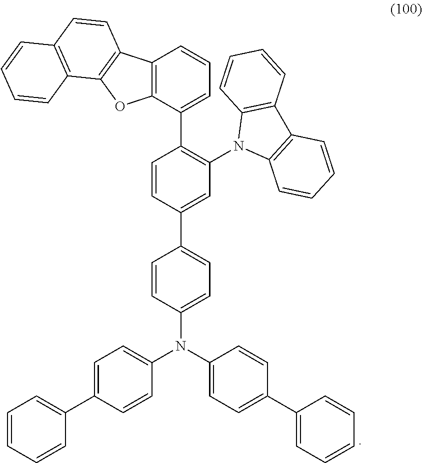 [SMILES: c1ccc(-c2ccc(N(c3ccc(-c4ccccc4)cc3)c3ccc(-c4ccc(-c5cccc6c5oc5c7ccccc7ccc65)c(-n5c6ccccc6c6ccccc65)c4)cc3)cc2)cc1]

11. A light-emitting device comprising the organic compound according to claim 7.

12. A light-receiving device comprising the organic compound according to claim 7.

13. An organic compound represented by General Formula (G5): 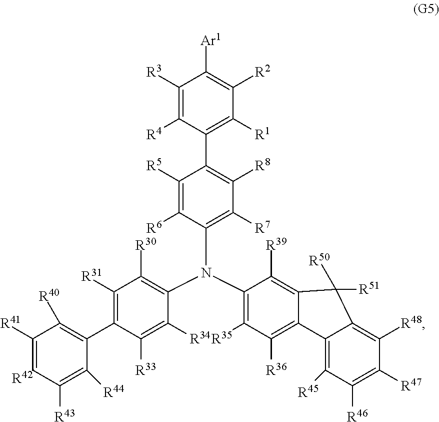
wherein:
Ar¹ represents any one of General Formulae (g1-1), (g1-2), and (g1-3);
each of R¹ to R⁸ and R³⁰ to R⁵¹ independently represents any one of hydrogen, an alkyl group having 1 to 6 carbon atoms, a cycloalkyl group having 3 to 10 carbon atoms, an alkoxy group having 1 to 6 carbon atoms, and a substituted or unsubstituted aryl group having 6 to 15 carbon atoms;
R² represents General Formula (g2-1) or (g2-3); 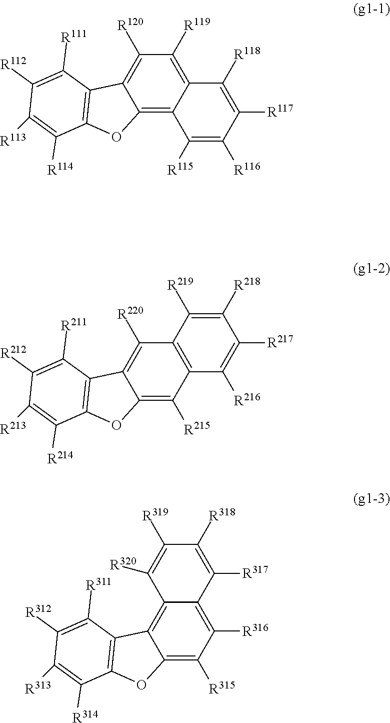
each of R¹¹¹ to R¹²⁰, R²¹¹ to R²²⁰, and R³¹¹ to R³²⁰ independently represents any one of hydrogen, an alkyl group having 1 to 6 carbon atoms, a cycloalkyl group having 3 to 10 carbon atoms, an alkoxy group having 1 to 6 carbon atoms, a substituted or unsubstituted aryl group having 6 to 30 carbon atoms, and a substituted or unsubstituted heteroaryl group having 1 to 30 carbon atoms;
any one of R¹¹¹, R¹¹², R¹¹⁴, and R¹¹⁹, any one of R²¹², R²¹⁴, and R²¹⁵, and any one of R³¹², R³¹⁴, and R³¹⁵ each represent a bond; 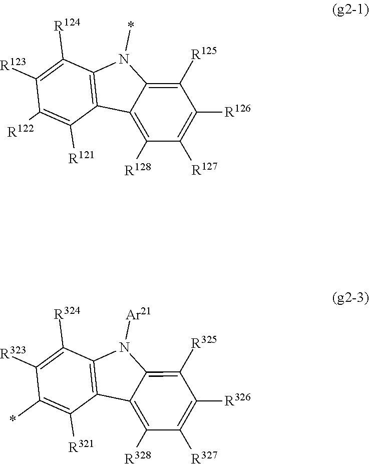
each of R¹²¹ to R¹²⁸ and R³²¹ to R³²⁸ independently represents any one of hydrogen, an alkyl group having 1 to 6 carbon atoms, an alkoxy group having 1 to 6 carbon atoms, and a substituted or unsubstituted aryl group having 6 to 30 carbon atoms;
an asterisk (*) represents a bond; and
Ar²¹ represents a substituted or unsubstituted aryl group having 6 to 30 carbon atoms or a substituted or unsubstituted heteroaryl group having 1 to 30 carbon atoms.

14. The organic compound according to claim 13, wherein when each of R⁵⁰ and R⁵¹ is a substituted or unsubstituted aryl group having 6 to 15 carbon atoms, R⁵⁰ and R⁵¹ are bonded to each other to form a ring.

15. The organic compound according to claim 13, wherein each of R¹¹⁴, R²¹⁴, and R³¹⁴ represents a bond.

16. The organic compound according to claim 13, wherein the organic compound is represented by Structural Formula (101):

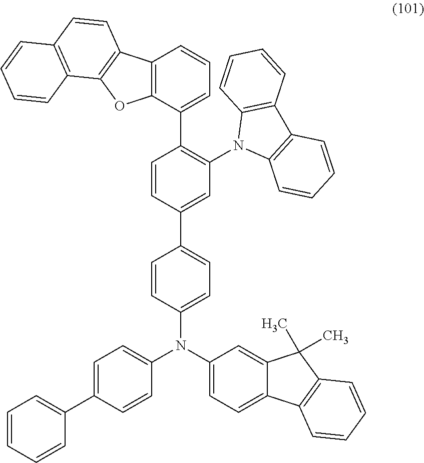 [SMILES: CC1(C)c2ccccc2-c2ccc(N(c3ccc(-c4ccccc4)cc3)c3ccc(-c4ccc(-c5cccc6c5oc5c7ccccc7ccc65)c(-n5c6ccccc6c6ccccc65)c4)cc3)cc21]

17. A light-emitting device comprising the organic compound according to claim 13.

18. A light-receiving device comprising the organic compound according to claim 13.
